(19) United States
(12) Patent Application Publication
(10) Pub. No.: US 2025/0042879 A1
(43) Pub. Date: Feb. 6, 2025

(54) HETEROCYCLIC SUBSTITUTED 1,3,4-THIADIAZOLE AND PYRIDAZINE COMPOUNDS AND METHODS OF USING THE SAME

(71) Applicant: Rgenta Therapeutics, Inc., Woburn, MA (US)

(72) Inventors: Travis T. Wager, Brookline, MA (US); Zhiping Weng, Wellesley, MA (US); Hualin Simon Xi, Lexington, MA (US)

(21) Appl. No.: 18/711,986

(22) PCT Filed: Nov. 22, 2022
(86) PCT No.: PCT/US2022/080352

§ 371 Date: May 21, 2024

Related U.S. Application Data
(60) Provisional application No. 63/281,988, filed on Nov. 22, 2021.

(51) Int. Cl. C07D 403/14; A61K 31/4985; A61K 31/501; A61K 31/5025; A61K 31/506; C07D 405/14; C07D 409/14; C07D 413/14; C07D 417/14; C07D 471/04; C07D 487/04

(52) U.S. Cl. CPC C07D 403/14; A61K 31/4985; A61K 31/501; A61K 31/5025; A61K 31/506; C07D 405/14; C07D 409/14; C07D 413/14; C07D 417/14; C07D 471/04; C07D 487/04

(57) ABSTRACT

The present disclosure relates to compounds of Formula (I); and to their prodrugs, pharmaceutically acceptable salts, pharmaceutical compositions, methods of use, and methods for their preparation. The compounds of the present disclosure may act as small molecule splicing modulator compounds that modulate splicing of mRNA, such as pre-mRNA, encoded genes, and methods of use of the compounds for modulating splicing and treating related diseases and conditions. The compounds disclosed herein may possess activity toward various genetic pathways and are accordingly useful in methods of treatment of the human or animal body.

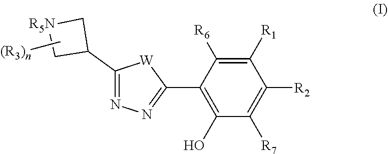

### CROSS REFERENCE TO RELATED APPLICATIONS

This application claims priority to and benefit of U.S. Provisional Application No. 63/281,988, filed Nov. 22, 2021, the contents of which are hereby incorporated by reference in their entirety.

### BACKGROUND

More than 90% of human genes produce multiple mature transcripts via alternative splicing. This process is essential for generating different transcripts in different cell and tissue types, during the developmental process, and in response to internal and external signals. Alternative splicing are prevalent not only for protein-coding genes but also for most other kinds of genes including microRNA genes and long noncoding genes. Splicing is carried out by the spliceosome. Small nuclear RNAs (snRNAs) are key components of the spliceosome. The major spliceosome comprises the U1, U2, U4, U5, and U6 snRNAs, and it catalyzes the removal of ˜95% of human introns, while the remaining introns (called the U12-type of introns) are removed by the minor spliceosome, comprising the U11, U12, U4atac, U5, and U6atac snRNAs. These snRNAs are in complex with their respective protein partners to form the functional unit of small nuclear ribonucleoproteins (snRNPs).

Splicing is a highly regulated process, with the regulation exerted by both cis-elements and trans-factors. The cis-elements that are recognized by the snRNAs include the 5′-splice site, 3′-splice site, and the branchpoint, each of these associating with a sequence motif that is recognized by a component of the spliceosome. In addition, there are intronic splicing enhancers (ISE), intronic splicing silencer (ISS), exonic splicing enhancer (ESE), and exonic splicing enhancer (ESS), which are recognized by a myriad of trans-factors commonly known as RNA-binding proteins (RBPs). Some of these RBPs directly bind to the cis-elements in a sequencing-specific way, while other RBPs recognize RNA structures (e.g., RNA duplex or unpaired loop region), yet others function via protein-protein interaction. There are ˜1600 RBPs annotated in the human genome, and they are expressed in a cell-type-specific manner and form an extensive regulatory network for splicing regulation.

Dysregulation of splicing is implicated in roughly half of human diseases. Some diseases are caused by mutations in the spliceosome components or RBPs, while others by mutations in the cis-elements such as splice sites, branchpoint, or the various splicing enhancers and silencers. Although current approaches to treating these diseases, such as CRISPR-based genome editing, virus-aided gene therapy, or a variety of oligonucleotide-based technologies, continue to improve, they still suffer major technical and clinical challenges. In particular, oligonucleotide-based therapeutics show unfavorable pharmacokinetics, cannot be orally administered, and cannot be delivered effectively to many tissues, especially the brain. Small-molecule drugs have excellent pharmacokinetics, effective delivery, and bioavailability, and have only recently become available for modulating RNA splicing. Yet, the currently available molecules come from a few limited chemical series. Thus, there is a great need to develop additional small molecule splicing modulators (SMSMs).

### SUMMARY

Here we describe a series of novel small molecule splicing modulators (SMSMs), which can be used to treat a wide variety of diseases, including neurological disorders. These SMSMs target regions of a primary RNA transcript that are cis-elements, such as splice sites, branch points, splicing enhancers, or splicing silencers. These regions may contain unpaired nucleotides in an RNA duplex, called bulges. The bulges may be naturally occurring or caused by diseases. When the SMSMs come into contact with the RNA transcript, it may be bound by the spliceosome or the other trans-factors, most notably RNA-binding proteins (RBPs). The SMSMs reported herein may cause an alteration in the sequence or abundance of the mature transcript, which may, in turn, cause a difference in the sequence or abundance of the functional protein should the transcript be protein-coding or the sequence or abundance of the functional RNA should the transcript be non-coding.

In some aspects, the present disclosure provides, inter alia, a compound of Formula (I), (II), or (II).

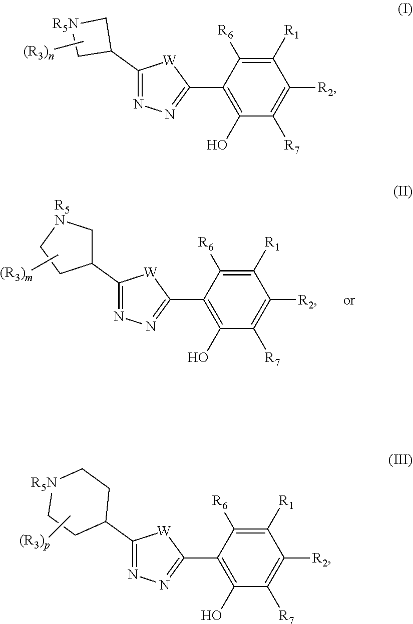

or a pharmaceutically acceptable salt, solvate, or prodrug thereof.

In some aspects, the present disclosure provides a compound obtainable by, or obtained by, a method for preparing a compound as described here (e.g., a method comprising one or more steps described in herein).

In some aspects, the present disclosure provides a pharmaceutical composition comprising a compound of the present disclosure, or a pharmaceutically acceptable salt, solvate, or prodrug thereof, and a pharmaceutically acceptable diluent or carrier.

In some aspects, the present disclosure provides an intermediate as described herein, being suitable for use in a method for preparing a compound as described herein (e.g., the intermediate is selected from the intermediates described herein).

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt, solvate, or prodrug thereof, or a pharmaceutical composition of the present disclosure.

In some aspects, the present disclosure provides a method of treating a disease or disorder disclosed herein in a subject in need thereof, comprising administering to the subject a therapeutically effective amount of a compound of the present disclosure or a pharmaceutically acceptable salt, solvate, or prodrug thereof, or a pharmaceutical composition of the present disclosure.

In some aspects, the present disclosure provides a compound of the present disclosure or a pharmaceutically acceptable salt, solvate, or prodrug thereof for use in treating or preventing a disease or disorder disclosed herein.

In some aspects, the present disclosure provides a compound of the present disclosure or a pharmaceutically acceptable salt, solvate, or prodrug thereof for use in treating a disease or disorder disclosed herein.

In some aspects, the present disclosure provides use of a compound of the present disclosure or a pharmaceutically acceptable salt, solvate, or prodrug thereof for treating or preventing a disease or disorder disclosed herein.

In some aspects, the present disclosure provides use of a compound of the present disclosure or a pharmaceutically acceptable salt, solvate, or prodrug thereof for treating a disease or disorder disclosed herein.

In some aspects, the present disclosure provides use of a compound of the present disclosure or a pharmaceutically acceptable salt, solvate, or prodrug thereof in the manufacture of a medicament for treating or preventing a disease or disorder disclosed herein.

In some aspects, the present disclosure provides use of a compound of the present disclosure or a pharmaceutically acceptable salt, solvate, or prodrug thereof in the manufacture of a medicament for treating a disease or disorder disclosed herein.

In some aspects, the present disclosure provides a method of preparing a compound of the present disclosure.

In some aspects, the present disclosure provides a method of preparing a compound, comprising one or more steps described herein.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the disclosure will be apparent from the following detailed description and claims.

### DETAILED DESCRIPTION

Compounds described herein are generally designed to treat diseases and disorders disclosed herein.

### Definitions

Unless otherwise stated, the following terms used in the specification and claims have the following meanings set out below.

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intends to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl, or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, “alkenyl” is intended to include straight-chain or branched hydrocarbon groups having from 2 to 6 carbon atoms, one or more carbon-carbon double bonds, and no triple bonds (“C₂-C₆alkenyl”). The one or more carbon-carbon double bonds can be internal (such as in 2-butenyl) or terminal (such as in 1-butenyl). Examples of C₂-C₆alkenyl groups include ethenyl (C₂), 1-propenyl (C₃), 2-propenyl (C₃), 1-butenyl (C₄), 2-butenyl (C₄), butadienyl (C₄), and the like.

As used herein, “alkynyl” is intended to include straight-chain or branched hydrocarbon groups having from 2 to 6 carbon atoms, one or more carbon-carbon triple bonds, and optionally one or more double bonds (“C₂-C₆ alkynyl”). The one or more carbon-carbon triple bonds can be internal (such as in 2-butynyl) or terminal (such as in 1-butynyl). Examples of C₂-C₄alkynyl groups include, without limitation, ethynyl (C₂), 1-propynyl (C₃), 2-propynyl (C₃), 1-butynyl (C₄), 2-butynyl (C₄), and the like.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphydryl, alkylthio, arylthio, thiocarboxylate, sulphates, alkylsulphinyl, sulphonato, sulphamoyl, sulphonamido, nitro, trifluoromethyl, cyano, azido, heterocycloalkyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, the term “alkoxy” or “alkoxyl” refers to the group —OR where R is alkyl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e., with between 1 and 6 carbon atoms.

As used herein, “heteroalkyl”, “C₁, C₂, C₃, C₄, C₅, or C₆ heteroalkyl” or “C₁-C₆ heteroalkyl” is intended to include C₁, C₂, C₃, C₄, C₅, or C₆ straight chain (linear) saturated aliphatic hydrocarbon groups and C₃, C₄, C₅, or C₆ branched saturated aliphatic hydrocarbon groups, in which at least one of the carbons has been replaced with N, O, or S. The heteroatom will be bonded to any required hydrogens to complete the heteroatom's valence (e.g., a CH₂ may be replaced with an “O” or a “NH”, a CH may be replaced with an N, etc.)). Such substituents can include, for example, —O—CH(CH₃)₂, —CH₂—N(CH₃)—CH₂CH₂OCH₃, —S—CH₂CH₂—O—CH₂CH₃, and so forth.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic.

As used herein, the term “heterocycloalkyl” or “heterocyclyl” refers to a saturated or partially unsaturated 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulphur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like. In the case of multicyclic heterocycloalkyl, only one of the rings in the heterocycloalkyl needs to be non-aromatic (e.g., 4,5,6,7-tetrahydrobenzo[c]isoxazolyl).

As used herein, the term “aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system. Examples of aryl groups include, but are not limited to, phenyl, naphthyl and the like. Conveniently, an aryl is phenyl.

As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulphur. The nitrogen atom may be substituted or unsubstituted (i.e., N or NR wherein R is H or other substituents, as defined). The nitrogen and sulphur heteroatoms may optionally be oxidised (i.e., N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like. Heteroaryl groups can also be fused or bridged with alicyclic or heterocyclic rings, which are not aromatic so as to form a multicyclic system (e.g., 4,5,6,7-tetrahydrobenzo[c]isoxazolyl).

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthrydine, indole, benzofuran, purine, benzofuran, deazapurine, indolizine.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocycloalkyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

As used herein, the term “hydroxy” or “hydroxyl” includes groups with an —OH or —O⁻.

As used herein, the term “cyano” refers to the group —CN.

As used herein, the term “nitro” refers to the radical —NO₂.

As used herein, the term “oxo” refers to ═O.

As used herein, the term “halo” or “halogen” refers to fluoro, chloro, bromo and iodo.

As used herein, the term “haloalkyl” refers to a branched or unbranched alkyl substituted with one or more halogens. For example, a C₁₋₆haloalkyl is an alkyl group of from one to seven carbons wherein at least one H is substituted by a halogen. Examples of haloalkyl include but are not limited to CFH₂, CF₂H, CF₃, CH₂CF₃, CF₂CF₃, C(F)(CH₃)₂, CH₂CH₂Br, CH(I)CH₂F, and CH₂Cl.

As used herein, the term “haloalkoxy” refers to alkoxy structures that are substituted with one or more halo groups or with combinations thereof. For example, the terms “fluoroalkyl” and “fluoro alkoxy” include haloalkyl and haloalkoxy groups, respectively, in which the halo is fluorine.

As used herein, the term “optionally substituted haloalkyl” refers to unsubstituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocycloalkyl, alkylaryl, or an aromatic or heteroaromatic moiety.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is to be understood that the present disclosure provides methods for the synthesis of the compounds of any of the Formulae described herein. The present disclosure also provides detailed methods for the synthesis of various disclosed compounds of the present disclosure according to the following schemes as well as those shown in the Examples.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognised reference textbooks of organic synthesis known to those in the art

One of ordinary skill in the art will note that, during the reaction sequences and synthetic schemes described herein, the order of certain steps may be changed, such as the introduction and removal of protecting groups. One of ordinary skill in the art will recognise that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

It is to be understood that, unless otherwise stated, any description of a method of treatment includes use of the compounds to provide such treatment or prophylaxis as is described herein, as well as use of the compounds to prepare a medicament to treat or prevent such condition. It is to be understood that, unless otherwise stated, any description of a method of treatment includes use of the compounds to provide such treatment or prophylaxis as is described herein, as well as use of the compounds to prepare a medicament to treat such condition. The treatment includes treatment of human or non-human animals including rodents and other disease models.

As used herein, the term “subject” is interchangeable with the term “subject in need thereof”, both of which refer to a subject having a disease or having an increased risk of developing the disease. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In one embodiment, the mammal is a human. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a subject for the purpose of combating a disease, condition, or disorder and includes the administration of a compound of the present disclosure, or a pharmaceutically acceptable salt, or solvate thereof, to alleviate the symptoms or complications of a disease, condition or disorder, or to eliminate the disease, condition or disorder. The term “treat” can also include treatment of a cell in vitro or an animal model.

It is to be appreciated that references to “treating” or “treatment” include the alleviation of established symptoms of a condition. “Treating” or “treatment” of a state, disorder or condition therefore includes: (1) delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

It is to be understood that a compound of the present disclosure, or a pharmaceutically acceptable salt, or solvate thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the disclosure.

It is to be understood that the present disclosure also provides pharmaceutical compositions comprising any compound described herein in combination with at least one pharmaceutically acceptable excipient or carrier.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the subject. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulphite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., a disease or disorder disclosed herein) and the health of the subject should preferably be closely monitored during and for a reasonable period after treatment.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

A “therapeutically effective amount” means the amount of a compound that, when administered to a subject for treating a disease, is sufficient to effect such treatment for the disease. The “therapeutically effective amount” will vary depending on the compound, the disease and its severity and the age, weight, etc., of the subject to be treated.

It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the subject, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilising processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilisation. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebuliser.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient subject, and the experience and judgment of the clinician or practitioner administering the therapy, among other factors affecting the selected dosage. Generally, the dose should be sufficient to result in slowing, and preferably regressing, the symptoms of the disease or disorder disclosed herein and also preferably causing complete regression of the disease or disorder. An effective amount of a pharmaceutical agent is that which provides an objectively identifiable improvement as noted by the clinician or other qualified observer. Improvement in survival and growth indicates regression. As used herein, the term “dosage effective manner” refers to amount of an active compound to produce the desired biological effect in a subject or cell.

It is to be understood that the pharmaceutical compositions can be included in a container, pack, or dispenser together with instructions for administration.

It is to be understood that, for the compounds of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulphonic, acetic, ascorbic, benzene sulphonic, benzoic, bicarbonic, carbonic, citric, edetic, ethane disulphonic, 1,2-ethane sulphonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulphonic, maleic, malic, mandelic, methane sulphonic, napsylic, nitric, oxalic, pamoic, pantothenic, phenylacetic, phosphoric, polygalacturonic, propionic, salicylic, stearic, subacetic, succinic, sulphamic, sulphanilic, sulphuric, tannic, tartaric, toluene sulphonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulphonic acid, 2-naphthalenesulphonic acid, 4-toluenesulphonic acid, camphorsulphonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3.

It is to be understood that all references to pharmaceutically acceptable salts include solvent addition forms (solvates) as defined herein, of the same salt.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally. One skilled in the art will recognise the advantages of certain routes of administration.

A salt, for example, can be formed between an anion and a positively charged group (e.g., amino) on a substituted compound disclosed herein. Suitable anions include chloride, bromide, iodide, sulphate, bisulphate, sulphamate, nitrate, phosphate, citrate, methanesulphonate, trifluoroacetate, glutamate, glucuronate, glutarate, malate, maleate, succinate, fumarate, tartrate, tosylate, salicylate, lactate, naphthalenesulphonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulphonamides, tetrazoles, sulphonates and phosphonates. See, e.g., Patani and LaVoie, Chem. Rev. 96, 3147-3176, 1996.

It is also to be understood that certain compounds of any one of the Formulae disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a ti-hydrate.

Compounds of any one of the Formulae disclosed herein may exist in a number of different tautomeric forms and references to compounds of Formula (I), (II), and (III) include all such forms. For the avoidance of doubt, where a compound can exist in one of several tautomeric forms, and only one is specifically described or shown, all others are nevertheless embraced by Formula (I), (II), and (III). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

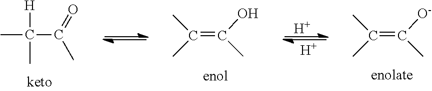

Compounds of any one of the Formulae disclosed herein containing an amine function may also form N-oxides. A reference herein to a compound of Formula (I), (II), and (III) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-oxides can be formed by treatment of the corresponding amine with an oxidising agent such as hydrogen peroxide or a peracid (e.g., a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of any one of the Formulae disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substitutents at the ester or amide group in any one of the Formulae disclosed herein.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterised by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterised by the absolute configuration of its asymmetric center and is described by the R- and S-sequencing rules of Cahn and Prelog, or by the manner in which the molecule rotates the plane of polarised light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centers (E- and Z-isomers).

Accordingly, the present disclosure includes those compounds of any one of the Formulae disclosed herein as defined hereinbefore when made available by organic synthesis and when made available within the human or animal body by way of cleavage of a prodrug thereof. Accordingly, the present disclosure includes those compounds of any one of the Formulae disclosed herein that are produced by organic synthetic means and also such compounds that are produced in the human or animal body by way of metabolism of a precursor compound, that is a compound of any one of the Formulae disclosed herein may be a synthetically-produced compound or a metabolically-produced compound.

A suitable pharmaceutically acceptable prodrug of a compound of any one of the Formulae disclosed herein is one that is based on reasonable medical judgment as being suitable for administration to the subject without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of any one of the Formulae disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the subject to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include a-acyloxyalkyl groups such as acetoxymethyl and pivaloyloxymethyl groups.

A suitable pharmaceutically acceptable prodrug of a compound of any one of the Formulae disclosed herein that possesses a carboxy group is, for example, an in vivo cleavable amide thereof, for example an amide formed with an amine such as ammonia, a C₁₋₄alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of any one of the Formulae disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl,morpholinomethyl,piperazin-1-ylmethyl and 4-(C₁-C₄ alkyl)piperazin-1-ylmethyl.

The dosage regimen utilising the compounds is selected in accordance with a variety of factors including type, species, age, weight, sex and medical condition of the subject; the severity of the condition to be treated; the route of administration; the renal and hepatic function of the subject; and the particular compound or salt thereof employed. An ordinarily skilled physician or veterinarian can readily determine and prescribe the effective amount of the drug required to prevent, counter, or arrest the progress of the condition. An ordinarily skilled physician or veterinarian can readily determine and prescribe the effective amount of the drug required to counter or arrest the progress of the condition.

Techniques for formulation and administration of the disclosed compounds of the disclosure can be found in Remington: the Science and Practice of Pharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

As use herein, the phrase “compound of the disclosure” refers to those compounds which are disclosed herein, both generically and specifically.

### Compounds of the Present Disclosure

In some aspects, the present disclosure provides, inter alia, a compound of Formula (I):

A compound of Formula (I):

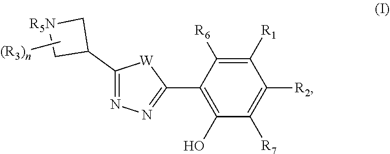

or a pharmaceutically acceptable salt, solvate, or prodrug thereof, wherein:
W is —S— or —HC═CH—;
   R₁ is H, halogen, hydroxyl, cyano, C₁-C₆alkyl, C₂-C₆alkenyl, C₂-C₆alkynyl, C₁-C₆haloalkyl, C₁-C₆alkoxyl, C₁-C₆haloalkoxyl, —(CH₂)₀₋₂—C₃-C₈cycloalkyl, NH₂, NH(C₁-C₆alkyl), N(C₁-C₆alkyl)₂, or —(CH₂)₀₋₂-heterocyclyl, wherein heterocyclyl is a 4- to 7-membered ring and comprises 1, 2, or 3 heteroatoms independently selected from N, O, and S, wherein the alkyl, alkenyl, alkynyl, alkoxyl, cycloalkyl, and heterocyclyl are optionally substituted with one or more C₃-C₈cycloalkyl, aryl, or 4- to 7-membered heterocyclyl comprising 1, 2, or 3 heteroatoms independently selected from N, O, and S;
   R₂ is aryl, 5- to 7-membered cycloalkyl, 5-, 6-, or 9-membered heterocyclyl comprising 1, 2, or 3 heteroatoms independently selected from N, O, and S, or 5-, 6-, or 9-membered heteroaryl comprising 2, 3, or 4 heteroatoms independently selected from N, O, and S, wherein the aryl, cycloalkyl, heterocyclyl, or heteroaryl is optionally substituted with one or more R₄;
   each R₃ is independently halogen, C₁-C₆alkyl, C₂-C₆alkenyl, C₂-C₆alkynyl, C₁-C₆alkoxyl, C₃-C₈cycloalkyl, NH₂, NH(C₁-C₆alkyl), or N(C₁-C₆alkyl)₂, wherein the alkyl, alkenyl, alkynyl, alkoxyl, and cycloalkyl are optionally substituted with one or more hydroxyl or NH₂;
   each R₄ is independently halogen, hydroxyl, cyano, nitro, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or C(O)NH₂, wherein the alkyl, alkenyl, alkynyl, alkoxyl, and cycloalkyl are optionally substituted with one or more hydroxyl, 4- to 7-membered heterocyclyl comprising 1, 2, or 3 heteroatoms independently selected from N, O, and S, NH₂, NH(C₁-C₆alkyl), or N(C₁-C₆alkyl)₂;
   R₅ is H, C₁-C₆alkyl, C₂-C₆alkenyl, C₂-C₆alkynyl, C₁-C₆alkoxyl, C₃-C₈cycloalkyl, —CH₂C₃-C₈cycloalkyl, heterocyclyl, —CH₂heterocycyl, —CH₂CH₂heterocycyl, —CH₂-(5-6 membered heteroaryl) wherein heterocyclyl is a 4- to 7-membered ring and comprises 1, 2, or 3 heteroatoms independently selected from N, O, and S, wherein R₅ is optionally substituted with one or more halogen, C₁-C₆alkyl, C₁-C₆haloalkyl, C₁-C₆ heteroalkyl, C₁-C₆alkoxyl, C₃-C₈cycloalkyl, spiro C₃-C₈cycloalkyl, spiro 4-7 membered heterocyclyl, 5-6 membered heteroaryl, oxo, cyano, or hydroxyl;
R₇ is H, halogen, C₁-C₆alkyl or C₁-C₆haloalkyl; and
n is 0, 1, 2, 3, 4, or 5.

In some aspects, the present disclosure provides, inter alia, a compound of Formula (Ic):

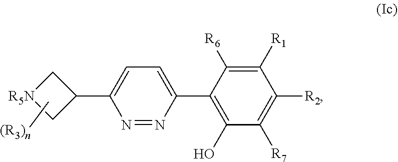

or a pharmaceutically acceptable salt, solvate, or prodrug thereof, wherein:
R₁ is H, halogen, hydroxyl, cyano, C₁-C₆alkyl, C₂-C₆alkenyl, C₂-C₆alkynyl, C₁-C₆haloalkyl, C₁-C₆alkoxyl, C₁-C₆haloalkoxyl, —(CH₂)₀₋₂—C₃-C₈cycloalkyl, NH₂, NH(C₁-C₆alkyl), N(C₁-C₆alkyl)₂, or —(CH₂)₀₋₂-heterocyclyl, wherein heterocyclyl is a 4- to 7-membered ring and comprises 1, 2, or 3 heteroatoms independently selected from N, O, and S, wherein the alkyl, alkenyl, alkynyl, alkoxyl, cycloalkyl, and heterocyclyl are optionally substituted with one or more C₃-C₈cycloalkyl, aryl, or 4- to 7-membered heterocyclyl comprising 1, 2, or 3 heteroatoms independently selected from N, O, and S;
R₂ is aryl, 5- to 7-membered cycloalkyl, 5-, 6-, or 9-membered heterocyclyl comprising 1, 2, or 3 heteroatoms independently selected from N, O, and S, or 5-, 6-, or 9-membered heteroaryl comprising 2, 3, or 4 heteroatoms independently selected from N, O, and S, wherein the aryl, cycloalkyl, heterocyclyl, or heteroaryl is optionally substituted with one or more R₄;
each R₃ is independently halogen, C₁-C₆alkyl, C₂-C₆alkenyl, C₂-C₆alkynyl, C₁-C₆alkoxyl, C₃-C₈cycloalkyl, NH₂, NH(C₁-C₆alkyl), or N(C₁-C₆alkyl)₂, wherein the alkyl, alkenyl, alkynyl, alkoxyl, and cycloalkyl are optionally substituted with one or more hydroxyl or NH₂;
each R₄ is independently halogen, hydroxyl, cyano, nitro, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or C(O)NH₂, wherein the alkyl, alkenyl, alkynyl, alkoxyl, and cycloalkyl are optionally substituted with one or more hydroxyl, 4- to 7-membered heterocyclyl comprising 1, 2, or 3 heteroatoms independently selected from N, O, and S, NH₂, NH(C₁-C₆alkyl), or N(C₁-C₆alkyl)₂;
R₅ is H, C₁-C₆alkyl, C₂-C₆alkenyl, C₂-C₆alkynyl, C₁-C₆alkoxyl, C₃-C₈cycloalkyl, —CH₂C₃-C₈cycloalkyl, heterocyclyl, —CH₂heterocycyl, —CH₂CH₂heterocycyl, —CH₂-(5-6 membered heteroaryl) wherein heterocyclyl is a 4- to 7-membered ring and comprises 1, 2, or 3 heteroatoms independently selected from N, O, and S, wherein R₅ is optionally substituted with one or more halogen, C₁-C₆alkyl, C₁-C₆haloalkyl, C₁-C₆ heteroalkyl, C₁-C₆alkoxyl, C₃-C₈cycloalkyl, spiro C₃-C₈cycloalkyl, spiro 4-7 membered heterocyclyl, 5-6 membered heteroaryl, oxo, cyano, or hydroxyl;
R₆ is H, halogen, C₁-C₆alkyl or C₁-C₆haloalkyl;
R₇ is H, halogen, C₁-C₆alkyl or C₁-C₆haloalkyl; and
n is 0, 1, 2, 3, 4, or 5.

In some aspects, the present disclosure provides, inter alia, a compound of Formula (Id):

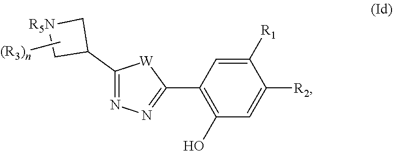

or a pharmaceutically acceptable salt, solvate, or prodrug thereof, wherein:
W is —S— or —HC═CH—;
R₁ is H, halogen, hydroxyl, cyano, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or 4- to 7-membered heterocyclyl comprising 1, 2, or 3 heteroatoms independently selected from N, O, and S, wherein the alkyl, alkenyl, alkynyl, alkoxyl, cycloalkyl, and heterocyclyl are optionally substituted with one or more C₃-C₈cycloalkyl, aryl, or 4- to 7-membered heterocyclyl comprising 1, 2, or 3 heteroatoms independently selected from N, O, and S;
R₂ is aryl, 5- to 7-membered cycloalkyl, 5-, 6-, or 9-membered heterocyclyl comprising 1, 2, or 3 heteroatoms independently selected from N, O, and S, or 5-, 6-, or 9-membered heteroaryl comprising 2, 3, or 4 heteroatoms independently selected from N, O, and S, wherein the aryl, cycloalkyl, heterocyclyl, or heteroaryl is optionally substituted with one or more R₄;
each R₃ is independently halogen, C₁-C₆alkyl, C₂-C₆alkenyl, C₂-C₆alkynyl, C₁-C₆alkoxyl, C₃-C₈cycloalkyl, NH₂, NH(C₁-C₆alkyl), or N(C₁-C₆alkyl)₂, wherein the alkyl, alkenyl, alkynyl, alkoxyl, and cycloalkyl are optionally substituted with one or more hydroxyl or NH₂;
each R₄ is independently halogen, hydroxyl, cyano, nitro, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or C(O)NH₂, wherein the alkyl, alkenyl, alkynyl, alkoxyl, and cycloalkyl are optionally substituted with one or more hydroxyl, 4- to 7-membered heterocyclyl comprising 1, 2, or 3 heteroatoms independently selected from N, O, and S, NH₂, NH(C₁-C₆alkyl), or N(C₁-C₆alkyl)₂;
R₅ is H, C₁-C₆alkyl, C₂-C₆alkenyl, C₂-C₆alkynyl, C₁-C₆alkoxyl, C₃-C₈cycloalkyl, —CH₂C₃-C₈cycloalkyl, heterocyclyl, —CH₂heterocycyl, —CH₂CH₂heterocycyl, —CH₂-(5-6 membered heteroaryl) wherein heterocyclyl is a 4- to 7-membered ring and comprises 1, 2, or 3 heteroatoms independently selected from N, O, and S, wherein R₅ is optionally substituted with one or more halogen, C₁-C₆alkyl, C₁-C₆haloalkyl, C₁-C₆ heteroalkyl, C₁-C₆alkoxyl, C₃-C₈cycloalkyl, spiro C₃-C₈cycloalkyl, spiro 4-7 membered heterocyclyl, 5-6 membered heteroaryl, oxo, cyano, or hydroxyl; and
n is 0, 1, 2, 3, 4, or 5.

In some aspects, the present disclosure provides, inter alia, a compound of Formula (II):

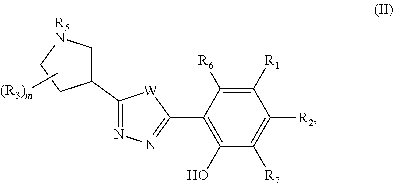

or a pharmaceutically acceptable salt, solvate, or prodrug thereof, wherein:
W is —S— or —HC═CH—;
R₁ is H, halogen, hydroxyl, cyano, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or 4- to 7-membered heterocyclyl comprising 1, 2, or 3 heteroatoms independently selected from N, O, and S, wherein the alkyl, alkenyl, alkynyl, alkoxyl, cycloalkyl, and heterocyclyl are optionally substituted with one or more C₃-C₈cycloalkyl, aryl, or 4- to 7-membered heterocyclyl comprising 1, 2, or 3 heteroatoms independently selected from N, O, and S;
R₂ is aryl, 5- to 7-membered cycloalkyl, 5-, 6-, or 9-membered heterocyclyl comprising 1, 2, or 3 heteroatoms independently selected from N, O, and S, or 5-, 6-, or 9-membered heteroaryl comprising 2, 3, or 4 heteroatoms independently selected from N, O, and S, wherein the aryl, cycloalkyl, heterocyclyl, or heteroaryl is optionally substituted with one or more R₄;
each R₃ is independently halogen, C₁-C₆alkyl, C₂-C₆alkenyl, C₂-C₆alkynyl, C₁-C₆alkoxyl, C₃-C₈cycloalkyl, NH₂, NH(C₁-C₆alkyl), or N(C₁-C₆alkyl)₂, wherein the alkyl, alkenyl, alkynyl, alkoxyl, and cycloalkyl are optionally substituted with one or more hydroxyl or NH₂;
each R₄ is independently halogen, hydroxyl, cyano, nitro, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or C(O)NH₂, wherein the alkyl, alkenyl, alkynyl, alkoxyl, and cycloalkyl are optionally substituted with one or more hydroxyl, 4- to 7-membered heterocyclyl comprising 1, 2, or 3 heteroatoms independently selected from N, O, and S, NH₂, NH(C₁-C₆alkyl), or N(C₁-C₆alkyl)₂;
R₅ is H, C₁-C₆alkyl, C₂-C₆alkenyl, C₂-C₆alkynyl, C₁-C₆alkoxyl, C₃-C₈cycloalkyl, —CH₂C₃-C₈cycloalkyl, heterocyclyl, —CH₂heterocycyl, —CH₂CH₂heterocycyl, —CH₂-(5-6 membered heteroaryl) wherein heterocyclyl is a 4- to 7-membered ring and comprises 1, 2, or 3 heteroatoms independently selected from N, O, and S, wherein R₅ is optionally substituted with one or more halogen, C₁-C₆alkyl, C₁-C₆haloalkyl, C₁-C₆ heteroalkyl, C₁-C₆alkoxyl, C₃-C₈cycloalkyl, spiro C₃-C₈cycloalkyl, spiro 4-7 membered heterocyclyl, 5-6 membered heteroaryl, oxo, cyano, or hydroxyl;
R₆ is H, halogen, C₁-C₆alkyl or C₁-C₆haloalkyl;
R₇ is H, halogen, C₁-C₆alkyl or C₁-C₆haloalkyl; and
m is 0, 1, 2, 3, 4, 5, 6, or 7.

In some aspects, the present disclosure provides, inter alia, a compound of Formula (III):

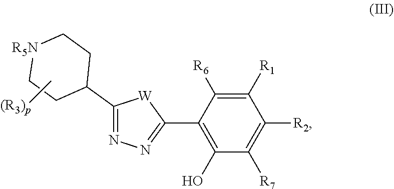

or a pharmaceutically acceptable salt, solvate, or prodrug thereof, wherein:
W is —S— or —HC═CH—;
R₁ is H, halogen, hydroxyl, cyano, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or 4- to 7-membered heterocyclyl comprising 1, 2, or 3 heteroatoms independently selected from N, O, and S, wherein the alkyl, alkenyl, alkynyl, alkoxyl, cycloalkyl, and heterocyclyl are optionally substituted with one or more C₃-C₈cycloalkyl, aryl, or 4- to 7-membered heterocyclyl comprising 1, 2, or 3 heteroatoms independently selected from N, O, and S;
R₂ is aryl, 5- to 7-membered cycloalkyl, 5-, 6-, or 9-membered heterocyclyl comprising 1, 2, or 3 heteroatoms independently selected from N, O, and S, or 6- or 9-membered heteroaryl comprising 2, 3, or 4 heteroatoms independently selected from N, O, and S, wherein the aryl, cycloalkyl, heterocyclyl, or heteroaryl is optionally substituted with one or more R₄;
each R₃ is independently halogen, C₁-C₆alkyl, C₂-C₆alkenyl, C₂-C₆alkynyl, C₁-C₆alkoxyl, C₃-C₈cycloalkyl, NH₂, NH(C₁-C₆alkyl), or N(C₁-C₆alkyl)₂, wherein the alkyl, alkenyl, alkynyl, alkoxyl, and cycloalkyl are optionally substituted with one or more hydroxyl or NH₂;
each R₄ is independently halogen, hydroxyl, cyano, nitro, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or C(O)NH₂, wherein the alkyl, alkenyl, alkynyl, alkoxyl, and cycloalkyl are optionally substituted with one or more hydroxyl, 4- to 7-membered heterocyclyl, NH₂, NH(C₁-C₆alkyl), or N(C₁-C₆alkyl)₂;
R₅ is H, C₁-C₆alkyl, C₂-C₆alkenyl, C₂-C₆alkynyl, C₁-C₆alkoxyl, C₃-C₈cycloalkyl, —CH₂C₃-C₈cycloalkyl, heterocyclyl, —CH₂heterocycyl, —CH₂CH₂heterocycyl, —CH₂-(5-6 membered heteroaryl) wherein heterocyclyl is a 4- to 7-membered ring and comprises 1, 2, or 3 heteroatoms independently selected from N, O, and S, wherein R₅ is optionally substituted with one or more halogen, C₁-C₆alkyl, C₁-C₆haloalkyl, C₁-C₆ heteroalkyl, C₁-C₆alkoxyl, C₃-C₈cycloalkyl, spiro C₃-C₈cycloalkyl, spiro 4-7 membered heterocyclyl, 5-6 membered heteroaryl, oxo, cyano, or hydroxyl;
R₆ is H, halogen, C₁-C₆alkyl or C₁-C₆haloalkyl;
R₇ is H, halogen, C₁-C₆alkyl or C₁-C₆haloalkyl; and
p is 0, 1, 2, 3, 4, 5, 6, 7, 8, or 9.

It is understood that, for a compound of Formula (I), (II) or (III), W, R₁, R₂, R₃, R₄, R₅, R₆, R₇, m, n, and p can each be, where applicable, selected from the groups described herein, and any group described herein for any of W, R₁, R₂, R₃, R₄, R₅, R₆, R₇, m, n, and p can be combined, where applicable, with any group described herein for one or more of the remainder of W, R₁, R₂, R₃, R₄, R₅, R₆, R₇, m, n, and p.

In some embodiments, a compound of Formula I is a compound of Formula (Ia):

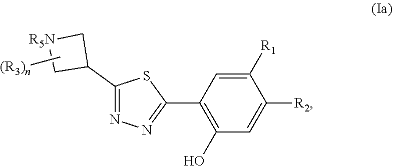

or a pharmaceutically acceptable salt, solvate, or prodrug thereof.

In some embodiments, a compound of Formula I is a compound of Formula (Ib):

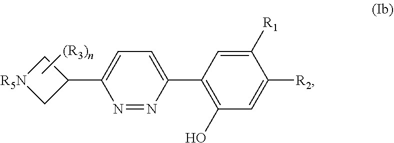

or a pharmaceutically acceptable salt, solvate, or prodrug thereof.

In some embodiments, a compound of Formula I is a compound of Formula (Ic):

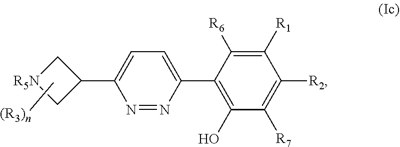

or a pharmaceutically acceptable salt, solvate, or prodrug thereof.

In some embodiments, a compound of Formula I is a compound of Formula (Id):

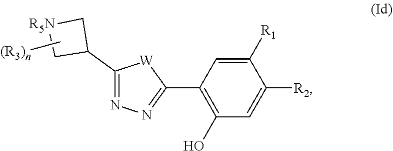

or a pharmaceutically acceptable salt, solvate, or prodrug thereof.

In some embodiments, a compound of Formula II is a compound of Formula (IIa):

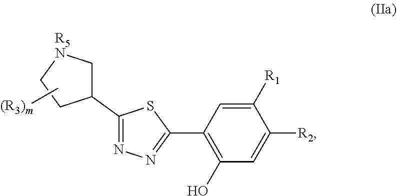

or a pharmaceutically acceptable salt, solvate, or prodrug thereof.

In some embodiments, a compound of Formula II is a compound of Formula (IIb):

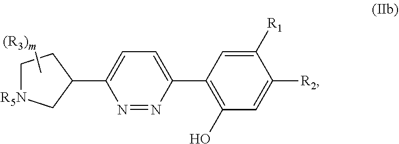

or a pharmaceutically acceptable salt, solvate, or prodrug thereof.

In some embodiments, a compound of Formula III is a compound of Formula (IIIa):

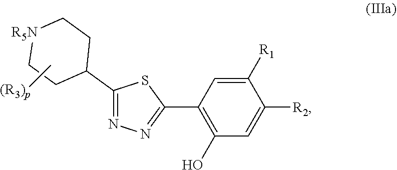

or a pharmaceutically acceptable salt, solvate, or prodrug thereof.

In some embodiments, a compound of Formula III is a compound of Formula (IIIb):

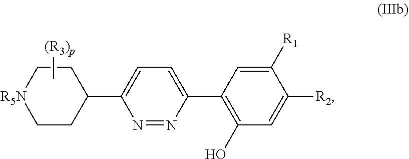

or a pharmaceutically acceptable salt, solvate, or prodrug thereof.

For a compound of Formula (I), (Ia), (Ib), (Ic), (Id), (II), (IIa), (IIb), (III), (IIIa), or (IIIb), where applicable:

In some embodiments, W is —S—.

In some embodiments, W is —HC═CH—.

In some embodiments, R₁ is H, halogen, hydroxyl, cyano, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or 4- to 7-membered heterocyclyl comprising 1, 2, or 3 heteroatoms independently selected from N, O, and S.

In some embodiments, R₁ is H.

In some embodiments, R₁ is halogen, hydroxyl, cyano, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or 4- to 7-membered heterocyclyl comprising 1, 2, or 3 heteroatoms independently selected from N, O, and S heterocyclyl, wherein the alkyl, alkenyl, alkynyl, alkoxyl, cycloalkyl, and heterocyclyl are optionally substituted with one or more C₃-C₈cycloalkyl, aryl, or 4- to 7-membered heterocyclyl comprising 1, 2, or 3 heteroatoms independently selected from N, O, and S.

In some embodiments, R₁ is halogen, hydroxyl, cyano, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or 4- to 7-membered heterocyclyl comprising 1, 2, or 3 heteroatoms independently selected from N, O, and S heterocyclyl.

In some embodiments, R₁ is halogen, hydroxyl, or cyano.

In some embodiments, R₁ is halogen. In some embodiments, R₁ is F, Cl, Br, or I. In some embodiments, R₁ is F, Cl, or Br. In some embodiments, R₁ is F or Cl. In some embodiments, R₁ is F. In some embodiments, R₁ is Cl. In some embodiments, R₁ is Br. In some embodiments, R₁ is I.

In some embodiments, R₁ is hydroxyl.

In some embodiments, R₁ is cyano.

In some embodiments, R₁ is C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or 4- to 7-membered heterocyclyl comprising 1, 2, or 3 heteroatoms independently selected from N, O, and S, wherein the alkyl, alkenyl, alkynyl, alkoxyl, cycloalkyl, and heterocyclyl are optionally substituted with one or more C₃-C₈cycloalkyl, aryl, or 4- to 7-membered heterocyclyl comprising 1, 2, or 3 heteroatoms independently selected from N, O, and S.

In some embodiments, R₁ is C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or 4- to 7-membered heterocyclyl comprising 1, 2, or 3 heteroatoms independently selected from N, O, and S.

In some embodiments, R₁ is C₁-C₆alkyl, C₂-C₆alkenyl, or C₂-C₆alkynyl.

In some embodiments, R₁ is C₁-C₆alkyl.

In some embodiments, R₁ is methyl. In some embodiments, R₁ is ethyl. In some embodiments, R₁ is propyl. In some embodiments, R₁ is butyl. In some embodiments, R₁ is iso-propyl. In some embodiments, R₁ is iso-butyl. In some embodiments, R₁ is sec-butyl. In some embodiments, R₁ is tert-butyl.

In some embodiments, R₁ is C₁-C₆alkyl optionally substituted with one or more C₃-C₈cycloalkyl, aryl, or 4- to 7-membered heterocyclyl comprising 1, 2, or 3 heteroatoms independently selected from N, O, and S. In some embodiments, R₁ is C₁-C₆alkyl substituted with one or more C₃-C₈cycloalkyl, aryl, or 4- to 7-membered heterocyclyl comprising 1, 2, or 3 heteroatoms independently selected from N, O, and S. In some embodiments, R₁ is C₁-C₆alkyl substituted with one C₃-C₈cycloalkyl, aryl, or 4- to 7-membered heterocyclyl comprising 1, 2, or 3 heteroatoms independently selected from N, O, and S. In some embodiments, R₁ is C₁-C₆alkyl substituted with two C₃-C₈cycloalkyl, aryl, or 4- to 7-membered heterocyclyl comprising 1, 2, or 3 heteroatoms independently selected from N, O, and S. In some embodiments, R₁ is C₁-C₆alkyl substituted with three C₃-C₈cycloalkyl, aryl, or 4- to 7-membered heterocyclyl comprising 1, 2, or 3 heteroatoms independently selected from N, O, and S.

In some embodiments, R₁ is C₂-C₆alkenyl. In some embodiments, R₁ is C₂alkenyl. In some embodiments, R₁ is C₃alkenyl. In some embodiments, R₁ is C₄alkenyl. In some embodiments, R₁ is C₅alkenyl. In some embodiments, R₁ is C₆alkenyl.

In some embodiments, R₁ is C₂-C₆alkenyl optionally substituted with one or more C₃-C₈cycloalkyl, aryl, or 4- to 7-membered heterocyclyl comprising 1, 2, or 3 heteroatoms independently selected from N, O, and S. In some embodiments, R₁ is C₂-C₆alkenyl substituted with one or more C₃-C₈cycloalkyl, aryl, or 4- to 7-membered heterocyclyl comprising 1, 2, or 3 heteroatoms independently selected from N, O, and S. In some embodiments, R₁ is C₂-C₆alkenyl substituted with one C₃-C₈cycloalkyl, aryl, or 4- to 7-membered heterocyclyl comprising 1, 2, or 3 heteroatoms independently selected from N, O, and S. In some embodiments, R₁ is C₂-C₆alkenyl substituted with two C₃-C₈cycloalkyl, aryl, or 4- to 7-membered heterocyclyl comprising 1, 2, or 3 heteroatoms independently selected from N, O, and S. In some embodiments, R₁ is C₂-C₆alkenyl substituted with three C₃-C₈cycloalkyl, aryl, or 4- to 7-membered heterocyclyl comprising 1, 2, or 3 heteroatoms independently selected from N, O, and S.

In some embodiments, R₁ is C₂-C₆alkynyl. In some embodiments, R₁ is C₂alkynyl. In some embodiments, R₁ is C₃alkynyl. In some embodiments, R₁ is C₄alkynyl. In some embodiments, R₁ is C₅alkynyl. In some embodiments, R₁ is C₆alkynyl.

In some embodiments, R₁ is C₂-C₆alkynyl optionally substituted with one or more C₃-C₈cycloalkyl, aryl, or 4- to 7-membered heterocyclyl comprising 1, 2, or 3 heteroatoms independently selected from N, O, and S. In some embodiments, R₁ is C₂-C₆alkynyl substituted with one or more C₃-C₈cycloalkyl, aryl, or 4- to 7-membered heterocyclyl comprising 1, 2, or 3 heteroatoms independently selected from N, O, and S. In some embodiments, R₁ is C₂-C₆alkynyl substituted with one C₃-C₈cycloalkyl, aryl, or 4- to 7-membered heterocyclyl comprising 1, 2, or 3 heteroatoms independently selected from N, O, and S. In some embodiments, R₁ is C₂-C₆alkynyl substituted with two C₃-C₈cycloalkyl, aryl, or 4- to 7-membered heterocyclyl comprising 1, 2, or 3 heteroatoms independently selected from N, O, and S. In some embodiments, R₁ is C₂-C₆alkynyl substituted with three C₃-C₈cycloalkyl, aryl, or 4- to 7-membered heterocyclyl comprising 1, 2, or 3 heteroatoms independently selected from N, O, and S.

In some embodiments, R₁ is C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or 4- to 7-membered heterocyclyl comprising 1, 2, or 3 heteroatoms independently selected from N, O, and S, wherein the alkoxyl, cycloalkyl, and heterocyclyl are optionally substituted with one or more C₃-C₈cycloalkyl, aryl, or 4- to 7-membered heterocyclyl comprising 1, 2, or 3 heteroatoms independently selected from N, O, and S.

In some embodiments, R₁ is C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or 4- to 7-membered heterocyclyl comprising 1, 2, or 3 heteroatoms independently selected from N, O, and S.

In some embodiments, R₁ is C₁-C₆haloalkyl, C₁-C₆alkoxyl, or C₁-C₆haloalkoxyl, wherein the alkoxyl is optionally substituted with one or more C₃-C₈cycloalkyl, aryl, or 4- to 7-membered heterocyclyl comprising 1, 2, or 3 heteroatoms independently selected from N, O, and S.

In some embodiments, R₁ is C₁-C₆haloalkyl, C₁-C₆alkoxyl, or C₁-C₆haloalkoxyl.

In some embodiments, R₁ is C₁-C₆haloalkyl. In some embodiments, R₁ is halomethyl. In some embodiments, R₁ is haloethyl. In some embodiments, R₁ is halopropyl. In some embodiments, R₁ is halobutyl. In some embodiments, R₁ is halopentyl. In some embodiments, R₁ is halohexyl.

In some embodiments, R₁ is C₁-C₆alkoxyl. In some embodiments, R₁ is methoxyl. In some embodiments, R₁ is ethoxyl. In some embodiments, R₁ is propoxyl. In some embodiments, R₁ is butyoxyl. In some embodiments, R₁ is pentyoxyl. In some embodiments, R₁ is hexoxyl.

In some embodiments, R₁ is C₁-C₆haloalkoxyl. In some embodiments, R₁ is halomethoxyl. In some embodiments, R₁ is haloethoxyl. In some embodiments, R₁ is halopropoxyl. In some embodiments, R₁ is halobutoxyl. In some embodiments, R₁ is halopentoxyl. In some embodiments, R₁ is halohexoxyl.

In some embodiments, R₁ is C₃-C₈cycloalkyl or 4- to 7-membered heterocyclyl comprising 1, 2, or 3 heteroatoms independently selected from N, O, and S, wherein the cycloalkyl and heterocyclyl are optionally substituted with one or more C₃-C₈cycloalkyl, aryl, or 4- to 7-membered heterocyclyl comprising 1, 2, or 3 heteroatoms independently selected from N, O, and S.

In some embodiments, R₁ is C₃-C₈cycloalkyl or 4- to 7-membered heterocyclyl comprising 1, 2, or 3 heteroatoms independently selected from N, O, and S.

In some embodiments, R₁ is C₃-C₈cycloalkyl. In some embodiments, R₁ is cyclopropyl. In some embodiments, R₁ is cyclobutyl. In some embodiments, R₁ is cyclopentyl. In some embodiments, R₁ is cyclohexyl.

In some embodiments, R₁ is bridged C₃-C₈cycloalkyl. In some embodiments, R₁ is fused C₃-C₈cycloalkyl. In some embodiments, R₁ is spiro C₃-C₈cycloalkyl.

In some embodiments, R₁ is C₃-C₈cycloalkyl optionally substituted with one or more C₃-C₈cycloalkyl, aryl, or 4- to 7-membered heterocyclyl comprising 1, 2, or 3 heteroatoms independently selected from N, O, and S. In some embodiments, R₁ is C₃-C₈cycloalkyl optionally substituted with one or more C₃-C₈cycloalkyl or aryl. In some embodiments, R₁ is C₃-C₈cycloalkyl substituted with one or more C₃-C₈cycloalkyl or aryl. In some embodiments, R₁ is C₃-C₈cycloalkyl substituted with one or more C₃-C₈cycloalkyl. In some embodiments, R₁ is C₃-C₈cycloalkyl substituted with one or more aryl.

In some embodiments, R₁ is 4- to 7-membered heterocyclyl comprising 1, 2, or 3 heteroatoms independently selected from N, O, and S.

In some embodiments, R₁ is 4-membered heterocyclyl comprising 1, 2, or 3 heteroatoms independently selected from N, O, and S.

In some embodiments, R₁ is 5-membered heterocyclyl comprising 1, 2, or 3 heteroatoms independently selected from N, O, and S.

In some embodiments, R₁ is 6-membered heterocyclyl comprising 1, 2, or 3 heteroatoms independently selected from N, O, and S.

In some embodiments, R₁ is 7-membered heterocyclyl comprising 1, 2, or 3 heteroatoms independently selected from N, O, and S.

In some embodiments, R₁ is 4- to 7-membered heterocyclyl comprising 1, 2, or 3 heteroatoms independently selected from N, O, and S optionally substituted with one or more C₃-C₈cycloalkyl, aryl, or 4- to 7-membered heterocyclyl comprising 1, 2, or 3 heteroatoms independently selected from N, O, and S. In some embodiments, R₁ is 4- to 7-membered heterocyclyl comprising 1, 2, or 3 heteroatoms independently selected from N, O, and S optionally substituted with one or more C₃-C₈cycloalkyl or aryl. In some embodiments, R₁ is 4- to 7-membered heterocyclyl comprising 1, 2, or 3 heteroatoms independently selected from N, O, and S substituted with one or more C₃-C₈cycloalkyl or aryl. In some embodiments, R₁ is 4- to 7-membered heterocyclyl comprising 1, 2, or 3 heteroatoms independently selected from N, O, and S substituted with one or more C₃-C₈cycloalkyl. In some embodiments, R₁ is 4- to 7-membered heterocyclyl comprising 1, 2, or 3 heteroatoms independently selected from N, O, and S substituted with one or more aryl.

In some embodiments, R₁ is NH₂, NH(C₁-C₆alkyl), or N(C₁-C₆alkyl)₂.

In some embodiments, R₁ is NH₂.

In some embodiments, R₁ is NH(C₁-C₆alkyl) or N(C₁-C₆alkyl)₂.

In some embodiments, R₁ is NH(C₁-C₆alkyl). In some embodiments, R₁ is NH(methyl). In some embodiments, R₁ is NH(ethyl). In some embodiments, R₁ is NH(propyl). In some embodiments, R₁ is NH(butyl). In some embodiments, R₁ is NH(pentyl). In some embodiments, R₁ is NH(hexyl).

In some embodiments, R₁ is N(C₁-C₆alkyl)₂. In some embodiments, R₁ is N(methyl)₂. In some embodiments, R₁ is N(ethyl)₂. In some embodiments, R₁ is N(propyl)₂. In some embodiments, R₁ is N(butyl)₂. In some embodiments, R₁ is N(pentyl)₂. In some embodiments, R₁ is N(hexyl)₂.

In some embodiments, R₁ is H or F.

In some embodiments, R₂ is aryl.

In some embodiments, R₂ is aryl optionally substituted with one or more R₄. In some embodiments, R₂ is aryl substituted with one or more R₄. In some embodiments, R₂ is aryl substituted with one R₄. In some embodiments, R₂ is aryl substituted with two R₄. In some embodiments, R₂ is aryl substituted with three R₄.

In some embodiments, R₂ is 5- to 7-membered cycloalkyl.

In some embodiments, R₂ is 5- to 7-membered saturated cycloalkyl. In some embodiments, R₂ is 5- to 7-membered partially saturated cycloalkyl.

In some embodiments, R₂ is 5- to 7-membered cycloalkyl optionally substituted with one or more R₄. In some embodiments, R₂ is 5- to 7-membered cycloalkyl substituted with one or more R₄. In some embodiments, R₂ is 5- to 7-membered cycloalkyl substituted with one R₄. In some embodiments, R₂ is 5- to 7-membered cycloalkyl substituted with two R₄. In some embodiments, R₂ is 5- to 7-membered cycloalkyl substituted with three R₄.

In some embodiments, R₂ is a 5-, 6-, or 9-membered heterocyclyl comprising 1, 2, or 3 heteroatoms independently selected from N, O, and S.

In some embodiments, R₂ is a 5-, 6-, or 9-membered heterocyclyl comprising 1, 2, or 3 heteroatoms independently selected from N and O.

In some embodiments, R₂ is a 5-, 6-, or 9-membered heterocyclyl comprising 1 or 2 heteroatoms independently selected from N, O, and S.

In some embodiments, R₂ is a 5-, 6-, or 9-membered heterocyclyl comprising 1 or 2 heteroatoms independently selected from N and O.

In some embodiments, R₂ is a 5-, 6-, or 9-membered heterocyclyl comprising 1, 2, or 3 heteroatoms independently selected from N, O, and S, wherein R₂ is optionally substituted with one or more R₄.

In some embodiments, R₂ is a 5-, 6-, or 9-membered heteroaryl comprising 2, 3, or 4 heteroatoms independently selected from N, O, and S.

In some embodiments, R₂ is a 5-, 6-, or 9-membered heteroaryl comprising 2, 3, or 4 heteroatoms independently selected from N and O.

In some embodiments, R₂ is a 5-, 6-, or 9-membered heteroaryl comprising 2, 3, or 4 heteroatoms independently selected from N, O, and S, wherein the heteroaryl is optionally substituted with one or more R₄.

In some embodiments, R₂ is a 5-, 6-, or 9-membered heteroaryl comprising 2, 3, or 4 heteroatoms independently selected from N and O, wherein the heteroaryl is optionally substituted with one or more R₄.

In some embodiments, R₂ is a 5-, 6-, or 9-membered heteroaryl comprising 2, 3, or 4 heteroatoms independently selected from N, O, and S, wherein the heteroaryl is substituted with one or more R₄.

In some embodiments, R₂ is a 5-, 6-, or 9-membered heteroaryl comprising 2, 3, or 4 heteroatoms independently selected from N and O, wherein the heteroaryl is substituted with one or more R₄.

In some embodiments, R₂ is a 5-, 6-, or 9-membered heteroaryl comprising 2, 3, or 4 heteroatoms independently selected from N, O, and S, wherein the heteroaryl is substituted with one R₄.

In some embodiments, R₂ is a 5-, 6-, or 9-membered heteroaryl comprising 2, 3, or 4 heteroatoms independently selected from N and O, wherein the heteroaryl is substituted with one R₄.

In some embodiments, R₂ is a 5-, 6-, or 9-membered heteroaryl comprising 2, 3, or 4 heteroatoms independently selected from N, O, and S, wherein the heteroaryl is substituted with two R₄.

In some embodiments, R₂ is a 5-, 6-, or 9-membered heteroaryl comprising 2, 3, or 4 heteroatoms independently selected from N and O, wherein the heteroaryl is substituted with two R₄.

In some embodiments, R₂ is a 5-, 6-, or 9-membered heteroaryl comprising 2, 3, or 4 heteroatoms independently selected from N, O, and S, wherein the heteroaryl is substituted with three R₄.

In some embodiments, R₂ is a 5-, 6-, or 9-membered heteroaryl comprising 2, 3, or 4 heteroatoms independently selected from N and O, wherein the heteroaryl is substituted with three R₄.

In some embodiments, R₂ is a 9-membered heteroaryl comprising 2, 3, or 4 heteroatoms independently selected from N, O, and S, wherein the heteroaryl is optionally substituted with one or more R₄.

In some embodiments, R₂ is a 9-membered heteroaryl comprising 2, 3, or 4 heteroatoms independently selected from N and O, wherein the heteroaryl is optionally substituted with one or more R₄.

In some embodiments, R₂ is a 9-membered heteroaryl comprising 2, 3, or 4 heteroatoms independently selected from N, O, and S, wherein the heteroaryl is substituted with one or more R₄.

In some embodiments, R₂ is a 9-membered heteroaryl comprising 2, 3, or 4 heteroatoms independently selected from N and O, wherein the heteroaryl is substituted with one or more R₄.

In some embodiments, R₂ is a 9-membered heteroaryl comprising 2, 3, or 4 heteroatoms independently selected from N, O, and S, wherein the heteroaryl is substituted with one R₄.

In some embodiments, R₂ is a 9-membered heteroaryl comprising 2, 3, or 4 heteroatoms independently selected from N and O, wherein the heteroaryl is substituted with one R₄.

In some embodiments, R₂ is a 9-membered heteroaryl comprising 2, 3, or 4 heteroatoms independently selected from N, O, and S, wherein the heteroaryl is substituted with two R₄.

In some embodiments, R₂ is a 9-membered heteroaryl comprising 2, 3, or 4 heteroatoms independently selected from N and O, wherein the heteroaryl is substituted with two R₄.

In some embodiments, R₂ is a 9-membered heteroaryl comprising 2, 3, or 4 heteroatoms independently selected from N, O, and S, wherein the heteroaryl is substituted with three R₄.

In some embodiments, R₂ is a 9-membered heteroaryl comprising 2, 3, or 4 heteroatoms independently selected from N and O, wherein the heteroaryl is substituted with three R₄.

In some embodiments, R₂ is a 9-membered heteroaryl comprising 2 heteroatoms independently selected from N, O, and S, wherein the heteroaryl is optionally substituted with one or more R₄.

In some embodiments, R₂ is a 9-membered heteroaryl comprising 2 heteroatoms independently selected from N and O, wherein the heteroaryl is optionally substituted with one or more R₄.

In some embodiments, R₂ is a 9-membered heteroaryl comprising 2 heteroatoms independently selected from N, O, and S, wherein the heteroaryl is substituted with one or more R₄.

In some embodiments, R₂ is a 9-membered heteroaryl comprising 2 heteroatoms independently selected from N and O, wherein the heteroaryl is substituted with one or more R₄.

In some embodiments, R₂ is a 9-membered heteroaryl comprising 2 heteroatoms independently selected from N, O, and S, wherein the heteroaryl is substituted with one R₄.

In some embodiments, R₂ is a 9-membered heteroaryl comprising 2 heteroatoms independently selected from N and O, wherein the heteroaryl is substituted with one R₄.

In some embodiments, R₂ is a 9-membered heteroaryl comprising 2 heteroatoms independently selected from N, O, and S, wherein the heteroaryl is substituted with two R₄.

In some embodiments, R₂ is a 9-membered heteroaryl comprising 2 heteroatoms independently selected from N and O, wherein the heteroaryl is substituted with two R₄.

In some embodiments, R₂ is a 9-membered heteroaryl comprising 2 heteroatoms independently selected from N, O, and S, wherein the heteroaryl is substituted with three R₄.

In some embodiments, R₂ is a 9-membered heteroaryl comprising 2 heteroatoms independently selected from N and O, wherein the heteroaryl is substituted with three R₄.

In some embodiments, R₂ is a 9-membered heteroaryl comprising 3 heteroatoms independently selected from N, O, and S, wherein the heteroaryl is optionally substituted with one or more R₄.

In some embodiments, R₂ is a 9-membered heteroaryl comprising 3 heteroatoms independently selected from N and O, wherein the heteroaryl is optionally substituted with one or more R₄.

In some embodiments, R₂ is a 9-membered heteroaryl comprising 3 heteroatoms independently selected from N, O, and S, wherein the heteroaryl is substituted with one or more R₄.

In some embodiments, R₂ is a 9-membered heteroaryl comprising 3 heteroatoms independently selected from N and O, wherein the heteroaryl is substituted with one or more R₄.

In some embodiments, R₂ is a 9-membered heteroaryl comprising 3 heteroatoms independently selected from N, O, and S, wherein the heteroaryl is substituted with one R₄.

In some embodiments, R₂ is a 9-membered heteroaryl comprising 3 heteroatoms independently selected from N and O, wherein the heteroaryl is substituted with one R₄.

In some embodiments, R₂ is a 9-membered heteroaryl comprising 3 heteroatoms independently selected from N, O, and S, wherein the heteroaryl is substituted with two R₄.

In some embodiments, R₂ is a 9-membered heteroaryl comprising 3 heteroatoms independently selected from N and O, wherein the heteroaryl is substituted with two R₄.

In some embodiments, R₂ is a 9-membered heteroaryl comprising 3 heteroatoms independently selected from N, O, and S, wherein the heteroaryl is substituted with three R₄.

In some embodiments, R₂ is a 9-membered heteroaryl comprising 3 heteroatoms independently selected from N and O, wherein the heteroaryl is substituted with three R₄.

In some embodiments, R₂ is a 9-membered heteroaryl comprising 4 heteroatoms independently selected from N, O, and S, wherein the heteroaryl is optionally substituted with one or more R₄.

In some embodiments, R₂ is a 9-membered heteroaryl comprising 4 heteroatoms independently selected from N and O, wherein the heteroaryl is optionally substituted with one or more R₄.

In some embodiments, R₂ is a 9-membered heteroaryl comprising 4 heteroatoms independently selected from N, O, and S, wherein the heteroaryl is substituted with one or more R₄.

In some embodiments, R₂ is a 9-membered heteroaryl comprising 4 heteroatoms independently selected from N and O, wherein the heteroaryl is substituted with one or more R₄.

In some embodiments, R₂ is a 9-membered heteroaryl comprising 4 heteroatoms independently selected from N, O, and S, wherein the heteroaryl is substituted with one R₄.

In some embodiments, R₂ is a 9-membered heteroaryl comprising 4 heteroatoms independently selected from N and O, wherein the heteroaryl is substituted with one R₄.

In some embodiments, R₂ is a 9-membered heteroaryl comprising 4 heteroatoms independently selected from N, O, and S, wherein the heteroaryl is substituted with two R₄.

In some embodiments, R₂ is a 9-membered heteroaryl comprising 4 heteroatoms independently selected from N and O, wherein the heteroaryl is substituted with two R₄.

In some embodiments, R₂ is a 9-membered heteroaryl comprising 4 heteroatoms independently selected from N, O, and S, wherein the heteroaryl is substituted with three R₄.

In some embodiments, R₂ is a 9-membered heteroaryl comprising 4 heteroatoms independently selected from N and O, wherein the heteroaryl is substituted with three R₄.

In some embodiments, R₂ is a bicyclic 9-membered heteroaryl.

In some embodiments, R₂ is

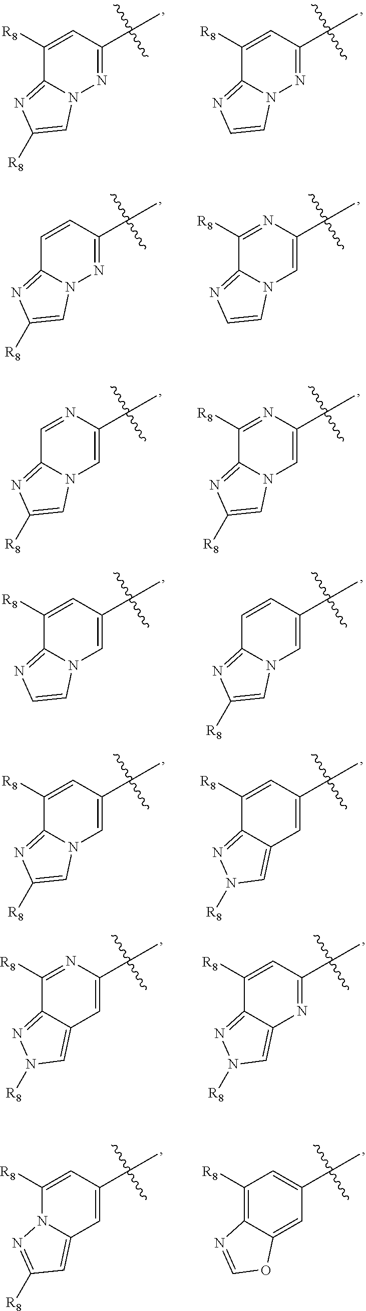

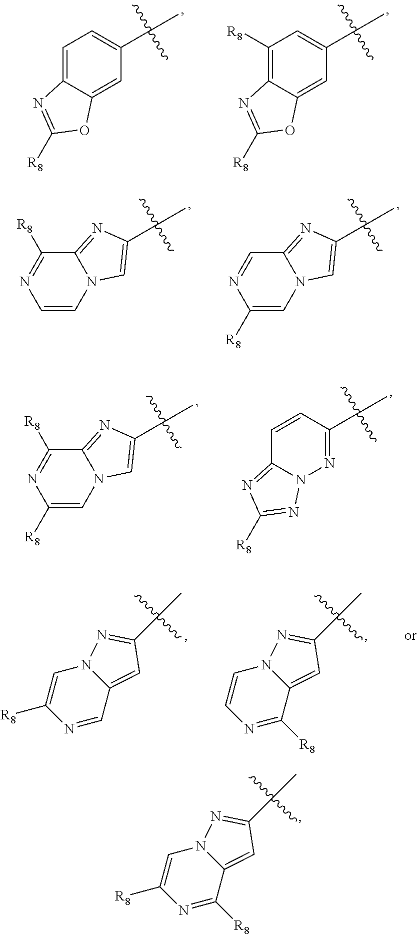

wherein each R₈ is independently R₄.

In some embodiments, R₂ is

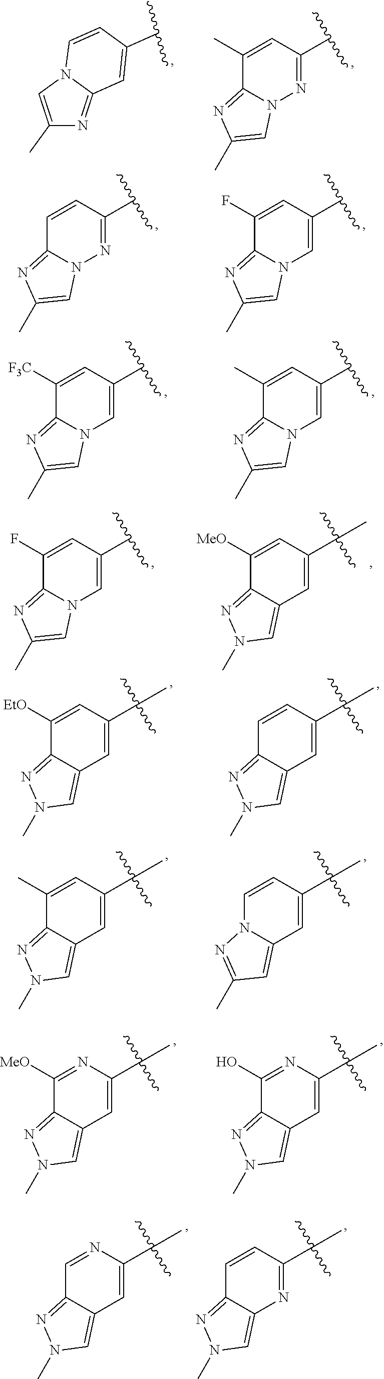

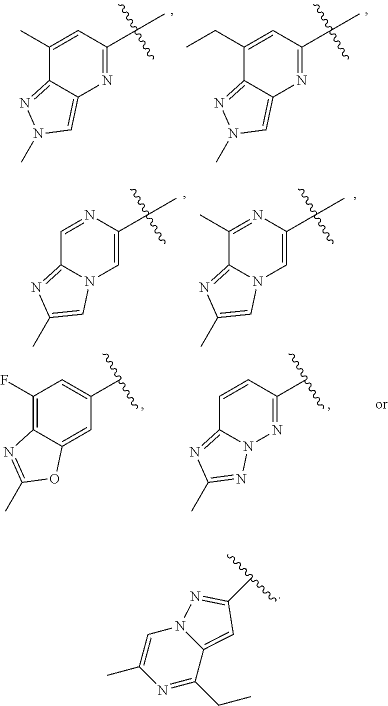

In some embodiments, each R₃ is independently halogen, C₁-C₆alkyl, C₂-C₆alkenyl, C₂-C₆alkynyl, C₁-C₆alkoxyl, C₃-C₈cycloalkyl, NH₂, NH(C₁-C₆alkyl), or N(C₁-C₆alkyl)₂.

In some embodiments, at least one R₃ is halogen. In some embodiments, at least one R₃ is F, Cl, Br, or I. In some embodiments, at least one R₃ is F, Cl, or Br. In some embodiments, at least one R₃ is F or Cl. In some embodiments, at least one R₃ is F. In some embodiments, at least one R₃ is Cl. In some embodiments, at least one R₃ is Br. In some embodiments, at least one R₃ is I.

In some embodiments, each R₃ is independently C₁-C₆alkyl, C₂-C₆alkenyl, C₂-C₆alkynyl, C₁-C₆alkoxyl, or C₃-C₈cycloalkyl.

In some embodiments, each R₃ is independently C₁-C₆alkyl, C₂-C₆alkenyl, C₂-C₆alkynyl, C₁-C₆alkoxyl, or C₃-C₈cycloalkyl, wherein the alkyl, alkenyl, alkynyl, alkoxyl, and cycloalkyl are optionally substituted with one or more hydroxyl or NH₂.

In some embodiments, each R₃ is independently C₁-C₆alkyl, C₂-C₆alkenyl, or C₂-C₆alkynyl.

In some embodiments, each R₃ is independently C₁-C₆alkyl, C₂-C₆alkenyl, or C₂-C₆alkynyl, wherein the alkyl, alkenyl, or alkynyl are optionally substituted with one or more hydroxyl or NH₂.

In some embodiments, at least one R₃ is C₁-C₆alkyl. In some embodiments, at least one R₃ is methyl. In some embodiments, at least one R₃ is ethyl. In some embodiments, at least one R₃ is propyl. In some embodiments, at least one R₃ is butyl. In some embodiments, R₃ is iso-propyl. In some embodiments, at least one R₃ is iso-butyl. In some embodiments, at least one R₃ is sec-butyl. In some embodiments, at least one R₃ is tert-butyl.

In some embodiments, each R₃ is C₁-C₆alkyl optionally substituted with one or more hydroxyl or NH₂.

In some embodiments, R₃ is C₂-C₆alkenyl. In some embodiments, R₃ is C₂alkenyl. In some embodiments, R₃ is C₃alkenyl. In some embodiments, R₃ is C₄alkenyl. In some embodiments, R₃ is C₅alkenyl. In some embodiments, R₃ is C₆alkenyl.

In some embodiments, R₃ is C₂-C₆alkenyl optionally substituted with one or more hydroxyl or NH₂.

In some embodiments, R₃ is C₂-C₆alkynyl. In some embodiments, R₃ is C₂alkynyl. In some embodiments, R₃ is C₃alkynyl. In some embodiments, R₃ is C₄alkynyl. In some embodiments, R₃ is C₅alkynyl. In some embodiments, R₃ is C₆alkynyl.

In some embodiments, R₃ is C₂-C₆alkynyl optionally substituted with one or more hydroxyl or NH₂.

In some embodiments, each R₃ is independently C₁-C₆alkoxyl or C₃-C₈cycloalkyl.

In some embodiments, each R₃ is independently C₁-C₆alkoxyl or C₃-C₈cycloalkyl, wherein the alkoxyl and cycloalkyl are optionally substituted with one or more hydroxyl or NH₂.

In some embodiments, each R₃ is independently C₁-C₆alkoxyl optionally substituted with one or more hydroxyl or NH₂.

In some embodiments, at least one R₃ is C₁-C₆alkoxyl. In some embodiments, at least one R₃ is methoxyl. In some embodiments, at least one R₃ is ethoxyl. In some embodiments, at least one R₃ is propoxyl. In some embodiments, at least one R₃ is butoxyl. In some embodiments, at least one R₃ is pentoxyl. In some embodiments, at least one R₃ is hexoxyl.

In some embodiments, each R₃ is independently C₃-C₈cycloalkyl optionally substituted with one or more hydroxyl or NH₂.

In some embodiments, at least one R₃ is independently C₃-C₈cycloalkyl. In some embodiments, at least one R₃ is independently cyclopropyl. In some embodiments, at least one R₃ is independently cyclobutyl. In some embodiments, at least one R₃ is independently cyclopentyl. In some embodiments, at least one R₃ is independently cyclohexyl. In some embodiments, at least one R₃ is independently cycloheptyl. In some embodiments, at least one R₃ is independently cyclooctyl.

In some embodiments, each R₃ is independently NH₂, NH(C₁-C₆alkyl), or N(C₁-C₆alkyl)₂.

In some embodiments, at least one R₃ is NH₂.

In some embodiments, each R₃ is NH(C₁-C₆alkyl), or N(C₁-C₆alkyl)₂.

In some embodiments, at least one R₃ is NH(C₁-C₆alkyl). In some embodiments, at least one R₃ is NH(methyl). In some embodiments, at least one R₃ is NH(ethyl). In some embodiments, at least one R₃ is NH(propyl). In some embodiments, at least one R₃ is NH(butyl). In some embodiments, at least one R₃ is NH(pentyl). In some embodiments, at least one R₃ is NH(hexyl).

In some embodiments, at least one R₃ is N(C₁-C₆alkyl)₂. In some embodiments, at least one R₃ is N(methyl)₂. In some embodiments, at least one R₃ is N(ethyl)₂. In some embodiments, at least one R₃ is N(propyl)₂. In some embodiments, at least one R₃ is N(butyl)₂. In some embodiments, at least one R₃ is N(pentyl)₂. In some embodiments, at least one R₃ is N(hexyl)₂.

In some embodiments, R₃ is F or methyl.

In some embodiments, each R₄ is independently halogen, hydroxyl, cyano, nitro, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or C(O)NH₂.

In some embodiments, each R₄ is independently halogen, hydroxyl, cyano, or nitro.

In some embodiments, at least one R₄ is halogen. In some embodiments, at least one R₄ is F, Cl, Br, or I. In some embodiments, at least one R₄ is F, C₁, or Br. In some embodiments, at least one R₄ is F or Cl. In some embodiments, at least one R₄ is F. In some embodiments, at least one R₄ is Cl. In some embodiments, at least one R₄ is Br. In some embodiments, at least one R₄ is I.

In some embodiments, each R₄ is independently hydroxyl, cyano, or nitro.

In some embodiments, at least one R₄ is independently hydroxyl.

In some embodiments, at least one R₄ is independently cyano.

In some embodiments, at least one R₄ is independently nitro.

In some embodiments, each R₄ is independently C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or C(O)NH₂, wherein the alkyl, alkenyl, alkynyl, alkoxyl, and cycloalkyl are optionally substituted with one or more hydroxyl, 4- to 7-membered heterocyclyl comprising 1, 2, or 3 heteroatoms independently selected from N, O, and S, NH₂, NH(C₁-C₆alkyl), or N(C₁-C₆alkyl)₂.

In some embodiments, each R₄ is independently C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or C(O)NH₂.

In some embodiments, each R₄ is independently C₁-C₆alkyl, C₂-C₆alkenyl, or C₂-C₆alkynyl, wherein the alkyl, alkenyl, or alkynyl are optionally substituted with one or more hydroxyl, 4- to 7-membered heterocyclyl comprising 1, 2, or 3 heteroatoms independently selected from N, O, and S, NH₂, NH(C₁-C₆alkyl), or N(C₁-C₆alkyl)₂.

In some embodiments, each R₄ is independently C₁-C₆alkyl, C₂-C₆alkenyl, or C₂-C₆alkynyl.

In some embodiments, R₄ is C₁-C₆alkyl. In some embodiments, R₄ is methyl. In some embodiments, R₄ is ethyl. In some embodiments, R₄ is propyl. In some embodiments, R₄ is butyl. In some embodiments, R₄ is iso-propyl. In some embodiments, R₄ is iso-butyl. In some embodiments, R₄ is sec-butyl. In some embodiments, R₄ is tert-butyl.

In some embodiments, R₄ is C₁-C₆alkyl optionally substituted with one or more hydroxyl, 4- to 7-membered heterocyclyl comprising 1, 2, or 3 heteroatoms independently selected from N, O, and S, NH₂, NH(C₁-C₆alkyl), or N(C₁-C₆alkyl)₂.

In some embodiments, R₄ is C₂-C₆alkenyl. In some embodiments, R₄ is C₂alkenyl. In some embodiments, R₄ is C₃alkenyl. In some embodiments, R₄ is C₄alkenyl. In some embodiments, R₄ is C₅alkenyl. In some embodiments, R₄ is C₆alkenyl.

In some embodiments, R₄ is C₂-C₆alkenyl optionally substituted with one or more hydroxyl, 4- to 7-membered heterocyclyl comprising 1, 2, or 3 heteroatoms independently selected from N, O, and S, NH₂, NH(C₁-C₆alkyl), or N(C₁-C₆alkyl)₂.

In some embodiments, R₄ is C₂-C₆alkynyl. In some embodiments, R₄ is C₂alkynyl. In some embodiments, R₄ is C₃alkynyl. In some embodiments, R₄ is C₄alkynyl. In some embodiments, R₄ is C₅alkynyl. In some embodiments, R₄ is C₆alkynyl.

In some embodiments, R₄ is C₂-C₆alkynyl optionally substituted with one or more hydroxyl, 4- to 7-membered heterocyclyl comprising 1, 2, or 3 heteroatoms independently selected from N, O, and S, NH₂, NH(C₁-C₆alkyl), or N(C₁-C₆alkyl)₂.

In some embodiments, each R₄ is independently C₁-C₆alkoxyl or C₃-C₈cycloalkyl.

In some embodiments, each R₄ is independently C₁-C₆alkoxyl or C₃-C₈cycloalkyl, wherein the alkoxyl and cycloalkyl are optionally substituted with one or more hydroxyl, 4- to 7-membered heterocyclyl, NH₂, NH(C₁-C₆alkyl), or N(C₁-C₆alkyl)₂.

In some embodiments, each R₄ is independently C₁-C₆alkoxyl. In some embodiments, each R₄ is independently methoxyl. In some embodiments, each R₄ is independently ethoxyl. In some embodiments, each R₄ is independently propoxyl. In some embodiments, each R₄ is independently butyoxyl. In some embodiments, each R₄ is independently pentyoxyl. In some embodiments, each R₄ is independently hexoxyl.

In some embodiments, each R₄ is independently C₁-C₆alkoxyl optionally substituted with one or more hydroxyl, 4- to 7-membered heterocyclyl, NH₂, NH(C₁-C₆alkyl), or N(C₁-C₆alkyl)₂.

In some embodiments, each R₄ is independently C₃-C₈cycloalkyl. In some embodiments, each R₄ is independently cyclopropyl. In some embodiments, each R₄ is independently cyclobutyl. In some embodiments, each R₄ is independently cyclopentyl. In some embodiments, each R₄ is independently cyclohexyl. In some embodiments, each R₄ is independently cycloheptyl. In some embodiments, each R₄ is independently cyclooctyl.

In some embodiments, each R₄ is independently C₃-C₈cycloalkyl optionally substituted with one or more hydroxyl, 4- to 7-membered heterocyclyl, NH₂, NH(C₁-C₆alkyl), or N(C₁-C₆alkyl)₂.

In some embodiments, each R₄ is independently C₁-C₆haloalkyl or C₁-C₆haloalkoxyl.

In some embodiments, each R₄ is independently C₁-C₆haloalkyl. In some embodiments, each R₄ is independently halomethyl. In some embodiments, each R₄ is independently haloethyl. In some embodiments, each R₄ is independently halopropyl. In some embodiments, each R₄ is independently halobutyl. In some embodiments, each R₄ is independently halopentyl. In some embodiments, each R₄ is independently halohexyl.

In some embodiments, each R₄ is independently CF₃, CHF₂, or CH₂F.

In some embodiments, each R₄ is independently C₁-C₆haloalkoxyl. In some embodiments, each R₄ is independently halomethoxyl. In some embodiments, each R₄ is independently haloethoxyl. In some embodiments, each R₄ is independently halopropoxyl. In some embodiments, each R₄ is independently halobutoxyl. In some embodiments, each R₄ is independently halopentoxyl. In some embodiments, each R₄ is independently halohexoxyl.

In some embodiments, each R₄ is independently NH₂, NH(C₁-C₆alkyl), N(C₁-C₆alkyl)₂, or C(O)NH₂.

In some embodiments, each R₄ is independently NH₂, NH(C₁-C₆alkyl), or N(C₁-C₆alkyl)₂.

In some embodiments, each R₄ is independently NH₂.

In some embodiments, each R₄ is independently C(O)NH₂.

In some embodiments, each R₄ is independently NH(C₁-C₆alkyl) or N(C₁-C₆alkyl)₂.

In some embodiments, each R₄ is independently NH(C₁-C₆alkyl). In some embodiments, each R₄ is independently H(methyl). In some embodiments, each R₄ is independently NH(ethyl). In some embodiments, each R₄ is independently NH(propyl). In some embodiments, each R₄ is independently NH(butyl). In some embodiments, each R₄ is independently NH(pentyl). In some embodiments, each R₄ is independently NH(hexyl).

In some embodiments, each R₄ is independently N(C₁-C₆alkyl)₂. In some embodiments, each R₄ is independently N(methyl)₂. In some embodiments, each R₄ is independently N(ethyl)₂. In some embodiments, each R₄ is independently N(propyl)₂. In some embodiments, each R₄ is independently N(butyl)₂. In some embodiments, each R₄ is independently N(pentyl)₂. In some embodiments, each R₄ is independently N(hexyl)₂.

In some embodiments, each R₄ is independently methyl, ethyl, F, or CF₃.

In some embodiments, each R₄ is independently methyl or ethyl.

In some embodiments, each R₄ is independently F or CF₃.

In some embodiments, R₅ is H.

In some embodiments, R₅ is C₁-C₆alkyl, C₁-C₆haloalkyl, C₂-C₆alkenyl, C₂-C₆alkynyl, C₁-C₆alkoxyl, C₃-C₈cycloalkyl, or heterocyclyl, wherein heterocyclyl is a 4- to 7-membered ring and comprises 1, 2, or 3 heteroatoms independently selected from N, O, and S, wherein the cycloalkyl and heterocyclyl are optionally substituted with one or more halogen, C₁-C₆alkyl or hydroxyl.

In some embodiments, R₅ is C₁-C₆alkyl, C₂-C₆alkenyl, or C₂-C₆alkynyl.

In some embodiments, R₅ is C₁-C₆alkyl. In some embodiments, R₅ is methyl. In some embodiments, R₅ is ethyl. In some embodiments, R₅ is propyl. In some embodiments, R₅ is butyl. In some embodiments, R₅ is iso-propyl. In some embodiments, R₅ is iso-butyl. In some embodiments, R₅ is sec-butyl. In some embodiments, R₅ is tert-butyl.

In some embodiments, R₅ is C₂-C₆alkenyl. In some embodiments, R₅ is C₂-C₆alkenyl. In some embodiments, R₅ is C₂alkenyl. In some embodiments, R₅ is C₃alkenyl. In some embodiments, R₅ is C₄alkenyl. In some embodiments, R₅ is C₅alkenyl. In some embodiments, R₅ is C₆alkenyl.

In some embodiments, R₅ is C₂-C₆alkynyl. In some embodiments, R₅ is C₂-C₆alkynyl. In some embodiments, R₅ is C₂alkynyl. In some embodiments, R₅ is C₃alkynyl. In some embodiments, R₅ is C₄alkynyl. In some embodiments, R₅ is C₅alkynyl. In some embodiments, R₅ is C₆alkynyl.

In some embodiments, R₅ is C₁-C₆alkoxyl or C₃-C₈cycloalkyl.

In some embodiments, R₅ is independently C₁-C₆alkoxyl. In some embodiments, R₅ is independently methoxyl. In some embodiments, R₅ is independently ethoxyl. In some embodiments, R₅ is independently propoxyl. In some embodiments, R₅ is independently butoxyl. In some embodiments, R₅ is independently pentoxyl. In some embodiments, R₅ is independently hexoxyl.

In some embodiments, R₅ is C₃-C₈cycloalkyl. In some embodiments, R₅ is cyclopropyl. In some embodiments, R₅ is cyclobutyl. In some embodiments, R₅ is cyclopentyl. In some embodiments, R₅ is cyclohexyl. In some embodiments, R₅ is heptyl. In some embodiments, R₅ is cyclooctyl.

In some embodiments, R₅ is heterocyclyl, wherein heterocyclyl is a 4- to 7-membered ring and comprises 1, 2, or 3 heteroatoms independently selected from N, O, and S, wherein the cycloalkyl and heterocyclyl are optionally substituted with one or more halogen, C₁-C₆alkyl or hydroxyl. In some embodiments, R₅ is heterocyclyl, wherein heterocyclyl is a 4- to 6-membered ring and comprises 1 or 2heteroatoms independently selected from N and O, wherein the cycloalkyl and heterocyclyl are optionally substituted with one or more halogen, C₁-C₆alkyl or hydroxyl. In some embodiments, R₅ is tetrahydropyranyl or tetrahydrofuranyl.

In some embodiments, R₅ is H, C₁₋₆alkyl, or heterocyclyl, wherein heterocyclyl is a 4- to 7-membered ring and comprises 1 or 2 heteroatoms independently selected from N and O.

In some embodiments, R₅ is H. In some embodiments, R₆ is H. In some embodiments both R₅ and R₆ are H.

In some embodiments of the compounds of Formula I, n is 0, 1, 2, 3, 4, or 5. In some embodiments of the compounds of Formula I, n is 0, 1, 2, 3, or 4. In some embodiments of the compounds of Formula I, n is 0, 1, 2, or 3. In some embodiments of the compounds of Formula I, n is 0, 1, or 2. In some embodiments of the compounds of Formula I, n is 0 or 1. In some embodiments of the compounds of Formula I, n is 0. In some embodiments of the compounds of Formula I, n is 1. In some embodiments of the compounds of Formula I, n is 2. In some embodiments of the compounds of Formula I, n is 3. In some embodiments of the compounds of Formula I, n is 4. In some embodiments of the compounds of Formula I, n is 5.

In some embodiments of the compounds of Formula II, m is 0, 1, 2, 3, 4, 5, 6, or 7. In some embodiments of the compounds of Formula II, m is 0, 1, 2, 3, 4, 5, or 6. In some embodiments of the compounds of Formula II, m is 0, 1, 2, 3, 4, or 5. In some embodiments of the compounds of Formula II, m is 0, 1, 2, 3, or 4. In some embodiments of the compounds of Formula II, m is 0, 1, 2, or 3. In some embodiments of the compounds of Formula II, m is 0, 1, or 2. In some embodiments of the compounds of Formula II, m is 0 or 1. In some embodiments of the compounds of Formula II, m is 0. In some embodiments of the compounds of Formula II, m is 1. In some embodiments of the compounds of Formula II, m is 2. In some embodiments of the compounds of Formula II, m is 3. In some embodiments of the compounds of Formula II, m is 4. In some embodiments of the compounds of Formula II, m is 5. In some embodiments of the compounds of Formula II, m is 6. In some embodiments of the compounds of Formula II, m is 7

In some embodiments of the compounds of Formula III, p is 0, 1, 2, 3, 4, 5, 6, 7, 8, or 9. In some embodiments of the compounds of Formula III, p is 0, 1, 2, 3, 4, 5, 6, 7, or 8. In some embodiments of the compounds of Formula III, p is 0, 1, 2, 3, 4, 5, 6, or 7. In some embodiments of the compounds of Formula III, p is 0, 1, 2, 3, 4, 5, or 6. In some embodiments of the compounds of Formula III, p is 0, 1, 2, 3, 4, or 5. In some embodiments of the compounds of Formula III, p is 0, 1, 2, 3, or 4. In some embodiments of the compounds of Formula III, p is 0, 1, 2, or 3. In some embodiments of the compounds of Formula III, p is 0, 1, or 2. In some embodiments of the compounds of Formula III, p is 0 or 1. In some embodiments of the compounds of Formula III, p is 0. In some embodiments of the compounds of Formula III, p is 1. In some embodiments of the compounds of Formula III, p is 2. In some embodiments of the compounds of Formula III, p is 3. In some embodiments of the compounds of Formula III, p is 4. In some embodiments of the compounds of Formula III, p is 5. In some embodiments of the compounds of Formula III, p is 6. In some embodiments of the compounds of Formula III, p is 7. In some embodiments of the compounds of Formula III, p is 8. In some embodiments of the compounds of Formula III, p is 9.

In some embodiments, the compound is selected from the compounds described in Table 1 and pharmaceutically acceptable salts, solvates, or prodrugs thereof.

In some embodiments, the compound is selected from the compounds described in Table 1 and pharmaceutically acceptable salts thereof.

In some embodiments, the compound is selected from the prodrugs of compounds described in Table 1 and pharmaceutically acceptable salts thereof.

For the avoidance of doubt it is to be understood that, where in this specification a group is qualified by “described herein”, the said group encompasses the first occurring and broadest definition as well as each and all of the particular definitions for that group.

The various functional groups and substituents making up the compounds of the Formula (I), (II) or (III) are typically chosen such that the molecular weight of the compound does not exceed 1000 daltons. More usually, the molecular weight of the compound will be less than 900, for example less than 800, or less than 750, or less than 700, or less than 650 daltons. More conveniently, the molecular weight is less than 600 and, for example, is 550 daltons or less.

It will be understood that the compounds of any one of the Formulae disclosed herein and any pharmaceutically acceptable salts thereof, comprise stereoisomers, or mixtures of stereoisomers of all isomeric forms of said compounds.

It is to be understood that the compounds of any Formula described herein include the compounds themselves, as well as their salts, and their solvates, if applicable.

The in vivo effects of a compound of any one of the Formulae disclosed herein may be exerted in part by one or more metabolites that are formed within the human or animal body after administration of a compound of any one of the Formulae disclosed herein. As stated hereinbefore, the in vivo effects of a compound of any one of the Formulae disclosed herein may also be exerted by way of metabolism of a precursor compound (a prodrug).

Suitably, the present disclosure excludes any individual compounds not possessing the biological activity defined herein.

### Methods of Synthesis

By way of example only, provided is a scheme for preparing the small molecule splicing modulators (SMSMs) described herein.

In some embodiments, a scheme for preparing an SMSM is described herein in

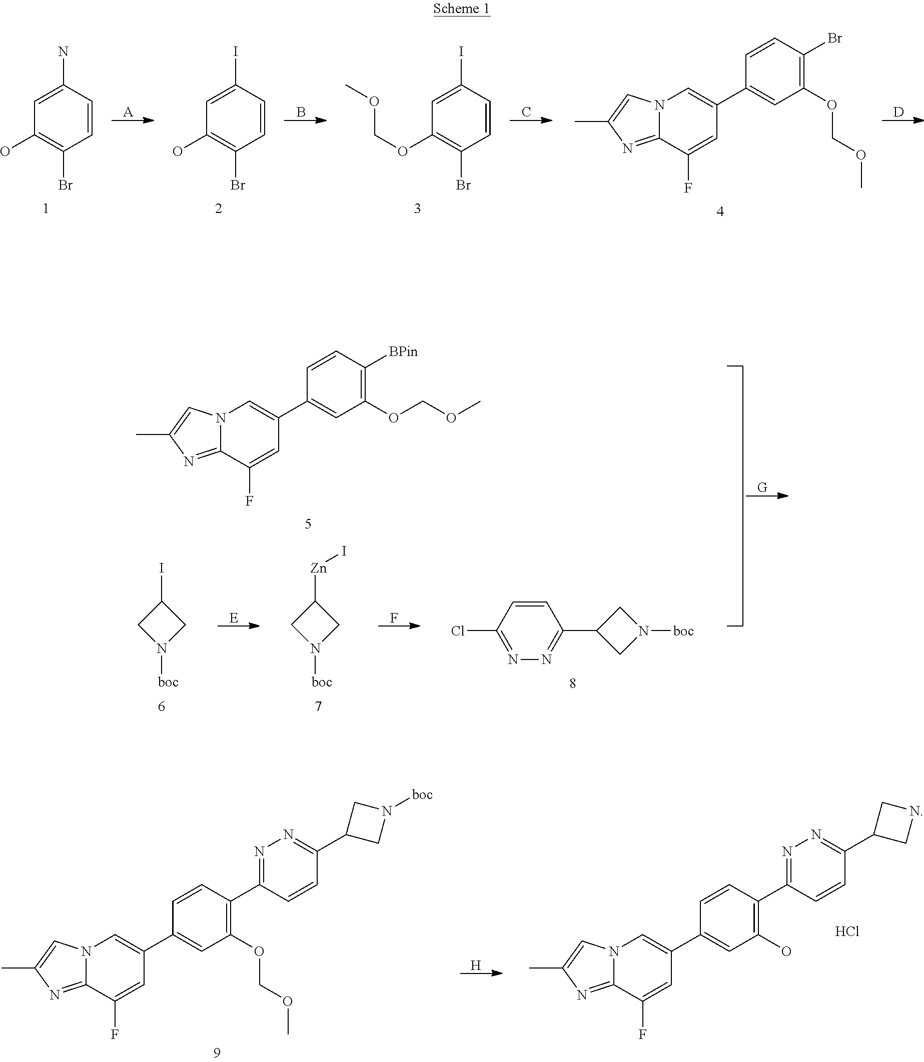

In some embodiments, a scheme for preparing an SMSM is described herein in

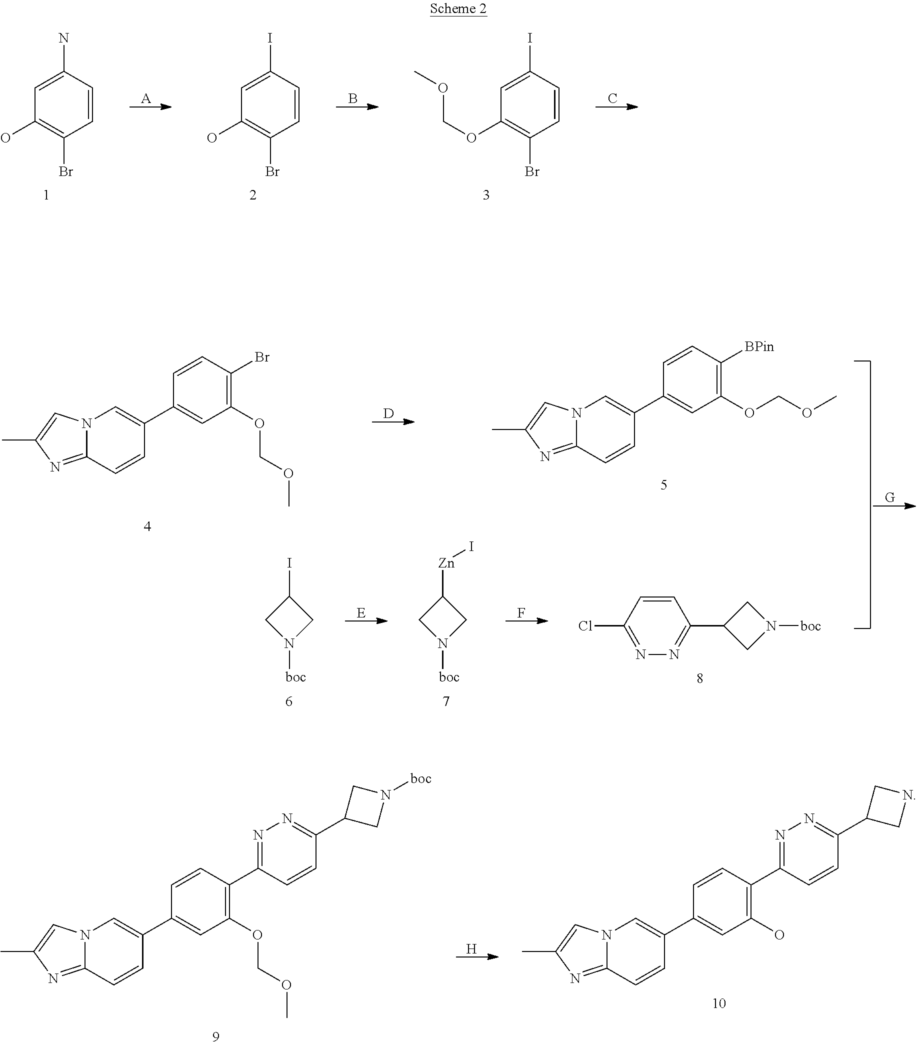

In some aspects, the present disclosure provides a method of preparing a compound of the present disclosure.

In some aspects, the present disclosure provides a method of preparing a compound, comprising one or more steps as described herein.

In some aspects, the present disclosure provides a compound obtainable by, or obtained by, or directly obtained by a method for preparing a compound as described herein.

In some aspects, the present disclosure provides an intermediate as described herein, being suitable for use in a method for preparing a compound as described herein.

The compounds of the present disclosure can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilized.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl, or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium on carbon.

Once a compound of Formula (I), (II), or (III) has been synthesised by any one of the processes defined herein, the processes may then further comprise the additional steps of: (i) removing any protecting groups present; (ii) converting the compound of Formula (I), (II), or (III) into another compound of Formula (I), (II) or (III); (iii) forming a pharmaceutically acceptable salt, hydrate or solvate thereof, and/or (iv) forming a prodrug thereof.

The resultant compounds of Formula (I), (II), or (III) can be isolated and purified using techniques well known in the art.

Conveniently, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIBK) or butanone; amides, such as acetamide, dimethylacetamide, dimethylformamide (DMF) or N-methylpyrrolidinone (NMP); nitriles, such as acetonitrile; sulphoxides, such as dimethyl sulphoxide (DMSO); nitro compounds, such as nitromethane or nitrobenzene; esters, such as ethyl acetate or methyl acetate, or mixtures of the said solvents or mixtures with water.

The reaction temperature is suitably between about −100° C. and 300° C., depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by utilising the procedures described herein, in conjunction with ordinary skills in the art, additional compounds of the present disclosure can be readily prepared. Those skilled in the art will readily understand that known variations of the conditions and processes of the following preparative procedures can be used to prepare these compounds. As will be understood by the person skilled in the art of organic synthesis, compounds of the present disclosure are readily accessible by various synthetic routes, some of which are exemplified in the accompanying examples. The skilled person will easily recognise which kind of reagents and reactions conditions are to be used and how they are to be applied and adapted in any particular instance—wherever necessary or useful—in order to obtain the compounds of the present disclosure. Furthermore, some of the compounds of the present disclosure can readily be synthesised by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply—whenever necessary or useful—synthetic protecting (or protective) groups; suitable protecting groups as well as methods for introducing and removing them are well-known to the person skilled in the art of chemical synthesis and are described, in more detail, in, e.g., P. G. M. Wuts, T. W. Greene, “Greene's Protective Groups in Organic Synthesis”, 4th edition (2006) (John Wiley & Sons).

General routes for the preparation of a compound of the application are described herein.

### Biological Assays

Compounds designed, selected and/or optimised by methods described above, once produced, can be characterised using a variety of assays known to those skilled in the art to determine whether the compounds have biological activity. For example, the molecules can be characterised by conventional assays, including but not limited to those assays described below, to determine whether they have a predicted activity, binding activity and/or binding specificity.

### Pharmaceutical Compositions

In some aspects, the present disclosure provides a pharmaceutical composition comprising a compound of the present disclosure as an active ingredient. In some embodiments, the present disclosure provides a pharmaceutical composition comprising at least one compound of each of the formulae described herein, or a pharmaceutically acceptable salt or solvate thereof, and one or more pharmaceutically acceptable carriers or excipients. In some embodiments, the present disclosure provides a pharmaceutical composition comprising at least one compound selected from Table 1.

As used herein, the term “composition” is intended to encompass a product comprising the specified ingredients in the specified amounts, as well as any product which results, directly or indirectly, from combination of the specified ingredients in the specified amounts.

The compounds of present disclosure can be formulated for oral administration in forms such as tablets, capsules (each of which includes sustained release or timed release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-o-cyclodextrin, glucosyl-β-cyclodextrin, sulphated β-cyclodextrin (S-β-CD), maltosyl-β-cyclodextrin, β-cyclodextrin sulphobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilise the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and E-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present disclosure for use in therapy is an amount sufficient to treat or prevent a disease or disorder referred to herein, slow its progression and/or reduce the symptoms associated with the condition.

An effective amount of a compound of the present disclosure for use in therapy is an amount sufficient to treat a disease or disorder referred to herein, slow its progression and/or reduce the symptoms associated with the condition.

The size of the dose for therapeutic or prophylactic purposes of a compound of Formula (I), (II), or (III) will naturally vary according to the nature and severity of the conditions, the age and sex of the animal or subject and the route of administration, according to well-known principles of medicine.

### Methods of Use

Provided herein is a method of treating a disease in a subject in need thereof, the method comprising administering to the subject a therapeutically effective amount of a compound or a pharmaceutically composition described herein, wherein the disease is selected from the group consisting of ALS, Alzheimer's disease, argyrophilic grain disease, corticobasal degeneration, cystic fibrosis, dilated cardiomyopathy, Duchenne muscular dystrophy, Ehlers-Danlos syndrome, Fabry's disease, familial dysautonomia, familial hypercholesterolemia, familial persistent hyperinsulinemic hypoglycemia, frontotemporal dementia, FTDP-17, Gaucher's disease, globular glial tauopathy, HIV-1, Huntington's disease, Hutchinson-Gilford progeria syndrome, hypercholesterolemia, Leber congenital amaurosis, migraine, multiple sclerosis, myelodysplastic syndromes, NASH, Niemann-Pick's, pain, Parkinson's disease, phenylketonuria, Pick's disease, progressive supranuclear palsy, spinal muscular atrophy, spinocerebellar ataxia type 2, Wilson's disease, Sickle cell anemia, Crohn's disease, ulcerative colitis, psoriasis, and rheumatoid arthritis

In some embodiments, the disease is Huntington's disease.

Provided herein is a method of treating a disease in a subject in need thereof, the method comprising administering to the subject a therapeutically effective amount of a compound or a pharmaceutically composition disclosed herein, wherein the disease is a pulmonary disease selected from the group consisting of chronic obstructive pulmonary disease (COPD), asthma, acute lung injury (ALI), pulmonary fibrosis, and pulmonary arterial hypertension (PAH).

### Routes of Administration

The compounds of the disclosure or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

The present disclosure includes the following embodiments numbered 1-27:
1. A compound of Formula (I):

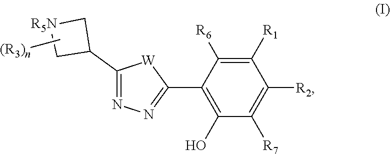

or a pharmaceutically acceptable salt, solvate, or prodrug thereof, wherein:
   W is —S— or —HC═CH—;
   R₁ is H, halogen, hydroxyl, cyano, C₁-C₆alkyl, C₂-C₆alkenyl, C₂-C₆alkynyl, C₁-C₆haloalkyl, C₁-C₆alkoxyl, C₁-C₆haloalkoxyl, —(CH₂)₀₋₂—C₃-C₈cycloalkyl, NH₂, NH(C₁-C₆alkyl), N(C₁-C₆alkyl)₂, or —(CH₂)₀₋₂-heterocyclyl, wherein heterocyclyl is a 4- to 7-membered ring and comprises 1, 2, or 3 heteroatoms independently selected from N, O, and S, wherein the alkyl, alkenyl, alkynyl, alkoxyl, cycloalkyl, and heterocyclyl are optionally substituted with one or more C₃-C₈cycloalkyl, aryl, or 4- to 7-membered heterocyclyl comprising 1, 2, or 3 heteroatoms independently selected from N, O, and S;
   R₂ is aryl, 5- to 7-membered cycloalkyl, 5-, 6-, or 9-membered heterocyclyl comprising 1, 2, or 3 heteroatoms independently selected from N, O, and S, or 5-, 6-, or 9-membered heteroaryl comprising 2, 3, or 4 heteroatoms independently selected from N, O, and S, wherein the aryl, cycloalkyl, heterocyclyl, or heteroaryl is optionally substituted with one or more R₄;
   each R₃ is independently halogen, C₁-C₆alkyl, C₂-C₆alkenyl, C₂-C₆alkynyl, C₁-C₆alkoxyl, C₃-C₈cycloalkyl, NH₂, NH(C₁-C₆alkyl), or N(C₁-C₆alkyl)₂, wherein the alkyl, alkenyl, alkynyl, alkoxyl, and cycloalkyl are optionally substituted with one or more hydroxyl or NH₂;
   each R₄ is independently halogen, hydroxyl, cyano, nitro, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or C(O)NH₂, wherein the alkyl, alkenyl, alkynyl, alkoxyl, and cycloalkyl are optionally substituted with one or more hydroxyl, 4- to 7-membered heterocyclyl comprising 1, 2, or 3 heteroatoms independently selected from N, O, and S, NH₂, NH(C₁-C₆alkyl), or N(C₁-C₆alkyl)₂;
   R₅ is H, C₁-C₆alkyl, C₁-C₆haloalkyl, C₂-C₆alkenyl, C₂-C₆alkynyl, C₁-C₆alkoxyl, C₃-C₈cycloalkyl, or heterocyclyl, wherein heterocyclyl is a 4- to 7-membered ring and comprises 1, 2, or 3 heteroatoms independently selected from N, O, and S, wherein the cycloalkyl and heterocyclyl are optionally substituted with one or more halogen, C₁-C₆alkyl or hydroxyl;
   R₆ is H, halogen, C₁-C₆alkyl or C₁-C₆haloalkyl;
   R₇ is H, halogen, C₁-C₆alkyl or C₁-C₆haloalkyl; and
   n is 0, 1, 2, 3, 4, or 5.
2. The compound of embodiment 1 of Formula (Ic):

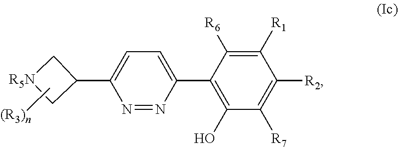

or a pharmaceutically acceptable salt, solvate, or prodrug thereof, wherein:
   R₁ is H, halogen, hydroxyl, cyano, C₁-C₆alkyl, C₂-C₆alkenyl, C₂-C₆alkynyl, C₁-C₆haloalkyl, C₁-C₆alkoxyl, C₁-C₆haloalkoxyl, —(CH₂)₀₋₂—C₃-C₈cycloalkyl, NH₂, NH(C₁-C₆alkyl), N(C₁-C₆alkyl)₂, or —(CH₂)₀₋₂-heterocyclyl, wherein heterocyclyl is a 4- to 7-membered ring and comprises 1, 2, or 3 heteroatoms independently selected from N, O, and S, wherein the alkyl, alkenyl, alkynyl, alkoxyl, cycloalkyl, and heterocyclyl are optionally substituted with one or more C₃-C₈cycloalkyl, aryl, or 4- to 7-membered heterocyclyl comprising 1, 2, or 3 heteroatoms independently selected from N, O, and S;
   R₂ is aryl, 5- to 7-membered cycloalkyl, 5-, 6-, or 9-membered heterocyclyl comprising 1, 2, or 3 heteroatoms independently selected from N, O, and S, or 5-, 6-, or 9-membered heteroaryl comprising 2, 3, or 4 heteroatoms independently selected from N, O, and S, wherein the aryl, cycloalkyl, heterocyclyl, or heteroaryl is optionally substituted with one or more R₄;
   each R₃ is independently halogen, C₁-C₆alkyl, C₂-C₆alkenyl, C₂-C₆alkynyl, C₁-C₆alkoxyl, C₃-C₈cycloalkyl, NH₂, NH(C₁-C₆alkyl), or N(C₁-C₆alkyl)₂, wherein the alkyl, alkenyl, alkynyl, alkoxyl, and cycloalkyl are optionally substituted with one or more hydroxyl or NH₂;
   each R₄ is independently halogen, hydroxyl, cyano, nitro, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or C(O)NH₂, wherein the alkyl, alkenyl, alkynyl, alkoxyl, and cycloalkyl are optionally substituted with one or more hydroxyl, 4- to 7-membered heterocyclyl comprising 1, 2, or 3 heteroatoms independently selected from N, O, and S, NH₂, NH(C₁-C₆alkyl), or N(C₁-C₆alkyl)₂;
   R₅ is H, C₁-C₆alkyl, C₁-C₆haloalkyl, C₂-C₆alkenyl, C₂-C₆alkynyl, C₁-C₆alkoxyl, C₃-C₈cycloalkyl, or heterocyclyl, wherein heterocyclyl is a 4- to 7-membered ring and comprises 1, 2, or 3 heteroatoms independently selected from N, O, and S, wherein the cycloalkyl and heterocyclyl are optionally substituted with one or more halogen, C₁-C₆alkyl or hydroxyl;
   R₆ is H, halogen, C₁-C₆alkyl or C₁-C₆haloalkyl;
   R₇ is H, halogen, C₁-C₆alkyl or C₁-C₆haloalkyl; and
   n is 0, 1, 2, 3, 4, or 5.
3. The compound of embodiment 1 of Formula (Id):

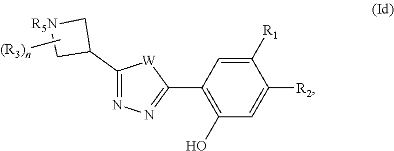

or a pharmaceutically acceptable salt, solvate, or prodrug thereof, wherein:
   W is —S— or —HC═CH—;
   R₁ is H, halogen, hydroxyl, cyano, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or 4- to 7-membered heterocyclyl comprising 1, 2, or 3 heteroatoms independently selected from N, O, and S, wherein the alkyl, alkenyl, alkynyl, alkoxyl, cycloalkyl, and heterocyclyl are optionally substituted with one or more C₃-C₈cycloalkyl, aryl, or 4- to 7-membered heterocyclyl comprising 1, 2, or 3 heteroatoms independently selected from N, O, and S;
   R₂ is aryl, 5- to 7-membered cycloalkyl, 5-, 6-, or 9-membered heterocyclyl comprising 1, 2, or 3 heteroatoms independently selected from N, O, and S, or 5-, 6-, or 9-membered heteroaryl comprising 2, 3, or 4 heteroatoms independently selected from N, O, and S, wherein the aryl, cycloalkyl, heterocyclyl, or heteroaryl is optionally substituted with one or more R₄;
   each R₃ is independently halogen, C₁-C₆alkyl, C₂-C₆alkenyl, C₂-C₆alkynyl, C₁-C₆alkoxyl, C₃-C₈cycloalkyl, NH₂, NH(C₁-C₆alkyl), or N(C₁-C₆alkyl)₂, wherein the alkyl, alkenyl, alkynyl, alkoxyl, and cycloalkyl are optionally substituted with one or more hydroxyl or NH₂;
   each R₄ is independently halogen, hydroxyl, cyano, nitro, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or C(O)NH₂, wherein the alkyl, alkenyl, alkynyl, alkoxyl, and cycloalkyl are optionally substituted with one or more hydroxyl, 4- to 7-membered heterocyclyl comprising 1, 2, or 3 heteroatoms independently selected from N, O, and S, NH₂, NH(C₁-C₆alkyl), or N(C₁-C₆alkyl)₂;
   R₅ is H, C₁-C₆alkyl, C₁-C₆haloalkyl, C₂-C₆alkenyl, C₂-C₆alkynyl, C₁-C₆alkoxyl, C₃-C₈cycloalkyl, or heterocyclyl, wherein heterocyclyl is a 4- to 7-membered ring and comprises 1, 2, or 3 heteroatoms independently selected from N, O, and S, wherein the cycloalkyl and heterocyclyl are optionally substituted with one or more halogen, C₁-C₆alkyl or hydroxyl; and
   n is 0, 1, 2, 3, 4, or 5.
4. A compound of Formula (II):

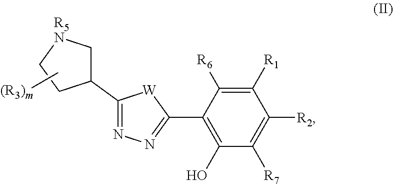

or a pharmaceutically acceptable salt, solvate, or prodrug thereof, wherein:
   W is —S— or —HC═CH—;
   R₁ is H, halogen, hydroxyl, cyano, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or 4- to 7-membered heterocyclyl comprising 1, 2, or 3 heteroatoms independently selected from N, O, and S, wherein the alkyl, alkenyl, alkynyl, alkoxyl, cycloalkyl, and heterocyclyl are optionally substituted with one or more C₃-C₈cycloalkyl, aryl, or 4- to 7-membered heterocyclyl comprising 1, 2, or 3 heteroatoms independently selected from N, O, and S;
   R₂ is aryl, 5- to 7-membered cycloalkyl, 5-, 6-, or 9-membered heterocyclyl comprising 1, 2, or 3 heteroatoms independently selected from N, O, and S, or 5-, 6-, or 9-membered heteroaryl comprising 2, 3, or 4 heteroatoms independently selected from N, O, and S, wherein the aryl, cycloalkyl, heterocyclyl, or heteroaryl is optionally substituted with one or more R₄;
   each R₃ is independently halogen, C₁-C₆alkyl, C₂-C₆alkenyl, C₂-C₆alkynyl, C₁-C₆alkoxyl, C₃-C₈cycloalkyl, NH₂, NH(C₁-C₆alkyl), or N(C₁-C₆alkyl)₂, wherein the alkyl, alkenyl, alkynyl, alkoxyl, and cycloalkyl are optionally substituted with one or more hydroxyl or NH₂;
   each R₄ is independently halogen, hydroxyl, cyano, nitro, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or C(O)NH₂, wherein the alkyl, alkenyl, alkynyl, alkoxyl, and cycloalkyl are optionally substituted with one or more hydroxyl, 4- to 7-membered heterocyclyl comprising 1, 2, or 3 heteroatoms independently selected from N, O, and S, NH₂, NH(C₁-C₆alkyl), or N(C₁-C₆alkyl)₂;
   R₅ is H, C₁-C₆alkyl, C₁-C₆alkyl, C₂-C₆alkenyl, C₂-C₆alkynyl, C₁-C₆alkoxyl, C₃-C₈cycloalkyl, or heterocyclyl, wherein heterocyclyl is a 4- to 7-membered ring and comprises 1, 2, or 3 heteroatoms independently selected from N, O, and S, wherein the cycloalkyl and heterocyclyl are optionally substituted with one or more halogen, C₁-C₆alkyl or hydroxyl;
   R₆ is H, halogen, C₁-C₆alkyl or C₁-C₆haloalkyl;
   R₇ is H, halogen, C₁-C₆alkyl or C₁-C₆haloalkyl; and
   m is 0, 1, 2, 3, 4, 5, 6, or 7.
5. A compound of Formula (III):

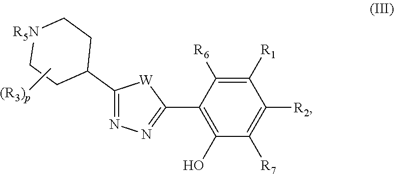

or a pharmaceutically acceptable salt, solvate, or prodrug thereof, wherein:
   W is —S— or —HC═CH—;
   R₁ is H, halogen, hydroxyl, cyano, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or 4- to 7-membered heterocyclyl comprising 1, 2, or 3 heteroatoms independently selected from N, O, and S, wherein the alkyl, alkenyl, alkynyl, alkoxyl, cycloalkyl, and heterocyclyl are optionally substituted with one or more C₃-C₈cycloalkyl, aryl, or 4- to 7-membered heterocyclyl comprising 1, 2, or 3 heteroatoms independently selected from N, O, and S;
   R₂ is aryl, 5- to 7-membered cycloalkyl, 5-, 6-, or 9-membered heterocyclyl comprising 1, 2, or 3 heteroatoms independently selected from N, O, and S, or 6-, or 9-membered heteroaryl comprising 2, 3, or 4 heteroatoms independently selected from N, O, and S, wherein the aryl, cycloalkyl, heterocyclyl, or heteroaryl is optionally substituted with one or more R₄;
   each R₃ is independently halogen, C₁-C₆alkyl, C₂-C₆alkenyl, C₂-C₆alkynyl, C₁-C₆alkoxyl, C₃-C₈cycloalkyl, NH₂, NH(C₁-C₆alkyl), or N(C₁-C₆alkyl)₂, wherein the alkyl, alkenyl, alkynyl, alkoxyl, and cycloalkyl are optionally substituted with one or more hydroxyl or NH₂;
   each R₄ is independently halogen, hydroxyl, cyano, nitro, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or C(O)NH₂, wherein the alkyl, alkenyl, alkynyl, alkoxyl, and cycloalkyl are optionally substituted with one or more hydroxyl, 4- to 7-membered heterocyclyl, NH₂, NH(C₁-C₆alkyl), or N(C₁-C₆alkyl)₂;
   R₅ is H, C₁-C₆alkyl, C₁-C₆haloalkyl, C₂-C₆alkenyl, C₂-C₆alkynyl, C₁-C₆alkoxyl, or C₃-C₈cycloalkyl, or heterocyclyl, wherein heterocyclyl is a 4- to 7-membered ring and comprises 1, 2, or 3 heteroatoms independently selected from N, O, and S, wherein the cycloalkyl and heterocyclyl are optionally substituted with one or more halogen, C₁-C₆alkyl or hydroxyl;
   R₆ is H, halogen, C₁-C₆alkyl or C₁-C₆haloalkyl;
   R₇ is H, halogen, C₁-C₆alkyl or C₁-C₆haloalkyl; and
   p is 0, 1, 2, 3, 4, 5, 6, 7, 8, or 9.
6. The compound of embodiment 1, wherein the compound is of Formula (Ia):

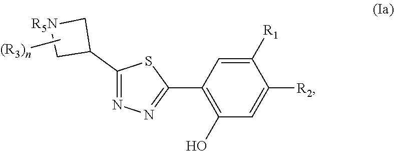

or a pharmaceutically acceptable salt, solvate, or prodrug thereof.
7. The compound of embodiment 1, wherein the compound is of Formula (Ib):

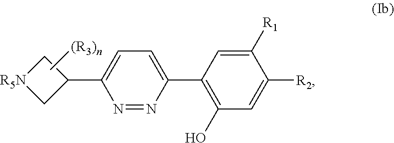

or a pharmaceutically acceptable salt, solvate, or prodrug thereof.
8. The compound of embodiment 1, wherein the compound is of Formula (Ie):

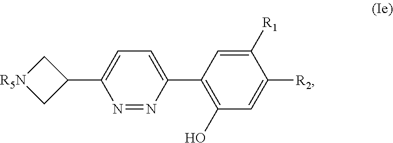

or a pharmaceutically acceptable salt, solvate, or prodrug thereof.
9. The compound of embodiment 1, wherein the compound is of Formula (If):

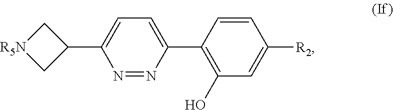

or a pharmaceutically acceptable salt, solvate, or prodrug thereof.
10. The compound of embodiment 4, wherein the compound is of Formula (IIa):

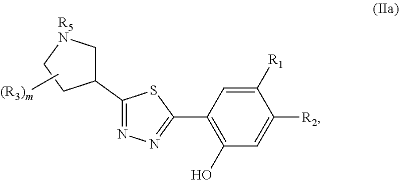

or a pharmaceutically acceptable salt, solvate, or prodrug thereof.
11. The compound of embodiment 4, wherein the compound is of Formula (IIb):

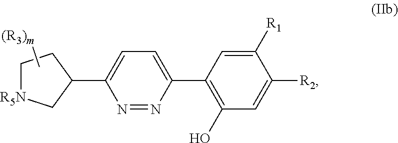

or a pharmaceutically acceptable salt, solvate, or prodrug thereof.
12. The compound of embodiment 5, wherein the compound is of Formula (IIIa):

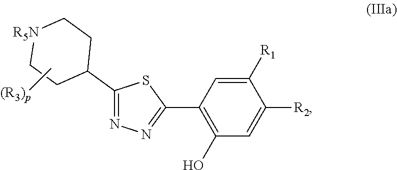

or a pharmaceutically acceptable salt, solvate, or prodrug thereof.
13. The compound of embodiment 5, wherein the compound is of Formula (IIIb):

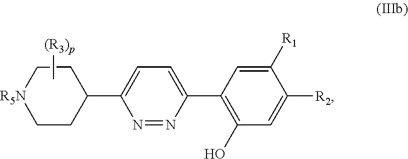

or a pharmaceutically acceptable salt thereof.
14. The compound according to any one of embodiments 1-13, wherein R₁ is H or F.
15. The compound according to any one of embodiments 1-14, wherein R₂ is

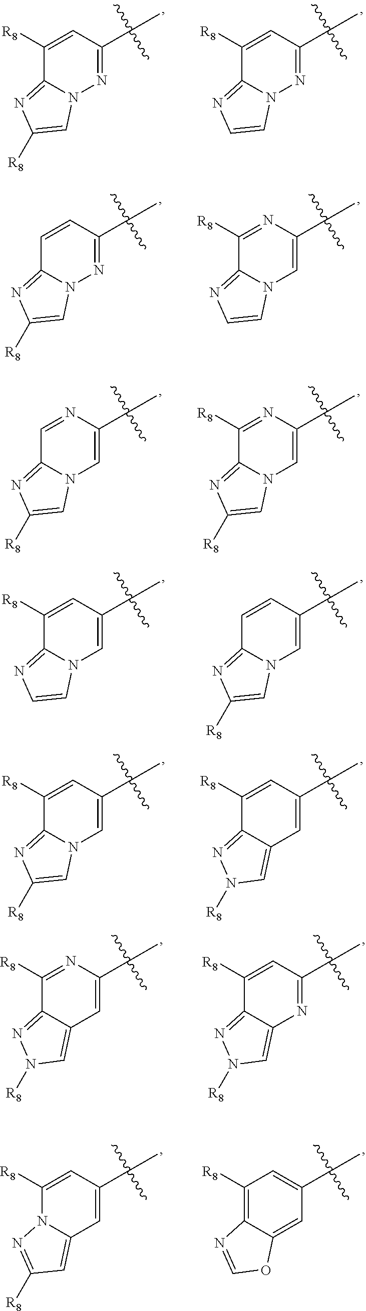

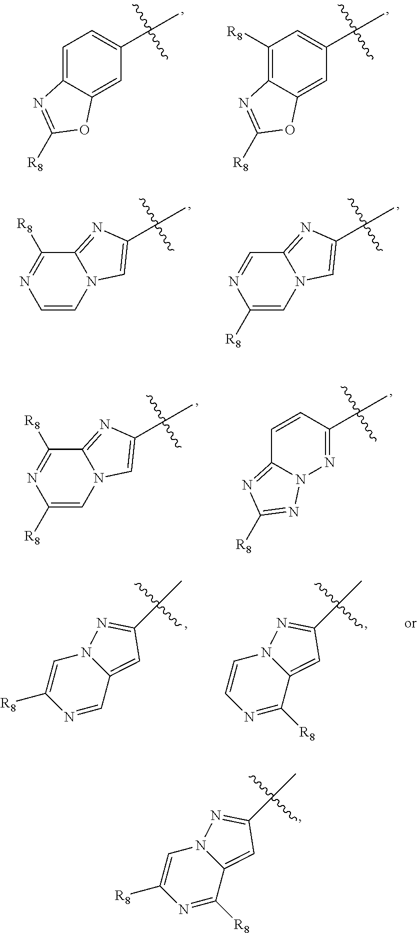

wherein each R₈ is independently R₄.
16. The compound of embodiment 15, wherein each R₈ is independently halo, C₁₋₆alkyl, C₁₋₆haloalkyl, or C₁₋₆alkoxy; preferably each R₈ is independently F, Me, Et, ClF₃, MeO, or EtO.
17. The compound according to any one of embodiments 1-16, wherein R₂ is

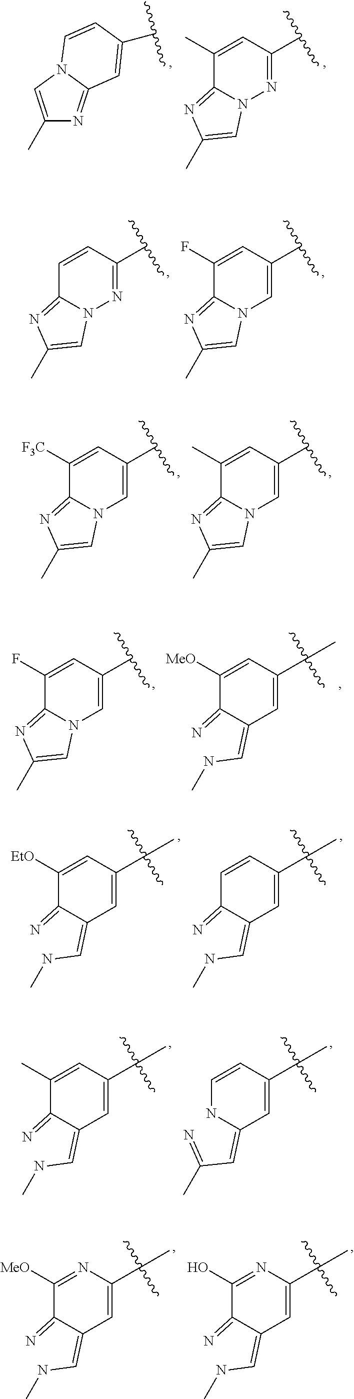

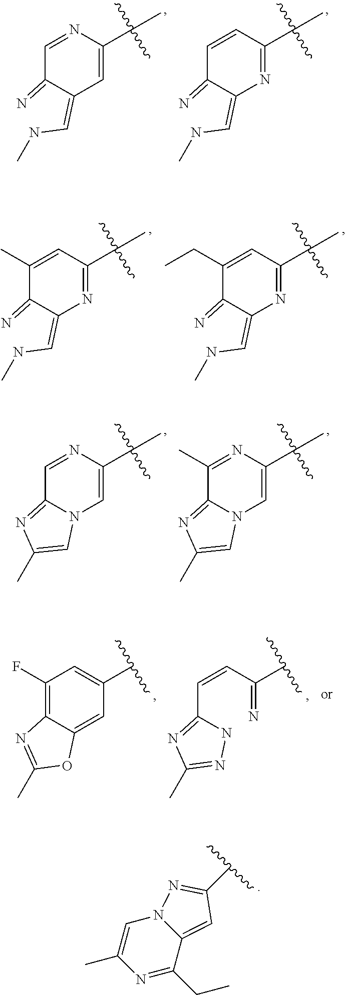

18. The compound according to any one of embodiments 1-16, wherein R₄ is methyl, ethyl, F, or CF₃.
19. A compound according to any one of embodiments 1-18, wherein R₅ is H, C₁₋₆alkyl, or heterocyclyl, wherein heterocyclyl is a 4- to 7-membered ring and comprises 1 or 2 heteroatoms independently selected from N and O.
20. A compound according to any one of embodiments 1-18, wherein R₅ is H.
21. A compound according to any one of embodiments 1-20, selected from a compound of Table 1.
22. A compound according to any one of embodiments 1-21, or a pharmaceutically acceptable salt, solvate, or prodrug thereof for use as a therapeutically active substance.
23. A compound according to any one of embodiments 1-22, or a pharmaceutically acceptable salt, solvate, or prodrug thereof for use as a small molecule splicing modulator.
24. A pharmaceutical composition comprising a compound according to any one of embodiments 1-23, or a pharmaceutically acceptable salt, solvate, or prodrug thereof and one or more pharmaceutically acceptable excipients.
25. A method of treating a disease in a subject in need thereof, the method comprising administering to the subject a therapeutically effective amount of a compound of any one of embodiments 1-23 or a pharmaceutically composition of embodiment 24, wherein the disease is selected from the group consisting of ALS, Alzheimer's disease, argyrophilic grain disease, corticobasal degeneration, cystic fibrosis, dilated cardiomyopathy, Duchenne muscular dystrophy, Ehlers-Danlos syndrome, Fabry's disease, familial dysautonomia, familial hypercholesterolemia, familial persistent hyperinsulinemic hypoglycemia, frontotemporal dementia, FTDP-17, Gacher's disease, globular glial tauopathy, HIV-1, Huntington's disease, Hutchinson-Gilford progeria syndrome, hypercholesterolemia, Leber congenital amaurosis, migraine, multiple sclerosis, myelodysplastic syndromes, NASH, Niemann-Pick's, pain, Parkinson's disease, phenylketonuria, Pick's disease, progressive supranuclear palsy, spinal muscular atrophy, spinocerebellar ataxia type 2, Wilson's disease, Sickle cell anemia, Crohn's disease, ulcerative colitis, psoriasis, and rheumatoid arthritis
26. The method of embodiment 25, wherein the disease is Huntington's disease.
27. A method of treating a disease in a subject in need thereof, the method comprising administering to the subject a therapeutically effective amount of a compound of any one of embodiments 1-23 or a pharmaceutically composition of embodiment 24, wherein the disease is a pulmonary disease selected from the group consisting of chronic obstructive pulmonary disease (COPD), asthma, acute lung injury (ALI), pulmonary fibrosis, and pulmonary arterial hypertension (PAH).

### EXAMPLES

For exemplary purpose, neutral compounds of Formula (I), (II) or (III) are synthesized and tested in the examples. It is understood that the neutral compounds of Formula (I), (II) or (III) may be converted to the corresponding pharmaceutically acceptable salts of the compounds using routine techniques in the art (e.g., by saponification of an ester to the carboxylic acid salt, or by hydrolyzing an amide to form a corresponding carboxylic acid and then converting the carboxylic acid to a carboxylic acid salt).

Nuclear magnetic resonance (NMR) spectra were recorded at 400 MHz or 300 MHz as stated; the chemical shifts (6) are reported in parts per million (ppm). Spectra were recorded using a Bruker or Varian instrument with 8, 16 or 32 scans.

LC-MS chromatograms and spectra were recorded using an Agilent 1200 or Shimadzu LC-20 AD&MS 2020 instrument using a C-18 column such as C18 2.1×30 mm, unless otherwise stated. Injection volumes were 0.7-8.0 μl and the flow rates were typically 0.8 or 1.2 ml/min. Detection methods were diode array (DAD) or evaporative light scattering (ELSD) as well as positive ion electrospray ionisation. MS range was 100-1000 Da. Solvents were gradients of water and acetonitrile both containing a modifier (typically 0.01-0.04%) such as trifluoroacetic acid or ammonium carbonate.

### Abbreviations

ACN Acetonitrile
BINAP 2,2′-bis(diphenylphosphino)-1,1′-binaphthyl
CDCl₃ Chloroform-d
DCM Dichloromethane
DMF N,N-dimethylformamide
DMSO dimethylsulphoxide
DMSO-d₆ Hexadeuterodimethylsulphoxide
DP desired product
eq. Equivalents
ESI Electrospray ionisation
EA ethyl acetate
FCC flash column chromatography
h hour(s)
¹H NMR Proton nuclear magnetic resonance spectroscopy
HPLC high performance liquid chromatography
LC-MS Liquid chromatography-mass spectrometry
MeOD Methanol-d₄
MeOH Methanol
min minute(s)
NaOAc Sodium acetate
NMP N-Methyl-2-Pyrrolidone
PE petroleum ether
ppm parts per million
RM reaction mixture
rt room temperature
SM starting material
TEA Triethylamine
TFA trifluoroacetic acid
THF Tetrahydrofuran
TLC thin layer chromatography
Y Yield

### Example A. tert-butyl 3-(6-chloropyridazin-3-yl)azetidine-1-carboxylate

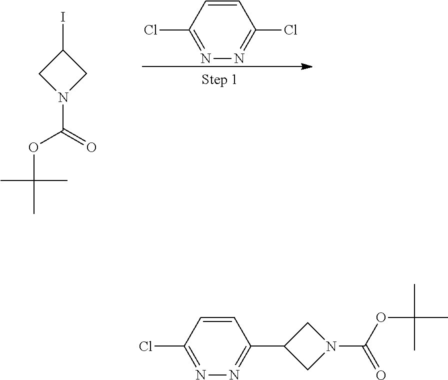 [SMILES: CC(C)(C)OC(=O)N1CC(I)C1 ; Clc1ccc(Cl)nn1 ; CC(C)(C)OC(=O)N1CC(c2ccc(Cl)nn2)C1]

### Step 1: Preparation of tert-butyl 3-(6-chloropyridazin-3-yl)azetidine-1-carboxylate

A 100 mL 3-neck round bottom flask fitted with a magnetic stirrer and flushed with Ar was charged with zinc (13.86 g, 211.94 mmol) and DMA (150 mL, anhydrous). 1,2-Dibromoethane (1.99 g, 10.6 mmol) was slowly added, followed by chlorotrimethylsilane (1.15 g, 10.6 mmol). The mixture was stirred for 15 min at RT and then a solution of tert-butyl 3-iodoazetidine-1-carboxylate (20.0 g, 70.65 mmol) in DMA (20 mL, anhydrous) was added dropwise. The suspension was stirred for 1 h at RT. A 100 mL 3-neck round bottom flask fitted with a mechanical stirrer was charged with copper(I) iodide (1.35 g, 7.06 mmol), [1,1′-bis(diphenylphosphino)ferrocene]dichloropalladium(II) dichloromethane adduct (2.88 g, 3.53 mmol), 3,6-dichloropyridazine (21.05 g, 141.29 mmol), and DMA (100 mL, anhydrous). The dark solution was degassed for 15 min. The clear zinc reagent solution above the residual solid zinc was transferred to the above 100 mL flask by cannulation. The dark solution was degassed and heated to 80° C. for 16 h. The resulting mixture was diluted with brine (500 mL) and extracted with EtOAc (3×150 mL). The combined organics were washed with water (2×200 mL) and brine (200 mL), followed by drying over sodium sulfate. The solution was concentrated and the residue was purified by flash column chromatography to provide tert-butyl 3-(6-chloropyridazin-3-yl)azetidine-1-carboxylate (8.0 g, Y.: 39.9%) as a light yellow solid. ESI-MS (M+Na)+: 292. ¹H NMR (500 MHz, DMSO-d6) δ 7.87 (d, J=8.9 Hz, 1H), 7.77 (d, J=8.9 Hz, 1H), 4.31-4.17 (m, 2H), 4.15-3.98 (m, 3H), 1.38 (s, 9H).

### Example B. 2-methyl-6-(4,4,5,5-tetramethyl-1,3,2-dioxaborolan-2-yl)-8-(trifluoromethyl)imidazo[1,2-a]pyridine

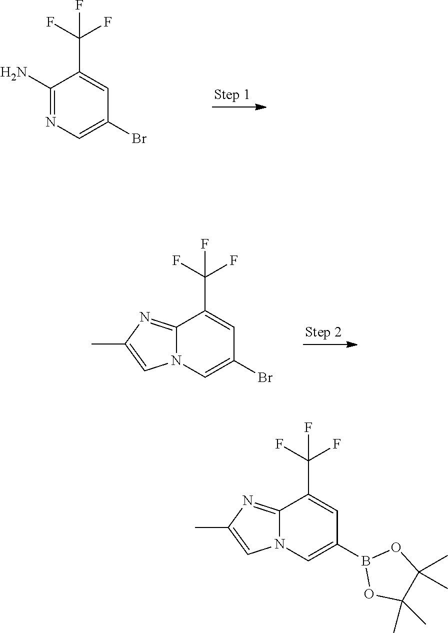

### Step 1: Preparation of 5-bromo-3-(trifluoromethyl)pyridin-2-amine

A suspension of 5-bromo-3-(trifluoromethyl)pyridin-2-amine (1.84 g, 7.64 mmol) in isopropanol (30 mL) was treated with pyridine (60.46 mg, 764.36 μmol, 60.0 μl, 0.1 equiv), 4-methylbenzene-1-sulfonic acid hydrate (145.39 mg, 764.36 μmol), 1-bromo-2,2-dimethoxypropane (1.68 g, 9.17 mmol, 1.24 ml, 1.2 equiv). The resulting mixture was heated at 80° C. for 21 h, then cooled to room temperature and concentrated under vacuum at 40° C. The residue triturated with MeCN and the precipitate formed was filtered to give 6-bromo-2-methyl-8-(trifluoromethyl)imidazo[1,2-a]pyridine (1.6 g, Y: 71.3%). ESI-MS (M+H)+: 280.0.

### Step 2: Preparation of 2-methyl-6-(4,4,5,5-tetramethyl-1,3,2-dioxaborolan-2-yl)-8-(trifluoromethyl)imidazo[1,2-a]pyridine

A solution of 6-bromo-2-methyl-8-(trifluoromethyl)imidazo[1,2-a]pyridine (1.8 g, 6.45 mmol), 4,4,5,5-tetramethyl-2-(4,4,5,5-tetramethyl-1,3,2-dioxaborolan-2-yl)-1,3,2-dioxaborolane (1.8 g, 7.1 mmol), potassium acetate (1.9 g, 19.35 mmol), and [1,1′-bis(diphenylphosphino)ferrocene]dichloropalladium (II) dichloromethane adduct (263.43 mg, 322.58 μmol) in dioxane (25 mL) was degassed and purged with Ar. The resulting mixture was heated at 100° C. overnight. The reaction mixture was cooled to rt, filtered through a pad of Celite, and concentrated in vacuo, and the crude 2-methyl-6-(4,4,5,5-tetramethyl-1,3,2-dioxaborolan-2-yl)-8-(trifluoromethyl)imidazo[1,2-a]pyridine (2.75 g) was used directly in the next step without further purification. ¹H NMR (400 MHz, DMSO-d6) δ 9.01 (s, 1H), 7.89 (s, 1H), 7.56 (s, 1H), 2.37 (s, 3H), 1.31 (s, 12H).

### Example C. 7-methoxy-2-methyl-5-(4,4,5,5-tetramethyl-1,3,2-dioxaborolan-2-yl)-2H-indazole

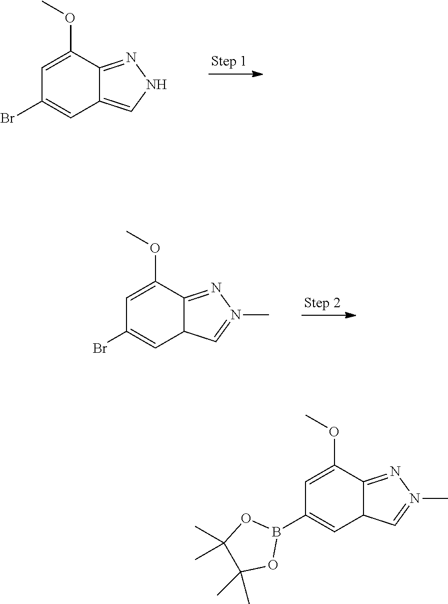

### Step 1: Preparation of 5-bromo-7-methoxy-2-methyl-2H-indazole

To a solution of 5-bromo-7-methoxy-1H-indazole (500.11 mg, 2.2 mmol) in 10 ml of EtOAc trimethyloxidanium tetrafluoroboranuide (977.32 mg, 6.61 mmol) was added at rt under stirring. The solution was stirred at rt for 2 days. The mixture was poured into sat. NaHCO₃ (15 ml) and organic layer was separated, dried under Na₂SO₄ and evaporated to dryness to give the crude 5-bromo-7-methoxy-2-methyl-2H-indazole (460.0 mg, Y: 85%). ESI-MS (M+H)+: 242.0. ¹H NMR (500 MHz, DMSO-d6) δ 8.24 (s, 1H), 7.45 (s, 1H), 6.66 (s, 1H), 4.11 (s, 3H), 3.89 (s, 3H).

### Step 2: Preparation of 7-methoxy-2-methyl-5-(4,4,5,5-tetramethyl-1,3,2-dioxaborolan-2-yl)-2H-indazole

A solution of 5-bromo-7-methoxy-2-methyl-2H-indazole (460.0 mg, 1.91 mmol), 4,4,5,5-tetramethyl-2-(4,4,5,5-tetramethyl-1,3,2-dioxaborolan-2-yl)-1,3,2-dioxaborolane (484.7 mg, 1.91 mmol), potassium acetate (561.98 mg, 5.73 mmol), and [1,1′-bis(diphenylphosphino)ferrocene]dichloropalladium (II) dichloromethane adduct (77.94 mg, 95.44 μmol) in dioxane (15 mL) was degassed and purged with Ar. The resulting mixture was heated at 90° C. overnight. The reaction mixture was diluted with EtOAc (30 mL) and filtered. The filtrate was evaporated to give crude give 7-methoxy-2-methyl-5-(4,4,5,5-tetramethyl-1,3,2-dioxaborolan-2-yl)-2H-indazole (600.0 mg, Y: 87.3%). ESI-MS (M+H)+: 289.2. ¹H NMR (400 MHz, DMSO-d6) δ 8.32 (s, 1H), 7.70 (s, 1H), 4.14 (s, 3H), 3.89 s, 3H), 1.30 (s, 12H).

### Example 1. 2-(6-(azetidin-3-yl)pyridazin-3-yl)-5-(8-fluoro-2-methylimidazo[1,2-a]pyridin-6-yl)phenol, HCl. (Compound 1)

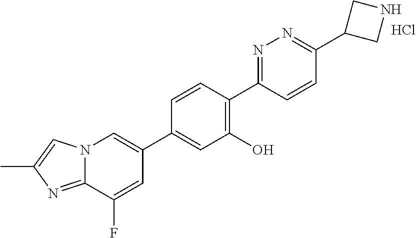 [SMILES: Cc1cn2cc(-c3ccc(-c4ccc(C5CNC5)nn4)c(O)c3)cc(F)c2n1 ; Cl]

### Step 1: Synthesis of 2-bromo-5-iodophenol

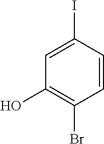

To a solution of 5-amino-2-bromophenol (50.0 g, 265.9 mmol) in 400 mL of 2.5 M hydrochloric acid, sodium nitrite (18.78 g, 272.2 mmol) in water (120 mL) was added dropwise at 0° C. After 15 min a solution of potassium iodide (49.3 g, 2967 mmol) in water (100 mL) was added dropwise and the whole mixture was heated at 60° C. for 1 h. The resulting mixture was extracted with MTBE (2×300 mL). The organic phase was dried over Na₂SO₄ and concentrated under reduced pressure, to give a brown liquid, which was purified by flash chromatography to give 2-bromo-5-iodophenol (60.0 g, 172.6 mmol, 75% yield).

### Step 2: Synthesis of 1-bromo-4-iodo-2-(methoxymethoxy)benzene

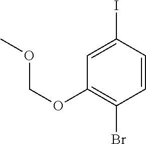

A mixture of 2-bromo-5-iodophenol (30 g, 100 mmol), potassium carbonate (41.4 g, 300 mmol) and MOM-Cl (24.15, 300 mmol) in DMF (400 mL) was stirred at rt for 48 h. Next, the reaction mixture was diluted with water (1000 mL) and extracted with MTBE (2×600 mL). The organic phases were washed with water and brine, dried over Na₂SO₄ and concentrated under reduced pressure, to give a brown liquid, which was purified by flash chromatography to give 1-bromo-4-iodo-2-(methoxymethoxy)benzene (21 g, 61 mmol, 61% yield).

### Step 3: Synthesis of 6-(4-bromo-3-(methoxymethoxy)phenyl)-8-fluoro-2-methylimidazo[1,2-a]pyridine

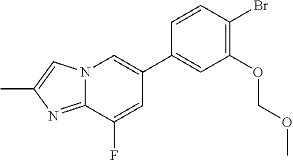

A portion of Pd(dppf)Cl₂ (950 mg, 1.2 mmol) was added to a suspension of 1-bromo-4-iodo-2-(methoxymethoxy)benzene (4 g, 11.7 mmol), 8-fluoro-2-methyl-6-(4,4,5,5-tetramethyl-1,3,2-dioxaborolan-2-yl)imidazo[1,2-a]pyridine (3.87 g, 14 mmol) and potassium carbonate (3.31 g, 24 mmol) in 100 mL of dioxane and 2 mL of water. The mixture was stirred at 80° C. for 12 h. After cooling, the solid was collected by filtration and washed with EtOAc. The filtrate was concentrated and partitioned between EtOAc and water. The organic layer was washed with brine, dried over Na₂SO₄, and concentrated to give crude product, which was purified by column chromatography to give 6-(4-bromo-3-(methoxymethoxy)phenyl)-8-fluoro-2-methylimidazo[1,2-a]pyridine (2 g, 5.5 mmol, 47% yield).

### Step 4: Synthesis of 8-fluoro-6-(3-(methoxymethoxy)-4-(4,4,5,5-tetramethyl-1,3,2-dioxaborolan-2-yl)phenyl)-2-methylimidazo[1,2-a]pyridine

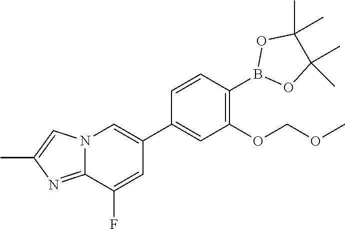

A mixture of 6-(4-bromo-3-(methoxymethoxy)phenyl)-8-fluoro-2-methylimidazo[1,2-a]pyridine (2 g, 5.5 mmol), bis(pinacolato)diboron (1.39 g, 5.5 mmol), potassium acetate (1.61 g, 16 mmol) and Pd(dppf)Cl₂ (450 mg, 0.55 mmol) in dioxane (20 mL) was stirred at 80° C. (under Ar atmosphere) for 12 h. Next it was concentrated, worked-up with EtOAc/water. The extract was dried and evaporated to give crude material, which was purified by column chromatography to afford 8-fluoro-6-(3-(methoxymethoxy)-4-(4,4,5,5-tetramethyl-1,3,2-dioxaborolan-2-yl)phenyl)-2-methylimidazo[1,2-a]pyridine (1.1 g, 2.6 mmol, 46% yield).

### Step 5: Synthesis of tert-butyl 3-(6-(4-(8-fluoro-2-methylimidazo[1,2-a]pyridin-6-yl)-2-(methoxymethoxy)phenyl)pyridazin-3-yl)azetidine-1-carboxylate

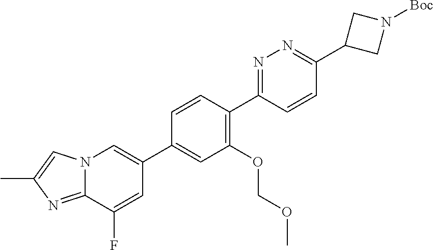 [SMILES: COCOc1cc(-c2cc(F)c3nc(C)cn3c2)ccc1-c1ccc(C2CN(C(=O)OC(C)(C)C)C2)nn1]

A portion of Pd(dppf)Cl₂ (70 mg, 85.77 μmol) was added to a suspension of 8-fluoro-6-(3-(methoxymethoxy)-4-(4,4,5,5-tetramethyl-1,3,2-dioxaborolan-2-yl)phenyl)-2-methylimidazo[1,2-a]pyridine (350 mg, 0.82 mmol), tert-butyl 3-(6-chloropyridazin-3-yl)azetidine-1-carboxylate (170 mg, 0.63 mmol) and potassium carbonate (355.6 mg, 2.57 mmol) in dioxane (10 mL) and water (1 mL). The mixture was stirred at 80° C. for 12 h. After cooling, the solid was collected by filtration and washed with EtOAc. The filtrate was concentrated and partitioned between EtOAc and water. The organic layer was washed with brine, dried over Na₂SO₄, and concentrated to give crude product, which was purified by column chromatography to give tert-butyl 3-(6-(4-(8-fluoro-2-methylimidazo[1,2-a]pyridin-6-yl)-2-(methoxymethoxy)phenyl)pyridazin-3-yl)azetidine-1-carboxylate (50.0 mg, 0.096 mmol, 15% yield).

### Step 6: 2-(6-(azetidin-3-yl)pyridazin-3-yl)-5-(8-fluoro-2-methylimidazo[1,2-a]pyridin-6-yl)phenol, HCl

Tert-butyl 3-(6-(4-(8-fluoro-2-methylimidazo[1,2-a]pyridin-6-yl)-2-(methoxymethoxy)phenyl)pyridazin-3-yl)azetidine-1-carboxylate (45 mg, 86.5 μmol) was suspended in 0.5 mL of dioxane/HCl (10%) and stirred for 12 h. Next, it was evaporated to dryness to give 2-(6-(azetidin-3-yl)pyridazin-3-yl)-5-(8-fluoro-2-methylimidazo[1,2-a]pyridin-6-yl)phenol (26 mg, 65.5 μmol, 76%). ¹H NMR (400 MHz, DMSO-d6) δ 2.44 (s, 3H), 4.35 (m, 4H), 4.44 (m, 1H), 7.44 (m, 2H), 7.96 (d, 1H), 8.06 (s, 1H), 8.11 (d, 1H), 8.20 (d, 1H), 8.60 (d, 1H), 9.04 (s, 1H), 9.14 (s, 1H), 9.30 (s, 1H). LC-HMRS calculated for C₂₁H₁₈FN₅O: m/z=375.15. found 374.1.0 (M−1).

### Example 2. 2-(6-(azetidin-3-yl)pyridazin-3-yl)-5-(2-methylimidazo[1,2-a]pyridin-6-yl)phenol, HCl. (Compound 2)

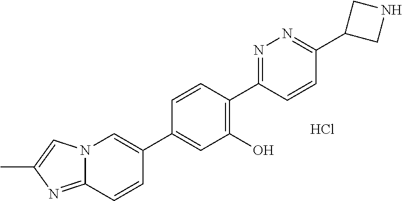 [SMILES: Cc1cn2cc(-c3ccc(-c4ccc(C5CNC5)nn4)c(O)c3)ccc2n1 ; Cl]

### Step 1: Synthesis of 6-(4-bromo-3-(methoxymethoxy)phenyl)-2-methylimidazo[1,2-a]pyridine

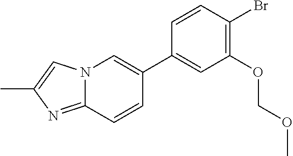

A portion of Pd(dppf)Cl₂ (0.95 g, 1.2 mmol) was added to a suspension of 1-bromo-4-iodo-2-(methoxymethoxy)benzene (4 g, 11.7 mmol), 2-methyl-6-(4,4,5,5-tetramethyl-1,3,2-dioxaborolan-2-yl)imidazo[1,2-a]pyridine (3.6 g, 14 mmol) and potassium carbonate (3.3 g, 24 mmol) in dioxane (100 mL) and water (2 mL). The mixture was stirred at 80° C. for 12 h. After cooling, the solid was collected by filtration and washed with EtOAc. The filtrate was concentrated and partitioned between EtOAc and water. The organic layer was washed with brine, dried over Na₂SO₄, and concentrated to give crude product, which was purified by column chromatography to give 6-(4-bromo-3-(methoxymethoxy)phenyl)-2-methylimidazo[1,2-a]pyridine (1.6 g, 4.6 mmol, 42% yield).

### Step 2: Synthesis of 6-(3-(methoxymethoxy)-4-(4,4,5,5-tetramethyl-1,3,2-dioxaborolan-2-yl)phenyl)-2-methylimidazo[1,2-a]pyridine

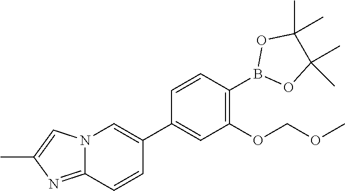

A mixture of 6-(4-bromo-3-(methoxymethoxy)phenyl)-2-methylimidazo[1,2-a]pyridine (1.6 g, 4.6 mmol), bis(pinacolato)diboron (1.17 g, 4.6 mmol), potassium acetate (1.37 g, 14 mmol) and Pd(dppf)Cl₂ (375 mg, 0.46 mmol) in dioxane (20 mL) was stirred at 80° C. (under Ar atmosphere) for 12 h. Next, the reaction mixture was concentrated, worked-up with EtOAc/water. The extract was dried and evaporated to give crude material, which was purified by column chromatography to afford 6-(3-(methoxymethoxy)-4-(4,4,5,5-tetramethyl-1,3,2-dioxaborolan-2-yl)phenyl)-2-methylimidazo[1,2-a]pyridine (0.9 g, 2.3 mmol, 50% yield).

### Step 3: Synthesis of tert-butyl 3-(6-(2-(methoxymethoxy)-4-(2-methylimidazo[1,2-a]pyridin-6-yl)phenyl)pyridazin-3-yl)azetidine-1-carboxylate

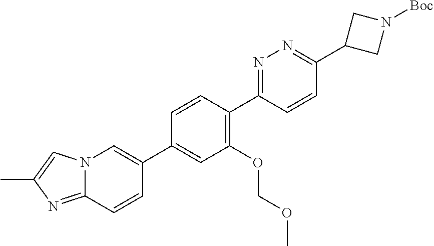 [SMILES: COCOc1cc(-c2ccc3nc(C)cn3c2)ccc1-c1ccc(C2CN(C(=O)OC(C)(C)C)C2)nn1]

A portion of Pd(dppf)Cl₂ (70 mg, 85.8 μmol) was added to a suspension of 6-(3-(methoxymethoxy)-4-(4,4,5,5-tetramethyl-1,3,2-dioxaborolan-2-yl)phenyl)-2-methylimidazo[1,2-a]pyridine (350 mg, 1 mmol), tert-butyl 3-(6-chloropyridazin-3-yl)azetidine-1-carboxylate (216 mg, 0.8 mmol) and potassium carbonate (355.6 mg, 2.57 mmol) in dioxane (10 mL) and water (1 mL). The mixture was stirred at 80° C. for 12 h. After cooling, the solid was collected by filtration and washed with EtOAc. The filtrate was concentrated and partitioned between EtOAc and water. The organic layer was washed with brine, dried over Na₂SO₄, and concentrated to give crude product, which was purified by column chromatography to give tert-butyl 3-(6-(2-(methoxymethoxy)-4-(2-methylimidazo[1,2-a]pyridin-6-yl)phenyl)pyridazin-3-yl)azetidine-1-carboxylate (98 mg, 0.195 mmol, 24% yield).

### Step 4: 2-(6-(azetidin-3-yl)pyridazin-3-yl)-5-(2-methylimidazo[1,2-a]pyridin-6-yl)phenol, HCl

Tert-butyl 3-(6-(2-(methoxymethoxy)-4-(2-methylimidazo[1,2-a]pyridin-6-yl)phenyl)pyridazin-3-yl)azetidine-1-carboxylate (61 mg, 121 μmol) was suspended in 0.5 ml of dioxane/HCl (10%) and stirred for 12 h. Next it was evaporated to dryness to give 2-(6-(azetidin-3-yl)pyridazin-3-yl)-5-(2-methylimidazo[1,2-a]pyridin-6-yl)phenol, HCl (59 mg, 149.7 μmol). ¹H NMR (400 MHz, DMSO-d6) δ 2.52 (s, 3H), 4.35 (m, 4H), 4.45 (m, 1H), 7.43 (d, 1H), 7.47 (s, 1H), 7.99 (d, 2H), 8.10 (s, 1H), 8.22 (d, 1H), 8.29 (d, 1H), 8.59 (d, 1H), 9.26 (s, 1H), 9.38 (s, 1H), 9.61 (s, 1H). LC-HRMS calcd for C₂₁H₁₉N₅O: m/z=357.18. found 358.0 (M+1).

### Example 3. 2-[6-(azetidin-3-yl)pyridazin-3-yl]-5-{2,8-dimethylimidazo[1,2-b]pyridazin-6-yl}phenol (Compound 3)

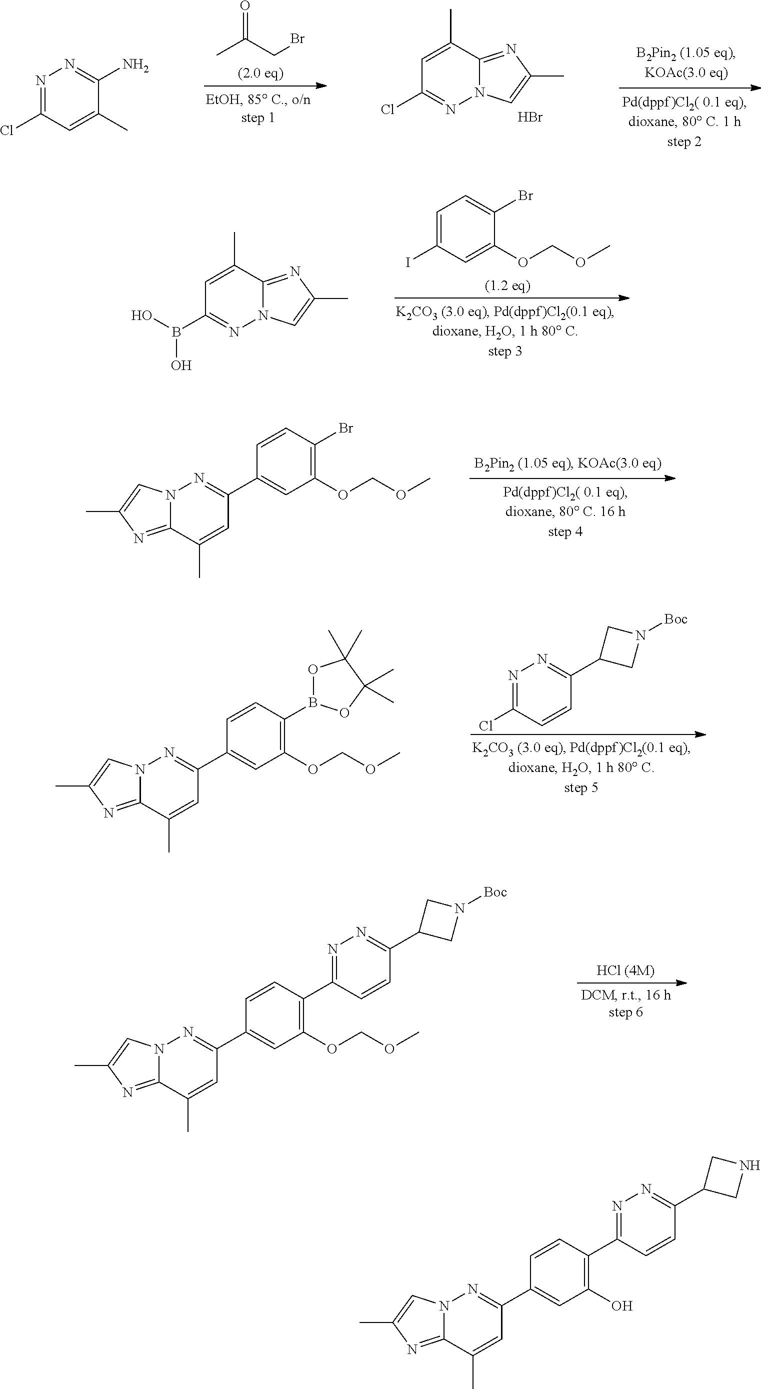

### Step 1: Preparation of 6-chloro-2,8-dimethylimidazo[1,2-b]pyridazine hydrobromide

To a suspension of 6-chloro-4-methylpyridazin-3-amine (1.35 g, 9.44 mmol) in EtOH (30 mL) was added 1-bromopropan-2-one (2.59 g, 18.88 mmol). The reaction mixture was stirred overnight at 85° C. The mixture was allowed to cool down to room temperature. After concentration, the mixture was diluted with EtOH (5 mL) and EtOAc (30 mL), the mixture was filtered and the solid was dried under vacuum to afford title product (750 mg, Y: 30%) as a light grey solid. ESI-MS (M+H)+: 182.0. ¹H NMR (400 MHz, DMSO-d6) δ 8.03 (s, 1H), 7.19 (s, 1H), 2.53 (s, 3H), 2.38 (s, 3H).

### Step 2: Preparation of (2,8-dimethylimidazo[1,2-b]pyridazin-6-yl)boronic acid

A mixture of 6-chloro-2,8-dimethylimidazo[1,2-b]pyridazine hydrobromide (590 mg, 3.26 mmol), 4,4,4′,4′,5,5,5′,5′-octamethyl-2,2′-bi(1,3,2-dioxaborolane) (869 mg, 3.42 mmol), KOAc (639 mg, 6.52 mmol) and Pd(dppf)Cl₂ (238 mg, 0.326 mmol) in dioxane (18 mL) at room temperature was purged with N₂ for three times. Then the reaction mixture was stirred at 100° C. for 3 h. After cooling to room temperature, the mixture was filtered and the filtration was used in the next step without further purification. ESI-MS (M+H)+: 192.0.

### Step 3: Preparation of 6-(4-bromo-3-(methoxymethoxy)phenyl)-2,8-dimethylimidazo[1,2-b]pyridazine

A mixture of (2,8-dimethylimidazo[1,2-b]pyridazin-6-yl)boronic acid (422 mg, 2.22 mmol), 1-bromo-4-iodo-2-(methoxymethoxy)benzene (777 mg, 2.26 mmol), K2CO3 (919 mg, 6.66 mmol) and Pd(dppf)Cl₂ (161 mg, 0.22 mmol) in H2O (4 mL) was stirred for 1 h at 80° C. under N₂. The mixture was allowed to cool down to room temperature and concentrated. The residue was purified by silica gel column chromatography (PE:EA=1:5) to give title product (1.0 g, Y: 84%) as a light yellow solid. ESI-MS (M+H)+: 362.0, 364.0.

### Step 4: Preparation of 6-(3-(methoxymethoxy)-4-(4,4,5,5-tetramethyl-1,3,2-dioxaborolan-2-yl)phenyl)-2,8-dimethylimidazo[1,2-b]pyridazine

A mixture of 6-(4-bromo-3-(methoxymethoxy)phenyl)-2,8-dimethylimidazo[1,2-b]pyridazine (500 mg, 1.385 mmol), bis(pinacolato)diboron (422 mg, 1.662 mmol), potassium acetate (272 mg, 2.770 mmol) and Pd(dppf)Cl₂ (102 mg, 0.139 mmol) in dioxane (10 mL) was stirred at 100° C. (under Ar atmosphere) for 16 h. The mixture was allowed to cool down to room temperature and filtered. The filtrate was used in the next step without further purification. ESI-MS (M+H)+: 410.2

### Step 5: Preparation of tert-butyl 3-(6-(4-(2,8-dimethylimidazo[1,2-b]pyridazin-6-yl)-2-(methoxymethoxy)phenyl)pyridazin-3-yl)azetidine-1-carboxylate

To the above solution was added H2O (1.5 mL), tert-butyl 3-(6-chloropyridazin-3-yl)azetidine-1-carboxylate and (403 mg, 1.5 mmol), K2CO3 (345 mg, 2.5 mmol) and Pd(dppf)Cl₂ (92 mg, 0.125 mmol). The resulting mixture was stirred for 2 h at 100° C. under N₂. The mixture was allowed to cool down to room temperature and concentrated. The residue was purified by silica gel column chromatography (EA:PE=1:10, v/v) to give title product (255 mg, Y: 36%) as a yellow solid. ESI-MS (M+H)+: 517.2.

### Step 6: Preparation of 6-(4-(6-(azetidin-3-yl)pyridazin-3-yl)-3-(methoxymethoxy)phenyl)-2,8-dimethylimidazo[1,2-b]pyridazine

To a solution of tert-butyl 3-(6-(4-(2,8-dimethylimidazo[1,2-b]pyridazin-6-yl)-2-(methoxymethoxy)phenyl)pyridazin-3-yl)azetidine-1-carboxylate (220 mg, 0.426 mmol) in DCM (10 mL) was added HCl in dioxane (1.1 mL) at 0° C. The mixture was stirred overnight at rt. After concentration, the residue was treated with DCM (10 mL) and removed the solvent again. The crude was purified by pre-HPLC (0.05% HCl in ACN) to give title product (3.0 mg, Y: 15%) as a light yellow solid. ESI-MS (M+H)+: 373.4. ¹H NMR (400 MHz, DMSO-d6) δ 9.64 (br s, 1H), 9.30 (br s, 1H), 8.59 (d, J=9.0 Hz, 1H), 8.55 (s, 1H), 8.39 (s, 1H), 8.24 (d, J=8.3 Hz, 1H), 8.01 (d, J=9.0 Hz, 1H), 7.83 (d, J=1.5 Hz, 1H), 7.76 (d, J=8.3 Hz, 1H), 4.47 (d, J=8.9 Hz, 2H), 4.36 (d, J=6.7 Hz, 3H), 2.77 (s, 3H), 2.59 (s, 3H).

### Example 4. Synthesis of 2-(6-(azetidin-3-yl)pyridazin-3-yl)-5-(2,8-dimethylimidazo[1,2-a]pyridin-6-yl)phenol hydrochloride (Compound 4)

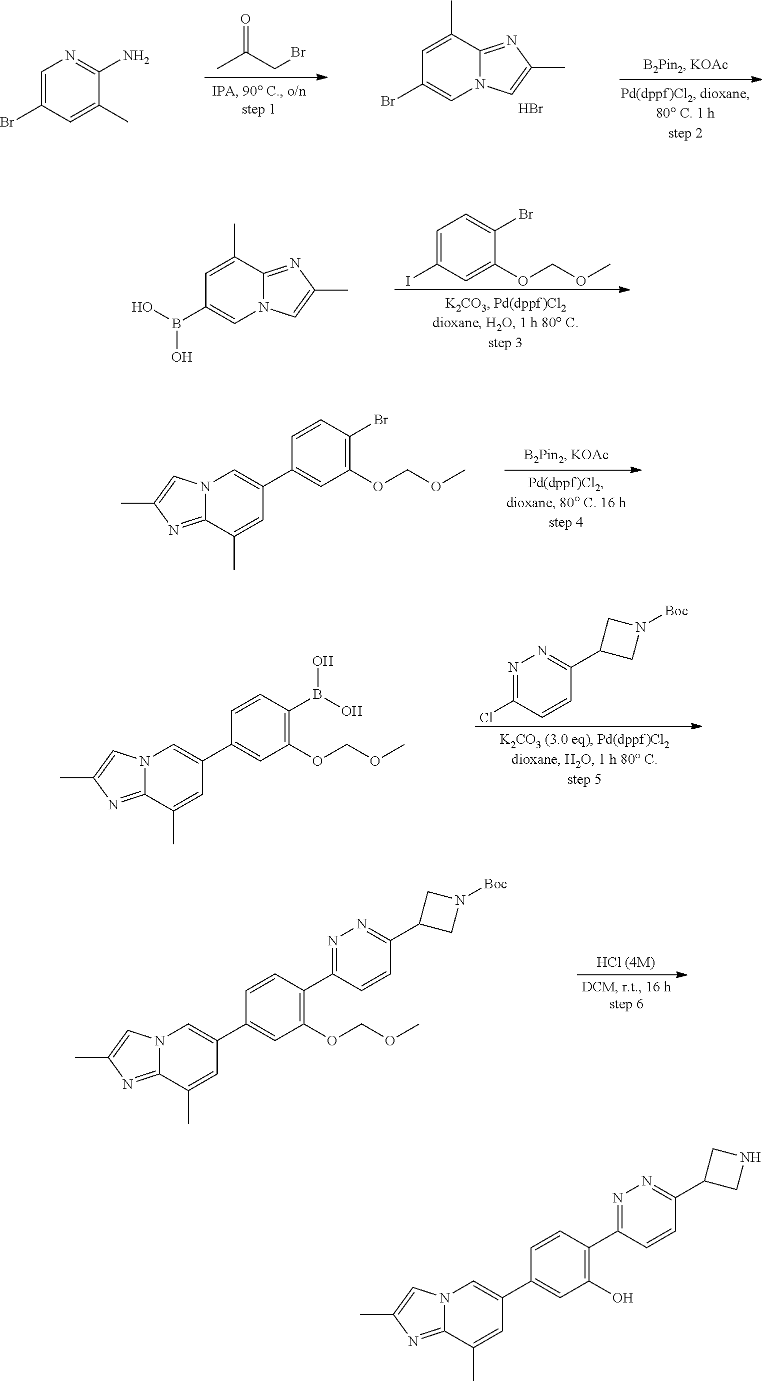

### Step 1: Preparation of 6-bromo-2,8-dimethylimidazo[1,2-a]pyridine HBr salt

To a suspension of 5-bromo-3-methylpyridin-2-amine (2.0 g, 10.70 mmol) in IPA (20 mL) was added 1-bromopropan-2-one (2.9 g, 21.39 mmol). The reaction mixture was stirred overnight at 90° C. The mixture was allowed to cool down to room temperature. After concentration, the mixture was added EtOH (5 mL) and EtOAc (30 mL), which was filtered to afford title product (2.8 g, Y: 86%) as a grey solid. ESI-MS (M+H)+: 226.8. ¹H NMR (400 MHz, DMSO-d6) δ 9.13 (s, 1H), 8.05 (d, J=0.8 Hz, 1H), 7.96 (s, 1H), 2.57 (s, 3H), 2.51 (d, J=0.4 Hz, 3H).

### Step 2: Preparation of (2,8-dimethylimidazo[1,2-a]pyridin-6-yl)boronic acid

A mixture of 6-bromo-2,8-dimethylimidazo[1,2-a]pyridine (680 mg, 2.22 mmol, HBr salt), B₂Pin₂ (593 mg, 2.33 mmol), KOAc (653 mg, 6.66 mmol) and Pd(dppf)Cl₂ (161 mg, 0.22 mmol) in dioxane (20 mL) at room temperature was purged with N₂ for three times. Then the reaction mixture was heated to 80° C. for 1 h. LCMS showed no starting material was remained and desired product was formed. The reaction was stopped and cooled to room temperature. The mixture was filtered and the filtration was used in the next step without further purification. ESI-MS (M+H)+: 191.2

### Step 3: Preparation of 6-(4-bromo-3-(methoxymethoxy)phenyl)-2,8-dimethylimidazo[1,2-a]pyridine

A mixture of (2,8-dimethylimidazo[1,2-a]pyridin-6-yl)boronic acid (crude from above step, 2.22 mmol), 1-bromo-4-iodo-2-(methoxymethoxy)benzene (777 mg, 2.26 mmol), K2CO3 (919 mg, 6.66 mmol) and Pd(dppf)Cl₂ (161 mg, 0.22 mmol) in H2O (4 mL) was stirred for 1 h at 80° C. under N₂. The mixture was allowed to cool down to room temperature and concentrated. The residue was purified by silica gel column chromatography (PE:EA=1:5) to give title product (350 mg, Y: 59%) as a red solid. ESI-MS (M+H)+: 363.1 ¹H NMR (400 MHz, CDCl₃) δ 8.08 (s, 1H), 7.60 (d, J=8.2 Hz, 1H), 7.37 (s, 1H), 7.31 (d, J=2.0 Hz, 1H), 7.12 (s, 1H), 7.12-7.05 (m, 1H), 5.33 (s, 2H), 3.56 (s, 3H), 2.65 (s, 3H), 2.49 (s, 3H).

### Step 4: Preparation of (4-(2,8-dimethylimidazo[1,2-a]pyridin-6-yl)-2-(methoxymethoxy)phenyl)boronic acid

A mixture of 6-(4-bromo-3-(methoxymethoxy)phenyl)-2,8-dimethylimidazo[1,2-a]pyridine (300 mg, 0.83 mmol), bis(pinacolato)diboron (232 mg, 0.91 mmol), potassium acetate (244 mg, 2.49 mmol) and Pd(dppf)Cl₂ (61 mg, 0.083 mmol) in dioxane (20 mL) was stirred at 80° C. (under Ar atmosphere) for 16 h. The mixture was allowed to cool down to room temperature and filtered. The filtrate was used in the next step without further purification. ESI-MS (M+H)+: 327.0

### Step 5: Preparation of tert-butyl 3-(6-(4-(2,8-dimethylimidazo[1,2-a]pyridin-6-yl)-2-(methoxymethoxy)phenyl)pyridazin-3-yl)azetidine-1-carboxylate

To the above solution was added H₂O (5 mL), tert-butyl 3-(6-chloropyridazin-3-yl)azetidine-1-carboxylate and (245 mg, 0.91 mM), K2CO3 (344 mg, 2.49 mmol) and Pd(dppf)Cl₂ (61 mg, 0.083 mmol). The resulting mixture was stirred for 1 h at 80° C. under N₂. The mixture was allowed to cool down to room temperature and concentration under reduced pressure. The residue was purified by silica gel column chromatography (DCM:MeOH=20:1) to give title product (90 mg, Y: 21% for two steps) as a yellow solid. ESI-MS (M+H)+: 516.2 ¹H NMR (400 MHz, CDCl₃) δ 8.18 (s, 1H), 8.08 (t, J=8.1 Hz, 2H), 7.52 (d, J=8.9 Hz, 1H), 7.44 (d, J=1.5 Hz, 1H), 7.41-7.36 (m, 2H), 7.23 (s, 1H), 5.30 (d, J=3.2 Hz, 2H), 4.44 (t, J=8.6 Hz, 2H), 4.29 (s, 1H), 4.12 (dd, J=14.3, 7.1 Hz, 2H), 3.49 (s, 3H), 2.68 (s, 3H), 2.51 (s, 3H), 1.48 (s, 9H).

### Step 6: Preparation of 2-(6-(azetidin-3-yl)pyridazin-3-yl)-5-(2,8-dimethylimidazo[1,2-a]pyridin-6-yl)phenol hydrochloride

To a solution of tert-butyl 3-(6-(4-(2,8-dimethylimidazo[1,2-a]pyridin-6-yl)-2-(methoxymethoxy)phenyl)pyridazin-3-yl)azetidine-1-carboxylate (80 mg, 0.155 mmol) in DCM (5 mL) was added HCl in dioxane (1 mL) at 0° C. The mixture was stirred overnight at 0° C.-rt. The reaction mixture was concentrated to dryness. The residue was treated with DCM (5 mL) and co-evaporated again. The crude was purified by pre-HPLC (0.05% HCl in ACN) to give title product (3.0 mg, Y: 5%) as a yellow solid. ESI-MS (M+H)+: 372.1. ¹H NMR (400 MHz, DMSO-d6) δ 8.81 (s, 1H), 8.57 (d, J=9.1 Hz, 1H), 8.37 (s, 1H), 8.14 (d, J=8.2 Hz, 1H), 7.97 (d, J=9.0 Hz, 1H), 7.70 (s, 1H), 7.43 (s, 1H), 7.35 (d, J=8.0 Hz, 2H), 4.35 (d, J=7.6 Hz, 1H), 4.17 (d, J=7.6 Hz, 4H), 2.53 (s, 3H), 2.36 (s, 3H).

### Example 5. 2-[6-(azetidin-3-yl)pyridazin-3-yl]-5-[2-methyl-8-(trifluoromethyl)imidazo[1,2-a]pyridin-6-yl]phenol (Compound 5)

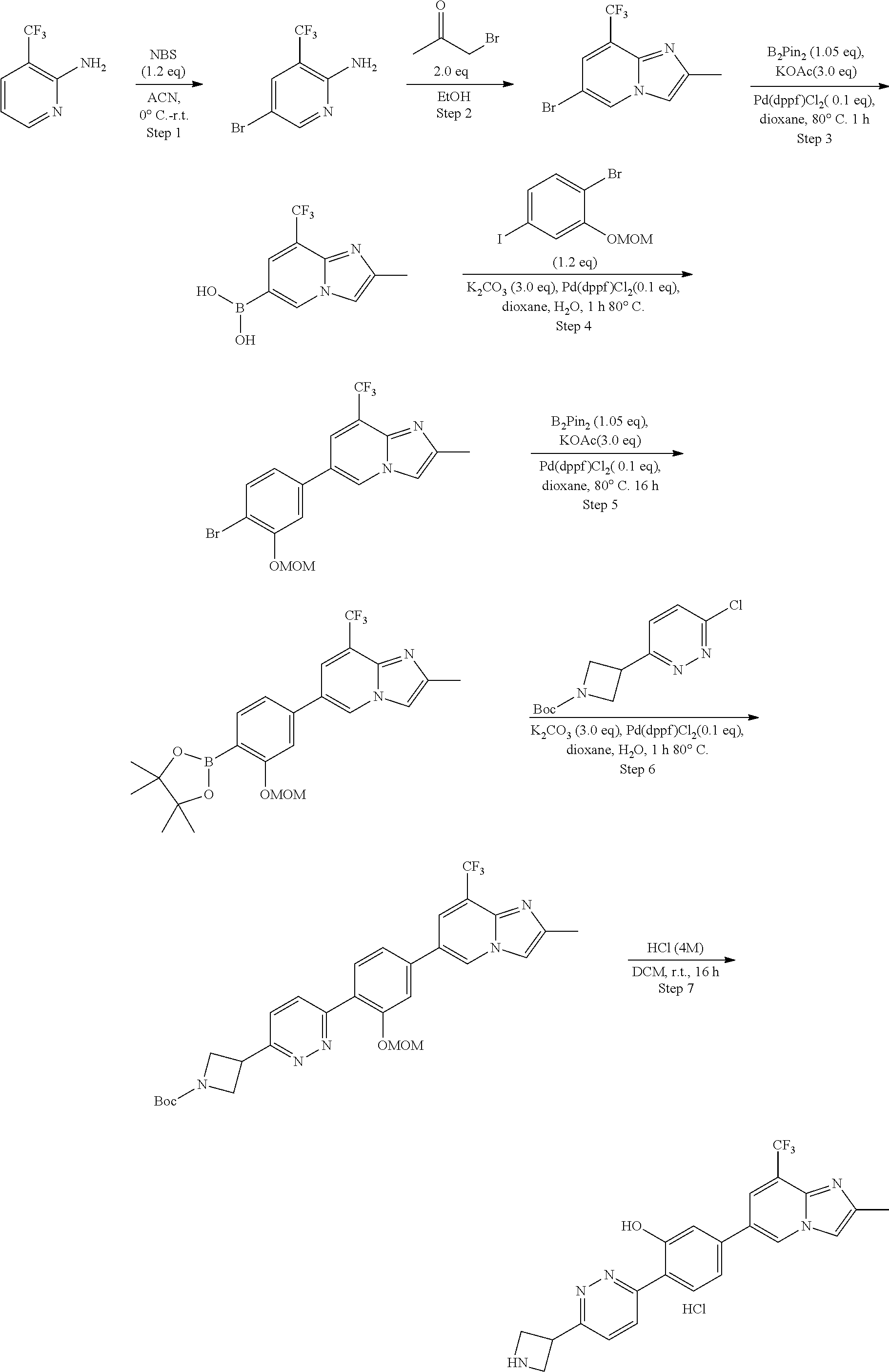

### Step 1: synthesis of 5-bromo-3-(trifluoromethyl)pyridin-2-amine

To a mixture of 3-(trifluoromethyl)pyridin-2-amine (3 g, 18.52 mmol) in ACN (30 mL) was added NBS (4.0 g, 22.22 mmol) at 0° C. The resulting mixture was stirred for 2 h at room temperature. The mixture was adjusted to pH=8 with saturated Na₂CO₃ aqueous solution, extracted with EtOAc (50 mL×3). The organic layers was concentrated. The residue was purified by silica gel column chromatography (PE:EA=10:1) to give title product (2.8 g, Y: 63%) as a yellow solid. ESI-MS (M+H)+: 241.0. ¹H NMR (400 MHz, DMSO) δ 8.28 (d, J=1.7 Hz, 1H), 7.91 (d, J=2.0 Hz, 1H), 6.73 (s, 2H).

### Step 2: synthesis of 6-bromo-2-methyl-8-(trifluoromethyl)imidazo[1,2-a]pyridine

To a suspension of 5-bromo-3-(trifluoromethyl)pyridin-2-amine (2.8 g, 11.62 mmol) in EtOH (25 mL) was added 1-bromopropan-2-one (3.2 g, 23.23 mmol). The reaction mixture was stirred overnight at 90° C. The mixture was allowed to cool down to room temperature and the precipitate was formed. The mixture was filtered to afford title product (2.4 g, Y: 74%) as a grey solid. ESI-MS (M+H)+: 278.9. ¹H NMR (400 MHz, DMSO-d6) δ 9.51 (s, 1H), 8.39 (s, 1H), 8.16 (d, J=0.8 Hz, 1H), 2.51 (d, J=0.7 Hz, 3H).

### Step 3: synthesis of (2-methyl-8-(trifluoromethyl)imidazo[1,2-a]pyridin-6-yl)boronic acid

A mixture of 6-bromo-2-methyl-8-(trifluoromethyl)imidazo[1,2-a]pyridine (500 mg, 1.79 mmol), B₂Pin₂ (477 mg, 1.88 mmol), KOAc (526 mg, 5.37 mmol) and Pd(dppf)Cl₂ (132 mg, 0.18 mmol) in dioxane (15 mL) at room temperature was purged with N₂ for three times. Then the reaction mixture was stirred at 80° C. for 1 h. The mixture was filtered and the filtration was used in the next step without further purification. ESI-MS (M+H)+: 245.0

### Step 4: synthesis of 6-(4-bromo-3-(methoxymethoxy)phenyl)-2-methyl-8-(trifluoromethyl)imidazo[1,2-a]pyridine

A mixture of (2-methyl-8-(trifluoromethyl)imidazo[1,2-a]pyridin-6-yl)boronic acid (solution from above step), 1-bromo-4-iodo-2-(methoxymethoxy)benzene (737 mg, 2.15 mmol), K₂CO₃ (741 mg, 5.37 mmol) and Pd(dppf)Cl₂ (132 mg, 0.18 mmol) in H₂O (4 ml) was stirred for 1 h at 80° C. under N₂. The mixture was allowed to cool down to room temperature and concentrated. The residue was purified by silica gel column chromatography (PE:EA=1:5) to give title product (420 mg, Y: 57%) as a yellow oil. ESI-MS (M+H)+: 414.9 ¹H NMR (400 MHz, DMSO-d6) δ 9.13 (s, 1H), 7.91-7.89 (m, 2H), 7.72 (d, J=8.3 Hz, 1H), 7.56 (d, J=2.0 Hz, 1H), 7.34 (dd, J=8.3, 2.0 Hz, 1H), 5.45 (s, 2H), 3.46 (s, 3H), 2.41 (s, 3H).

### Step 5: synthesis of 6-(3-(methoxymethoxy)-4-(4,4,5,5-tetramethyl-1,3,2-dioxaborolan-2-yl)phenyl)-2-methyl-8-(trifluoromethyl)imidazo[1,2-a]pyridine

A mixture of 6-(4-bromo-3-(methoxymethoxy)phenyl)-2-methyl-8-(trifluoromethyl)imidazo[1,2-a]pyridine (400 mg, 0.96 mmol), bis(pinacolato)diboron (257 mg, 1.01 mmol), potassium acetate (282 mg, 2.88 mmol) and Pd(dppf)Cl₂ (70 mg, 0.1 mmol) in dioxane (20 mL) was stirred at 80° C. (under Ar atmosphere) for 16 h. The mixture was allowed to cool down to room temperature and filtered. The filtrate was used in the next step without further purification. ESI-MS (M+H)+: 463.0.

### Step 6: synthesis of tert-butyl 3-(6-(2-(methoxymethoxy)-4-(2-methyl-8-(trifluoromethyl)imidazo[1,2-a]pyridin-6-yl)phenyl)pyridazin-3-yl)azetidine-1-carboxylate

To the above solution was added H₂O (2 mL), tert-butyl 3-(6-chloropyridazin-3-yl)azetidine-1-carboxylate and (259 mg, 0.96 mmol), K₂CO₃ (397 mg, 2.88 mmol) and Pd(dppf)Cl₂ (70 mg, 0.096 mmol). The resulting mixture was stirred for 1 h at 80° C. under N₂. The mixture was allowed to cool down to room temperature and concentration under reduced pressure. The residue was purified by silica gel column chromatography (DCM:MeOH=20:1) to give title product (130 mg, Y: 24% for two steps) as a yellow solid. ESI-MS (M+H)+: 570.2.

### Step 7: synthesis of 2-(6-(azetidin-3-yl)pyridazin-3-yl)-5-(2-methyl-8-(trifluoromethyl)imidazo[1,2-a]pyridin-6-yl)phenol hydrochloride

To a solution of tert-butyl 3-(6-(2-(methoxymethoxy)-4-(2-methyl-8-(trifluoromethyl)imidazo[1,2-a]pyridin-6-yl)phenyl)pyridazin-3-yl)azetidine-1-carboxylate (120 mg, 0.21 mmol) in DCM (10 mL) was added HCl in dioxane (2 mL) at 0° C. The mixture was stirred for 16 h at rt. After concentration, the residue was purified by pre-HPLC to afforded title product (6.4 mg, Y: 10%) as white solid. ESI-MS (M+H)+: 426.4 ¹H NMR (400 MHz, MeOD) δ 9.46 (s, 1H), 8.72-8.62 (m, 2H), 8.24 (d, J=0.8 Hz, 1H), 8.14-8.12 (m, 2H), 7.56-7.50 (m, 2H), 4.63-4.52 (m, 5H), 2.65 (s, 3H).

### Example 6. Synthesis of 2-(6-(azetidin-3-yl)pyridazin-3-yl)-5-(2,7-dimethyl-2H-indazol-5-yl)phenol hydrochloride (Compound 6)

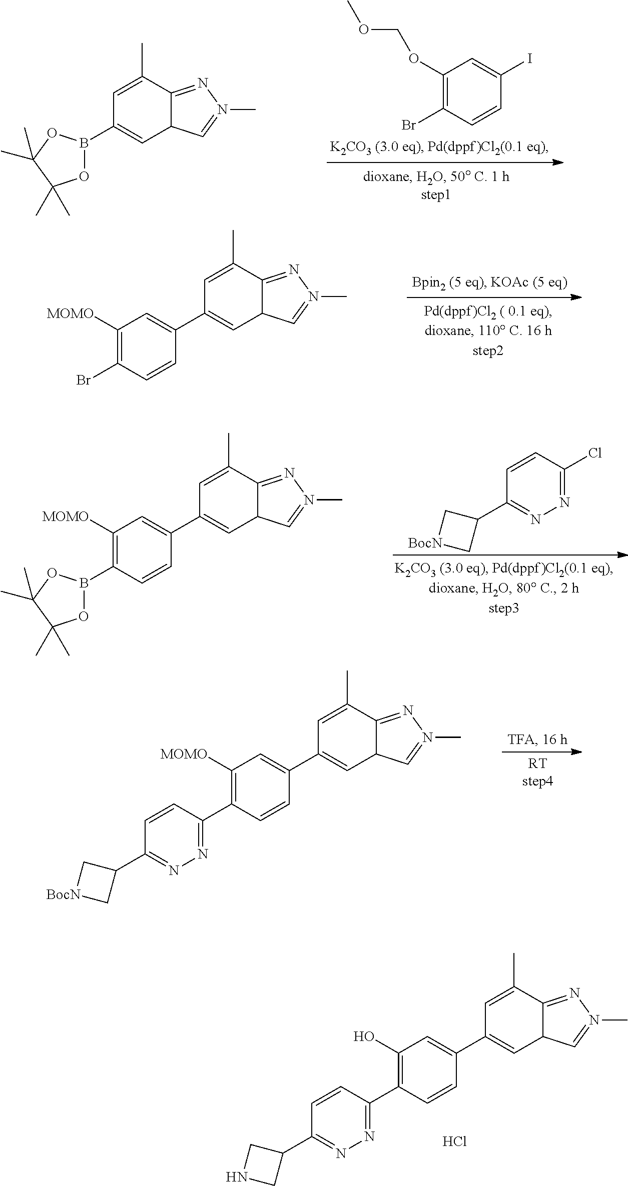

### Step 1: Preparation of 5-(4-bromo-3-(methoxymethoxy)phenyl)-2,7-dimethyl-2H-indazole

To a mixture of 2,7-dimethyl-5-(4,4,5,5-tetramethyl-1,3,2-dioxaborolan-2-yl)-2H-indazole (5.30 g, 19.48 mmol) and 1-bromo-4-iodo-2-(methoxymethoxy) benzene (6.60 g, 19.48 mmol) in dioxane: H₂O (90 mL:15 mL) were added Pd(dppf)Cl₂ (712 mg, 0.97 mmol) and K₂CO₃ (8.00 g, 58.34 mmol). The mixture was stirred at 50° C. for 1 h. LCMS showed the starting material was consumed completely. The mixture was filtered and the filtrate was concentrate in vacuo. The residue was purified by silica gel column chromatography (PE:EA=1:1) to give title product (4.0 g, Y: 58%) as a brown solid. ESI-MS (M+H+): 363.0. ¹H NMR (400 MHz, DMSO-d6) δ 8.36 (s, 1H), 7.85-7.69 (d, J=13.9 Hz, 1H), 7.70-7.59 (s, 1H), 7.51-7.45 (s, 1H), 7.33 (s, 1H), 7.28-7.19 (m, 1H), 5.38 (s, 2H), 4.18 (s, 3H), 3.43 (s, 3H), 2.55 (s, 3H).

### Step 2: Preparation of 5-(3-(methoxymethoxy)-4-(4,4,5,5-tetramethyl-1,3,2-dioxaborolan-2-yl)phenyl)-2,7-dimethyl-2H-indazole

To a mixture of 5-(4-bromo-3-(methoxymethoxy)phenyl)-2,7-dimethyl-2H-indazole (4.00 g, 11.11 mmol) and B₂pin₂ (28.00 g, 111.11 mmol) in dioxane (200 mL) were added Pd(dppf)Cl₂ (406 mg, 0.55 mmol) and KOAc (8.70 g, 88.88 mmol). The mixture was stirred at 110° C. for 16 h. LCMS showed the starting material was consumed completely. The mixture was filtered and the filtrate was concentrate in vacuo. The residue was purified by Flash chromatography (PE:EA=1:1) to give title product (1.6 g, Y: 35.5%) as a brown solid. ESI-MS (M+H+): 409.0. ¹H NMR (400 MHz, CDCl₃) δ 7.91 (s, 1H), 7.76 (d, J=7.5 Hz, 1H), 7.68 (s, 1H), 7.31 (d, J=1.5 Hz, 1H), 7.29 (d, 1H), 7.26 (s, 1H), 5.28 (s, 2H), 4.25 (s, 3H), 3.56 (s, 3H), 2.68 (s, 3H), 1.37 (s, 12H).

### Step 3: Preparation of tert-butyl 3-(6-(4-(2,7-dimethyl-2H-indazol-5-yl)-2-(methoxymethoxy)phenyl)pyridazin-3-yl)azetidine-1-carboxylate

To a mixture of 5-(3-(methoxymethoxy)-4-(4,4,5,5-tetramethyl-1,3,2-dioxaborolan-2-yl)phenyl)-2,7-dimethyl-2H-indazole (1.60 g, 3.92 mmol) and tert-butyl 3-(6-chloropyridazin-3-yl) azetidine-1-carboxylate (1.06 g, 3.92 mmol) in dioxane: H₂O (63 mL:9 mL) were added Pd(dppf)Cl₂ (196 mg, 0.20 mmol) and K₂CO₃ (1.62 g, 11.76 mmol). The mixture was stirred at 80° C. for 2 h. LCMS showed the starting material was consumed completely. The mixture was filtered and the filtrate was concentrate in vacuo. The residue was purified by silica gel column chromatography (DCM:MeOH=40:1) to give title product (1.40 g, Y: 70%) as a yellow solid. ESI-MS (M+H+): 516.2. ¹H NMR (400 MHz, CDCl₃) δ 8.10-8.05 (m, 2H), 7.95 (d, J=4.1 Hz, 1H), 7.75-7.72 (m, 1H), 7.51 (d, J=8.8 Hz, 2H), 7.39-7.34 (m, 2H), 5.31 (s, 2H), 4.48-4.37 (m, 3H), 4.27 (s, 3H), 4.21-4.09 (m, 2H), 3.49 (s, 3H), 2.70 (s, 3H), 1.48 (s, 9H).

### Step 4: Preparation of 2-(6-(azetidin-3-yl)pyridazin-3-yl)-5-(2-methyl-2H-pyrazolo[3,4-c]pyridin-5-yl)phenol hydrochloride

A mixture of tert-butyl 3-(6-(4-(2,7-dimethyl-2H-indazol-5-yl)-2-(methoxymethoxy)phenyl)pyridazin-3-yl)azetidine-1-carboxylate (1.30 g, 2.52 mmol) in TFA (15 mL) was stirred at rt for 16 h. LCMS showed the starting material was consumed completely. The mixture was concentrated in vacuo and the crude was purified by pre-HPLC to give title product (500 mg, Y: 54%) as a yellow solid. ESI-MS (M+H+): 372.1. ¹H NMR (400 MHz, MeOD-d4) δ 9.02-8.88 (m, 2H), 8.51 (d, J=8.9 Hz, 1H), 8.17 (s, 1H), 8.00 (d, J=8.2 Hz, 1H), 7.91 (s, 1H), 7.53 (d, J=8.1 Hz, 1H), 7.47 (s, 1H), 4.75-4.67 (m, 1H), 4.63-4.54 (m, 4H), 4.47 (s, 3H), 2.72 (s, 3H).

### Example 7. 5-{4-[6-(azetidin-3-yl)pyridazin-3-yl]-3-hydroxyphenyl}-1,3-dimethyl-1,2-dihydropyridin-2-one (Compound 7)

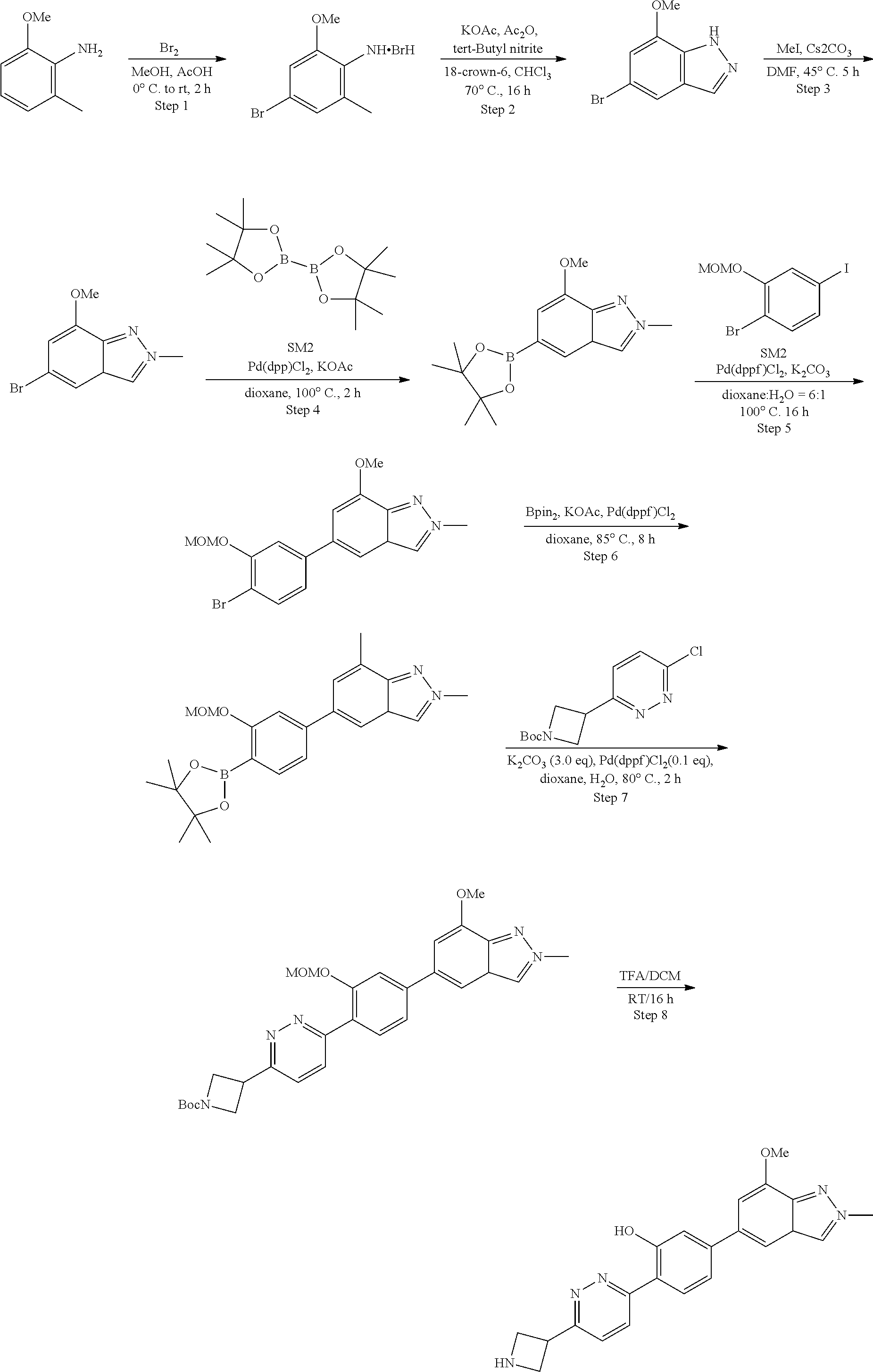

### Step 1: Preparation of 4-bromo-2-methoxy-6-methylaniline hydrobromide

To a solution of 2-methoxy-6-methylaniline (25 g, 182 mmol) in MeOH (250 mL) was added Br₂ (29.25 g, 182 mmol) in AcOH (50 mL) dropwised at RT, the mixture was stirred for 2 h at RT. The reaction mixture was diluted with EtOAc (1000 mL) and stirred for 5 min. The solid was collected by filtration, washed with EtOAc (100 mL×2) to afford title compound (44.4 g, 83% yield) as a pink solid. ESI-MS (M+H)+: 216.0.

### Step 2: Preparation of 5-bromo-7-methoxy-1H-indazole

To a solution of 4-bromo-2-methoxy-6-methylaniline hydrobromide (10 g, 46.512 mmol) in CHCl₃ (200 mL) was added KOAc (5.47 g, 55.814 mmol), Ac₂O (18.977 g, 186.048 mmol), the mixture was stirred for 1 h at RT, then stirred at 70° C. for 2 h. ter-Butyl-nitrile (9.6 g, 93.024 mmol) and 18-crown-6 (982 mg, 3.721 mmol) was added, the solution was stirred at 70° C. for 16 h. Then the mixture was added aqueous K₂CO₃ (38.512 g, 279.072 mmol) solution and stirred at RT for 5 h. The reaction mixture was diluted with water (100 mL) and extracted with DCM (200 mL×3). The organic layer was washed with brine, dried over Na₂SO₄ and evaporated to give title compound (8.7 g, Y: 83%) as a gray solid. ESI-MS (M+H)+: 226.9.

### Step 3: Preparation of 5-bromo-7-methoxy-2-methyl-2H-indazole

To a solution of 5-bromo-7-methoxy-1H-indazole (45 g, crude) in DMF (250 mL) was added Cs₂CO₃ (108 g, 331.85 mmol), after 15 min, Mel (24 g, 165.93 mmol) was added, the mixture was stirred for 5 h at 45° C. The reaction mixture was diluted with water (1300 mL) and extracted with EtOAc (1300 mL×3). The organic layer was washed with brine, dried over Na₂SO₄ and evaporated to give crude title compound. The crude was purified by silica gel column chromatography eluted with (EtOAc/PE=50% to 100%) to give title compound (6.03 g, 11% yield) as a brown solid, ESI-MS (M+H)+: 241.9. ¹H NMR (400 MHz, CDCl₃) δ 7.79 (s, 1H), 7.37 (d, J=1.2 Hz, 1H), 6.63 (d, J=1.0 Hz, 1H), 4.19 (s, 3H), 4.00 (s, 3H).

### Step 4: Preparation of 7-methoxy-2-methyl-5-(4,4,5,5-tetramethyl-1,3,2-dioxaborolan-2-yl)-2H-indazole

To a mixture of 5-bromo-7-methoxy-2-methyl-2H-indazole (6.03 g, 25 mmol), B₂pin₂ (16 g, 62.5 mmol) in 1,4-dioxane (250 mL) were added KOAc (8.6 g, 87.5 mmol) and Pd(dppf)Cl₂ (1.83 g, 2.5 mmol), the mixture was charged with N₂ for three times and stirred at 100° C. for 2 h under N₂. The reaction mixture was used to next step without purification further. ESI-MS (M+H)+: 288.7.

### Step 5: Preparation of 5-(4-bromo-3-(methoxymethoxy)phenyl)-7-methoxy-2-methyl-2H-indazole

To a mixture of 7-methoxy-2-methyl-5-(4,4,5,5-tetramethyl-1,3,2-dioxaborolan-2-yl)-2H-indazole (6.5 g, 22.56 mmol) in 1,4-dioxane (230 mL) were added 1-bromo-4-iodo-2-(methoxymethoxy)benzene (10 g, 29.34 mmol), Pd(dppf)Cl₂ (1.642 g, 2.25 mmol), K₂CO₃ (9.344 g, 67.70 mmol) and water (38 mL), the mixture was charged with N₂ for three times and stirred at 100° C. for 16 h. The reaction mixture was diluted with water (150 mL) and extracted with EtOAc (200 mL×3). The organic layer was washed with brine, dried over Na₂SO₄ and evaporated to give crude title compound. The crude was purified by silica gel column chromatography (PE:EA=5:1-1:1) to give title product (5 g, Y: 8.8% for two steps) as a brown solid. ESI-MS (M+H)+: 378.9. ¹H NMR (400 MHz, CDCl₃) δ 7.89 (s, 1H), 7.59 (d, J=8.2 Hz, 1H), 7.40 (d, J=2.1 Hz, 1H), 7.36 (d, J=1.3 Hz, 1H), 7.17-7.14 (m, 1H), 6.73 (d, J=1.2 Hz, 1H), 5.33 (s, 2H), 4.23 (s, 3H), 4.07 (s, 3H), 3.57 (s, 3H).

### Step 6: Preparation of 7-methoxy-5-(3-(methoxymethoxy)-4-(4,4,5,5-tetramethyl-1,3,2-dioxaborolan-2-yl)phenyl)-2-methyl-2H-indazole

To a mixture of 5-(4-bromo-3-(methoxymethoxy)phenyl)-7-methoxy-2-methyl-2H-indazole (2.27 g, 6.04 mmol), B₂pin₂ (3.06 g, 12.07 mmol) in 1,4-dioxane (80 mL) were added Pd(dppf)Cl₂ (438 mg, 0.604 mmol) and KOAc (1.78 g, 18.12 mmol), the mixture was charged with N₂ for three times and stirred at 85° C. for 8 h under N₂. The reaction mixture was diluted with water (100 mL) and extracted with EtOAc (100 mL×3). The organic layer was washed with brine, dried over Na₂SO₄ and evaporated to give crude title compound. The crude was purified by silica gel column chromatography (PE:EA=4:1-1:1) to give title product (2.88 g, crude) as a brown oil. ESI-MS (M+H)+: 425.2

### Step 7: Preparation of tert-butyl 3-(6-(4-(7-methoxy-2-methyl-2H-indazol-5-yl)-2-(methoxymethoxy)phenyl)pyridazin-3-yl)azetidine-1-carboxylate

To a mixture of 7-methoxy-5-(3-(methoxymethoxy)-4-(4,4,5,5-tetramethyl-1,3,2-dioxaborolan-2-yl)phenyl)-2-methyl-2H-indazole (2.68 g, 6.32 mmol) and tert-butyl 3-(6-chloropyridazin-3-yl)azetidine-1-carboxylate (1.87 g, 6.95 mmol) and K₂CO₃ (2.62 g, 18.96 mmol) in 1,4-dioxane: H2O (67 mL:13 mL) was added Pd(dppf)Cl₂ (461 mg, 0.632 mmol). The mixture was stirred at 80° C. under N₂ for 2 h. The mixture was allowed to cooling down to room temperature, concentrated under reduced pressure, added water (100 mL), extracted with EA (100 mL×3), the combined organic layer was washed brine (100 mL), dried over sodium sulfate, filtered and concentrated in vacuo. The residue was purified by silica gel column chromatography (PE:EA=2:1-1:3) to give title product (2.43 g, Y: 68%) as a white solid. ESI-MS (M+H)+: 532.2. ¹H NMR (400 MHz, CDCl₃) δ 8.09 (d, J=1.2 Hz, 1H), 8.07 (s, 1H), 7.92 (s, 1H), 7.51 (d, J=8.7 Hz, 2H), 7.47-7.45 (m, 2H), 6.84 (d, J=0.9 Hz, 1H), 5.31 (s, 2H), 4.43 (t, J=8.6 Hz, 2H), 4.32-4.28 (m, 2H), 4.25 (s, 3H), 4.17-4.13 (m, 1H), 4.10 (s, 3H), 3.48 (s, 3H), 1.48 (s, 9H).

### Step 8: Preparation of 2-(6-(azetidin-3-yl)pyridazin-3-yl)-5-(7-methoxy-2-methyl-2H-indazol-5-yl)phenol

To a solution of tert-butyl 3-(6-(4-(7-methoxy-2-methyl-2H-indazol-5-yl)-2-(methoxymethoxy)phenyl)pyridazin-3-yl)azetidine-1-carboxylate (2.2 g, 4.143 mmol) in DCM (15 mL) was added TFA (15 mL). The resulting mixture was stirred at RT for 16 h. The mixture was diluted with DCM (60 mL) and concentrated in vacuo and lyophilized to give title product (3.0 g, crude) as an orange solid. 200 mg crude was purified by prep-HPLC (0.05% FA in water/CH₃CN) to give title product (30 mg, 25%) as a yellow solid. ESI-MS (M+H)+: 388.2. ¹H NMR (400 MHz, DMSO-d6) δ 8.55 (d, J=8.0 Hz, 1H), 8.38-8.36 (m, 2H), 8.10 (d, J=8.0 Hz, 1H), 7.95 (d, J=8.0 Hz, 1H), 7.60 (s, 1H), 7.37-7.36 (m, 2H), 6.93 (s, 1H), 4.45-4.32 (m, 1H), 4.16 (s, 3H), 4.15-4.13 (m, 4H), 4.02 (s, 3H).

### Example 8. 2-[6-(azetidin-3-yl)pyridazin-3-yl]-4-fluoro-5-{8-fluoro-2-methylimidazo[1,2-a]pyridin-6-yl}phenol (Compound 8)

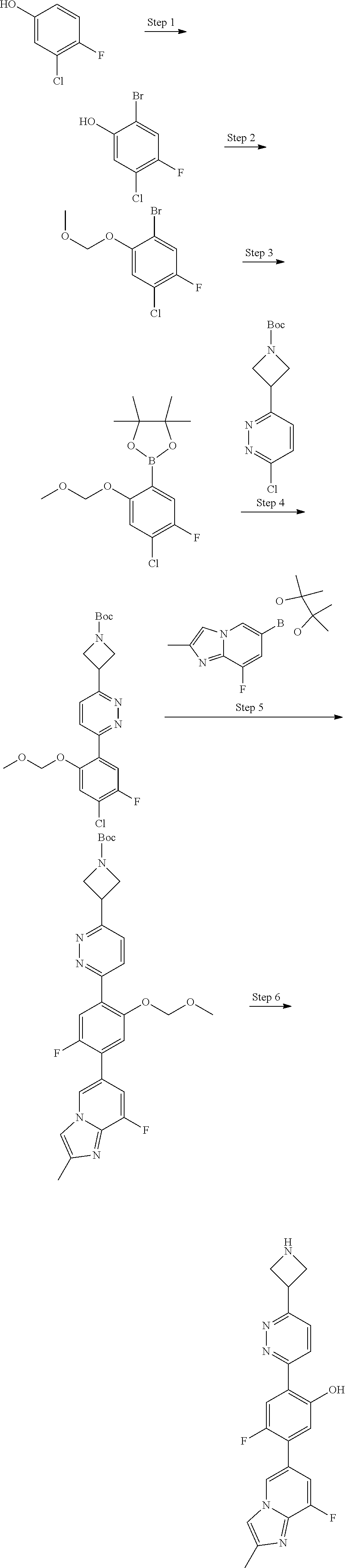

### Step 1: Preparation of 2-bromo-5-chloro-4-fluorophenol

In a 0.5 L reaction flask, 3-chloro-4-fluorophenol (10.0 g, 68.24 mmol) and chloroform (200 mL) were added, and the mixture was cooled to −10° C. Bromine (12 g, 75 mmol, 3.9 mL) was added dropwise maintaining the temperature of the solution in the reaction flask below 0° C. After the addition was completed, the reaction mixture was stirred at 0° C. for 3 hours. After that a saturated NaHSO₃ aqueous solution (100 mL) was added to the reaction solution and the organic layer was separated, washed with a saturated aqueous NaCl solution (100 mL) dried over anhydrous Na₂SO₄ and concentrated to dryness under reduced pressure to give crude 2-bromo-5-chloro-4-fluorophenol (12.46 g, Y: 99.7%) which was used in the next step without purification. ¹H NMR (500 MHz, DMSO-d6) δ 10.57 (s, 1H), 7.57 (d, J=8.8 Hz, 1H), 7.00 (d, J=6.8 Hz, 1H).

### Step 2: Preparation of 1-bromo-4-chloro-5-fluoro-2-(methoxymethoxy)benzene

2-Bromo-5-chloro-4-fluorophenol (5.9 g, 26.17 mmol) was dissolved in anhydrous CH₂C₁₂ (150 mL). Ethylbis(propan-2-yl)amine (4.06 g, 31.41 mmol) was then added at r.t. The mixture was cooled to 0° C. and chloro(methoxy)methane (2.53 g, 31.41 mmol) was added dropwise. The reaction mixture was stirred at 0° C. for 30 min, then allowed to warm to r.t. and stirred for 11 h. After that the resulting mixture was quenched with sat. aq. NaHCO₃ (250 mL), and the organic layer was separated, washed with brine (200 mL) and dried over anhydrous Na₂SO₄. The solvent was removed under reduced pressure to afford a yellow oil which was purified by column chromatography (eluent Hex/EtOAc 30/1, Rf˜0.4) to provide 1-bromo-4-chloro-5-fluoro-2-(methoxymethoxy)benzene (1.58 g, Y: 23.1% yield) as white solid. ¹H NMR (500 MHz, Chloroform-d) δ 7.36 (d, J=8.2 Hz, 1H), 7.22 (d, J=6.7 Hz, 1H), 5.19 (s, 2H), 3.52 (s, 3H).

### Step 3: Preparation of 2-[4-chloro-5-fluoro-2-(methoxymethoxy)phenyl]-4,4,5,5-tetramethyl-1,3,2-dioxaborolane

A solution of 1-bromo-4-chloro-5-fluoro-2-(methoxymethoxy)benzene (1.58 g, 5.86 mmol), 4,4,5,5-tetramethyl-2-(4,4,5,5-tetramethyl-1,3,2-dioxaborolan-2-yl)-1,3,2-dioxaborolane (1.49 g, 5.86 mmol), potassium acetate (1.73 g, 17.59 mmol), and [1,1′-bis(diphenylphosphino)ferrocene]dichloropalladium (II) dichloromethane adduct (239.4 mg, 293.15 μmol) in DMSO (20 mL) was degassed and purged with Ar. The reaction mixture was heated overnight at 90° C. The resulting mixture was poured into water with ice (40 mL) and extracted with EtOAc (20 mL). The organic layer was washed with brine (15 mL), dried over Na₂SO₄ and evaporated under reduced pressure to give the crude which was purified by column chromatography using Hex/EtOAc (8/1) as an eluent to give 2-[4-chloro-5-fluoro-2-(methoxymethoxy)phenyl]-4,4,5,5-tetramethyl-1,3,2-dioxaborolane (900.0 mg, Y: 43.6%) as yellow oil. ¹H NMR (500 MHz, DMSO-d6) δ 7.37 (d, J=8.2, 1H), 7.28 (d, J=6.7, 1H), 5.15 (s, 2H), 3.39 (s, 3H), 1.26 (s, 12H).

### Step 4: Preparation of tert-butyl 3-6-[4-chloro-5-fluoro-2-(methoxymethoxy)phenyl]pyridazin-3-yl-azetidine-1-carboxylate

A solution of 2-[4-chloro-5-fluoro-2-(methoxymethoxy)phenyl]-4,4,5,5-tetramethyl-1,3,2-dioxaborolane (450.35 mg, 1.42 mmol), tert-butyl 3-(6-chloropyridazin-3-yl)azetidine-1-carboxylate (383.72 mg, 1.42 mmol), [1,1′-bis(diphenylphosphino)ferrocene]dichloropalladium(II) dichloromethane adduct (58.09 mg, 71.13 μmol), and potassium carbonate (393.23 mg, 2.85 mmol) in dioxane (15 mL) and water (2 mL) was degassed and purged with Ar. The reaction mixture was heated overnight at 90° C. The resulting mixture was cooled to r.t. and filtered through a pad of Celite. The filtrate was concentrated in vacuo and the residue was purified by column chromatography using Hex/EtOAc 1/1 as an eluent to give tert-butyl 3-6-[4-chloro-5-fluoro-2-(methoxymethoxy)phenyl]pyridazin-3-ylazetidine-1-carboxylate (440.0 mg, Y: 65.7%). ESI-MS (M+H)+: 424.2. ¹H NMR (400 MHz, Chloroform-d) δ 8.01 (d, J=8.8 Hz, 1H), 7.84 (d, J=9.6 Hz, 1H), 7.50 (d, J=8.8 Hz, 1H), 7.33 (d, J=6.0 Hz, 1H), 5.15 (s, 2H), 4.40 (t, J=8.6 Hz, 2H), 4.27-4.18 (m, 2H), 4.15-4.07 (m, 1H), 3.43 (s, 3H), 1.45 (s, 9H).

### Step 5: Preparation of tert-butyl 3-[6-(5-fluoro-4-8-fluoro-2-methylimidazo[1,2-a]pyridin-6-yl-2-(methoxymethoxy)phenyl)pyridazin-3-yl]azetidine-1-carboxylate

A solution of tert-butyl 3-6-[4-chloro-5-fluoro-2-(methoxymethoxy)phenyl]pyridazin-3-ylazetidine-1-carboxylate (439.98 mg, 1.04 mmol), 8-fluoro-2-methyl-6-(tetramethyl-1,3,2-dioxaborolan-2-yl)imidazo[1,2-a]pyridine (343.94 mg, 1.25 mmol), [1,1′-bis(diphenylphosphino)ferrocene]dichloropalladium (II) dichloromethane adduct (42.38 mg, 51.9 μmol), and cesium carbonate (1.01 g, 3.11 mmol) in dioxane (15 mL) and water (2 mL) was degassed and purged with Ar. The reaction mixture was heated overnight at 90° C. The resulting mixture was cooled to r.t., diluted with MTBE (15 mL) and filtered through a pad of Celite. The filtrate was concentrated in vacuo to give the crude, which was purified by preparative HPLC to give tert-butyl 3-[6-(5-fluoro-4-8-fluoro-2-methylimidazo[1,2-a]pyridin-6-yl-2-(methoxymethoxy)phenyl)pyridazin-3-yl]azetidine-1-carboxylate (92.0 mg, Y: 14.8%). ESI-MS (M+H)+: 538.0.

### Step 6: Preparation of 2-[6-(azetidin-3-yl)pyridazin-3-yl]-4-fluoro-5-8-fluoro-2-methylimidazo[1,2-a]-pyridin-6-ylphenol as trifluoroacetate salt

tert-Butyl 3-[6-(5-fluoro-4-8-fluoro-2-methylimidazo[1,2-a]pyridin-6-yl-2-(methoxymethoxy)phenyl)pyridazin-3-yl]azetidine-1-carboxylate (91.96 mg, 171.06 μmol) was dissolved in CH₂Cl₂ (3 mL), and 2,2,2-trifluoroacetic acid (195.05 mg, 1.71 mmol) was added. The reaction mixture was stirred overnight at r.t. and then evaporated to dryness to provide the crude material. The residue was triturated with MeCN/MTBE (1/4, 2 mL) and the resulting precipitate was filtered and dried in vacuo to give pure 2-[6-(azetidin-3-yl)pyridazin-3-yl]-4-fluoro-5-8-fluoro-2-methylimidazo[1,2-a]pyridin-6-ylphenol as trifluoroacetate salt (35.0 mg, Y: 40.3%). ESI-MS (M+H)+: 394.2. ¹H NMR (500 MHz, DMSO-d6) δ 12.68 (s, 1H), 9.1-8.5 (m, 3H), 8.2-7.8 (m, 3H), 7.5-.7.3 (m, 1H), 7.3-7.2 (m, 1H), 4.55-4.25 (m, 5H), 2.39 (s, 3H).

### Example 9. 2-[6-(azetidin-3-yl)pyridazin-3-yl]-5-{2,8-dimethylimidazo[1,2-a]pyrazin-6-yl}phenol (Compound 9)

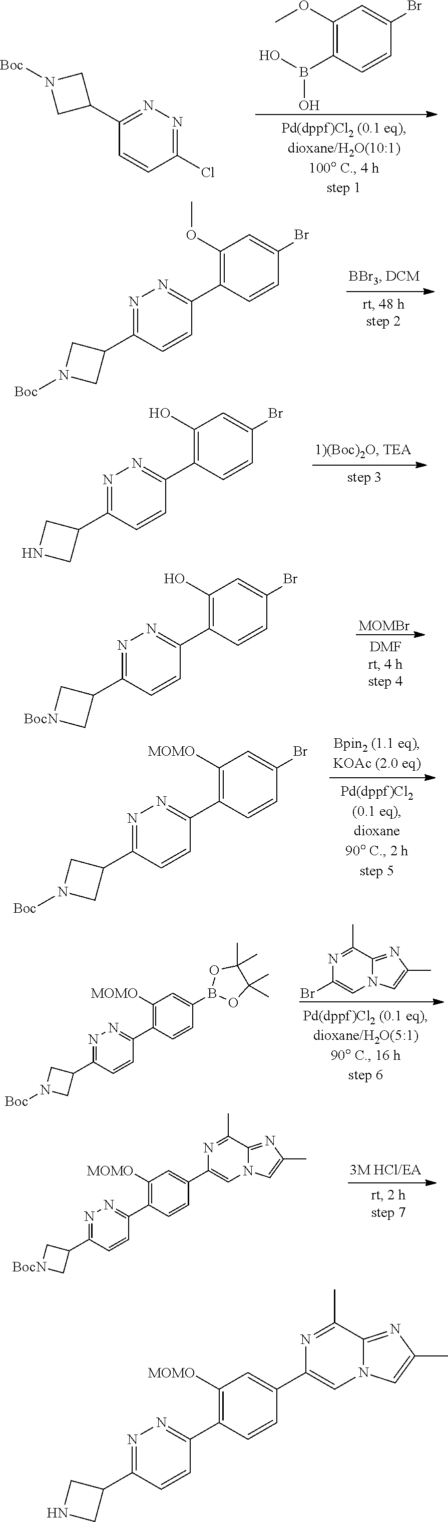

### Step 1: Preparation of tert-butyl 3-(6-(4-bromo-2-methoxyphenyl)pyridazin-3-yl)azetidine-1-carboxylate

To a mixture of tert-butyl 3-(6-chloropyridazin-3-yl)azetidine-1-carboxylate (2.0 g, 7.43 mmol) in 1,4-dioxane (50 mL) was added (4-bromo-2-methoxyphenyl)boronic acid (1.88 g, 8.18 mmol), Pd(dppf)Cl₂ (540 mg, 0.74 mmol), K₂CO₃ (3.08 g, 22.3 mmol) and water (5 mL), the mixture was charged with N₂ for three times and stirred at 100° C. for 4 h. The reaction mixture was diluted with water (50 mL) and extracted with EtOAc (50 mL×3). The organic layer was washed with brine, dried over Na₂SO₄ and evaporated to give crude title compound. The crude was purified column chromatography eluted with (PE/EA=1:1) to give title compound (1.2 g, 38.7% yield) as a yellow solid, ESI-MS (M+H)+: 422.1. ¹H NMR (400 MHz, CDCl₃) δ 8.00 (d, J=8.8 Hz, 1H), 7.89 (d, J=8.3 Hz, 1H), 7.47 (d, J=8.8 Hz, 1H), 7.28 (dd, J=8.3, 1.8 Hz, 1H), 7.17 (d, J=1.7 Hz, 1H), 4.41 (t, J=8.6 Hz, 2H), 4.33-4.20 (m, 2H), 4.15-4.10 (m, 1H), 3.88 (s, 3H), 1.47 (s, 9H).

### Step 2: Preparation of 2-(6-(azetidin-3-yl)pyridazin-3-yl)-5-bromophenol

A mixture of tert-butyl 3-(6-(4-bromo-2-methoxyphenyl)pyridazin-3-yl)azetidine-1-carboxylate (1.2 g, 2.88 mmol) in BBr₃ (28.8 mL, 1.0 M in DCM) was stirred at rt for 48 h. The mixture was concentrated in vacuo to give title compound (900 mg, crude) as a yellow solid, ESI-MS (M+H)+: 305.8.

### Step 3: Preparation of tert-butyl 3-(6-(4-bromo-2-hydroxyphenyl)pyridazin-3-yl)azetidine-1-carboxylate

To a mixture of 2-(6-(azetidin-3-yl)pyridazin-3-yl)-5-bromophenol (900 mg, 2.95 mmol) in DCM (20 mL) was added TEA (5.96 g, 59.0 mmol), Boc₂O (3.22 g, 14.75 mmol) was added dropwise to the mixture and stirred at rt overnight. The reaction mixture was diluted with water (30 mL) and extracted with DCM (20 mL×3). The organic layer was washed with brine, dried over Na₂SO₄ and evaporated to give crude title compound. The crude was purified by column chromatography eluted with (PE/EA=2:1) to give title compound (500 mg, 41.7% yield) as a yellow solid, ESI-MS (M+H)+: 407.8. ¹H NMR (400 MHz, CDCl₃) δ 13.74 (s, 1H), 8.05 (d, J=9.2 Hz, 1H), 7.66 (d, J=9.1 Hz, 1H), 7.57 (d, J=8.6 Hz, 1H), 7.30 (d, J=2.0 Hz, 1H), 7.10 (dd, J=8.5, 2.0 Hz, 1H), 4.44 (t, J=8.7 Hz, 2H), 4.27-4.18 (m, 2H), 4.14-4.08 (m, 1H), 1.48 (s, 9H).

### Step 4: Preparation of tert-butyl 3-(6-(4-bromo-2-(methoxymethoxy)phenyl)pyridazin-3-yl)azetidine-1-carboxylate

To a mixture of tert-butyl 3-(6-(4-bromo-2-hydroxyphenyl)pyridazin-3-yl)azetidine-1-carboxylate (500 mg, 1.23 mmol) in DMF (20 mL) was added K₂CO₃ (509 mg, 3.69 mmol), MOMBr (306 mg, 2.46 mmol) was added dropwise to the mixture and stirred at rt overnight. The reaction mixture was diluted with water (30 mL) and extracted with EtOAc (20 mL×3). The organic layer was washed with brine, dried over Na₂SO₄ and evaporated to give crude title compound. The crude was purified column chromatography eluted with (PE/EA=2:1) to give title compound (100 mg, 18.1% yield) as a yellow solid, ESI-MS (M+H)+: 452.1. ¹H NMR (400 MHz, CDCl₃) δ 7.99 (s, 1H), 7.86 (d, J=8.3 Hz, 1H), 7.48 (s, 1H), 7.44 (d, J=1.8 Hz, 1H), 7.33 (dd, J=8.3, 1.8 Hz, 1H), 5.21 (s, 2H), 4.41 (d, J=8.6 Hz, 2H), 4.30-4.25 (m, 2H), 4.12 (d, J=5.1 Hz, 1H), 3.45 (s, 3H), 1.47 (s, 9H).

### Step 5: Preparation of tert-butyl 3-(6-(2-(methoxymethoxy)-4-(4,4,5,5-tetramethyl-1,3,2-dioxaborolan-2-yl)phenyl)pyridazin-3-yl)azetidine-1-carboxylate

To a mixture of) tert-butyl 3-(6-(4-bromo-2-(methoxymethoxy)phenyl)pyridazin-3-yl)azetidine-1-carboxylate (100 mg, 0.22 mmol), B₂pin₂ (145 mg, 0.572 mmol) in 1,4-dioxane (5 mL) was added KOAc (84.1 mg, 0.858 mmol), Pd(dppf)Cl₂ (21 mg, 0.029 mmol), the mixture was charged with N₂ for three times and stirred at 90° C. for 2 h under N₂. The reaction mixture was used to next step without purification further. ESI-MS (M+H)+: 498.3.

### Step 6: Preparation of tert-butyl 3-(6-(4-(2,8-dimethylimidazo[1,2-a]pyrazin-6-yl)-2-(methoxymethoxy)phenyl)pyridazin-3-yl)azetidine-1-carboxylate

To a mixture of tert-butyl 3-(6-(2-(methoxymethoxy)-4-(4,4,5,5-tetramethyl-1,3,2-dioxaborolan-2-yl)phenyl) pyridazin-3-yl)azetidine-1-carboxylate (0.286 mmol) in 1,4-dioxane (5 mL) was added 6-bromo-2,8-dimethylimidazo[1,2-a]pyrazine (64 mg, 0.286 mmol), Pd(dppf)Cl₂ (20.4 mg, 0.029 mmol), K₂CO₃ (118 mg, 0.858 mmol) and water (0.5 mL), the mixture was charged with N₂ for three times and stirred at 90° C. for 16 h. The reaction mixture was diluted with water (20 mL) and extracted with EtOAc (20 mL×3). The organic layer was washed with brine, dried over Na₂SO₄ and evaporated to give crude title compound. The crude was purified C₁₈ column chromatography eluted with (MeCN:H2O=30% to 60%) to give title compound (40 mg, 27% yield for two steps) as a yellow solid, ESI-MS (M+H)+: 517.3.

### Step 7: Preparation of 2-(6-(azetidin-3-yl)pyridazin-3-yl)-5-(2,8-dimethylimidazo[1,2-a]pyrazin-6-yl)phenol

To a solution of tert-butyl 3-(6-(4-(2,8-dimethylimidazo[1,2-a]pyrazin-6-yl)-2-(methoxymethoxy)phenyl)pyridazin-3-yl)azetidine-1-carboxylate (40 mg, 0.077 mmol) in EtOAc (1 mL) was added 3M HCl/EtOAc (2 mL) and the mixture was stirred for 2 h at RT. The mixture was concentrated in vacuo, the residue was purified by prep-HPLC (0.05% NH₃·H₂O in water/CH₃CN) afford title compound (10 mg, 35.7%) as a yellow solid. ¹H NMR (400 MHz, MeOD-d4) δ 8.78 (s, 1H), 8.40 (d, J=9.2 Hz, 1H), 8.00 (d, J=8.4 Hz, 1H), 7.83 (d, J=9.1 Hz, 1H), 7.77 (s, 1H), 7.63 (d, J=1.7 Hz, 1H), 7.59 (dd, J=8.3, 1.8 Hz, 1H), 4.36 (t, J=8.1 Hz, 1H), 4.16-4.07 (m, 4H), 2.85 (s, 3H), 2.47 (s, 3H). ESI-MS (M+H)+: 373.0

### Example 10. 6-[6-(azetidin-3-yl)pyridazin-3-yl]-2-fluoro-3-{8-fluoro-2-methylimidazo[1,2-a]pyridin-6-yl}phenol (Compound 10)

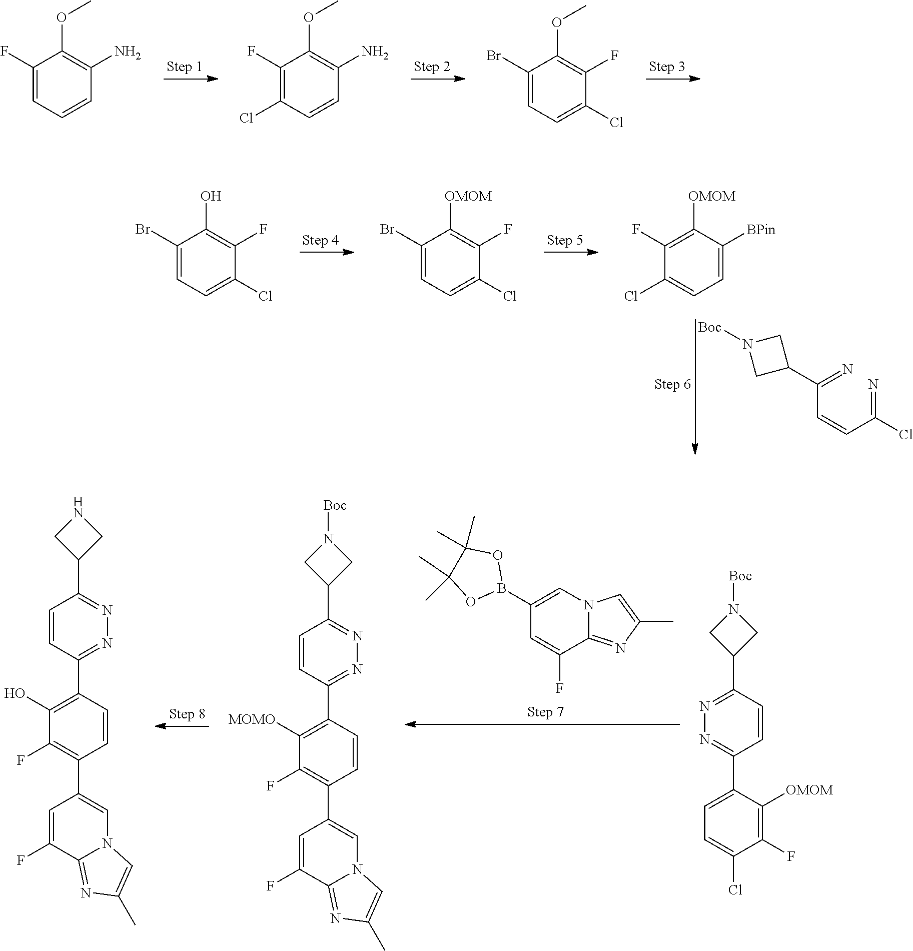

### Step 1: Preparation of 4-chloro-3-fluoro-2-methoxyaniline

To a solution of 3-fluoro-2-methoxybenzenenamine (15.0 g, 106.28 mmol) in DMF (50 mL) at room temperature was added in portions 1-chloropyrrolidine-2,5-dione (NCS) (14.19 g, 106.28 mmol) over 10 min. The reaction mixture was stirred for 1 day. After that the resulting mixture was diluted with water (100 mL) and the residue was extracted with EtOAc (100 mL). The organic layer was separated, washed with H₂O (50 mL), brine (50 mL), dried and evaporated under reduced pressure. The obtained mixture of isomers was purified by column chromatography using Hex/EtOAc (4/1) as an eluent to afford pure 4-chloro-3-fluoro-2-methoxyaniline (5.75 g, Y: 29.3%). ¹H NMR (400 MHz, Chloroform-d) δ 6.84 (t, J=8.5 Hz, 1H), 6.40 (d, J=9.8, 1.9 Hz, 1H), 3.90 (s, 3H).

### Step 2: Preparation of 1-bromo-4-chloro-3-fluoro-2-methoxybenzene

4-Chloro-3-fluoro-2-methoxyaniline (5.54 g, 31.56 mmol) was dissolved in HBr (48% in water, 50 mL) and the solution was cooled to 0° C. A solution of CuBr (5.43 g, 37.87 mmol) in water (30 mL) was added slowly (˜0.5 h) to the stirred slurry maintaining the temperature of the reaction mixture below 5° C. A purple solution of sodium nitrite (2.4 g, 34.71 mmol) in HBr (48% in water, 20 mL) was added dropwise to the reaction mixture, maintaining the temperature of the below 5° C. The resulting reaction mixture was heated at 60° C. until the evolution of gas ceased (˜1.5 h). The resulting mixture was cooled to r.t., and the product was extracted with DCM (2×50 mL). The combined organic extracts were washed with brine (50 mL), dried over MgSO₄, and evaporated under reduced pressure to give 1-bromo-4-chloro-3-fluoro-2-methoxybenzene (6.8 g, Y: 81%). ¹H NMR (500 MHz, DMSO-d6) δ 7.26 (t, J=8.5, 1H), 7.01 (d, J=9.5, 7.0, 1H), 3.98 (s, 3H).

### Step 3: Preparation of 6-bromo-3-chloro-2-fluorophenol

1-Bromo-4-chloro-3-fluoro-2-methoxybenzene (6.8 g, 28.4 mmol) was dissolved in dichloromethane (200 mL) and cooled to −78° C. Tribromoborane (14.23 g, 56.79 mmol) was added, and the reaction mixture was stirred at room temperature for 18 hours. The resulting mixture was quenched with ice water (200 mL); the organic layer was separated, washed with 5% aqueous NaHCO₃, (100 mL), dried over Na₂SO₄, and evaporated under reduced pressure to give 6-bromo-3-chloro-2-fluorophenol (4.15 g, Y: 61.6%). ¹H NMR (400 MHz, Chloroform-d) δ 7.24 (t, J=8.5, 1H), 6.88 (d, J=9.5, 7.0, 1H), 5.75-5.55 (br s, 1H).

### Step 4: Preparation of 1-bromo-4-chloro-3-fluoro-2-(methoxymethoxy)benzene

6-Bromo-3-chloro-2-fluorophenol (4.15 g, 18.41 mmol) was dissolved in anhydrous CH₂Cl₂ (100 mL). Ethylbis(propan-2-yl)amine (3.57 g, 27.61 mmol) was then added at r.t. The mixture was cooled to 0° C. and chloro(methoxy)methane (2.22 g, 27.61 mmol) was added dropwise. The reaction mixture was stirred at 0° C. for 30 min, then allowed to warm to r.t. and stirred for 11 h. After that the resulting mixture was quenched with sat. aq. NaHCO₃ (100 mL), and the organic layer was separated, washed with brine (200 mL) and dried over anhydrous Na₂SO₄. The solvent was removed under reduced pressure to afford 1-bromo-4-chloro-3-fluoro-2-(methoxy-methoxy)benzene (4.05 g, Y: 77.6%). ¹H NMR (400 MHz, Chloroform-d) δ 7.29-7.22 (m, 1H), 7.08-6.97 (m, 1H), 5.19 (s, 2H), 3.63 (s, 3H).

### Step 5: Preparation of 2-[4-chloro-3-fluoro-2-(methoxymethoxy)phenyl]-4,4,5,5-tetramethyl-1,3,2-dioxaborolane

A solution of 1-bromo-4-chloro-3-fluoro-2-(methoxymethoxy)benzene (4.0 g, 14.84 mmol), 4,4,5,5-tetramethyl-2-(4,4,5,5-tetramethyl-1,3,2-dioxaborolan-2-yl)-1,3,2-dioxaborolane (3.77 g, 14.84 mmol), potassium acetate (4.37 g, 44.53 mmol), and [1,1′-bis(diphenylphosphino)ferrocene]dichloropalladium (II) dichloromethane adduct (606.1 mg, 742.2 μmol) in dioxane (50 mL) was degassed and purged with Ar. The reaction mixture was heated overnight at 90° C. The resulting mixture was diluted with EtOAc (40 mL) and filtered. The filtrate was evaporated under reduced pressure to give the crude which was purified by column chromatography using Hex/EtOAc (8/1) as an eluent to give 2-[4-chloro-3-fluoro-2-(methoxymethoxy)phenyl]-4,4,5,5-tetramethyl-1,3,2-dioxaborolane (1.3 g, Y: 26.3%) as yellow oil. ¹H NMR (500 MHz, DMSO-d6) δ 7.43 (d, J=8.2, 1H), 7.13 (dd, J=7.0, 1H), 5.15 (s, 2H), 3.62 (s, 3H), 1.34 (s, 12H).

### Step 6: Preparation of tert-butyl 3-6-[4-chloro-3-fluoro-2-(methoxymethoxy)phenyl]pyridazin-3-yl-azetidine-1-carboxylate

A solution of 2-[4-chloro-3-fluoro-2-(methoxymethoxy)phenyl]-4,4,5,5-tetramethyl-1,3,2-dioxaborolane (305.46 mg, 964.93 μmol), tert-butyl 3-(6-chloropyridazin-3-yl)azetidine-1-carboxylate (260.27 mg, 964.93 μmol), [1,1′-bis(diphenylphosphino)ferrocene]dichloropalladium (II) dichloromethane adduct (39.4 mg, 48.25 μmol), and potassium carbonate (400.08 mg, 2.89 mmol) in dioxane (10 mL) and water (2 mL) was degassed and purged with Ar. The reaction mixture was heated overnight at 90° C. The resulting mixture was cooled to r.t. and filtered through a pad of Celite. The filtrate was concentrated in vacuo and the residue was purified by column chromatography using Hex/EtOAc (1/1) as an eluent to give tert-butyl 3-6-[4-chloro-3-fluoro-2-(methoxy-methoxy)phenyl]pyridazin-3-ylazetidine-1-carboxylate (269.0 mg, Y: 65.8%). ESI-MS (M+H)+: 424.2.

### Step 7: Preparation of tert-butyl 3-[6-(3-fluoro-4-8-fluoro-2-methylimidazo[1,2-a]pyridin-6-yl-2-(methoxymethoxy)phenyl)pyridazin-3-yl]azetidine-1-carboxylate

A solution of tert-butyl 3-6-[4-chloro-3-fluoro-2-(methoxymethoxy)phenyl]pyridazin-3-ylazetidine-1-carboxylate (230.24 mg, 543.2 μmol), 8-fluoro-2-methyl-6-(tetramethyl-1,3,2-dioxaborolan-2-yl)imidazo[1,2-a]pyridine (157.48 mg, 570.36 μmol), Pd₂(dba)₃ (24.87 mg, 27.16 μmol), and dicyclohexyl[2′,4′,6′-tris(propan-2-yl)-[1,1′-biphenyl]-2-yl]phosphane (38.84 mg, 81.48 μmol) in dioxane (10 mL) and water (2 mL) was degassed and purged with Ar. The reaction mixture was heated overnight at 100° C. The resulting mixture was cooled to r.t., diluted with EtOAc (10 mL) and filtered through a pad of Celite with Na₂SO₄. The filtrate was concentrated in vacuo to give the crude residue, which was triturated with MTBE (5 mL) and the precipitate was filtered to give pure tert-butyl 3-[6-(3-fluoro-4-8-fluoro-2-methylimidazo[1,2-a]pyridin-6-yl-2-(methoxymethoxy)phenyl)pyridazin-3-yl]azetidine-1-carboxylate (200.0 mg, Y: 65.8%). ESI-MS (M+H)+: 538.2.

### Step 8: Preparation of 6-[6-(azetidin-3-yl)pyridazin-3-yl]-2-fluoro-3-8-fluoro-2-methylimidazo[1,2-a]-pyridin-6-ylphenol as trifluoroacetate salt

tert-Butyl 3-[6-(3-fluoro-4-8-fluoro-2-methylimidazo[1,2-a]pyridin-6-yl-2-(methoxymethoxy)phenyl)pyridazin-3-yl]azetidine-1-carboxylate (200.23 mg, 372.48 μmol) was dissolved in CH₂Cl₂ (5 mL) and 2,2,2-trifluoroacetic acid (424.71 mg, 3.72 mmol) was added. The resulting mixture was stirred overnight at r.t. and then evaporated to dryness to provide the crude material. The residue was triturated with MeCN/MTBE (1/4, 2 mL) and the resulting precipitate was filtered and dried in vacuo to give pure 6-[6-(azetidin-3-yl)pyridazin-3-yl]-2-fluoro-3-8-fluoro-2-methylimidazo[1,2-a]pyridin-6-ylphenol as trifluoroacetate salt (183.0 mg, Y: 92%). ESI-MS (M+H)+: 394.0. ¹H NMR (500 MHz, DMSO-d6) δ 9.07 (br s, 1H), 8.9-8.8 (m, 2H), 8.6 (d, J=8.8 Hz, 1H), 8.0-7.9 (m, 3H), 7.68 (d, J=10.8 Hz, 1H), 7.26 (t, J=7.2 Hz, 1H), 4.45-4.38 (m, 5H), 2.4 (s, 3H). HzH

### Example 11. 2-[6-(azetidin-3-yl)pyridazin-3-yl]-3-fluoro-5-{8-fluoro-2-methylimidazo[1,2-a]pyridin-6-yl}phenol (Compound 11)

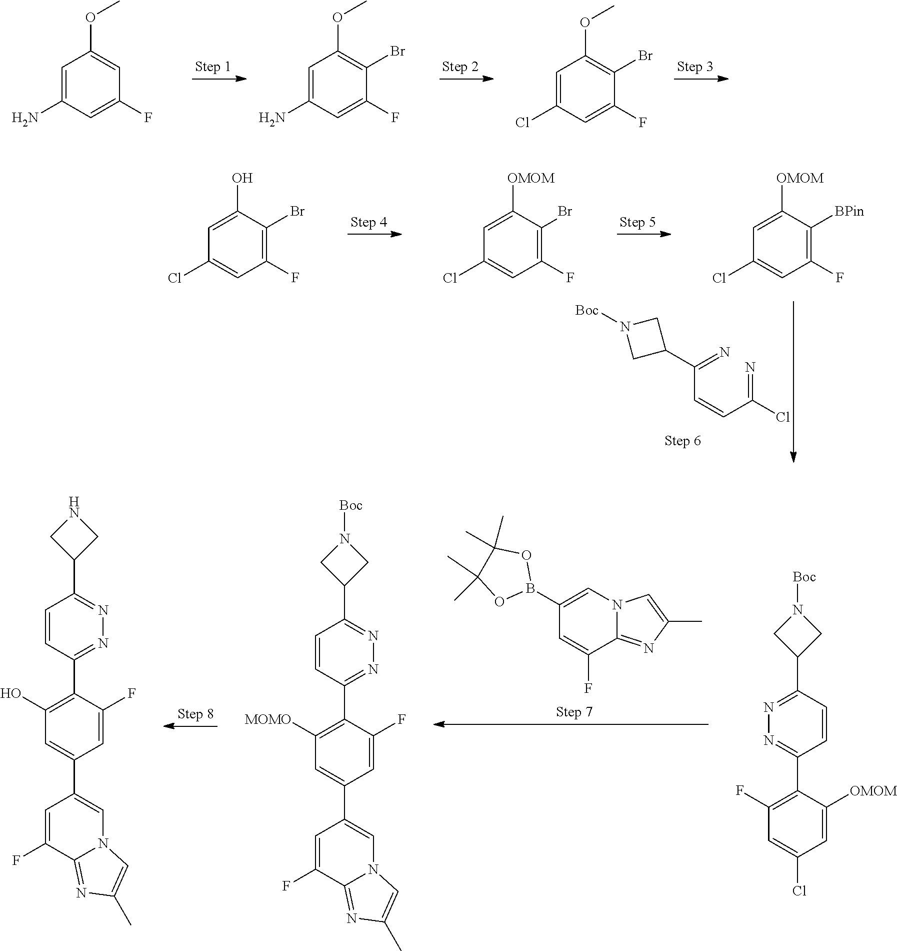

### Step 1: Preparation of 4-bromo-3-fluoro-5-methoxyaniline

A solution of 3-fluoro-5-methoxyaniline (10.0 g, 70.85 mmol) in DMF (100 mL) was treated with N-bromosuccinimide (12.61 g, 70.85 mmol) and the reaction mixture was stirred at r.t. for 1 h. The resulting mixture was diluted with water (150 mL) and ethyl acetate (150 mL). The phases were separated, and the aqueous phase extracted with additional ethyl acetate (100 mL). The combined organic phases were washed with water (150 mL) and brine (150 mL), dried over sodium sulfate and concentrated under reduced pressure to give 4-bromo-3-fluoro-5-methoxyaniline (14.7 g, Y: 89.6%). ¹H NMR (400 MHz, DMSO-d6) δ 6.16-6.08 (m, 1H), 6.06 (dd, J=11.2, 2.3 Hz, 1H) 3.71 (s, 3H).

### Step 2: Preparation of 2-bromo-5-chloro-1-fluoro-3-methoxybenzene

To a mixture of dichlorocopper dihydrate (12.01 g, 70.44 mmol) and tert-butyl nitrite (7.26 g, 70.44 mmol) in acetonitrile (100 mL) was added dropwise a solution of 4-bromo-3-fluoro-5-methoxyaniline (10.0 g, 45.45 mmol) in acetonitrile (20 mL) at 0° C. After all the aniline had been added, the reaction mixture was stirred for 1 h at room temperature, poured into 0.5 N HCl and extracted with EtOAc (2×150 mL). The combined organic layers were washed with brine (100 mL) and dried over Na₂SO₄. The suspension was filtered, and the filtrate was concentrated in vacuo. The residue was purified by column chromatography to afford 2-bromo-5-chloro-1-fluoro-3-methoxybenzene (10.6 g, Y: 92.5%). ¹H NMR (400 MHz, DMSO-d6) δ 7.17 (dt, J=8.5, 1.8 Hz, 1H), 7.06 (s, 1H), 3.87 (s, 3H).

### Step 3: Preparation of 2-bromo-5-chloro-3-fluorophenol

2-Bromo-5-chloro-1-fluoro-3-methoxybenzene (10.6 g, 44.26 mmol) was dissolved in dichloromethane (200 mL) and cooled to −78° C. Tribromoborane (22.18 g, 88.53 mmol) was added, and the reaction mixture was stirred at room temperature for 18 hours. The resulting mixture was quenched with ice-water (300 mL) and extracted with DCM (200 mL). The organic layer was washed with brine (150 mL), dried over sodium sulfate and concentrated under reduced pressure to afford 2-bromo-5-chloro-3-fluorophenol (8.5 g, Y: 80.9%). ¹H NMR (400 MHz, Chloroform-d) δ 6.85 (s, 1H), 6.75 (dd, J=8.1, 2.4 Hz, 1H), 5.71 (s, 1H).

### Step 4: Preparation of 2-bromo-5-chloro-1-fluoro-3-(methoxymethoxy)benzene

2-Bromo-5-chloro-3-fluorophenol (8.5 g, 37.7 mmol) was dissolved in anhydrous CH₂Cl₂ (100 mL). Ethylbis(propan-2-yl)amine (5.85 g, 45.24 mmol) was then added at r.t. The mixture was cooled to 0° C. and chloro(methoxy)methane (3.64 g, 45.24 mmol) was added dropwise. The reaction mixture was stirred at 0° C. for 30 min, then allowed to warm to r.t. and stirred for 11 h. After that the resulting mixture was quenched with sat. aq. NaHCO₃ (250 mL), and the organic layer was separated, washed with brine (200 mL) and dried over anhydrous Na₂SO₄. The solvent was removed under reduced pressure to afford 2-bromo-5-chloro-1-fluoro-3-(methoxymethoxy)benzene (9.0 g, Y: 84.1%). ¹H NMR (400 MHz, DMSO-d6) δ 7.22 (dt, J=8.6, 2.7 Hz, 1H), 7.13 (s, 1H), 5.33 (s, 2H), 3.37 (s, 3H).

### Step 5: Preparation of 2-[4-chloro-2-fluoro-6-(methoxymethoxy)phenyl]-4,4,5,5-tetramethyl-1,3,2-dioxaborolane

A solution of 2-bromo-5-chloro-1-fluoro-3-(methoxymethoxy)benzene (9.85 g, 36.55 mmol), 4,4,5,5-tetramethyl-2-(4,4,5,5-tetramethyl-1,3,2-dioxaborolan-2-yl)-1,3,2-dioxaborolane (9.28 g, 36.55 mmol), potassium acetate (10.76 g, 109.65 mmol), and [1,1′-bis(diphenylphosphino)ferrocene]dichloropalladium (II) dichloromethane adduct (1.49 g, 1.83 mmol) in dioxane (150 mL) was degassed and purged with Ar. The reaction mixture was heated overnight at 90° C. The reaction mixture was diluted with EtOAc (100 mL) and filtered. The filtrate was evaporated under reduced pressure to give the crude which was purified by column chromatography using Hex/EtOAc (8/1) as an eluent to give 2-[4-chloro-2-fluoro-6-(methoxymethoxy)phenyl]-4,4,5,5-tetramethyl-1,3,2-dioxaborolane (4.0 g, Y: 27.7%) as yellow oil. ¹H NMR (500 MHz, Chloroform-d) δ 6.84 (s, 1H), 6.72 (dt, J=8.3, 1.8 Hz, 1H), 5.13 (s, 2H), 3.47 (s, 3H), 1.36 (s, 12H).

### Step 6: Preparation of tert-butyl 3-6-[4-chloro-2-fluoro-6-(methoxymethoxy)phenyl]pyridazin-3-yl-azetidine-1-carboxylate

A solution of 2-[4-chloro-2-fluoro-6-(methoxymethoxy)phenyl]-4,4,5,5-tetramethyl-1,3,2-dioxaborolane (845.57 mg, 2.67 mmol), tert-butyl 3-(6-chloropyridazin-3-yl)azetidine-1-carboxylate (400.26 mg, 1.48 mmol), [1,1′-bis(diphenylphosphino)ferrocene]dichloropalladium (II) dichloromethane adduct (60.59 mg, 74.2 μmol), and potassium carbonate (410.18 mg, 2.97 mmol) in dioxane (10 mL) and water (2 mL) was degassed and purged with Ar. The reaction mixture was heated overnight at 90° C. The resulting mixture was cooled to r.t. and filtered through a pad of Celite. The filtrate was concentrated in vacuo and the residue was purified by column chromatography using Hex/EtOAc 1/1 as an eluent to give tert-butyl 3-6-[4-chloro-2-fluoro-6-(methoxymethoxy)phenyl]pyridazin-3-ylazetidine-1-carboxylate (100.0 mg, Y: 15.1%). ESI-MS (M+H)+: 424.0.

### Step 7: Preparation of tert-butyl 3-[6-(2-fluoro-4-8-fluoro-2-methylimidazo[1,2-a]pyridin-6-yl-6-(methoxymethoxy)phenyl)pyridazin-3-yl]azetidine-1-carboxylate

A solution of tert-butyl 3-6-[4-chloro-2-fluoro-6-(methoxymethoxy)phenyl]pyridazin-3-ylazetidine-1-carboxylate (100.14 mg, 236.25 μmol), 8-fluoro-2-methyl-6-(tetramethyl-1,3,2-dioxaborolan-2-yl)imidazo[1,2-a]pyridine (65.23 mg, 236.25 μmol), Pd₂(dba)₃ (10.82 mg, 11.81 μmol), and dicyclohexyl[2′,4′,6′-tris(propan-2-yl)-[1,1′-biphenyl]-2-yl]phosphane (16.89 mg, 35.44 μmol) in dioxane (15 mL) and water (3 mL) was degassed and purged with Ar. The resulting mixture was heated overnight at 100° C. The resulting mixture was cooled to r.t., diluted with EtOAc (20 mL) and filtered through a pad of Celite with Na₂SO₄. The filtrate was concentrated in vacuo to give the crude residue which was purified by HPLC to provide pure tert-butyl 3-[6-(2-fluoro-4-8-fluoro-2-methylimidazo[1,2-a]pyridin-6-yl-6-(methoxymethoxy)phenyl)pyridazin-3-yl]azetidine-1-carboxylate (66.0 mg, Y: 52%). ESI-MS (M+H)+: 538.2.

### Step 8: Preparation of 2-[6-(azetidin-3-yl)pyridazin-3-yl]-3-fluoro-5-8-fluoro-2-methylimidazo[1,2-a]-pyridin-6-ylphenol as trifluoroacetate salt

tert-Butyl 3-[6-(2-fluoro-4-8-fluoro-2-methylimidazo[1,2-a]pyridin-6-yl-6-(methoxymethoxy)phenyl)pyridazin-3-yl]azetidine-1-carboxylate (66.0 mg, 122.78 μmol) was dissolved in CH₂C₂ (3 mL) and 2,2,2-trifluoroacetic acid (140.0 mg, 1.23 mmol) was added. The reaction mixture was stirred overnight at r.t. and then evaporated to dryness to provide crude material. The residue was triturated with MeCN/MTBE (1/4, 2 mL) and the resulting precipitate was filtered and dried in vacuo to give pure 2-[6-(azetidin-3-yl)pyridazin-3-yl]-3-fluoro-5-8-fluoro-2-methylimidazo[1,2-a]pyridin-6-ylphenol as trifluoroacetate salt (56.0 mg, Y: 87.2%). ESI-MS (M+H)+: 394.0. ¹H NMR (400 MHz, DMSO-d6) δ 9.00-8.90 (m, 2H), 8.85-8.75 (m, 1H), 7.99 (d, J=8.8 Hz, 1H), 7.96-7.92 (m, J=8.8 Hz, 1H), 7.81 (d, 1H), 7.77-7.69 (m, 1H), 7.25 (dd, J=7.8 Hz, 1.1 Hz, 1H), 7.20-7.15 (m, 1H), 4.45-4.30 (m, 5H), 2.40 (s, 3H).

### Example 12. 5-{8-fluoro-2-methylimidazo[1,2-a]pyridin-6-yl}-2-[6-(1-methylazetidin-3-yl)pyridazin-3-yl]phenol (Compound 12)

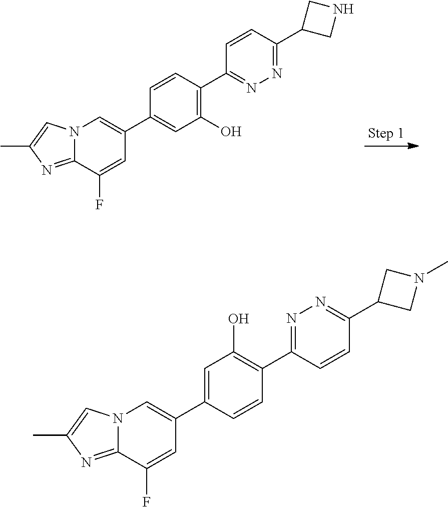

### Step 1: Preparation of 5-8-fluoro-2-methylimidazo[1,2-a]pyridin-6-yl-2-[6-(1-methyl-azetidin-3-yl)-pyridazin-3-yl]phenol hydrochloride

To a stirred solution of 2-[6-(azetidin-3-yl)pyridazin-3-yl]-5-8-fluoro-2-methylimidazo[1,2-a]pyridin-6-ylphenol hydrochloride (100.05 mg, 242.92 μmol) in EtOH (5 mL), formaldehyde (191.95 mg, 6.39 mmol) was added followed by acetic acid (58.35 mg, 971.69 μmol). The mixture was stirred for 1 h and then sodium cyanoborohydride (45.8 mg, 728.76 μmol) was added and the reaction mixture was stirred overnight. After that the solvent was evaporated and the crude residue was triturated with H₂O (5 mL) and EtOAc (10 mL). The precipitate was filtered and dried in vacuo. The residue was dissolved in MeOH (2 mL), and HCl/dioxane (1 mL) was added at 0° C. The resulting solution was evaporated under high vacuum to give 5-8-fluoro-2-methylimidazo[1,2-a]pyridin-6-yl-2-[6-(1-methyl-azetidin-3-yl)-pyridazin-3-yl]phenol hydrochloride (124.0 mg, Y: 95% yield). ESI-MS (M+H)+: 390.2. ¹H NMR (500 MHz, DMSO-d₆) δ 9.21 (s, 1H), 8.54 (t, J=9.9 Hz, 1H), 8.31 (d, J=10.8 Hz, 1H), 8.16 (s, 2H), 7.92 (dd, J=16.6, 9.0 Hz, 1H), 7.48-7.38 (m, 2H), 4.63-4.55 (m, 2H), 4.45-4.30 (m, 3H), 3.16 (s, 3H), 2.51 (s, 3H).

### Example 13. 2-[6-(azetidin-3-yl)pyridazin-3-yl]-5-{2-methylpyrazolo[1,5-a]pyridin-5-yl}phenol (Compound 13)

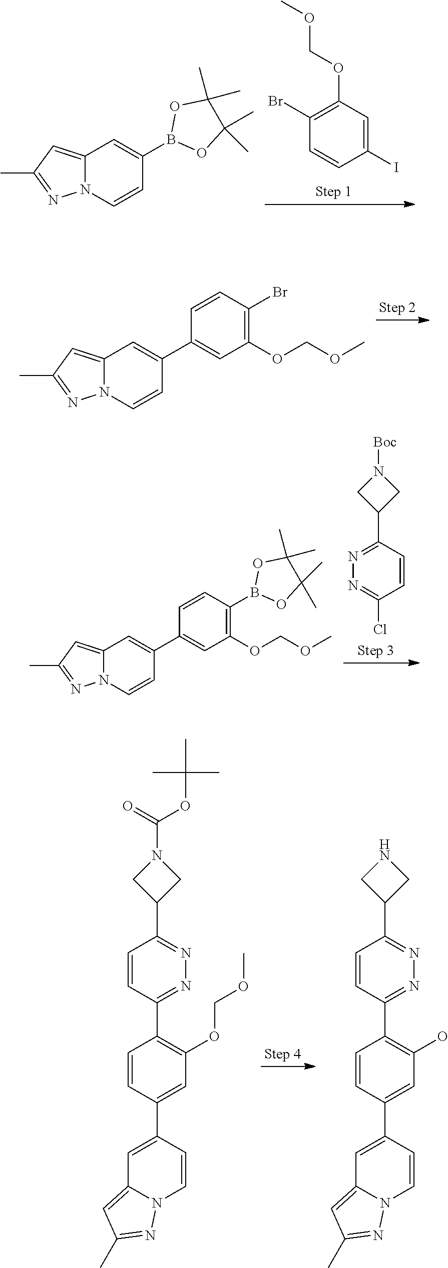

### Step 1: Preparation of 5-[4-bromo-3-(methoxymethoxy)phenyl]-2-methylpyrazolo[1,5-a]pyridine

2-Methyl-5-(tetramethyl-1,3,2-dioxaborolan-2-yl)pyrazolo[1,5-a]pyridine (2.62 g, 10.15 mmol), 1-bromo-4-iodo-2-(methoxymethoxy)benzene (2.78 g, 8.12 mmol), [1,1′-bis(diphenylphosphino)ferrocene]-dichloropalladium(II) dichloromethane adduct (663.03 mg, 811.91 μmol), and potassium carbonate (3.37 g, 24.36 mmol) were mixed in dioxane/water under an argon atmosphere. The reaction mixture was heated to 90° C. overnight. After work-up with EtOAc and crystallization from MTBE 5-[4-bromo-3-(methoxymethoxy)phenyl]-2-methylpyrazolo[1,5-a]pyridine (2.8 g, Y: 79.5%) was obtained and used in the next step without further purification. ESI-MS (M+H)+: 348.0.

### Step 2: Preparation of 5-[3-(methoxymethoxy)-4-(4,4,5,5-tetramethyl-1,3,2-dioxaborolan-2-yl)phenyl]-2-methylpyrazolo[1,5-a]pyridine

5-[4-Bromo-3-(methoxymethoxy)phenyl]-2-methylpyrazolo[1,5-a]pyridine (2.8 g, 8.06 mmol), 4,4,5,5-tetramethyl-2-(4,4,5,5-tetramethyl-1,3,2-dioxaborolan-2-yl)-1,3,2-dioxaborolane (1.54 g, 6.05 mmol), [1,1′-bis(diphenylphosphino)ferrocene]dichloropalladium(II) dichloromethane adduct (658.66 mg, 806.55 μmol), and potassium acetate (2.37 g, 24.2 mmol) were mixed in dioxane under an argon atmosphere. The reaction mixture was heated to 100° C. overnight and concentrated under reduced pressure. Purification of the residue via flash column chromatography afforded 5-[3-(methoxymethoxy)-4-(4,4,5,5-tetramethyl-1,3,2-dioxaborolan-2-yl)-phenyl]-2-methylpyrazolo[1,5-a]pyridine (500.0 mg, 1.27 mmol, Y: 15.7%) which was used in the next step without further purification. ESI-MS (M+H)+: 395.2.

### Step 3: Preparation of tert-butyl 3-6-[2-(methoxymethoxy)-4-2-methylpyrazolo[1,5-a]pyridin-5-ylphenyl]pyridazin-3-ylazetidine-1-carboxylate

5-[3-(Methoxymethoxy)-4-(4,4,5,5-tetramethyl-1,3,2-dioxaborolan-2-yl)phenyl]-2-methylpyrazolo[1,5-a]pyridine (179.85 mg, 456.16 μmol), tert-butyl 3-(6-chloropyridazin-3-yl)azetidine-1-carboxylate (111.85 mg, 414.69 μmol), [1,1′-bis(diphenylphosphino)ferrocene]dichloropalladium(II) dichloromethane adduct (33.87 mg, 41.47 μmol), and potassium carbonate (171.94 mg, 1.24 mmol) were mixed in dioxane/water under an argon atmosphere. The reaction mixture was heated to 90° C. overnight. After work-up with EtOAc tert-butyl 3-6-[2-(methoxymethoxy)-4-2-methylpyrazolo[1,5-a]pyridin-5-ylphenyl]pyridazin-3-ylazetidine-1-carboxylate (200.0 mg, Y.: 67.3% yield) was obtained and used in the next step without further purification. ESI-MS (M+H)+: 502.2.

### Step 4: Preparation of 2-[6-(azetidin-3-yl)pyridazin-3-yl]-5-2-methylpyrazolo[1,5-a]pyridin-5-ylphenol

tert-Butyl 3-6-[2-(methoxymethoxy)-4-2-methylpyrazolo[1,5-a]pyridin-5-ylphenyl]pyridazin-3-ylazetidine-1-carboxylate (200.0 mg, 398.74 μmol) was dissolved in DCM (1 mL) and TFA (1 mL) and the reaction mixture was stirred overnight at r.t. After full evaporation and HPLC pure 2-[6-(azetidin-3-yl)pyridazin-3-yl]-5-2-methylpyrazolo[1,5-a]pyridin-5-ylphenol as TFA (25.5 mg, Y: 13.5%) was obtained. ESI-MS (M+H)+: 358.2. ¹H NMR (500 MHz, DMSO-d₆) δ 12.95 (s, 1H), 8.95 (br s, 1H), 8.82 (br s, 1H), 8.62 (d, J=8.2 Hz, 1H), 8.55 (d, J=8.2 Hz, 1H), 8.14 (d, J=8.3 Hz, 1H), 7.96 (s, 1H), 7.89 (d, J=9.0 Hz, 1H), 7.46-7.37 (m, 2H), 7.17 (dd, J=7.3, 2.3 Hz, 1H), 6.45 (s, 1H), 4.45-4.30 (m, 5H), 2.40 (s, 3H).

### Example 14. 2-[6-(1-methylazetidin-3-yl)pyridazin-3-yl]-5-{2-methylimidazo[1,2-a]pyridin-6-yl}phenol (Compound 14)

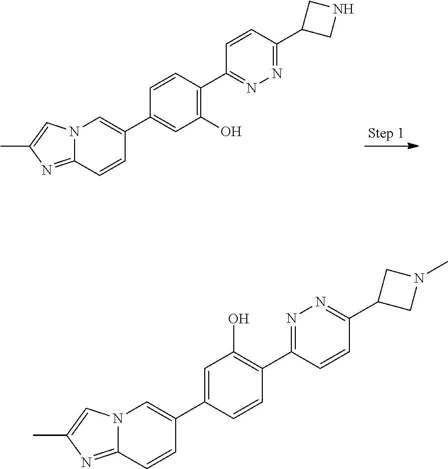

### Step 1: Preparation of 2-[6-(I-methylazetidin-3-yl)pyridazin-3-yl]-5-2-methylimidazo[1,2-a]pyridin-6-ylphenol as trifluoroacetate salt

A solution of 2-[6-(azetidin-3-yl)pyridazin-3-yl]-5-2-methylimidazo[1,2-a]pyridin-6-ylphenol hydrochloride (20.04 mg, 50.88 μmol) and formaldehyde (40.21 mg, 1.34 mmol) in methanol was stirred at ambient temperature for 2 h. After that sodium cyanoborohydride (9.59 mg, 152.65 μmol) was added followed by acetic acid (17.5 mg, 291.48 μmol) and the reaction mixture was stirred at ambient temperature for 16 h. The resulting mixture was diluted with water and the precipitate formed was filtered and dried in vacuo. The residue was dissolved in MeOH (2 mL), and TFA (0.1 mL) was added at 0° C. The resulting solution was evaporated under high vacuum to give 2-[6-(1-methylazetidin-3-yl)pyridazin-3-yl]-5-2-methylimidazo[1,2-a]pyridin-6-ylphenol as trifluoroacetate salt (20.0 mg, Y: 97.4%). ESI-MS (M+H)+: 372.0. ¹H NMR (400 MHz, DMSO-d₆) δ 10.27-10.07 (br s, 1H), 9.34 (s, 1H), 8.65-8.57 (m, 1H), 8.28 (d, J=9.1 Hz, 1H), 8.22 (d, J=8.1 Hz, 1H), 8.07 (s, 1H), 7.99 (d, J=9.1 Hz, 1H), 7.93 (d, J=9.1 Hz, 1H), 7.44 (d, J=11.6 Hz, 2H), 4.65-4.55 (m, 2H), 4.46-4.29 (m, 3H), 2.95 (s, 3H), 2.49 (s, 3H).

### Example 15. 5-{8-fluoro-2-methylimidazo[1,2-a]pyridin-6-yl}-2-{6-[1-(propan-2-yl)azetidin-3-yl]pyridazin-3-yl}phenol (Compound 15)

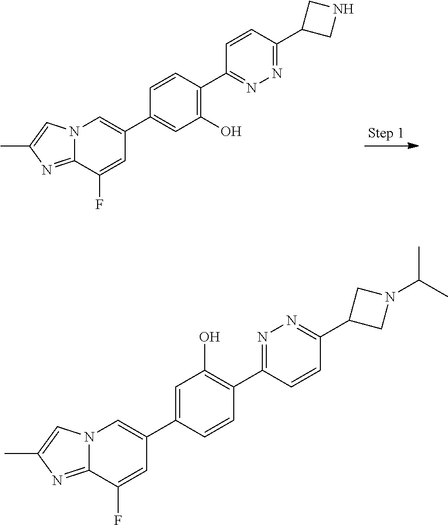 [SMILES: Cc1cn2cc(-c3ccc(-c4ccc(C5CNC5)nn4)c(O)c3)cc(F)c2n1 ; Cc1cn2cc(-c3ccc(-c4ccc(C5CN(C(C)C)C5)nn4)c(O)c3)cc(F)c2n1]

### Step 1: Preparation of 5-8-fluoro-2-methylimidazo[1,2-a]pyridin-6-yl-2-6-[I-(propan-2-yl)azetidin-3-yl]pyridazin-3-ylphenol trifluoroacetate

A solution of 2-[6-(azetidin-3-yl)pyridazin-3-yl]-5-8-fluoro-2-methylimidazo[1,2-a]pyridin-6-ylphenol hydrochloride (29.99 mg, 72.82 μmol) and propan-2-one (42.29 mg, 728 μmol) in methanol was stirred at ambient temperature for 2 h. After that sodium cyanoborohydride (13.73 mg, 218 μmol) was added followed by acetic acid (17.5 mg, 291.48 μmol) and the reaction mixture was stirred at ambient temperature for 16 h. The resulting mixture was diluted with water and the precipitate formed was filtered and dried in vacuo. The residue was dissolved in MeOH (2 mL), and TFA (0.1 mL) was added at 0° C. The resulting solution was evaporated under high vacuum to give 5-8-fluoro-2-methylimidazo[1,2-a]pyridin-6-yl-2-6-[1-(propan-2-yl)azetidin-3-yl]pyridazin-3-ylphenol trifluoroacetate (18.0 mg, Y: 56.3%). ESI-MS (M+H)+: 418.2. ¹H NMR (400 MHz, DMSO-d₆) δ 10.34-10.11 (br s, 1H), 8.95 (s, 1H), 8.63-8.59 (m, 1H), 8.20-8.14 (m, 1H), 7.99-7.92 (m, 1H), 7.90 (s, 1H), 7.75-7.70 (m, 1H), 7.40 (s, 2H), 4.60-4.30 (m, 5H), 3.55-3.45 (m, 1H), 2.41 (s, 3H), 1.20 (d, J=5.6 Hz, 6H).

### Example 16. 2-[6-(azetidin-3-yl)pyridazin-3-yl]-5-(2-methyl-2H-indazol-5-yl)phenol (Compound 16)

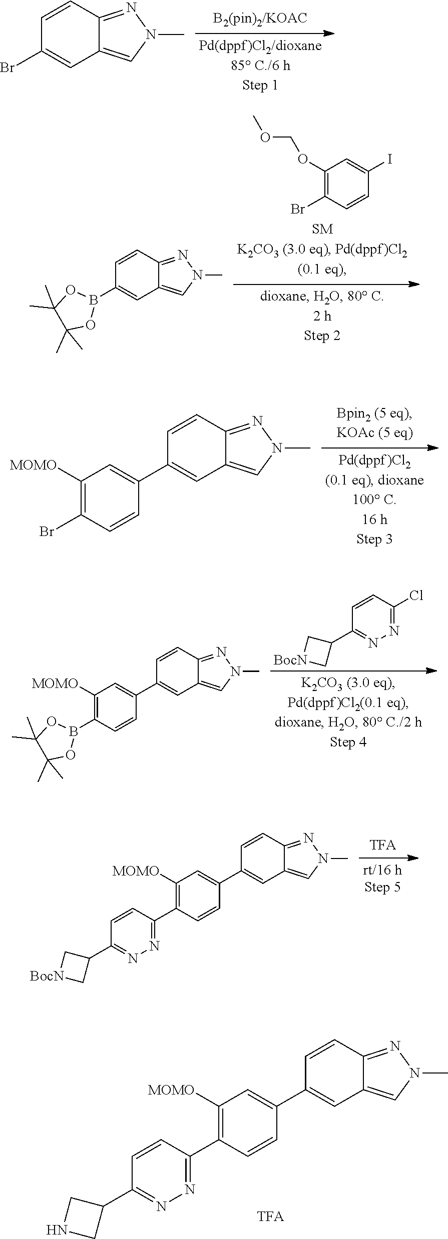

### Step 1: Preparation of 2-methyl-5-(4,4,5,5-tetramethyl-1,3,2-dioxaborolan-2-yl)-2H-indazole

To a solution of 5-bromo-2-methyl-2H-indazole (800 mg, 3.79 mmol), B₂(Pin)₂ (1.925 g, 7.58 mmol) and KOAc (1.114 g, 11.37 mmol) in 1,4-dioxane (25 mL) was added Pd(dppf)Cl₂ (309 mg, 0.379 mmol). The mixture was stirred at 85° C. under N₂ for 6 h. The mixture was concentrated under reduced pressure and the residue was purified by silica gel column chromatography (PE:EA=4: 1-1:1) to give title product (732 mg, Y: 75%) as a white solid. ESI-MS (M+H)+: 259.2. ¹H NMR (400 MHz, CDCl₃) δ 8.22 (s, 1H), 7.91 (s, 1H), 7.66-7.65 (m, 2H), 4.21 (s, 3H), 1.36 (s, 12H).

### Step 2: Preparation of 5-(4-bromo-3-(methoxymethoxy)phenyl)-2-methyl-2H-indazole

To a solution of 2-methyl-5-(4,4,5,5-tetramethyl-1,3,2-dioxaborolan-2-yl)-2H-indazole (1 g, 3.88 mmol), 1-bromo-4-iodo-2-(methoxymethoxy)benzene (1.6 g, 4.656 mmol) and K₂CO₃ (1.6 g, 11.64 mmol) in 1,4-dioxane: H₂O (40 mL:8 mL) was added Pd(dppf)Cl₂ (317 mg, 0.388 mmol). The mixture was stirred at 80° C. under N₂ for 2 h. The mixture was concentrated under reduced pressure, the residue was purified by silica gel column chromatography (PE:EA=4:1-1:2) to give title product (1.2 g, Y: 92%) as a brown solid. ESI-MS (M+H)+: 347.1. ¹H NMR (400 MHz, CDCl₃) δ 7.94 (s, 1H), 7.81-7.80 (m, 1H), 7.75 (d, J=9.0 Hz, 1H), 7.59 (d, J=8.2 Hz, 1H), 7.52-7.49 (m, 1H), 7.41 (d, J=2.1 Hz, 1H), 7.17-7.14 (m, 1H), 5.33 (s, 2H), 4.24 (s, 3H), 3.56 (s, 3H).

### Step 3: Preparation of 5-(3-(methoxymethoxy)-4-(4,4,5,5-tetramethyl-1,3,2-dioxaborolan-2-yl)phenyl)-2-methyl-2H-indazole

To a mixture of 5-(4-bromo-3-(methoxymethoxy)phenyl)-2-methyl-2H-indazole (1.6 g, 4.62 mmol), B₂(Pin)₂ (5.9 g, 23.12 mmol) and KOAc (2.3 g, 23.12 mmol) in 1,4-dioxane (100 mL) was added Pd(dppf)Cl₂ (377 mg, 0.462 mmol). The mixture was stirred at 100° C. under N₂ for 16 h. The mixture was allowed to cooling down to room temperature and concentrated under reduced pressure, the residue was purified by silica gel column chromatography (PE:EA=2:1-1:3) to give title product (2 g, crude) as a brown oil. ESI-MS (M+H)+: 395.4.

### Step 4: Preparation of tert-butyl 3-(6-(2-(methoxymethoxy)-4-(2-methyl-2H-indazol-5-yl)phenyl)pyridazin-3-yl)azetidine-1-carboxylate

To a mixture of 5-(3-(methoxymethoxy)-4-(4,4,5,5-tetramethyl-1,3,2-dioxaborolan-2-yl)phenyl)-2-methyl-2H-indazole (1.9 g, 4.8 mmol) and tert-butyl 3-(6-chloropyridazin-3-yl)azetidine-1-carboxylate (1.29 g, 4.8 mmol) and K₂CO₃ (2.0 g, 14.5 mmol) in 1,4-dioxane: H₂O (50 mL:10 mL) was added Pd(dppf)Cl₂ (392 mg, 0.48 mmol). The mixture was stirred at 80° C. under N₂ for 2 h. The mixture was concentrated in vacuo and the residue was purified by silica gel column chromatography (PE:EA=2:1-1:3) to give title product (450 mg, Y: 19%) as a white solid. ESI-MS (M+H)+: 502.4.

### Step 5: Preparation of 2-(6-(azetidin-3-yl)pyridazin-3-yl)-5-(imidazo[1,2-a]pyridin-6-yl)phenol TFA salt

A solution of tert-butyl 3-(6-(2-(methoxymethoxy)-4-(2-methyl-2H-indazol-5-yl)phenyl)pyridazin-3-yl)azetidine-1-carboxylate (50 mg, 0.10 mmol) in TFA (5 mL) was stirred at RT for 16 h. The mixture was concentrated in vacuo and lyophilized to give title product (38 mg, Y: 81%) as a yellow solid. ESI-MS (M+H)+: 358.0. ¹H NMR (400 MHz, MeOD-d₄) δ 8.44 (d, J=9.1 Hz, 1H), 8.29 (s, 1H), 8.01 (d, J=8.5 Hz, 2H), 7.77 (d, J=9.0 Hz, 1H), 7.72-7.64 (m, 2H), 7.38-7.31 (m, 2H), 4.61-4.52 (m, 4H), 4.50-4.45 (m, 1H), 4.24 (s, 3H).

### Example 17. 2-[6-(1-ethylazetidin-3-yl)pyridazin-3-yl]-5-{8-fluoro-2-methylimidazo[1,2-a]pyridin-6-yl}phenol (Compound 17)

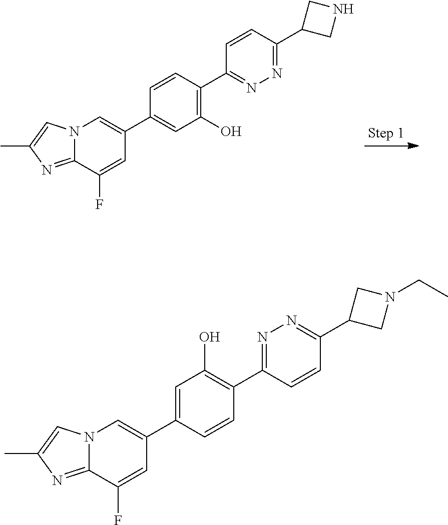 [SMILES: Cc1cn2cc(-c3ccc(-c4ccc(C5CNC5)nn4)c(O)c3)cc(F)c2n1 ; CCN1CC(c2ccc(-c3ccc(-c4cc(F)c5nc(C)cn5c4)cc3O)nn2)C1]

### Step 1: Preparation of 2-[6-(I-ethylazetidin-3-yl)-pyridazin-3-yl]-5-8-fluoro-2-methylimidazo[1,2-a]pyridin-6-ylphenol as trifluoroacetate salt

A solution of 2-[6-(azetidin-3-yl)pyridazin-3-yl]-5-8-fluoro-2-methylimidazo[1,2-a]pyridin-6-yl-phenol hydrochloride (30.01 mg, 72.87 μmol) and acetaldehyde (32.1 mg, 728.71 μmol) in methanol (2 mL) was stirred at ambient temperature for 2 h. After that sodium cyanoborohydride (13.74 mg, 218.61 μmol) was added followed by acetic acid (17.5 mg, 291.48 μmol) and the reaction mixture was stirred at ambient temperature for 16 h. The resulting mixture was diluted with water (3 mL) and the precipitate formed was filtered and purified by HPLC. The residue was dissolved in a mixture of MeCN/TFA (1/0.1 mL) and evaporated under reduced pressure to afford 2-[6-(1-ethylazetidin-3-yl)-pyridazin-3-yl]-5-8-fluoro-2-methylimidazo[1,2-a]pyridin-6-ylphenol as trifluoroacetate salt (17.0 mg, Y: 57.8%). ESI-MS (M+H)+: 404.2. ¹H NMR (500 MHz, Methanol-d₄) δ 8.98 (s, 1H), 8.47 (t, J=8.7 Hz, 1H), 8.19-8.04 (m, 3H), 7.82 (dd, J=9.2, 3.7 Hz, 1H), 7.46-7.32 (m, 2H), 4.77-4.62 (m, 2H), 4.58-4.35 (m, 3H), 3.42 (q, J=7.5 Hz, 2H), 2.59 (s, 3H), 1.29 (t, J=7.3, 3H).

### Example 18. 5-{8-fluoro-2-methylimidazo[1,2-a]pyridin-6-yl}-2-{6-[1-(oxan-4-yl)azetidin-3-yl]pyridazin-3-yl}phenol (Compound 18)

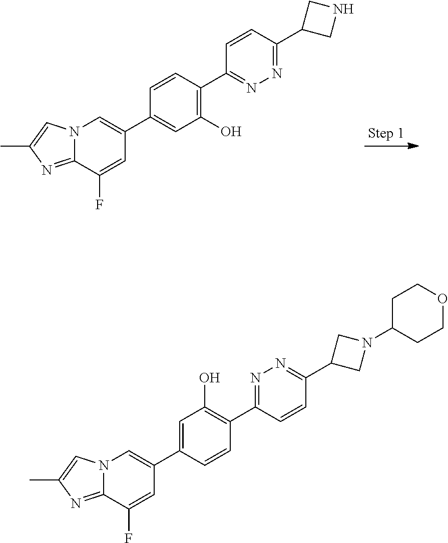 [SMILES: Cc1cn2cc(-c3ccc(-c4ccc(C5CNC5)nn4)c(O)c3)cc(F)c2n1 ; Cc1cn2cc(-c3ccc(-c4ccc(C5CN(C6CCOCC6)C5)nn4)c(O)c3)cc(F)c2n1]

### Step 1: Preparation of 5-8-fluoro-2-methylimidazo[1,2-a]pyridin-6-yl-2-6-[1-(oxan-4-yl)azetidin-3-yl]pyridazin-3-ylphenol as trifluoroacetate salt

A solution of 2-[6-(azetidin-3-yl)pyridazin-3-yl]-5-8-fluoro-2-methylimidazo[1,2-a]pyridin-6-ylphenol hydrochloride (30.0 mg, 72.84 μmol) and oxan-4-one (14.6 mg, 145.81 μmol, 10.0 μL) in ethanol was stirred at 45° C. for 2 h. After that sodium cyanoboranuide (13.74 mg, 218.71 μmol) was added followed by acetic acid (17.5 mg, 291.48 μmol) and the reaction mixture was stirred at 80° C. for 16 h. The resulting mixture was diluted with water and the precipitate formed was filtered and dried in vacuo. The residue was dissolved in MeOH (2 mL), and TFA (0.1 mL) was added at 0° C. The resulting solution was evaporated under high vacuum to give 5-8-fluoro-2-methylimidazo[1,2-a]pyridin-6-yl-2-6-[1-(oxan-4-yl)azetidin-3-yl]pyridazin-3-ylphenol as trifluoroacetate salt (20.0 mg, Y: 59.7%). ESI-MS (M+H)+: 460.2. ¹H NMR (500 MHz, Methanol-d₄) δ 8.98 (s, 1H), 8.47 (t, J=8.7 Hz, 1H), 8.19-8.04 (m, 3H), 7.82 (dd, J=9.2, 3.7 Hz, 1H), 7.46-7.32 (m, 2H), 4.79-4.53 (m, 5H), 4.13-4.00 (m, 2H), 3.69-3.61 (m, 1H), 3.46 (t, J=11.7 Hz, 2H), 2.58 (s, 3H), 2.10-2.00 (m, 2H), 1.62-1.52 (m, 2H).

### Example 19. 5-(2-methyl-2H-indazol-5-yl)-2-[6-(1-methylazetidin-3-yl)pyridazin-3-yl]phenol (Compound 19)

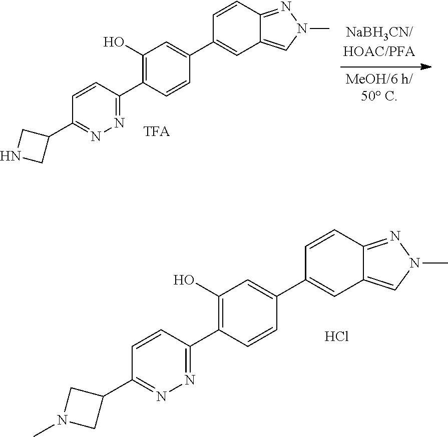

### Step 1: Preparation of 5-(2-methyl-2H-indazol-5-yl)-2-(6-(I-methylazetidin-3-yl)pyridazin-3-yl)phenol hydrochloride

To a solution of 2,2,2-trifluoroacetaldehyde-2-(6-(azetidin-3-yl)pyridazin-3-yl)-5-(2-methyl-2H-indazol-5-yl)phenol (600 mg, 1.68 mmol) in MeOH (80 mL) was added (HCHO)ₙ (252 mg, 8.4 mmol) and acetic acid (302 mg, 5.04 mmol). The mixture was stirred at RT for 1 h, then sodium cyanoborohydride (318 mg, 5.04 mmol) was added h and the mixture was stirred at 50° C. for 5 h. Water (10 mL) was added and the solution was concentrated in vacuo. The crude was added MeOH (10 mL) and stirred for 3 h, the precipitate was filtered and washed with MeOH (30 mL), then diluted with 8 mL water (0.1% HCl in H2O) and lyophilized to give title product (185 mg, yield: 27%) as a yellow solid. ESI-MS (M+H)+: 372.1. ¹H NMR (400 MHz, MeOD-d₄) δ 8.49 (d, J=9.0 Hz, 1H), 8.35 (s, 1H), 8.05 (s, 1H), 8.03-7.96 (m, 1H), 7.91-7.82 (m, 1H), 7.71 (s, 2H), 7.41-7.30 (m, 2H), 4.80-4.76 (m, 1H), 4.74-4.68 (m, 1H), 4.55-4.41 (m, 3H), 4.26 (s, 3H), 3.09 (d, J=4.8 Hz, 3H).

### Example 20. 5-{2,8-dimethylimidazo[1,2-a]pyrazin-6-yl}-2-[6-(1-methylazetidin-3-yl)pyridazin-3-yl]phenol (Compound 20)

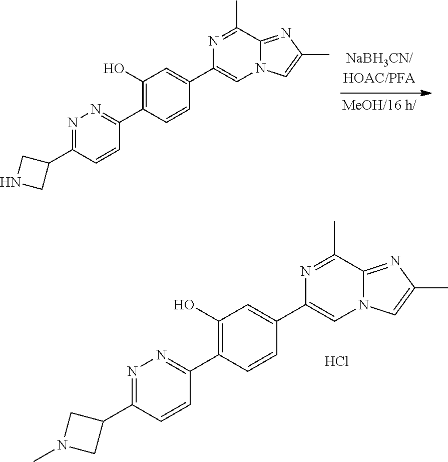

### Step 1: 5-(2,8-dimethylimidazo[1,2-a]pyrazin-6-yl)-2-(6-(1-methylazetidin-3-yl)pyridazin-3-yl)phenol hydrochloride

To a mixture of 2-(6-(azetidin-3-yl)pyridazin-3-yl)-5-(2,8-dimethylimidazo[1,2-a]pyrazin-6-yl)phenol (8 mg, 0.02 mmol) in MeOH (5 mL) were added (HCHO)ₙ (3.2 mg, 0.108 mmmol) and HOAc (0.1 ml) at RT. The mixture was stirred at this temperature for 0.5 hour, NaBH₃CN (4 mg, 0.064 mmol) was added. The mixture was stirred for 16 h. The mixture was concentrated in vacuo and the residue was purified by prep-HPLC (0.05% HCl in water/CH₃CN) to give 5-(2,8-dimethylimidazo[1,2-a]pyrazin-6-yl)-2-(6-(1-methylazetidin-3-yl)pyridazin-3-yl)phenol hydrochloride (2.4 mg, Y:28.8%) as a white solid. ¹H NMR (400 MHz, MeOD-d₄) δ 9.22 (s, 1H), 8.55-8.46 (m, 1H), 8.17 (s, 1H), 8.13 (d, J=7.1 Hz, 1H), 7.86 (d, J=8.9 Hz, 1H), 7.81 (d, J=1.5 Hz, 1H), 7.76 (d, J=8.3 Hz, 1H), 4.76-4.66 (m, 2H), 4.55-4.41 (m, 3H), 3.09 (d, J=7.6 Hz, 3H), 2.66 (s, 3H). ESI-MS (M+H)+: 387.1

### Example 21. 2-[6-(1-methylazetidin-3-yl)pyridazin-3-yl]-5-{2-methylpyrazolo[1,5-a]pyridin-5-yl}phenol (Compound 21)

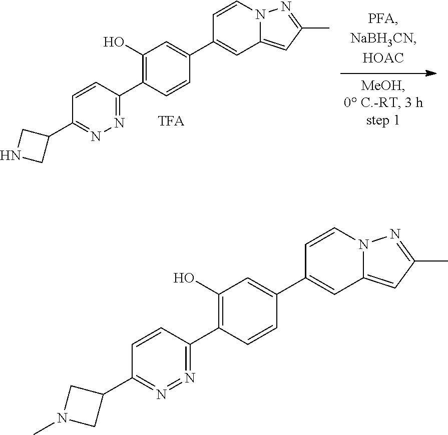

### Step 1: Preparation of 2-(6-(1-methylazetidin-3-yl)pyridazin-3-yl)-5-(2-methylpyrazolo[1,5-a]pyridin-5-yl)phenol

To a solution of 2-(6-(azetidin-3-yl)pyridazin-3-yl)-5-(2-methylpyrazolo[1,5-a]pyridin-5-yl)phenol (30 mg, 0.08 mmol) and (HCHO)ₙ (13 mg, 0.40 mmol) in MeOH (3 mL) was added HOAc (0.05 mL) at RT. After stirred at RT for 1 h, NaBH₃CN (15 mg, 0.24 mmol) in MeOH (1 mL) was added to the mixture at 0° C., the mixture was stirred at RT for 2 h. The resulting reaction mixture was concentrated under reduced pressure and purified by prep-HPLC (0.05% NH₃·H₂O in H2O/MeCN) to give title product (5 mg, Y: 17%) as a yellow solid. ESI-MS (M+H)+: 372.0. ¹H NMR (400 MHz, CDCl₃+CD₃OD) δ 8.45 (d, J=7.2 Hz, 1H), 8.37 (d, J=9.1 Hz, 1H), 8.01 (d, J=8.1 Hz, 1H), 7.87-7.76 (m, 3H), 7.36 (s, 1H), 7.13 (d, J=7.1 Hz, 1H), 6.44 (s, 1H), 4.06 (d, J=7.6 Hz, 1H), 3.88 (t, J=7.6 Hz, 2H), 3.59 (t, J=7.5 Hz, 2H), 3.31 (s, 3H), 2.47 (s, 3H).

### Example 22. 5-(2-methyl-2H-indazol-5-yl)-2-{6-[1-(oxan-4-yl)azetidin-3-yl]pyridazin-3-yl}phenol (Compound 22)

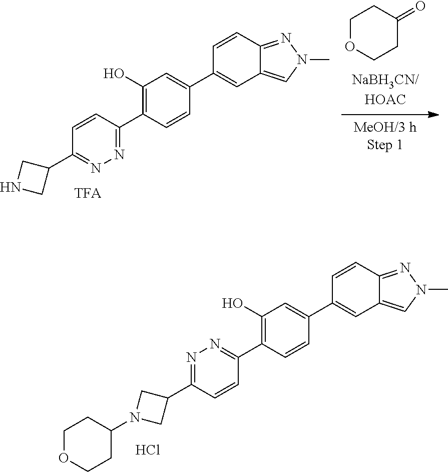

### Step 1: Preparation of 5-(2-methyl-2H-indazol-5-yl)-2-(6-(1-(tetrahydro-2H-pyran-4-yl)azetidin-3-yl)pyridazin-3-yl)phenol hydrochloride

To a solution of 2-(6-(azetidin-3-yl)pyridazin-3-yl)-5-(2-methyl-2H-indazol-5-yl)phenol (1.2 g, 3.36 mmol) in MeOH (100 mL) was added tetrahydro-4H-pyran-4-one (1.68 g, 16.807 mmol) and acetic acid (1 g, 16.807 mmol). The mixture was stirred at RT for 1 h, then sodium cyanoborohydride (635 mg, 10.08 mmol) was added under ice bath and the mixture was stirred at rt for 2 h, then water (10 mL) was added and the solution was concentrated in vacuo. The crude was purified by prep-HPLC (0.05% HCl in water/CH₃CN) to give title compound (657 mg, yield: 44.3%) as a yellow solid. ESI-MS (M+H)+: 442.2. ¹H NMR (400 MHz, DMSO-d₆) δ 12.13-11.31 (m, 1H), 8.64-8.56 (m, 1H), 8.45 (s, 1H), 8.13-8.07 (m, 2H), 7.99 (d, J=9.1 Hz, 1H), 7.71 (d, J=9.0 Hz, 1H), 7.65-7.60 (m, 1H), 7.39-7.36 (m, 2H), 4.64-4.31 (m, 5H), 4.21 (s, 3H), 4.02-3.92 (m, 2H), 3.67-3.44 (m, 1H), 3.34-3.23 (m, 2H), 1.98-1.88 (m, 2H), 1.63-1.49 (m, 2H).

### Example 23. 2-(6-(1-isopropylazetidin-3-yl)pyridazin-3-yl)-5-(2-methyl-2H-indazol-5-yl)phenol (Compound 23)

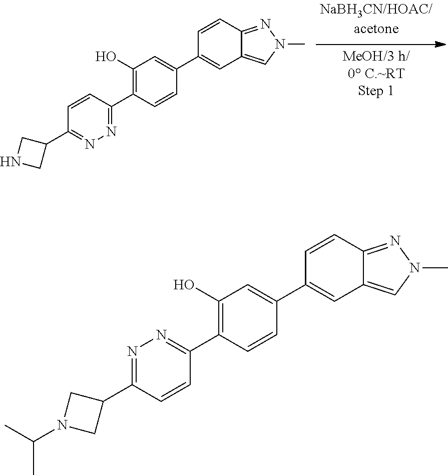

### Step 1: Preparation of 2-(6-(1-isopropylazetidin-3-yl)pyridazin-3-yl)-5-(2-methyl-2H-indazol-5-yl)phenol

To a solution of 2-(6-(azetidin-3-yl)pyridazin-3-yl)-5-(2-methyl-2H-indazol-5-yl)phenol (50 mg, 0.14 mmol) and HOAc (0.09 mL) in MeOH (5 mL) was added acetone (40 mg, 0.7 mmol) in MeOH (5 mL), the reaction mixture was stirred at RT for 1 h. NaBH₃CN (26 mg, 0.42 mmol) in MeOH (2 mL) was added to the mixture at 0° C., the mixture was stirred at RT for 2 h. The resulting reaction mixture diluted with water (5 mL) and extracted with DCM (30 mL×3) The combined organic layer was concentrated under reduced pressure and purified by prep-HPLC (0.05% NH₃·H₂O/CH₃CN) to give title product (7.11 mg, Y: 12%) as a white solid. ESI-MS (M+H)+: 400.1. ¹H NMR (400 MHz, DMSO-d₆) δ 13.35 (s, 1H), 8.58 (d, J=9.2 Hz, 1H), 8.48 (s, 1H), 8.17-8.13 (m, 2H), 8.02 (d, J=9.1 Hz, 1H), 7.75 (d, J=9.0 Hz, 1H), 7.70-7.67 (m, 1H), 7.42-7.38 (m, 2H), 4.26 (s, 3H), 3.96-3.92 (m, 1H), 3.70 (t, J=7.0 Hz, 2H), 2.56 (br.s, 2H), 2.45 (br.s, 1H), 0.96 (d, J=6.2 Hz, 6H).

### Example 24. 2-[6-(1-ethylazetidin-3-yl)pyridazin-3-yl]-5-(2-methyl-2H-indazol-5-yl)phenol (Compound 24)

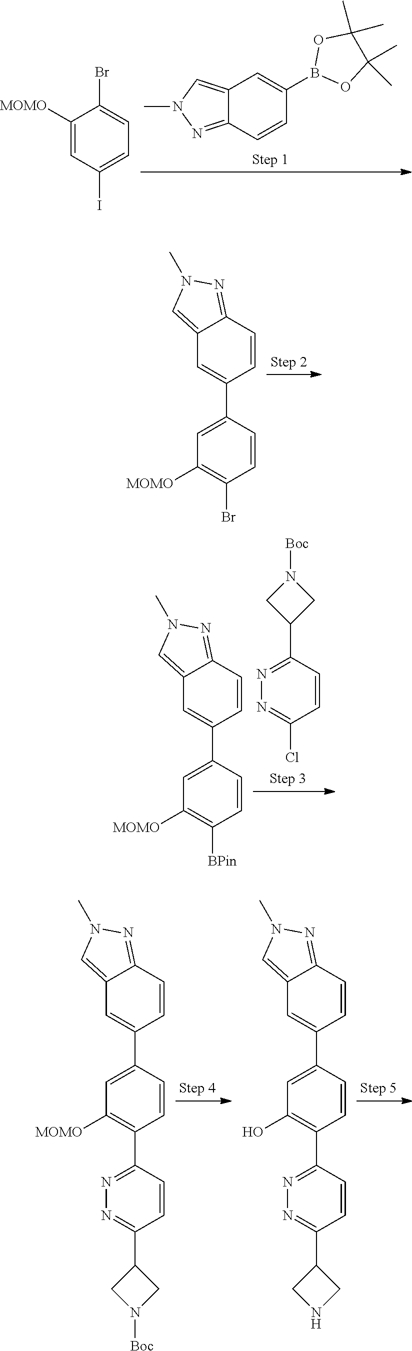

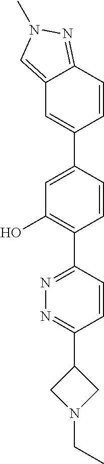 [SMILES: CCN1CC(c2ccc(-c3ccc(-c4ccc5nn(C)cc5c4)cc3O)nn2)C1]

### Step 1: Preparation of 5-(4-bromo-3-(methoxymethoxy)phenyl)-2-methyl-2H-indazole

A solution of 2-methyl-5-(tetramethyl-1,3,2-dioxaborolan-2-yl)-2H-indazole (500.33 mg, 1.94 mmol), 1-bromo-4-iodo-2-(methoxymethoxy)benzene (664.76 mg, 1.94 mmol), [1,1′-bis(diphenylphosphino)ferrocene]dichloropalladium (II) dichloromethane adduct (79.15 mg, 96.92 μmol), and potassium carbonate (535.78 mg, 3.88 mmol) in dioxane (10 mL) and water (2 ml) was degassed and purged with Ar. The resulting mixture was heated at 90° C. overnight. The reaction mixture was cooled to rt EtOAc (15 mL) was added and the mixture was filtered through a pad of Celite. The filtrate was concentrated in vacuo to give crude which was purified by flash column chromatography (Hex/EtOAc 3/1) to give 5-[4-bromo-3-(methoxymethoxy)phenyl]-2-methyl-2H-indazole (380.0 mg, Y: 50.8%). ESI-MS (M+H)+: 347.0 ¹H NMR (400 MHz, DMSO-d₆) δ 8.41 (s, 1H), 7.99 (s, 1H), 7.66 (dd, J=13.2, 8.6 Hz, 2H), 7.58-7.41 (m, 2H), 7.27 (dd, J=8.3, 2.1 Hz, 1H), 5.42 (s, 2H), 4.19 (s, 3H), 3.45 (s, 3H).

### Step 2: Preparation of 5-[3-(methoxymethoxy)-4-(4,4,5,5-tetramethyl-1,3,2-dioxaborolan-2-yl)phenyl]-2-methyl-2H-indazole

A solution of 5-[4-bromo-3-(methoxymethoxy)phenyl]-2-methyl-2H-indazole (380.0 mg, 1.09 mmol), 4,4,5,5-tetramethyl-2-(4,4,5,5-tetramethyl-1,3,2-dioxaborolan-2-yl)-1,3,2-dioxaborolane (278.24 mg, 1.1 mmol), [1,1′-bis(diphenylphosphino)ferrocene]dichloropalladium (II) dichloromethane adduct (89.48 mg, 109.57 μmol), and potassium acetate (215.07 mg, 2.19 mmol) in TolH (10 mL) was degassed and purged with Ar. The resulting mixture was heated at 110° C. overnight. The reaction mixture was diluted with EtOAc (10 mL) and filtered. The filtrate was evaporated to give crude 5-[3-(methoxymethoxy)-4-(4,4,5,5-tetramethyl-1,3,2-dioxaborolan-2-yl)phenyl]-2-methyl-2H-indazole (430.0 mg, Y: 51.8%) which was used in the next step without purification. ESI-MS (M+H)+: 395.2.

### Step 3: Preparation of tert-butyl 3-6-[2-(methoxymethoxy)-4-(2-methyl-2H-indazol-5-yl)phenyl]pyridazin-3-ylazetidine-1-carboxylate

A solution of of 5-[3-(methoxymethoxy)-4-(4,4,5,5-tetramethyl-1,3,2-dioxaborolan-2-yl)phenyl]-2-methyl-2H-indazole (432.0 mg, 1.1 mmol), tert-butyl 3-(6-chloropyridazin-3-yl)azetidine-1-carboxylate (295.77 mg, 1.1 mmol), [1,1′-bis(diphenylphosphino)ferrocene]dichloropalladium (II) dichloromethane adduct (44.77 mg, 54.83 μmol), and potassium carbonate (303.1 mg, 2.19 mmol) in dioxane (10 mL) and water (2 ml) was degassed and purged with Ar. The resulting mixture was heated at 90° C. overnight. The reaction mixture was cooled to rt diluted with MTBE (15 ml) and filtered through a pad of Celite. The filtrate was concentrated in vacuo to give crude, which was purified by preparative HPLC to give tert-butyl 3-6-[2-(methoxymethoxy)-4-(2-methyl-2H-indazol-5-yl)phenyl]pyridazin-3-ylazetidine-1-carboxylate (207.0 mg, Y: 37.6%). ESI-MS (M+H)+: 502.2.

### Step 4: Preparation of 2-[6-(azetidin-3-yl)pyridazin-3-yl]-5-(2-methyl-2H-indazol-5-yl)phenol trifluoroacetate

To a solution of tert-butyl 3-6-[2-(methoxymethoxy)-4-(2-methyl-2H-indazol-5-yl)phenyl]pyridazin-3-ylazetidine-1-carboxylate (207.47 mg, 413.63 μmol) in dichloromethane (5 mL) 2,2,2-trifluoroacetic acid (472.64 mg, 4.15 mmol, 320.0 μl) was added and the mixture was stirred overnight. Then the mixture was evaporated to dryness under reduced pressure to give crude 2-[6-(azetidin-3-yl)pyridazin-3-yl]-5-(2-methyl-2H-indazol-5-yl)phenol trifluoroacetate (135.0 mg, Y: 69.2%) which was used in the next step without purification. ESI-MS (M+H)+: 358.2

### Step 5: Preparation of 2-[6-(I-ethylazetidin-3-yl)pyridazin-3-yl]-5-(2-methyl-2H-indazol-5-yl)phenol trifluoroacetate

2-[6-(azetidin-3-yl)pyridazin-3-yl]-5-(2-methyl-2H-indazol-5-yl)phenol trifluoroacetate (134.02 mg, 284.29 μmol) and acetaldehyde (124.8 mg, 2.83 mmol, 160.0 μl) in methanol was stirred at ambient temperature for 2 h. Sodium cyanoborohydride (89.33 mg, 1.42 mmol) was added, and the mixture was stirred at ambient temperature for 16 h. The reaction mixture was diluted with water and precipitate formed was filtered and purified by HPLC to afford the 2-[6-(1-ethylazetidin-3-yl)pyridazin-3-yl]-5-(2-methyl-2H-indazol-5-yl)phenol trifluoroacetate (2.2 mg, Y: 1.4%). ESI-MS (M+H)+: 386.2.

### Example 25. 2-[6-(1-ethylazetidin-3-yl)pyridazin-3-yl]-5-{2-methylpyrazolo[1,5-a]pyridin-5-yl}phenol (Compound 25)

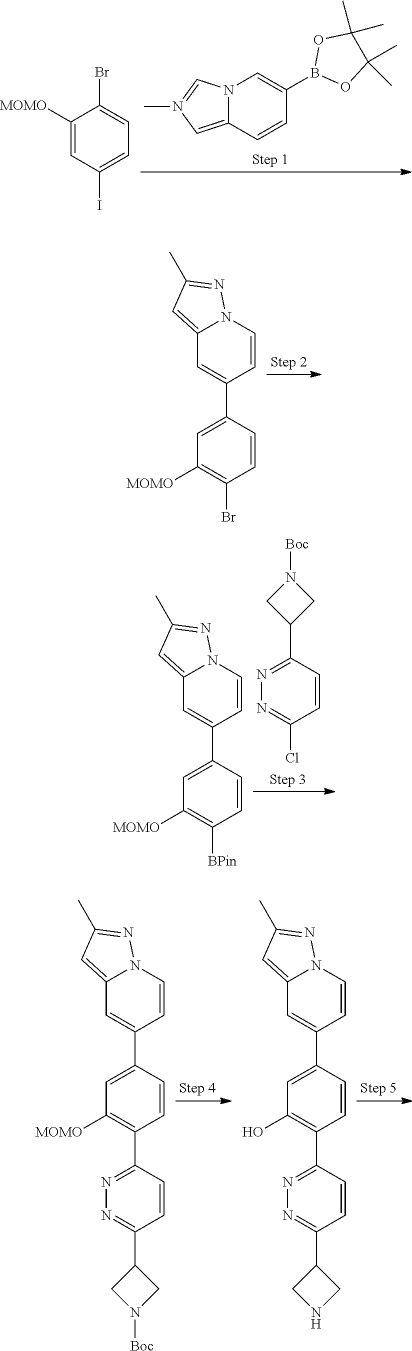

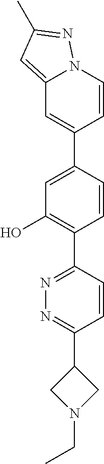 [SMILES: CCN1CC(c2ccc(-c3ccc(-c4ccn5nc(C)cc5c4)cc3O)nn2)C1]

### Step 1: Preparation of 5-[4-bromo-3-(methoxymethoxy)phenyl]-2-methylpyrazolo[1,5-a]pyridine

A solution of 2-methyl-5-(tetramethyl-1,3,2-dioxaborolan-2-yl)pyrazolo[1,5-a]pyridine (399.97 mg, 1.55 mmol), 1-bromo-4-iodo-2-(methoxymethoxy)benzene (531.41 mg, 1.55 mmol), [1,1′-bis(diphenylphosphino)ferrocene]dichloropalladium (II) dichloromethane adduct (63.27 mg, 77.48 μmol), and potassium carbonate (428.3 mg, 3.1 mmol) in dioxane (10 mL) and water (2 ml) was degassed and purged with Ar. The resulting mixture was heated at 90° C. overnight. The reaction mixture was cooled to rt EtOAc (15 mL) was added and the mixture was filtered through a pad of Celite. The filtrate was concentrated in vacuo to give crude which was purified by CC (Hex/EtOAc 1/1) to provide 5-[4-bromo-3-(methoxymethoxy)phenyl]-2-methylpyrazolo[1,5-a]pyridine (356.0 mg, Y: 66.2%). ESI-MS (M+H)+: 347.2. ¹H NMR (400 MHz, DMSO-d₆) δ 8.60 (d, J=7.6, 1H), 7.89 (s, 1H), 7.67 (d, J=8.3, 1H), 7.55 (s, 1H), 7.33 (d, J=8.5, 1H), 7.11 (d, J=7.2, 1H), 6.43 (s, 1H), 5.43 (s, 2H), 3.43 (s, 3H), 2.39 (s, 3H).

### Step 2: Preparation of 5-[3-(methoxymethoxy)-4-(4,4,5,5-tetramethyl-1,3,2-dioxaborolan-2-yl)phenyl]-2-methylpyrazolo[1,5-a]pyridine

A solution of 5-[4-bromo-3-(methoxymethoxy)phenyl]-2-methylpyrazolo[1,5-a]pyridine (199.9 mg, 575.74 μmol), 4,4,5,5-tetramethyl-2-(4,4,5,5-tetramethyl-1,3,2-dioxaborolan-2-yl)-1,3,2-dioxaborolane (146.2 mg, 575.75 μmol), [1,1′-bis(diphenylphosphino)ferrocene]dichloropalladium (II) dichloromethane adduct (23.51 mg, 28.79 μmol), and potassium acetate (169.51 mg, 1.73 mmol) in dioxane (10 mL) was degassed and purged with Ar. The resulting mixture was heated at 90° C. overnight. The reaction mixture was diluted with EtOAc (20 mL) and filtered. The filtrate was evaporated to give crude 5-[3-(methoxymethoxy)-4-(4,4,5,5-tetramethyl-1,3,2-dioxaborolan-2-yl)phenyl]-2-methylpyrazolo[1,5-a]pyridine (400.0 mg) which was used in the next step without purification. ESI-MS (M+H)+: 395.4

### Step 3: Preparation of tert-butyl 3-6-[2-(methoxymethoxy)-4-2-methylpyrazolo[1,5-a]pyridin-5-ylphenyl]pyridazin-3-ylazetidine-1-carboxylate

A solution of of 5-[3-(methoxymethoxy)-4-(4,4,5,5-tetramethyl-1,3,2-dioxaborolan-2-yl)phenyl]-2-methylpyrazolo[1,5-a]pyridine (230.32 mg, 584.16 μmol), tert-butyl 3-(6-chloropyridazin-3-yl)azetidine-1-carboxylate (157.56 mg, 584.16 μmol), [1,1′-bis(diphenylphosphino)ferrocene]dichloropalladium (II) dichloromethane adduct (47.7 mg, 58.42 μmol), and potassium carbonate (161.47 mg, 1.17 mmol) in dioxane (10 mL) and water (2 ml) was degassed and purged with Ar. The resulting mixture was heated at 90° C. overnight. The reaction mixture was cooled to rt diluted with MTBE (15 ml) and filtered through a pad of Celite. The filtrate was concentrated in vacuo to give crude, which was purified by preparative HPLC to give tert-butyl 3-6-[2-(methoxymethoxy)-4-2-methylpyrazolo[1,5-a]pyridin-5-ylphenyl]pyridazin-3-ylazetidine-1-carboxylate (54.0 mg, Y: 18.4%). ESI-MS (M+H)+: 502.4.

### Step 4: Preparation of 2-[6-(azetidin-3-yl)pyridazin-3-yl]-5-2-methylpyrazolo[1,5-a]pyridin-5-ylphenol trifluoroacetate

To a solution of tert-butyl 3-6-[2-(methoxymethoxy)-4-2-methylpyrazolo[1,5-a]pyridin-5-ylphenyl]pyridazin-3-ylazetidine-1-carboxylate (54.05 mg, 107.76 μmol) in dichloromethane (3 mL) 2,2,2-trifluoroacetic acid (122.87 mg, 1.08 mmol) was added and the mixture was stirred overnight. Then the mixture was evaporated to dryness under reduced pressure to give crude 2-[6-(azetidin-3-yl)pyridazin-3-yl]-5-2-methylpyrazolo[1,5-a]pyridin-5-ylphenol trifluoroacetate (50.0 mg, Y: 73.8%) which was used in the next step without purification. ESI-MS (M+H)+: 358.2.

### Step 5: Preparation of 2-[6-(1-ethylazetidin-3-yl)pyridazin-3-yl]-5-2-methylpyrazolo[1,5-a]pyridin-5-ylphenol trifluoroacetate

A solution of 2-[6-(azetidin-3-yl)pyridazin-3-yl]-5-2-methylpyrazolo[1,5-a]pyridin-5-ylphenol trifluoroacetate (50.0 mg, 106.06 μmol) and acetaldehyde (46.74 mg, 1.06 mmol) in methanol was stirred at ambient temperature for 2 h. Sodium cyanoborohydride (33.34 mg, 530.53 μmol) was added, and the mixture was stirred at ambient temperature for 16 hrs. The reaction mixture was diluted with water and precipitate formed was filtered and purified by HPLC to afford the 2-[6-(1-ethylazetidin-3-yl)pyridazin-3-yl]-5-2-methylpyrazolo[1,5-a]pyridin-5-ylphenol trifluoroacetate (25.0 mg, Y: 61.1%). ESI-MS (M+H)+: 386.4. ¹H NMR (400 MHz, Acetonitrile-d₃) δ 8.66 (d, J=7.4 Hz, 1H), 8.48 (br s, 1H), 8.34-8.25 (m, 1H), 7.96 (dd, J=8.3, 1.9 Hz, 1H), 7.92 (s, 1H), 7.75-7.67 (m, 1H), 7.36-7.33 (m, 1H), 7.32-7.26 (m, 1H), 4.71-4.54 (m, 2H), 4.46-4.27 (m, 3H), 3.34 (q, J=6.9, 2H), 2.47 (s, 3H), 1.95 (d, J=2.3 Hz, 11H), 1.21 (t, J=7.4 Hz, 3H).

### Example 26. 5-[2-methyl-8-(trifluoromethyl)imidazo[1,2-a]pyridin-6-yl]-2-[6-(1-methylazetidin-3-yl)pyridazin-3-yl]phenol (Compound 26)

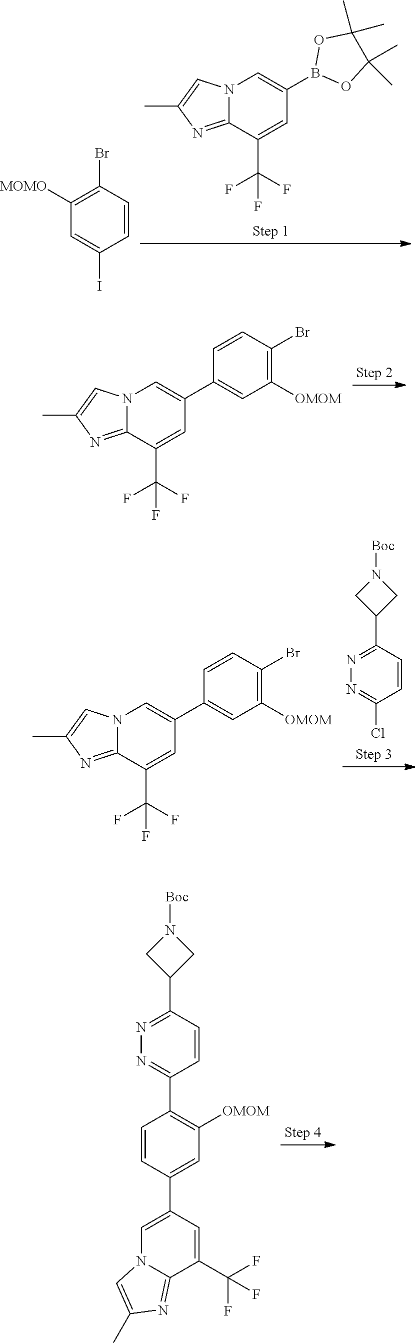

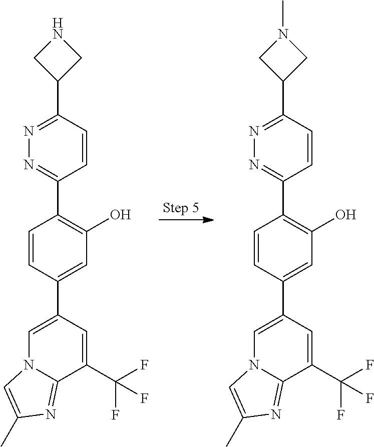 [SMILES: Cc1cn2cc(-c3ccc(-c4ccc(C5CNC5)nn4)c(O)c3)cc(C(F)(F)F)c2n1 ; Cc1cn2cc(-c3ccc(-c4ccc(C5CN(C)C5)nn4)c(O)c3)cc(C(F)(F)F)c2n1]

### Step 1: Preparation of 6-[4-bromo-3-(methoxymethoxy)phenyl]-2-methyl-8-(trifluoromethyl)imidazo[1,2-a]pyridine

A solution of 2-methyl-6-(4,4,5,5-tetramethyl-1,3,2-dioxaborolan-2-yl)-8-(trifluoromethyl)imidazo[1,2-a]pyridine (500.0 mg, 1.53 mmol), 1-bromo-4-iodo-2-(methoxymethoxy)benzene (526.16 mg, 1.53 mmol), [1,1′-bis(diphenylphosphino)ferrocene]dichloropalladium (II) dichloromethane adduct (62.64 mg, 76.71 μmol), and potassium carbonate (424.07 mg, 3.07 mmol) in dioxane (10 mL) and water (2 ml) was degassed and purged with Ar. The resulting mixture was heated at 90° C. overnight. The reaction mixture was cooled to rt EtOAc (20 ml) was added and the mixture was filtered through a pad of Celite. The filtrate was concentrated in vacuo to give crude which was purified by flash column chromatography (Hex/EtOAc 1/1) to provide 6-[4-bromo-3-(methoxymethoxy)phenyl]-2-methyl-8-(trifluoromethyl)imidazo[1,2-a]pyridine (283.0 mg, Y: 41.8%). ESI-MS (M+H)+: 415.0.

### Step 2: Preparation of 6-[3-(methoxymethoxy)-4-(4,4,5,5-tetramethyl-1,3,2-dioxaborolan-2-yl)phenyl]-2-methyl-8-(trifluoromethyl)imidazo[1,2-a]pyridine

A solution of 6-[4-bromo-3-(methoxymethoxy)phenyl]-2-methyl-8-(trifluoromethyl)imidazo[1,2-a]pyridine (283.0 mg, 681.59 μmol), 4,4,5,5-tetramethyl-2-(4,4,5,5-tetramethyl-1,3,2-dioxaborolan-2-yl)-1,3,2-dioxaborolane (190.34 mg, 749.56 μmol), potassium acetate (200.63 mg, 2.04 mmol), and [1,1′-bis(diphenylphosphino)ferrocene]dichloropalladium (II) dichloromethane adduct (27.82 mg, 34.07 μmol) in dioxane (8 mL) was degassed and purged with Ar. The resulting mixture was heated at 90° C. overnight. The reaction mixture was cooled to rt, filtered through a pad of Celite, and concentrated in vacuo, and the residue (315 mg) was used directly in the next step without further purification. ESI-MS (M+H)+: 463.2.

### Step 3: Preparation of tert-butyl 3-6-[2-(methoxymethoxy)-4-[2-methyl-8-(trifluoromethyl)imidazo[1,2-a]pyridin-6-yl]phenyl]pyridazin-3-ylazetidine-1-carboxylate

A solution of 6-[3-(methoxymethoxy)-4-(4,4,5,5-tetramethyl-1,3,2-dioxaborolan-2-yl)phenyl]-2-methyl-8-(trifluoromethyl)imidazo[1,2-a]pyridine (315.0 mg, 681.42 μmol), tert-butyl 3-(6-chloropyridazin-3-yl)azetidine-1-carboxylate (183.74 mg, 681.21 μmol), [1,1′-bis(diphenylphosphino)ferrocene]dichloropalladium(II) dichloromethane adduct (27.82 mg, 34.06 μmol), and potassium carbonate (188.29 mg, 1.36 mmol) in dioxane (10 mL) and water (2 ml) was degassed and purged with Ar. The resulting mixture was heated at 95° C. overnight. The reaction mixture was cooled to rt. EtOAc (20 mL) was added and the mixture was filtered through a pad of Celite. The filtrate was concentrated in vacuo to give crude which was purified by HPLC to provide tert-butyl 3-6-[2-(methoxymethoxy)-4-[2-methyl-8-(trifluoromethyl)imidazo[1,2-a]pyridin-6-yl]phenyl]pyridazin-3-ylazetidine-1-carboxylate (125.9 mg, Y: 32.4%). ESI-MS (M+H)+: 570.2.

### Step 4: Preparation of 2-[6-(azetidin-3-yl)pyridazin-3-yl]-5-[2-methyl-8-(trifluoromethyl)imidazo[1,2-a]pyridin-6-yl]phenol trifluoroacetate

To a solution of tert-butyl 3-6-[2-(methoxymethoxy)-4-[2-methyl-8-(trifluoromethyl)imidazo[1,2-a]pyridin-6-yl]phenyl]pyridazin-3-ylazetidine-1-carboxylate (125.65 mg, 220.6 μmol) in dichloromethane (4 mL) 2,2,2-trifluoroacetic acid (251.09 mg, 2.2 mmol, 170.0 μl) was added and the mixture was stirred overnight. Then the mixture was evaporated to dryness under reduced pressure to obtain 2-[6-(azetidin-3-yl)pyridazin-3-yl]-5-[2-methyl-8-(trifluoromethyl)imidazo[1,2-a]pyridin-6-yl]phenol trifluoroacetate (135.0 mg, Y: 98%). ESI-MS (M+H)+: 426.2.

### Step 5: Preparation of 5-[2-methyl-8-(trifluoromethyl)imidazo[1,2-a]pyridin-6-yl]-2-[6-(1-methylazetidin-3-yl)pyridazin-3-yl]phenol trifluoroacetate

A solution of 2-[6-(azetidin-3-yl)pyridazin-3-yl]-5-[2-methyl-8-(trifluoromethyl)imidazo[1,2-a]pyridin-6-yl]phenol trifluoroacetate (135.0 mg, 250.26 μmol), acetic acid (150.32 mg, 2.5 mmol) and formaldehyde (75.16 mg, 2.5 mmol) in methanol was stirred at ambient temperature for 2 h. Sodium cyanoborohydride (157.31 mg, 2.5 mmol) was added, and the mixture was stirred at ambient temperature for 16 h. The reaction mixture was diluted with water and precipitate formed was filtered and dried to afford the 5-[2-methyl-8-(trifluoromethyl)imidazo[1,2-a]pyridin-6-yl]-2-[6-(1-methylazetidin-3-yl)pyridazin-3-yl]phenol trifluoroacetate (78.0 mg, Y: 64.5%). ESI-MS (M+H)+: 440.2. ¹H NMR (400 MHz, Methanol-d₄) δ 8.86 (s, 1H), 8.31 (d, J=9.1 Hz, 1H), 7.95 (d, J=8.7 Hz, 1H), 7.85 (s, 1H), 7.77-7.68 (m, 2H), 7.26-7.22 (m, 2H), 4.56-4.49 (m, 2H), 4.45-4.39 (m, 2H), 4.36-4.29 (m, 1H), 2.99 (s, 3H), 2.40 (s, 3H).

### Example 27. 5-(2,7-dimethyl-2H-indazol-5-yl)-2-[6-(1-methylazetidin-3-yl)pyridazin-3-yl]phenol (Compound 27)

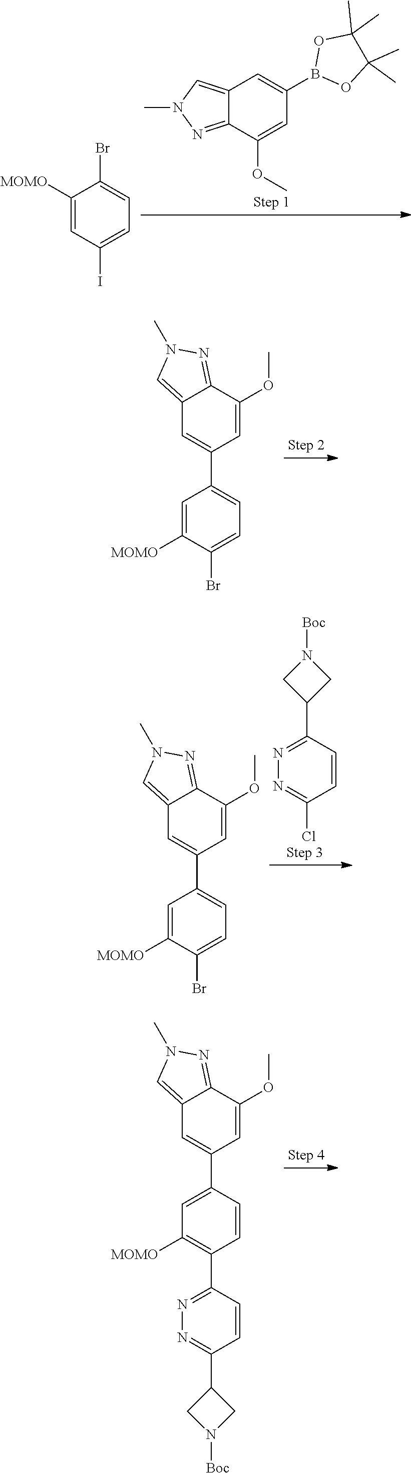

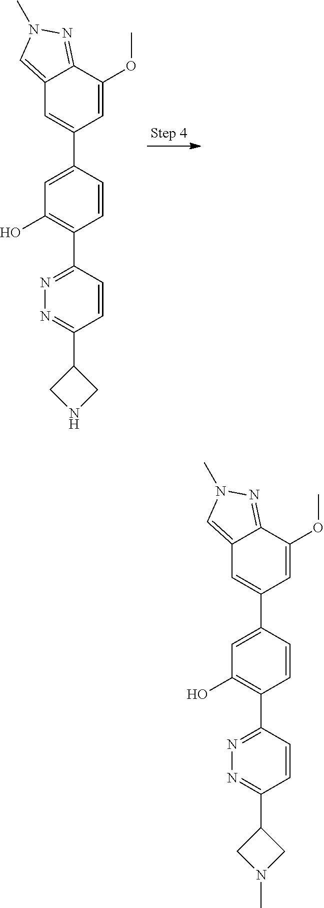 [SMILES: COc1cc(-c2ccc(-c3ccc(C4CNC4)nn3)c(O)c2)cc2cn(C)nc12 ; COc1cc(-c2ccc(-c3ccc(C4CN(C)C4)nn3)c(O)c2)cc2cn(C)nc12]

### Step 1: Preparation of 5-[4-bromo-3-(methoxymethoxy)phenyl]-2,7-dimethyl-2H-indazole

A solution of 2,7-dimethyl-5-(tetramethyl-1,3,2-dioxaborolan-2-yl)-2H-indazole (400.0 mg, 1.47 mmol), 1-bromo-4-iodo-2-(methoxymethoxy)benzene (504.13 mg, 1.47 mmol), [1,1′-bis(diphenylphosphino)ferrocene]dichloropalladium (II) dichloromethane adduct (60.02 mg, 73.5 μmol), and potassium carbonate (406.31 mg, 2.94 mmol) in dioxane (15 mL) and water (4 ml) was degassed and purged with Ar. The resulting mixture was heated at 90° C. overnight. The reaction mixture was cooled to rt, MTBE (25 ml) was added and precipitate formed was filtered through a pad of Celite. The precipitate was washed with MeCN/MTBE(1/1, 5 ml) and the filtrate was concentrated in vacuo to give pure 5-[4-bromo-3-(methoxymethoxy)phenyl]-2,7-dimethyl-2H-indazole (400.0 mg, Y: 66.3%). ESI-MS (M+H)+: 361.0.

### Step 2: Preparation of 5-[3-(methoxymethoxy)-4-(4,4,5,5-tetramethyl-1,3,2-dioxaborolan-2-yl)phenyl]-2,7-dimethyl-2H-indazole

A solution of 5-[4-bromo-3-(methoxymethoxy)phenyl]-2,7-dimethyl-2H-indazole (236.22 mg, 653.93 μmol), 4,4,5,5-tetramethyl-2-(4,4,5,5-tetramethyl-1,3,2-dioxaborolan-2-yl)-1,3,2-dioxaborolane (166.06 mg, 653.93 μmol), [1,1′-bis(diphenylphosphino)ferrocene]dichloropalladium (II) dichloromethane adduct (53.4 mg, 65.39 μmol), and potassium acetate (128.36 mg, 1.31 mmol) in TolH (10 mL) was degassed and purged with Ar. The resulting mixture was heated at 120° C. overnight. The reaction mixture was diluted with EtOAc (10 mL) and filtered. The filtrate was evaporated to give crude 5-[3-(methoxymethoxy)-4-(4,4,5,5-tetramethyl-1,3,2-dioxaborolan-2-yl)phenyl]-2,7-dimethyl-2H-indazole (300.0 mg) which was used in the next step without purification. ESI-MS (M+H)+: 409.4.

### Step 3: Preparation of tert-butyl 3-6-[4-(2,7-dimethyl-2H-indazol-5-yl)-2-(methoxymethoxy)phenyl]pyridazin-3-ylazetidine-1-carboxylate

A solution of 5-[3-(methoxymethoxy)-4-(4,4,5,5-tetramethyl-1,3,2-dioxaborolan-2-yl)phenyl]-2,7-dimethyl-2H-indazole (299.33 mg, 733.12 μmol), tert-butyl 3-(6-chloropyridazin-3-yl)azetidine-1-carboxylate (98.87 mg, 366.56 μmol), [1,1′-bis(diphenylphosphino)ferrocene]dichloropalladium (II) dichloromethane adduct (29.93 mg, 36.66 μmol), and potassium carbonate (101.32 mg, 733.12 μmol) in dioxane (10 mL) and water (2 ml) was degassed and purged with Ar. The resulting mixture was heated at 90° C. overnight. The reaction mixture was cooled to rt diluted with MTBE (15 ml) and filtered through a pad of Celite. The filtrate was concentrated in vacuo to give crude, which was purified by preparative HPLC to give tert-butyl 3-6-[4-(2,7-dimethyl-2H-indazol-5-yl)-2-(methoxymethoxy)phenyl]pyridazin-3-ylazetidine-1-carboxylate (80.0 mg, Y: 34.7%). ESI-MS (M+H)+: 516.2.

### Step 4: Preparation of 2-[6-(azetidin-3-yl)pyridazin-3-yl]-5-(2,7-dimethyl-2H-indazol-5-yl)phenol trifluoroacetate

To a solution of tert-butyl 3-6-[4-(2,7-dimethyl-2H-indazol-5-yl)-2-(methoxymethoxy)phenyl]-pyridazin-3-ylazetidine-1-carboxylate (79.98 mg, 155.11 μmol) in dichloromethane (3 mL) 2,2,2-trifluoroacetic acid (176.86 mg, 1.55 mmol) was added and the mixture was stirred overnight. Then the mixture was evaporated to dryness under reduced pressure to give 2-[6-(azetidin-3-yl)pyridazin-3-yl]-5-(2,7-dimethyl-2H-indazol-5-yl)phenol trifluoroacetate (80.0 mg, Y: 95.6%) which was used in the next step without purification. ESI-MS (M+H)+: 372.0.

### Step 5: Preparation of 5-(2,7-dimethyl-2H-indazol-5-yl)-2-[6-(I-methylazetidin-3-yl)pyridazin-3-yl]phenol trifluoroacetate

A solution 2-[6-(azetidin-3-yl)pyridazin-3-yl]-5-(2,7-dimethyl-2H-indazol-5-yl)phenol trifluoroacetate (119.55 mg, 246.25 μmol) and formaldehyde (73.94 mg, 2.46 mmol) in methanol was stirred at ambient temperature for 2 h. Sodium cyanoborohydride (46.43 mg, 738.76 μmol) was added, and the mixture was stirred at ambient temperature for 16 hrs. The reaction mixture was diluted with water and precipitate formed was filtered and purified by HPLC to afford the 5-(2,7-dimethyl-2H-indazol-5-yl)-2-[6-(1-methylazetidin-3-yl)pyridazin-3-yl]phenol trifluoroacetate (22.0 mg, Y: 17.9%). ESI-MS (M+H)+: 386.2. ¹H NMR (400 MHz, DMSO-d₆) δ 8.54 (d, J=9.2 Hz, 1H), 8.36 (s, 1H), 8.07 (d, J=8.2 Hz, 1H), 7.91 (d, J=9.1 Hz, 1H), 7.85 (s, 1H), 7.39 (s, 1H), 7.31-7.22 (m, 2H), 4.18 (s, 3H), 4.11-3.82 (m, 2H), 3.70-3.60 (m, 3H), 2.29 (s, 3H).

### Example 28. 5-(7-methoxy-2-methyl-2H-indazol-5-yl)-2-[6-(1-methylazetidin-3-yl)pyridazin-3-yl]phenol (Compound 28)

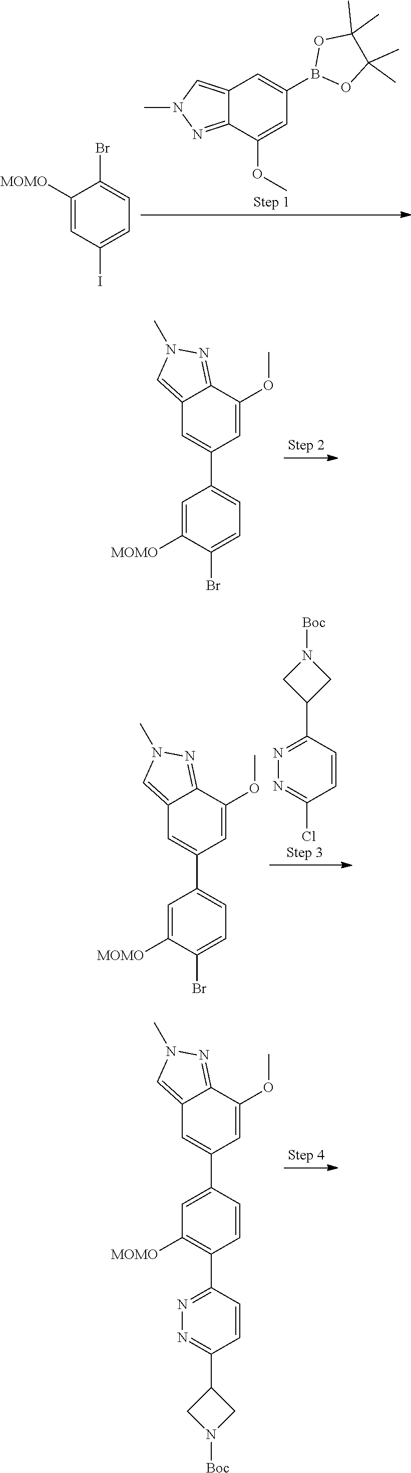

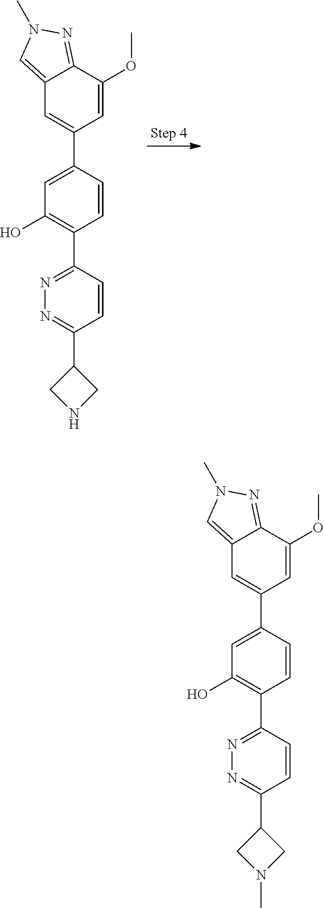 [SMILES: COc1cc(-c2ccc(-c3ccc(C4CNC4)nn3)c(O)c2)cc2cn(C)nc12 ; COc1cc(-c2ccc(-c3ccc(C4CN(C)C4)nn3)c(O)c2)cc2cn(C)nc12]

### Step 1: Preparation of 5-[4-bromo-3-(methoxymethoxy)phenyl]-7-methoxy-2-methyl-2H-indazole

1-bromo-4-iodo-2-(methoxymethoxy)benzene (761.86 mg, 2.22 mmol), 7-methoxy-2-methyl-5-(4,4,5,5-tetramethyl-1,3,2-dioxaborolan-2-yl)-2H-indazole (800.0 mg, 2.78 mmol), [1,1′-bis(diphenylphosphino)ferrocene]dichloropalladium (II) dichloromethane adduct (90.71 mg, 111.07 μmol), and potassium carbonate (614.03 mg, 4.44 mmol) in dioxane (10 mL) and water (2 ml) was degassed and purged with Ar. The resulting mixture was heated at 90° C. overnight. The reaction mixture was cooled to rt EtOAc (15 mL) was added and the mixture was filtered through a pad of Celite. The filtrate was concentrated in vacuo to give crude which was purified by CC (Hex/EtOAc 1/1) to provide 5-[4-bromo-3-(methoxymethoxy)phenyl]-7-methoxy-2-methyl-2H-indazole (370.0 mg, 980.83 μmol, 44.2% yield). ESI-MS (M+H)+: 378.2. ¹H NMR (400 MHz, DMSO-d₆) δ 8.34 (s, 1H), 7.64 (d, J=8.3, 1H), 7.49 (d, J=9.1, 2H), 7.36-7.20 (m, 1H), 6.85 (s, 1H), 5.42 (s, 2H), 4.15 (s, 3H), 3.99 (s, 3H), 3.41 (s, 3H), 3.32 (s, 1H).

### Step 2: Preparation of 7-methoxy-5-[3-(methoxymethoxy)-4-(4,4,5,5-tetramethyl-1,3,2-dioxaborolan-2-yl)phenyl]-2-methyl-2H-indazole

A solution of 5-[4-bromo-3-(methoxymethoxy)phenyl]-7-methoxy-2-methyl-2H-indazole (365.0 mg, 967.57 μmol), 4,4,5,5-tetramethyl-2-(4,4,5,5-tetramethyl-1,3,2-dioxaborolan-2-yl)-1,3,2-dioxaborolane (245.98 mg, 968.66 μmol), [1,1′-bis(diphenylphosphino)ferrocene]dichloropalladium (II) dichloromethane adduct (79.1 mg, 96.87 μmol), and potassium acetate (190.13 mg, 1.94 mmol) in dioxane (10 mL) was degassed and purged with Ar. The resulting mixture was heated at 90° C. overnight. The reaction mixture was diluted with EtOAc (20 mL) and filtered. The filtrate was evaporated to give crude 7-methoxy-5-[3-(methoxymethoxy)-4-(4,4,5,5-tetramethyl-1,3,2-dioxaborolan-2-yl)phenyl]-2-methyl-2H-indazole (290.0 mg) which was used in the next step without purification. ESI-MS (M+H)+: 425.2.

### Step 3: Preparation of tert-butyl 3-6-[4-(7-methoxy-2-methyl-2H-indazol-5-yl)-2-(methoxymethoxy)phenyl]pyridazin-3-ylazetidine-1-carboxylate

A solution of of 7-methoxy-5-[3-(methoxymethoxy)-4-(4,4,5,5-tetramethyl-1,3,2-dioxaborolan-2-yl)phenyl]-2-methyl-2H-indazole (290.53 mg, 684.72 μmol), tert-butyl 3-(6-chloropyridazin-3-yl)azetidine-1-carboxylate (92.34 mg, 342.36 μmol), [1,1′-bis(diphenylphosphino)ferrocene]dichloropalladium (II) dichloromethane adduct (27.96 mg, 34.24 μmol), and potassium carbonate (94.63 mg, 684.72 μmol) in dioxane (10 mL) and water (2 ml) was degassed and purged with Ar. The resulting mixture was heated at 90° C. overnight. The reaction mixture was cooled to rt diluted with MTBE (15 ml) and filtered through a pad of Celite. The filtrate was concentrated in vacuo to give crude, which was purified by preparative HPLC to give tert-butyl 3-6-[4-(7-methoxy-2-methyl-2H-indazol-5-yl)-2-(methoxymethoxy)phenyl]pyridazin-3-ylazetidine-1-carboxylate (29.0 mg, Y: 12.7%). ESI-MS (M+H)+: 532.2.

### Step 4: Preparation of 2-[6-(azetidin-3-yl)pyridazin-3-yl]-5-(7-methoxy-2-methyl-2H-indazol-5-yl)phenol trifluoroacetate

To a solution of tert-butyl 3-6-[4-(7-methoxy-2-methyl-2H-indazol-5-yl)-2-(methoxymethoxy)phenyl]pyridazin-3-ylazetidine-1-carboxylate (29 mg, 54.64 μmol) in dichloromethane (3 mL) 2,2,2-trifluoroacetic acid (122.87 mg, 1.08 mmol) was added and the mixture was stirred overnight. Then the mixture was evaporated to dryness under reduced pressure to give crude 2-[6-(azetidin-3-yl)pyridazin-3-yl]-5-(7-methoxy-2-methyl-2H-indazol-5-yl)phenol trifluoroacetate (28.0 mg, 96.0% purity, 53.6 μmol, 98.1% yield) which was used in the next step without purification. ESI-MS (M+H)+: 388.2.

### Step 5: Preparation of 5-(7-methoxy-2-methyl-2H-indazol-5-yl)-2-[6-(I-methylazetidin-3-yl)pyridazin-3-yl]phenol trifluoroacetate

A solution of 2-[6-(azetidin-3-yl)pyridazin-3-yl]-5-(7-methoxy-2-methyl-2H-indazol-5-yl)phenol trifluoroacetate (28.0 mg, 53.6 μmol) and formaldehyde (45.68 mg, 1.52 mmol, 40.0 μl, 10.0 equiv) in methanol was stirred at ambient temperature for 2 h. Sodium cyanoborohydride (10.9 mg, 173.43 μmol) was added, and the mixture was stirred at ambient temperature for 16 hrs. The reaction mixture was diluted with water and precipitate formed was filtered and purified by HPLC to afford the 5-(7-methoxy-2-methyl-2H-indazol-5-yl)-2-[6-(1-methylazetidin-3-yl)pyridazin-3-yl]phenol trifluoroacetate (6.7 mg, Y: 21.4%). ESI-MS (M+H)+: 402.0.

### Example 29. 2-[6-(azetidin-3-yl)pyridazin-3-yl]-5-{2,7-dimethylpyrazolo[1,5-a]pyridin-5-yl}phenol (Compound 29)

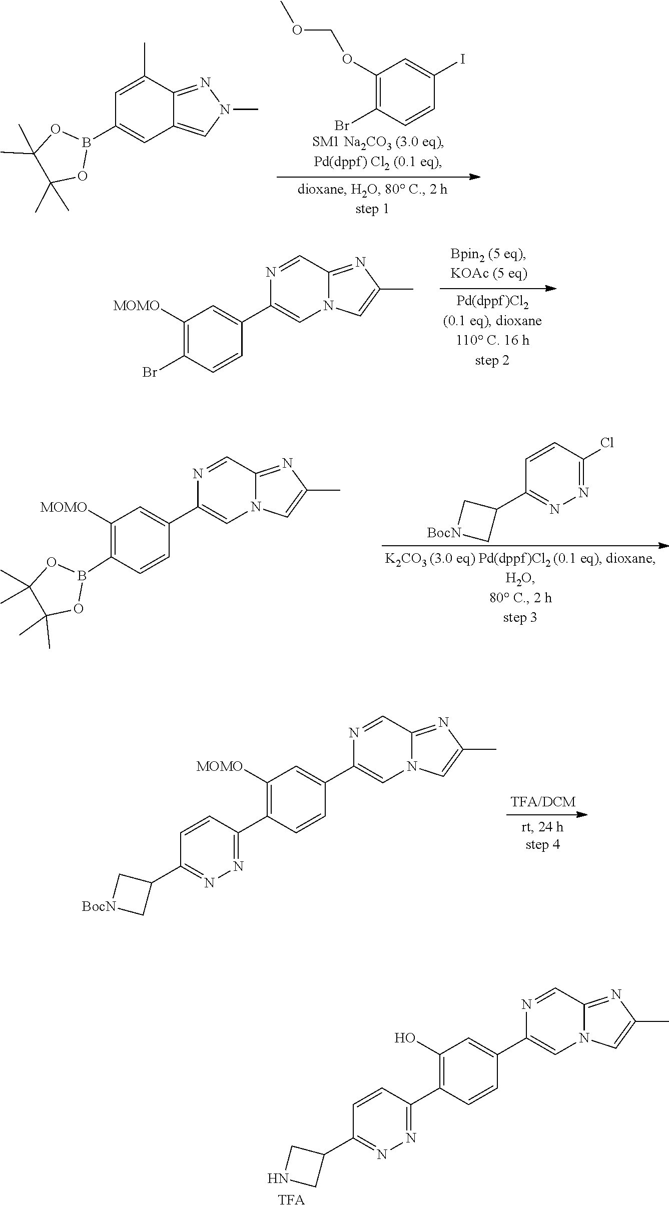

### Step 1: Preparation of 5-(4-bromo-3-(methoxymethoxy)phenyl)-2,7-dimethylpyrazolo[1,5-a]pyridine

A mixture of 1-bromo-4-iodo-2-(methoxymethoxy)benzene (1.23 g, 3.6 mmol), 2,7-dimethyl-5-(4,4,5,5-tetramethyl-1,3,2-dioxaborolan-2-yl)pyrazolo[1,5-a]pyridine (956 mg, 3.6 mmol), Na₂CO₃ (1.13 g, 1.08 mmol) and Pd(dppf)Cl₂ (262 mg, 0.36 mmol) in 1,4-dioxane/H₂O (110 mL/11 mL) was stirred at 80° C. for 2 h. The reaction mixture was diluted with water (150 mL) and extracted with EtOAc (150 mL×3). The organic layer was washed with brine, dried over Na₂SO₄ and evaporated to give crude title compound. The crude was purified by silica gel column chromatography (PE/EA=5:1) to give title compound (360 mg, 30% yield) as a yellow solid. ESI-MS (M+H)+: 361.1. ¹H NMR (400 MHz, CDCl₃) δ 7.60 (d, J=8.2 Hz, 1H), 7.49 (s, 1H), 7.39 (d, J=1.8 Hz, 1H), 7.15 (dd, J=8.2, 1.9 Hz, 1H), 6.74 (s, 1H), 6.37 (s, 1H), 5.33 (s, 2H), 3.57 (s, 3H), 2.78 (s, 3H), 2.54 (s, 3H).

### Step 2: Preparation of 5-(3-(methoxymethoxy)-4-(4,4,5,5-tetramethyl-1,3,2-dioxaborolan-2-yl)phenyl)-2,7-dimethylpyrazolo[1,5-a]pyridine

A mixture of 5-(4-bromo-3-(methoxymethoxy)phenyl)-2,7-dimethylpyrazolo[1,5-a]pyridine (360 mg, 9.7 mmol), B₂Pin₂ (2 g, 7.76 mmol), KOAc (480 mg, 4.85 mmol) and Pd(dppf)Cl₂ (140 mg, 0.196 mmol) in 1,4-dioxane (35 mL) was stirred at 110° C. for 16 h. The reaction mixture was diluted with water (50 mL) and extracted with EtOAc (50 mL×3). The organic layer was washed with brine, dried over Na₂SO₄ and evaporated to give crude title compound. The crude was purified by silica gel column chromatography (PE/EA=2:1) to give title compound (360 mg, 90% yield) as a yellow solid. ESI-MS (M+H)+: 409.1. ¹H NMR (400 MHz, CDCl₃) δ 7.78 (d, J=8.2 Hz, 1H), 7.60-7.47 (m, 1H), 7.29 (dd, J=11.3, 4.4 Hz, 2H), 6.80 (s, 1H), 6.37 (s, 1H), 5.28 (s, 2H), 3.56 (s, 3H), 2.78 (s, 3H), 2.54 (s, 3H), 1.37 (s, 12H).

### Step 3: Preparation of tert-butyl 3-(6-(4-(2,7-dimethylpyrazolo[1,5-a]pyridin-5-yl)-2-(methoxymethoxy)phenyl)pyridazin-3-yl)azetidine-1-carboxylate

A mixture of 5-(3-(methoxymethoxy)-4-(4,4,5,5-tetramethyl-1,3,2-dioxaborolan-2-yl)phenyl)-2,7-dimethylpyrazolo[1,5-a]pyridine (360 mg, 0.88 mmol), tert-butyl 3-(6-chloropyridazin-3-yl)azetidine-1-carboxylate (236.7 mg, 0.88 mmol), K₂CO₃ (364.32 mg, 2.64 mmol) and Pd(dppf)Cl₂ (64 mg, 0.088 mmol) in 1,4-dioxane/H₂O (60 mL/10 mL) was stirred at 80° C. for 2 h. The reaction mixture was diluted with water (50 mL) and extracted with EtOAc (50 mL×3). The organic layer was washed with brine, dried over Na₂SO₄ and evaporated to give crude title compound. The crude was purification by C₁₈-flash (0.1% FA in water/CH₃CN) to give title compound (240 mg, 52.8% yield) as a yellow solid. ESI-MS (M+H)+: 516.4. ¹H NMR (400 MHz, CDCl₃) δ 8.09 (d, J=2.1 Hz, 1H), 8.07 (d, J=2.9 Hz, 1H), 7.60 (s, 1H), 7.53 (d, J=8.8 Hz, 2H), 7.47 (dd, J=8.1, 1.6 Hz, 1H), 6.85 (s, 1H), 6.41 (s, 1H), 5.31 (s, 2H), 4.47-4.39 (m, 2H), 4.35-4.23 (m, 2H), 4.20-4.06 (m, 1H), 3.50 (s, 3H), 2.81 (s, 3H), 2.55 (s, 3H), 1.48 (s, 9H).

### Step 4: Preparation of 2-(6-(azetidin-3-yl)pyridazin-3-yl)-5-(2,7-dimethylpyrazolo[1,5-a]pyridin-5-yl)phenol

To a solution of tert-butyl 3-(6-(4-(2,7-dimethylpyrazolo[1,5-a]pyridin-5-yl)-2-(methoxymethoxy)phenyl)pyridazin-3-yl)azetidine-1-carboxylate (240 mg, 0.48 mmol) in DCM (10 mL) was added TFA (10 ml) at room temperature. The reaction mixture was stirred for 24 h. The reaction mixture was diluted with DCM (100 mL) and concentrated in vacuo. The residue was purified by pre-HPLC (0.05% TFA in water/CH₃CN) to give title compound (180 mg, Y: 79%) as a yellow solid. ESI-MS (M+H)+: 372.2. ¹H NMR (400 MHz, DMSO-d₆) δ 8.94-8.92 (m, 2H), 8.59 (d, J=9.1 Hz, 1H), 8.16 (d, J=8.2 Hz, 1H), 7.92 (d, J=9.1 Hz, 1H), 7.90 (s, 1H), 7.47-7.44 (m, 2H), 7.18 (s, 1H), 6.52 (s, 1H), 4.45-4.31 (m, 5H), 2.73 (s, 3H), 2.45 (s, 3H).

### Example 30. 5-{2,8-dimethylimidazo[1,2-b]pyridazin-6-yl}-2-[6-(1-methylazetidin-3-yl)pyridazin-3-yl]phenol (Compound 30)

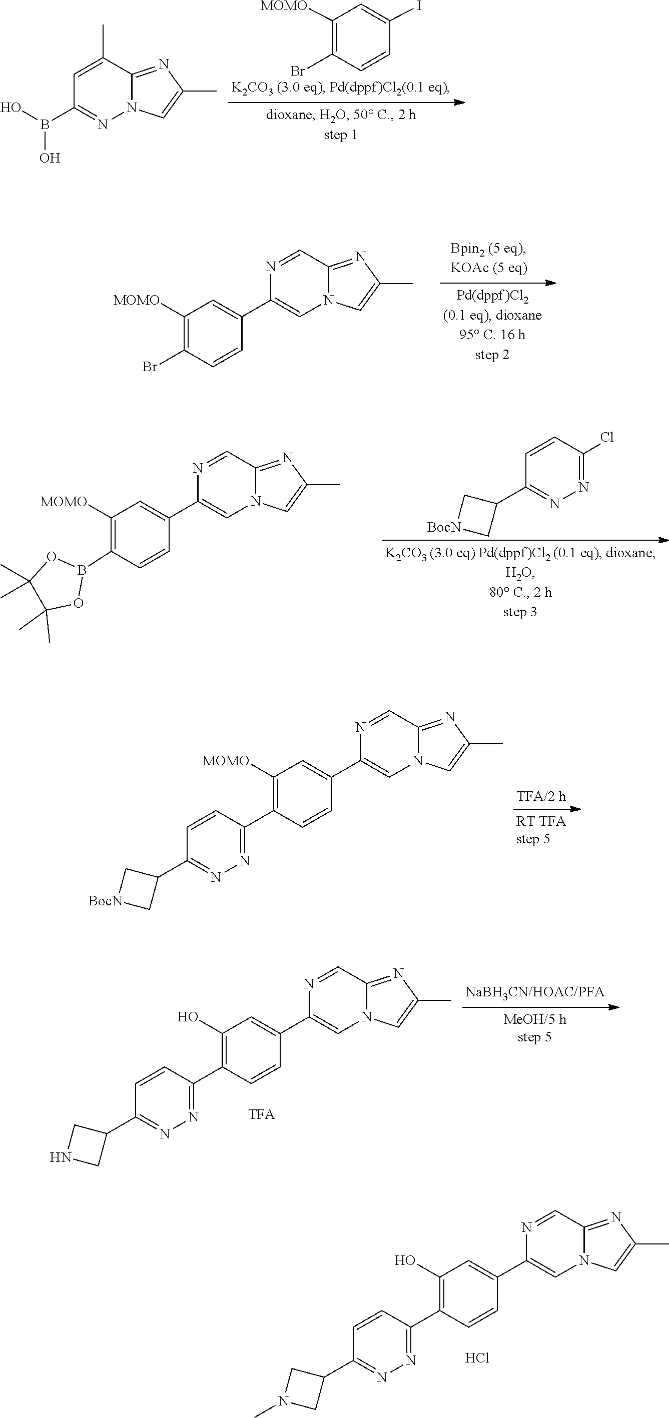

### Step 1: Preparation of 6-(4-bromo-3-(methoxymethoxy) phenyl)-2,8-dimethylimidazo[1,2-b]pyridazine

To a mixture of (2,8-dimethylimidazo[1,2-b]pyridazin-6-yl) boronic acid (1.47 g, 4.29 mmol) and 1-bromo-4-iodo-2-(methoxymethoxy) benzene (836 mg, 4.31 mmol) in dioxane:H₂O (20 mL:5 mL) were added Pd (dppf) Cl₂ (315 mg, 0.43 mmol) and K₂CO₃ (1.77 g, 12.80 mmol). The mixture was stirred at 50° C. for 2 h. The mixture was filtered and the filtrate was concentrate in vacuo. The residue was purified by silica gel column chromatography (PE:EA=1:1) to give title product (0.7 g, Y: 45.22%) as a brown solid. ESI-MS (M+H+): 363.9. ¹H NMR (400 MHz, CDCl₃) δ 7.75 (d, J=6.8 Hz, 1H), 7.73 (s, 1H), 7.66 (d, J=8.3 Hz, 1H), 7.44 (d, J=8.2 Hz, 1H), 7.19 (s, 1H), 5.37 (s, 2H), 3.57 (s, 3H), 2.70 (s, 3H), 2.53 (s, 3H).

### Step 2: Preparation of 6-(3-(methoxymethoxy)-4-(4,4,5,5-tetramethyl-1,3,2-dioxaborolan-2-yl) phenyl)-2,8-dimethylimidazo [1,2-b]pyridazine

To a mixture of 6-(4-bromo-3-(methoxymethoxy) phenyl)-2,8-dimethylimidazo [1,2-b]pyridazine (605 mg, 1.68 mmol) and B₂pin₂ (5.12 g, 20.17 mmol) in dioxane (20 mL) were added Pd (dppf) C₁₂ (409 mg, 0.56 mmol) and KOAc (2.74 g, 27.98 mmol). The mixture was charged with Ar for three times and stirred at 95° C. for 16 h. The mixture was concentrated in vacuo, the residue was purified by column chromatography (PE:EA=1:1) to give title product (425 mg, Y: 59.52%) as a brown solid. ESI-MS (M+H+): 410.1. ¹H NMR (400 MHz, CDCl₃) δ 7.80 (d, J=7.7 Hz, 1H), 7.76 (s, 1H), 7.61 (s, 1H), 7.55 (d, J=8.5 Hz, 1H), 7.24 (s, 1H), 5.31 (s, 2H), 3.57 (s, 3H), 2.70 (s, 3H), 2.53 (s, 3H), 1.38 (s, 12H).

### Step 3: Preparation of tert-butyl 3-(6-(4-(2,8-dimethylimidazo [1,2-b]pyridazin-6-yl)-2-(methoxymethoxy) phenyl) pyridazin-3-yl) azetidine-1-carboxylate

To a mixture of 6-(3-(methoxymethoxy)-4-(4,4,5,5-tetramethyl-1,3,2-dioxaborolan-2-yl) phenyl)-2,8-dimethylimidazo [1,2-b]pyridazine (425 mg, 1.04 mmol) and tert-butyl 3-(6-chloropyridazin-3-yl) azetidine-1-carboxylate (300 mg, 1.12 mmol) in dioxane: H₂O (20 mL:5 mL) were added Pd (dppf) C₁₂ (80 mg, 0.11 mmol) and K₂CO₃ (431 mg, 3.12 mmol). The mixture was stirred at 80° C. for 2 h. The mixture was concentrate in vacuo and the residue was purified by silica gel column chromatography (DCM:MeOH=40:1) to give title product (200 mg, Y: 38.76%) as a yellow solid. ESI-MS (M+H+): 517.2.

### Step 4: Preparation of 2-(6-(azetidin-3-yl) pyridazin-3-yl)-5-(2,8-dimethylimidazo [1,2-b]pyridazin-6-yl) phenol

A mixture of tert-butyl 3-(6-(4-(2,8-dimethylimidazo [1,2-b]pyridazin-6-yl)-2-(methoxymethoxy) phenyl) pyridazin-3-yl) azetidine-1-carboxylate (200 mg, 0.39 mmol) in TFA (10 mL) was stirred at rt for 2 h. The mixture was concentrated in vacuo to give title product (125 mg, Y: 86.21%) as a yellow solid. ESI-MS (M+H+): 373.1. ¹H NMR (400 MHz, MeOD-d₄) δ 8.48 (d, J=9.1 Hz, 1H), 8.25 (s, 1H), 8.17-8.11 (m, 2H), 7.81 (d, J=9.0 Hz, 1H), 7.77 (d, J=1.6 Hz, 1H), 7.76-7.72 (m, 1H), 4.55-4.49 (m, 5H), 2.77 (s, 3H), 2.62 (s, 3H).

### Step 5: Preparation of 5-(2,8-dimethylimidazo [1,2-b]pyridazin-6-yl)-2-(6-(I-methylazetidin-3-yl) pyridazin-3-yl) phenol hydrochloride

To a mixture of 2-(6-(azetidin-3-yl) pyridazin-3-yl)-5-(2,8-dimethylimidazo [1,2-b]pyridazin-6-yl) phenol (40 mg, 0.11 mmol) and (HCHO)ₙ (28 mg, 0.93 mmol) in MeOH (10 mL) and HOAc (1 drops) was added NaBH₃CN (35 mg, 0.56 mmol). The mixture was stirred at RT for 5 h. The mixture was diluted with water (0.5 mL) and concentrated in vacuo. The residue was purified by prep-HPLC (0.05% HCl in water/CH₃CN) to give the title product (7 mg, Y: 16.49%) as a yellow solid. ESI-MS (M+H+): 387.2. ¹H NMR (400 MHz, MeOD-d₄) δ 8.73-8.67 (m, 1H), 8.34 (s, 1H), 8.29 (s, 1H), 8.20-8.12 (m, 2H), 7.85 (s, 1H), 7.71 (s, 1H), 4.59-4.48 (m, 3H), 3.10 (s, 3H), 3.06-3.00 (m, 2H), 2.82 (s, 3H), 2.67 (s, 3H).

### Example 31. 2-[6-(azetidin-3-yl)pyridazin-3-yl]-5-{2-methylimidazo[1,2-b]pyridazin-6-yl}phenol (Compound 31)

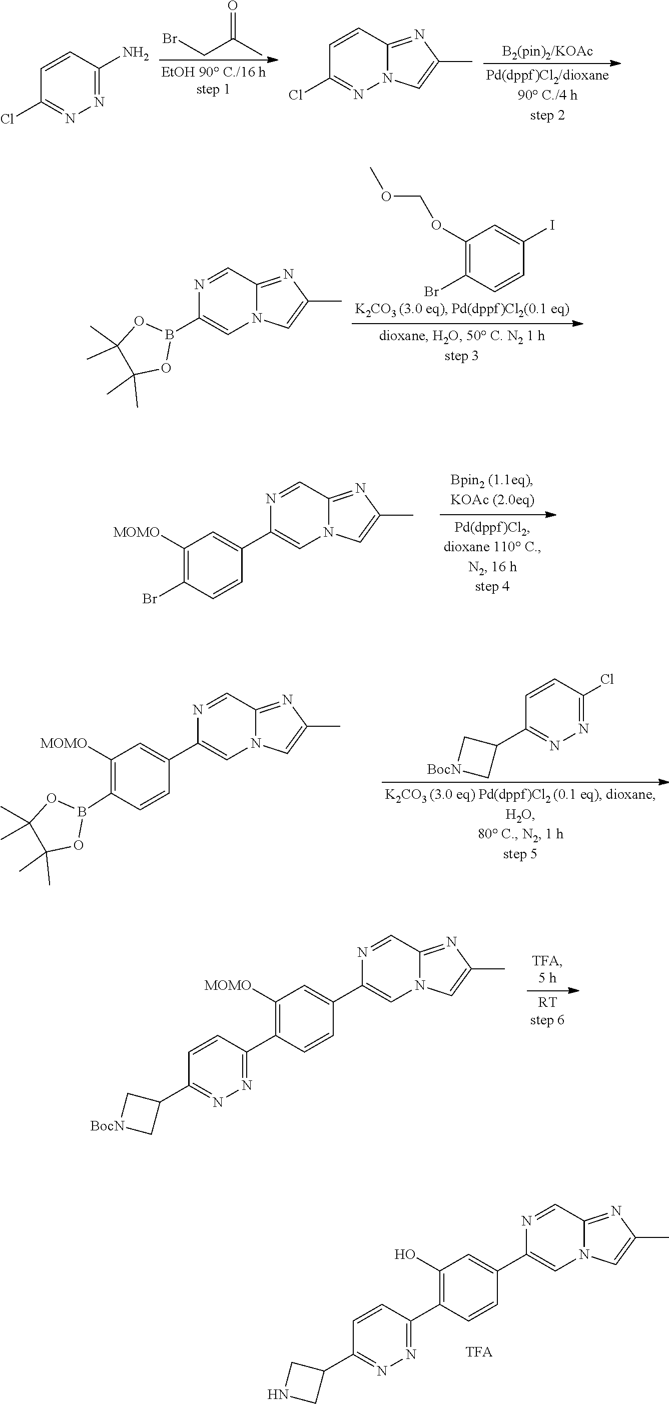

### Step 1: Preparation of 6-chloro-2-methylimidazo[1,2-b]pyridazine

To a solution of 6-chloropyridazin-3-amine (9.3 g, 72 mmol) in EtOH (200 mL) was added 1-bromopropan-2-one (19.8 g, 144 mmol) in EtOH (20 mL). The mixture was stirred at 90° C. for 16 h. The reaction mixture was concentrated, to the residue was added 2M NaOH aqueous solution (150 mL), then stirred at RT for 2 h. The precipitate was filtered and concentrated in vacuo. The filter cake was dispersed in MTBE (200 mL), stirred at RT for 2 h and filtered with PE:MTBE=1:1 (200 mL:200 mL), dried in vacuo to give the title product (8.2 g, Y: 68.0%) as a gray solid. ESI-MS (M+H)+: 168.1. ¹H NMR (400 MHz, CDCl₃) δ 7.79 (d, J=9.4 Hz, 1H), 7.71 (s, 1H), 7.00 (d, J=9.4 Hz, 1H), 2.50 (s, 3H).

### Step 2: Preparation of 2-methyl-6-(4,4,5,5-tetramethyl-1,3,2-dioxaborolan-2-yl)imidazo[1,2-b]pyridazine

To a solution of 6-chloro-2-methylimidazo[1,2-b]pyridazine (5.2 g, 31 mmol), B₂(Pin)₂ (15.8 g, 62 mmol) and KOAc (9.1 g, 93 mmol) in 1,4-dioxane (250 mL) was added Pd(dppf)Cl₂ (2.5 g, 3.1 mmol). The mixture was stirred at 90° C. under N₂ for 4 h. The reaction mixture was used directly without purification. ESI-MS (M+H)+: 178.1.

### Step 3: Preparation of 6-(4-bromo-3-(methoxymethoxy)phenyl)-2-methylimidazo[1,2-b]pyridazine

To a solution of 2-methyl-6-(4,4,5,5-tetramethyl-1,3,2-dioxaborolan-2-yl)imidazo[1,2-b]pyridazine (2.50 g, 14 mmol), 1-bromo-4-iodo-2-(methoxymethoxy)benzene (5.00 g, 14 mmol) and K₂CO₃ (5.70 g, 42 mmol) in 1,4-dioxane: H₂O (250 mL:25 mL) was added Pd(dppf)Cl₂ (1.14 g, 1.4 mmol). The mixture was stirred at 50° C. under N₂ for 1 h. The mixture was concentrated under reduced pressure, the residue was purified by silica gel column chromatography (PE:EA=10:1) to give title product (2.0 g, Y: 40.8%) as a black solid. ESI-MS (M+H)+: 349.7.

### Step 4: Preparation of 6-(3-(methoxymethoxy)-4-(4,4,5,5-tetramethyl-1,3,2-dioxaborolan-2-yl)phenyl)-2-methylimidazo[1,2-b]pyridazine

To a mixture of 6-(4-bromo-3-(methoxymethoxy)phenyl)-2-methylimidazo[1,2-b]pyridazine (1.90 g, 5.40 mmol), B₂(Pin)₂ (27.6 g, 109 mmol) and KOAc (5.29 g, 54 mmol) in 1,4-dioxane (150 mL) was added Pd(dppf)Cl₂ (310 mg, 0.38 mmol). The mixture was stirred at 110° C. under N₂ for 16 h. The mixture was allowed to cooling down to room temperature and concentrated under reduced pressure. The crude was purified by silica gel column chromatography (PE:EA=10:1) to give title product (912 mg, Y: 42.4%) as a yellow solid. ESI-MS (M+H)+: 396.2. ¹H NMR (400 MHz, CDCl₃) δ 7.91 (d, J=9.4 Hz, 1H), 7.83-7.79 (m, 2H), 7.64 (d, J=1.3 Hz, 1H), 7.57-7.54 (m, 1H), 7.44 (s, 1H), 5.31 (s, 2H), 3.56 (s, 3H), 2.52 (s, 3H), 1.38 (s, 12H).

### Step 5: Preparation of tert-butyl 3-(6-(2-(methoxymethoxy)-4-(2-methylimidazo[1,2-b]pyridazin-6-yl)phenyl)pyridazin-3-yl)azetidine-1-carboxylate

To a mixture of 6-(3-(methoxymethoxy)-4-(4,4,5,5-tetramethyl-1,3,2-dioxaborolan-2-yl)phenyl)-2-methylimidazo[1,2-b]pyridazine (900 mg, 2.27 mmol), tert-butyl 3-(6-chloropyridazin-3-yl)azetidine-1-carboxylate (610 mg, 2.27 mmol) and K₂CO₃ (939 mg, 6.81 mmol) in 1,4-dioxane:H₂O (100 mL:10 mL) was added Pd(dppf)Cl₂ (179 mg, 0.22 mmol). The mixture was stirred at 80° C. under N₂ for 1 h. The mixture was allowed to cooling down to room temperature, and concentrated under reduced pressure. The residue was purified by silica gel column chromatography (PE:EA=5:1) to give title product (450 mg, Y: 39.4%) as a yellow solid. ESI-MS (M+H)+: 503.1.

### Step 6: Preparation of 2-(6-(azetidin-3-yl)pyridazin-3-yl)-5-(2-methylimidazo[1,2-b]pyridazin-6-yl)phenol trifluoroacetate

A solution of tert-butyl 3-(6-(2-(methoxymethoxy)-4-(2-methylimidazo[1,2-b]pyridazin-6-yl)phenyl)pyridazin-3-yl)azetidine-1-carboxylate (50 mg, 0.09 mmol) in TFA (2.5 mL) was stirred at RT for 5 h. The mixture was concentrated and purified by pre-HPLC (0.05% TFA in water/CH₃CN) to give title product (25.28 mg, Y: 70.9%) as a gray solid. ESI-MS (M+H)+: 359.0. ¹H NMR (400 MHz, MeOD-d₄) δ 8.49 (d, J=9.1 Hz, 1H), 8.32 (d, J=9.6 Hz, 1H), 8.28-8.23 (m, 2H), 8.17 (d, J=8.4 Hz, 1H), 7.81 (d, J=9.2 Hz, 2H), 7.78-7.75 (m, 1H), 4.56-4.51 (m, 5H), 2.62 (d, J=0.7 Hz, 3H).

### Example 32. 5-{2,8-dimethylimidazo[1,2-a]pyrazin-6-yl}-2-[6-(1-ethylazetidin-3-yl)pyridazin-3-yl]phenol (Compound 32)

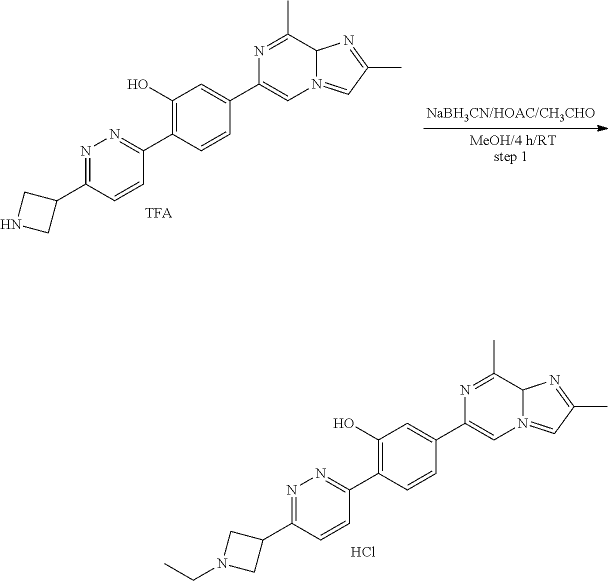

### Step 1: Preparation of 5-(2,8-dimethylimidazo[1,2-a]pyrazin-6-yl)-2-(6-(1-ethylazetidin-3-yl)pyridazin-3-yl)phenol

To a solution of 2-(6-(azetidin-3-yl)pyridazin-3-yl)-5-(2,8-dimethylimidazo[1,2-a]pyrazin-6-yl)phenol (20 mg, 0.054 mmol) in MeOH (4 mL) was added acetaldehyde (12 mg, 0.27 mmol) and acetic acid (16 mg, 0.27 mmol). The mixture was stirred at RT for 1 h, then sodium cyanoborohydride (10 mg, 0.161 mmol) was added under ice bath and the mixture was stirred at rt for 3 h, then water (1 mL) was added and the solution was concentrated in vacuo. The residue was purified by prep-HPLC (0.05% NH₃·H₂O in H₂O/CH₃CN) to give title compound (8.6 mg, yield: 26.6%) as a white solid. ESI-MS (M+H)+: 401.1. ¹H NMR (400 MHz, CDCl₃) δ 13.66 (s, 1H), 8.33 (s, 1H), 8.10 (d, J=9.1 Hz, 1H), 7.82 (d, J=8.2 Hz, 1H), 7.72 (d, J=9.0 Hz, 1H), 7.62 (d, J=8.0 Hz, 2H), 7.49 (s, 1H), 4.17-4.10 (m, 1H), 4.02-4.01 (m, 2H), 3.65-3.64 (m, 2H), 2.95 (s, 3H), 2.75 (q, J=7.1 Hz, 2H), 2.54 (s, 3H), 1.11 (t, J=7.1 Hz, 3H).

### Example 33. 2-[6-(azetidin-3-yl)pyridazin-3-yl]-5-{2-methylimidazo[1,2-a]pyrazin-6-yl}phenol (Compound 33)

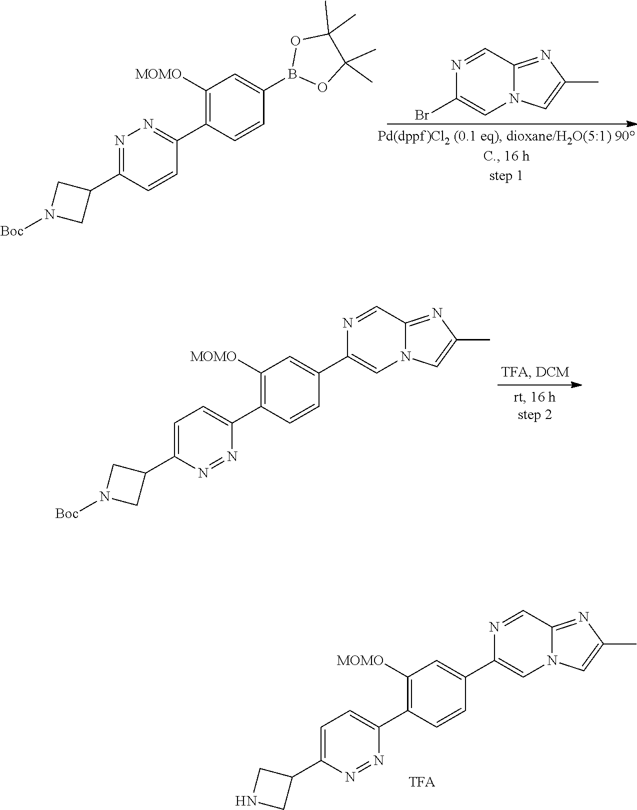

### Step 1: Preparation of tert-butyl 3-(6-(2-(methoxymethoxy)-4-(2-methylimidazo[1,2-a]pyrazin-6-yl)phenyl)pyridazin-3-yl)azetidine-1-carboxylate

A mixture of tert-butyl 3-(6-(2-(methoxymethoxy)-4-(4,4,5,5-tetramethyl-1,3,2-dioxaborolan-2-yl)phenyl)pyridazin-3-yl)azetidine-1-carboxylate (100 mg, 0.2 mmol), 6-bromo-2-methylimidazo[1,2-a]pyrazine (100 mg, 0.48 mmol), K₂CO₃ (82.8 mg, 0.6 mmol) and Pd(dppf)Cl₂ (14.6 mg, 0.02 mmol) in 1,4-dioxane/H₂O (10 mL/2 mL) was stirred at 90° C. for 16 h. The reaction mixture was diluted with water (45 mL) and extracted with EtOAc (45 mL*3). The organic layer was washed with brine, dried over Na₂SO₄ and evaporated to give crude title compound. The crude was purified by C₁₈-flash (0.1% FA in water/CH₃CN) to give title compound (90 mg, Y: 90%) as a yellow solid. ESI-MS (M+H)+: 503.2. ¹H NMR (400 MHz, CDCl₃) δ 9.08 (s, 1H), 8.47 (d, J=1.4 Hz, 1H), 8.11-8.07 (m, 2H), 7.69 (dd, J=8.1, 1.6 Hz, 1H), 7.54 (d, J=3.4 Hz, 2H), 7.46 (d, J=8.8 Hz, 1H), 5.35 (s, 2H), 4.48-4.37 (m, 2H), 4.35-4.22 (m, 2H), 4.20-4.07 (m, 1H), 3.49 (s, 3H), 2.56 (s, 3H), 1.48 (s, 9H).

### Step 2: Preparation of 2-(6-(azetidin-3-yl)pyridazin-3-yl)-5-(2-methylimidazo[1,2-a]pyrazin-6-yl)phenol

To a solution of tert-butyl 3-(6-(2-(methoxymethoxy)-4-(2-methylimidazo[1,2-a]pyrazin-6-yl)phenyl)pyridazin-3-yl)azetidine-1-carboxylate (90 mg, 0.18 mmol) in DCM (5 mL) was added TFA (5 ml) at room temperature. The reaction mixture was stirred for 16 h. The reaction mixture was concentrated in vacuo and the residue was purified by prep-HPLC (0.05% NH₃·H₂O in water/CH₃CN) to give title compound (5 mg, Y: 7%) as a yellow solid. ESI-MS (M+H)+: 359.0. ¹H NMR (400 MHz, MeOD-d₄) δ 8.96 (d, J=8.4 Hz, 2H), 8.43 (d, J=9.2 Hz, 1H), 8.04 (d, J=8.4 Hz, 1H), 7.87-7.74 (m, 2H), 7.70-7.46 (m, 2H), 4.44-4.34 (m, 1H), 4.30-4.11 (m, 4H), 2.50 (s, 3H).

### Example 34. 5-(2,7-dimethyl-2H-indazol-5-yl)-2-[6-(1-ethylazetidin-3-yl)pyridazin-3-yl]phenol (Compound 34)

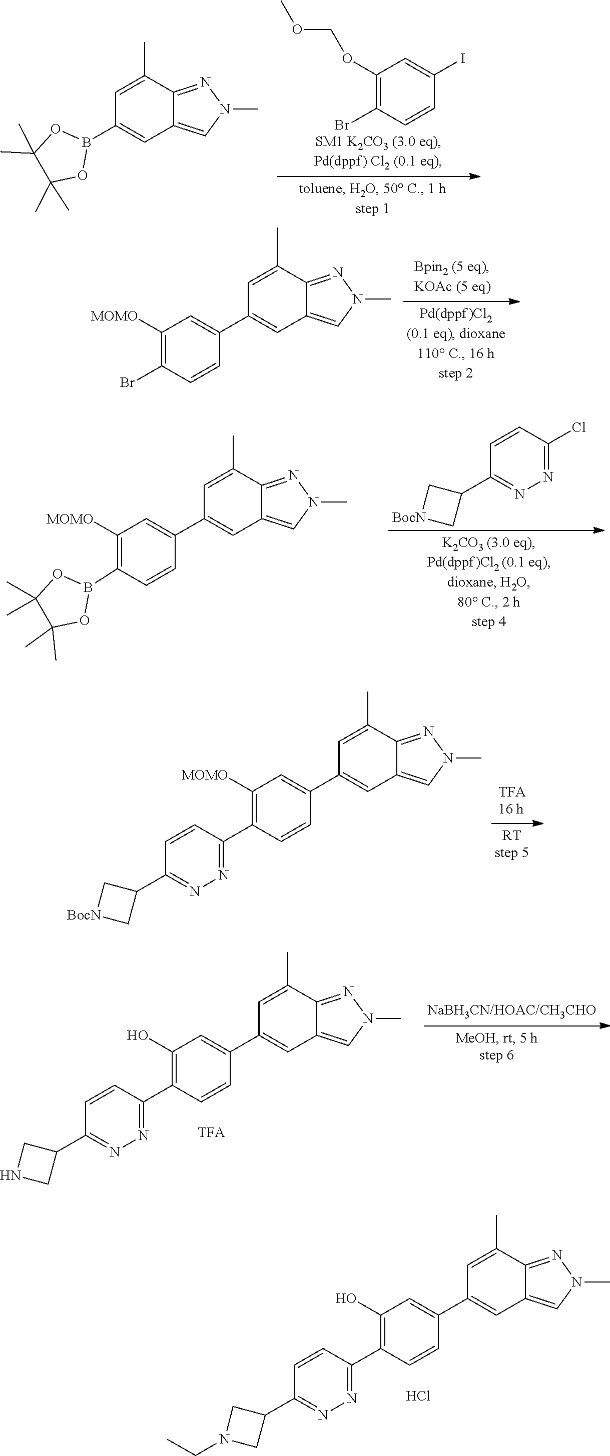

### Step 1: Preparation of 5-(4-bromo-3-(methoxymethoxy)phenyl)-2,7-dimethyl-2H-indazole

To a mixture of 2,7-dimethyl-5-(4,4,5,5-tetramethyl-1,3,2-dioxaborolan-2-yl)-2H-indazole (5.30 g, 19.48 mmol) and 1-bromo-4-iodo-2-(methoxymethoxy) benzene (6.60 g, 19.48 mmol) in dioxane: H2O (90 mL:15 mL) were added Pd(dppf)Cl₂ (712 mg, 0.97 mmol) and K₂CO₃ (8.00 g, 58.34 mmol). The mixture was stirred at 50° C. for 1 h. LCMS showed the starting material was consumed completely. The mixture was filtered and the filtrate was concentrate in vacuo. The residue was purified by silica gel column chromatography (PE:EA=1:1) to give title product (4.0 g, Y: 58%) as a brown solid. ESI-MS (M+H+): 363.0. ¹H NMR (400 MHz, DMSO-d₆) δ 8.36 (s, 1H), 7.85-7.69 (d, J=13.9 Hz, 1H), 7.70-7.59 (s, 1H), 7.51-7.45 (s, 1H), 7.33 (s, 1H), 7.28-7.19 (m, 1H), 5.38 (s, 2H), 4.18 (s, 3H), 3.43 (s, 3H), 2.55 (s, 3H).

### Step 2: Preparation of 5-(3-(methoxymethoxy)-4-(4,4,5,5-tetramethyl-1,3,2-dioxaborolan-2-yl)phenyl)-2,7-dimethyl-2H-indazole

To a mixture of 5-(4-bromo-3-(methoxymethoxy)phenyl)-2,7-dimethyl-2H-indazole (4.00 g, 11.11 mmol) and B₂pin₂ (28.00 g, 111.11 mmol) in dioxane (200 mL) were added Pd(dppf)Cl₂ (406 mg, 0.55 mmol) and KOAc (8.70 g, 88.88 mmol). The mixture was stirred at 110° C. for 16 h. LCMS showed the starting material was consumed completely. The mixture was filtered and the filtrate was concentrate in vacuo. The residue was purified by Flash chromatography (PE:EA=1:1) to give title product (1.6 g, Y: 35.5%) as a brown solid. ESI-MS (M+H+): 409.0. ¹H NMR (400 MHz, CDCl₃) δ 7.91 (s, 1H), 7.76 (d, J=7.5 Hz, 1H), 7.68 (s, 1H), 7.31 (d, J=1.5 Hz, 1H), 7.29 (d, 1H), 7.26 (s, 1H), 5.28 (s, 2H), 4.25 (s, 3H), 3.56 (s, 3H), 2.68 (s, 3H), 1.37 (s, 12H).

### Step 3: Preparation of tert-butyl 3-(6-(4-(2,7-dimethyl-2H-indazol-5-yl)-2-(methoxymethoxy)phenyl)pyridazin-3-yl)azetidine-1-carboxylate

To a mixture of 5-(3-(methoxymethoxy)-4-(4,4,5,5-tetramethyl-1,3,2-dioxaborolan-2-yl)phenyl)-2,7-dimethyl-2H-indazole (1.60 g, 3.92 mmol) and tert-butyl 3-(6-chloropyridazin-3-yl) azetidine-1-carboxylate (1.06 g, 3.92 mmol) in dioxane: H₂O (63 mL:9 mL) were added Pd(dppf)Cl₂ (196 mg, 0.20 mmol) and K₂CO₃ (1.62 g, 11.76 mmol). The mixture was stirred at 80° C. for 2 h. LCMS showed the starting material was consumed completely. The mixture was filtered and the filtrate was concentrate in vacuo. The residue was purified by silica gel column chromatography (DCM:MeOH=40:1) to give title product (1.40 g, Y: 70%) as a yellow solid. ESI-MS (M+H+): 516.2. ¹H NMR (400 MHz, CDCl₃) δ 8.10-8.05 (m, 2H), 7.95 (d, J=4.1 Hz, 1H), 7.75-7.72 (m, 1H), 7.51 (d, J=8.8 Hz, 2H), 7.39-7.34 (m, 2H), 5.31 (s, 2H), 4.48-4.37 (m, 3H), 4.27 (s, 3H), 4.21-4.09 (m, 2H), 3.49 (s, 3H), 2.70 (s, 3H), 1.48 (s, 9H).

### Step 4: 2-(6-(azetidin-3-yl)pyridazin-3-yl)-5-(2-methyl-2H-pyrazolo[3,4-c]pyridin-5-yl)phenol hydrochloride

A mixture of tert-butyl 3-(6-(4-(2,7-dimethyl-2H-indazol-5-yl)-2-(methoxymethoxy)phenyl)pyridazin-3-yl)azetidine-1-carboxylate (1.30 g, 2.52 mmol) in TFA (15 mL) was stirred at rt for 16 h. LCMS showed the starting material was consumed completely. The mixture was concentrated in vacuo and the crude was purified by pre-HPLC to give title product (500 mg, Y: 54%) as a yellow solid. ESI-MS (M+H+): 372.1. ¹H NMR (400 MHz, MeOD-d₄) δ 9.02-8.88 (m, 2H), 8.51 (d, J=8.9 Hz, 1H), 8.17 (s, 1H), 8.00 (d, J=8.2 Hz, 1H), 7.91 (s, 1H), 7.53 (d, J=8.1 Hz, 1H), 7.47 (s, 1H), 4.75-4.67 (m, 1H), 4.63-4.54 (m, 4H), 4.47 (s, 3H), 2.72 (s, 3H).

### Step 5: Preparation of 5-(2,7-dimethyl-2H-indazol-5-yl)-2-(6-(I-ethylazetidin-3-yl)pyridazin-3-yl)phenol hydrochloride

To a mixture of 2-(6-(azetidin-3-yl) pyridazin-3-yl)-5-(2,8-dimethylimidazo [1,2-b]pyridazin-6-yl) phenol (500 mg, 1.35 mmol) and CH₃CHO (296 mg, 6.75 mmol) in MeOH (20 mL) and HOAc (500 mg, 8.34 mmol) were added NaBH₃CN (258 mg, 4.04 mmol). The mixture was stirred at RT for 5 h. LCMS showed the starting material was consumed completely. The mixture was concentrated in vacuo. The residue was purified by prep-HPLC (0.05% NH₃·H₂O in/CH₃CN) to give the solid. Dissolved in 0.05% HCl in water (20 mL) and lyophilized to give title product (185 mg, Y: 34.5%) as a yellow solid. ESI-MS (M+H+): 400.2. ¹H NMR (400 MHz, MeOD-d₄) δ 8.93-8.87 (m, 2H), 8.43 (t, J=8.1 Hz, 1H), 8.15 (s, 1H), 8.04-7.97 (m, 1H), 7.88 (s, 1H), 7.52 (d, J=8.3 Hz, 1H), 7.46 (s, 1H), 4.79-4.68 (m, 2H), 4.67-4.46 (m, 3H), 4.45 (s, 3H), 3.50-3.39 (m, 2H), 2.71 (s, 3H), 1.34-1.27 (m, 3H).

### Example 35. 5-{2,8-dimethylimidazo[1,2-a]pyridin-6-yl}-2-[6-(1-methylazetidin-3-yl)pyridazin-3-yl]phenol (Compound 35)

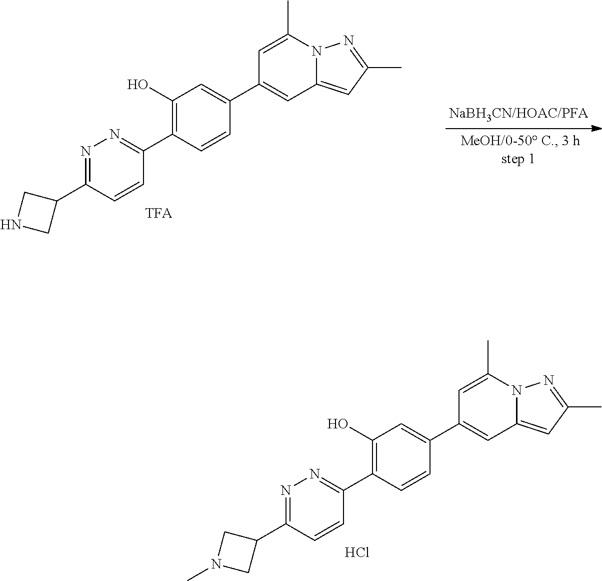

### Step 1: Preparation of 5-(2,8-dimethylimidazo[1,2-a]pyridin-6-yl)-2-(6-(1-methylazetidin-3-yl)pyridazin-3-yl)phenol hydrochloride

To a solution of 2-(6-(azetidin-3-yl)pyridazin-3-yl)-5-(2,8-dimethylimidazo[1,2-a]pyridin-6-yl)phenol trifluoroacetate (150 mg, 0.40 mmol) and AcOH (0.76 mL) in MeOH (30 mL) was added (HCHO)ₙ (60 mg, 2.02 mmol). The mixture was stirred at RT for 1 h, then added NaBH₃CN (75.6 mg, 1.20 mmol) at 0° C. and stirred at 50° C. for 3 h. The mixture was quenched with H2O, concentrated under reduced pressure and purified by prep-HPLC (0.05% HCl in water/ACN) to give title product (15.84 mg, Y: 10.1%) as a yellow solid. ESI-MS (M+H)+: 386.1. ¹H NMR (400 MHz, MeOD-d₄) δ 9.03 (s, 1H), 8.74 (s, 1H), 8.26 (s, 1H), 8.09-8.07 (m, 3H), 7.51-7.42 (m, 2H), 4.82-4.80 (m, 1H), 4.67-4.42 (m, 4H), 3.10 (s, 3H), 2.72 (s, 3H), 2.60 (s, 3H).

### Example 36. 5-{2,8-dimethylimidazo[1,2-a]pyridin-6-yl}-2-[6-(1-ethylazetidin-3-yl)pyridazin-3-yl]phenol (Compound 36)

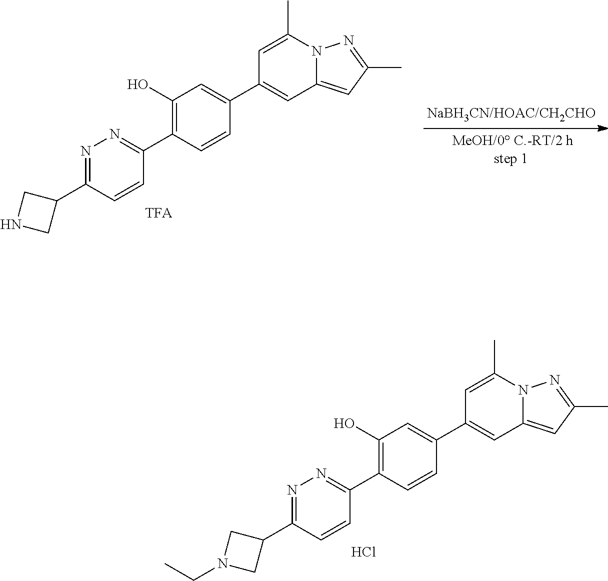

### Step 1: Preparation of 5-(2,8-dimethylimidazo[1,2-a]pyridin-6-yl)-2-(6-(1-ethylazetidin-3-yl)pyridazin-3-yl)phenol hydrochloride

To a solution of 2-(6-(azetidin-3-yl)pyridazin-3-yl)-5-(2,8-dimethylimidazo[1,2-a]pyridin-6-yl)phenol trifluoroacetate (150 mg, 0.40 mmol) and AcOH (0.76 mL) in MeOH (30 mL) was added CH₃CHO (88 mg, 2.00 mmol). The mixture was stirred at RT for 1 h, then added NaBH₃CN (75.6 mg, 1.20 mmol) at 0° C. and stirred at RT for 1 h. The mixture was quenched with H2O (20 mL), concentrated under reduced pressure and purified by prep-HPLC (0.05% HCl in water/ACN) to give title product (52.45 mg, Y: 32.5%) as a yellow solid. ESI-MS (M+H)+: 400.3. ¹H NMR (400 MHz, MeOD-d₄) δ 8.97 (s, 1H), 8.58-8.54 (m, 1H), 8.14-8.09 (m, 2H), 8.01 (s, 1H), 7.95 (d, J=9.0 Hz, 1H), 7.43 (d, J=7.3 Hz, 2H), 4.73-4.64 (m, 2H), 4.56-4.44 (m, 3H), 3.46-3.40 (m, 2H), 2.72 (s, 3H), 2.60 (s, 3H), 1.32-1.28 (m, 3H).

### Example 37. 2-[6-(1-methylazetidin-3-yl)pyridazin-3-yl]-5-{2-methylimidazo[1,2-b]pyridazin-6-yl}phenol (Compound 37)

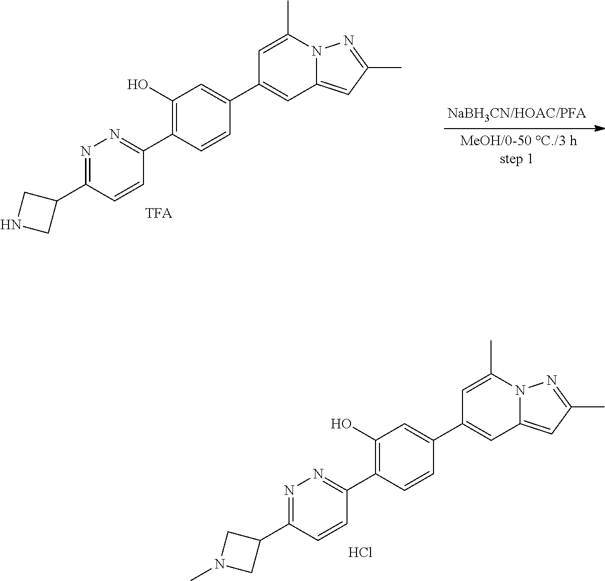

### Step 1: Preparation of 2-(6-(1-methylazetidin-3-yl)pyridazin-3-yl)-5-(2-methylimidazo[1,2-b]pyridazin-6-yl)phenol hydrochloride

To a solution of 2-(6-(azetidin-3-yl)pyridazin-3-yl)-5-(2-methylimidazo[1,2-b]pyridazin-6-yl)phenol trifluoroacetate (30 mg, 0.80 mmol) and AcOH (0.15 mL) in MeOH (6 mL) was added polymer of HCHO (12 mg, 0.40 mmol). The mixture was stirred at RT for 1 h, then added NaBH₃CN (15 mg, 0.23 mmol) at 0° C. and stirred at 50° C. for 2 h. The mixture was quenched with H₂O (1 mL), concentrated under reduced pressure and purified by prep-HPLC (0.05% HCl in water/ACN) to give title product (1.98 mg, Y: 6.3%) as a yellow solid. ESI-MS (M+H)+: 373.1. ¹H NMR (400 MHz, MeOD-d₄) δ 8.59-8.54 (m, 1H), 8.47-8.41 (m, 2H), 8.38 (s, 1H), 8.21-8.15 (m, 1H), 7.93 (d, J=8.9 Hz, 1H), 7.86-7.78 (m, 2H), 4.82-4.80 (m, 2H), 4.75-4.71 (m, 1H), 4.50-4.48 (m, 2H), 3.10 (d, J=5.5 Hz, 3H), 2.66 (s, 3H).

### Example 38. 2-[6-(1-ethylazetidin-3-yl)pyridazin-3-yl]-5-{2-methylimidazo[1,2-b]pyridazin-6-yl}phenol (Compound 38)

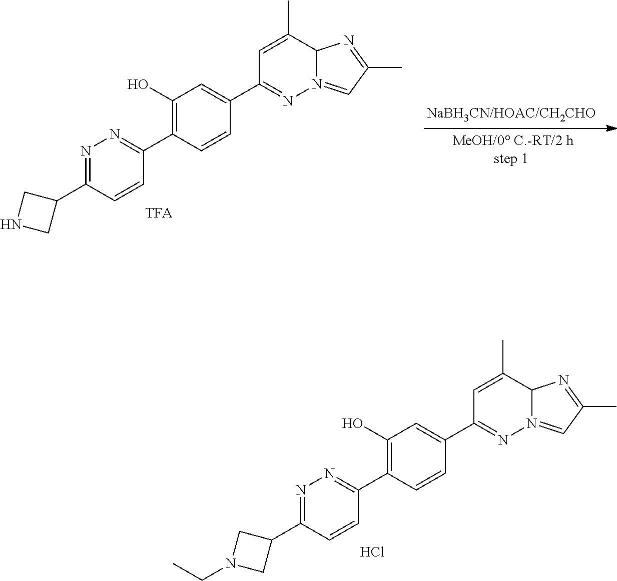

### Step 1: Preparation of 2-(6-(1-ethylazetidin-3-yl)pyridazin-3-yl)-5-(2-methylimidazo[1,2-b]pyridazin-6-yl)phenol hydrochloride

To a solution of 2-(6-(azetidin-3-yl)pyridazin-3-yl)-5-(2-methylimidazo[1,2-b]pyridazin-6-yl)phenol trifluoroacetate (30 mg, 0.80 mmol) and AcOH (0.15 mL) in MeOH (6 mL) was added CH₃CHO (17 mg, 0.40 mmol). The mixture was stirred at RT for 1 h, then added NaBH₃CN (15 mg, 0.23 mmol) at 0° C. and stirred at RT for 1 h. The mixture was quenched with H₂O (1 mL), concentrated under reduced pressure and purified by prep-HPLC (0.05% HCl in water/ACN) to give title product (7.28 mg, Y: 22.5%) as a white solid. ESI-MS (M+H)+: 388.0. ¹H NMR (400 MHz, MeOD-d₄) δ 8.50 (d, J=8.8 Hz, 1H), 8.32-8.14 (m, 4H), 7.84 (d, J=9.0 Hz, 1H), 7.80-7.73 (m, 2H), 4.69-4.67 (m, 2H), 4.52-4.50 (m, 3H), 3.45-3.43 (m, 2H), 2.61 (s, 3H), 1.34-1.25 (m, 3H).

### Example 39. 2-[6-(1-methylazetidin-3-yl)pyridazin-3-yl]-5-{2-methylimidazo[1,2-a]pyrazin-6-yl}phenol (Compound 39)

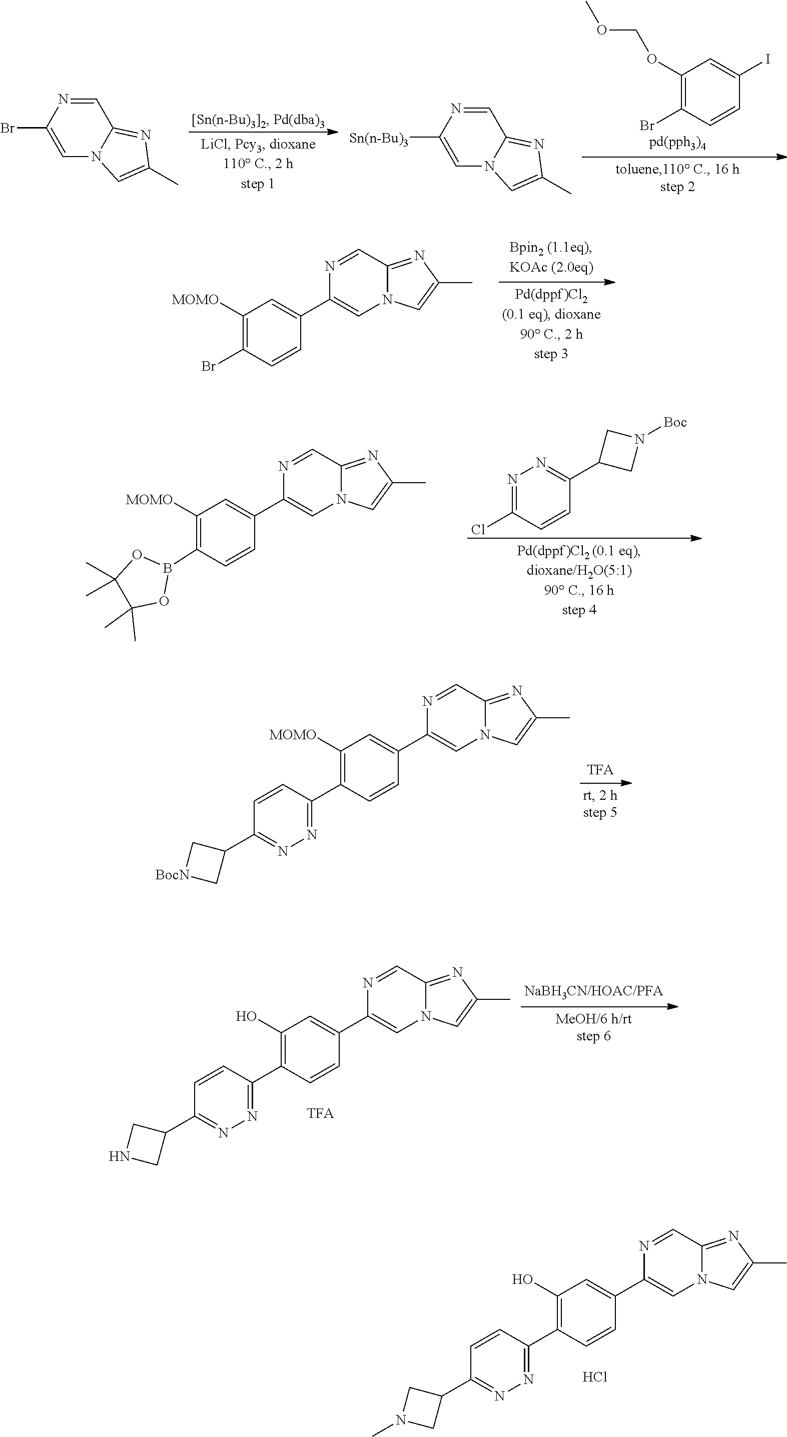

### Step 1: Preparation of 6-butyl-2-methylimidazo[1,2-a]pyrazine-dibutyl-l2-stannane

To a mixture of 6-bromo-2-methylimidazo[1,2-a]pyrazine (2.5 g, 11.8 mmol) and [Sn(n-Bu)_{3]2} (20.6 g, 35.4 mmol) in dioxane (250 mL) were added Pd₂(dba)₃ (1 g, 1.18 mmol), LiCl (2.5 g, 59 mmol) and Pcy₃ (664 mg, 2.36 mmol). The mixture was stirred at 110° C. for 2 h. The mixture was poured into sat. KF (100 mL), extracted with EA (300 mL), washed with brine (50 mL*3). The combined organic layer was dried over Na₂SO₄. filtered and concentrate in vacuo. The residue was purified by column chromatography (PE:EA=20:1-5:1) to give title product (3 g, Y: 61%) as a yellow oil. ESI-MS (M+H+): 424.0.

### Step 2: Preparation of 6-(4-bromo-3-(methoxymethoxy)phenyl)-2-methylimidazo[1,2-a]pyrazine

To a mixture of 1-bromo-4-iodo-2-(methoxymethoxy)benzene (2.4 g, 7.0 mmol) and 6-butyl-2-methylimidazo[1,2-a]pyrazine-dibutyl-l2-stannane (3 g, 10.5 mmol) in toluene (100 mL) were added Pd(PPh₃)₄ (1.6 g, 0.7 mmol). The mixture was stirred at 110° C. for 16 h. The mixture was concentrated in vacuo. The residue was purified by column chromatography (PE:EA=20:1-5:1) to give title product (1.5 g, Y: 62.5%) as a yellow oil. ESI-MS (M+H+): 349.9.

### Step 3: Preparation of 6-(3-(methoxymethoxy)-4-(4,4,5,5-tetramethyl-1,3,2-dioxaborolan-2-yl)phenyl)-2-methylimidazo[1,2-a]pyrazine

To a mixture of 6-(4-bromo-3-(methoxymethoxy)phenyl)-2-methylimidazo[1,2-a]pyrazine (1.4 g, 4.0 mmol) in dioxane (150 mL) were added B₂pin₂ (5.2 g, 32 mmol), Pd (dppf) Cl₂ (589 mg, 0.8 mmol) and KOAc (2 g, 20 mmol). The mixture was stirred at 90° C. under N₂ atmosphere for 2 h. LCMS showed the starting material was consumed completely. The mixture was concentrated and purified by column chromatography (PE:EA=20:1-5:1) to give title product (1.4 g, Y: 93%) as a yellow oil. ESI-MS (M+H+): 396.1. ¹H NMR (400 MHz, CDCl₃) δ 9.06 (d, J=0.8 Hz, 1H), 8.40 (d, J=1.5 Hz, 1H), 7.79 (d, J=7.7 Hz, 1H), 7.65 (d, J=1.3 Hz, 1H), 7.54 (dd, J=7.7, 1.5 Hz, 1H), 7.50 (s, 1H), 5.31 (s, 2H), 3.56 (s, 3H), 2.54 (s, 3H), 1.37 (s, 12H).

### Step 4: Preparation of tert-butyl 3-(6-(2-(methoxymethoxy)-4-(2-methylimidazo[1,2-a]pyrazin-6-yl)phenyl)pyridazin-3-yl)azetidine-1-carboxylate

To a mixture of 6-(3-(methoxymethoxy)-4-(4,4,5,5-tetramethyl-1,3,2-dioxaborolan-2-yl)phenyl)-2-methylimidazo[1,2-a]pyrazine (500 mg, 1.25 mmol) and tert-butyl 3-(6-chloropyridazin-3-yl)azetidine-1-carboxylate (340 mg, 1.25 mmol) in dioxane: H₂O (50 mL: 10 mL) were added Pd(dppf)Cl₂ (105 mg, 0.125 mmol) and K₂CO₃ (520 mg, 3.75 mmol). The mixture was stirred at 90° C. under N₂ atmosphere for 16 h. The mixture was concentrated and the crude was purified by column chromatography (PE:EA=50:1-5:1) to give title product (250 mg, Y: 39%) as a brown solid. ESI-MS (M+H+): 503.3.

### Step 5: Preparation of 2-(6-(azetidin-3-yl)pyridazin-3-yl)-5-(2-methylimidazo[1,2-a]pyrazin-6-yl)phenol

A mixture of tert-butyl 3-(6-(2-(methoxymethoxy)-4-(2-methylimidazo[1,2-a]pyrazin-6-yl)phenyl)pyridazin-3-yl)azetidine-1-carboxylate (200 mg, 0.52 mmol) in TFA (15 mL) was stirred at rt for 2 h. The mixture was diluted with water (10 mL), washed with DCM (5 mL*3). The aqueous phase was lyophilized to give title product (250 mg, crude) as a yellow solid. ESI-MS (M+H+): 359.2.

### Step 6: Preparation of 2-(6-(I-methylazetidin-3-yl)pyridazin-3-yl)-5-(2-methylimidazo[1,2-a]pyrazin-6-yl)phenol hydrochloride

To a solution of 2-(6-(azetidin-3-yl)pyridazin-3-yl)-5-(2-methylimidazo[1,2-a]pyrazin-6-yl)phenol (50 mg, 0.14 mmol) and (HCHO)ₙ (30 mg, 0.70 mmol) in MeOH (25 mL) was added HOAc (1.5 mL) at RT, after stirred at RT for 1 h, NaBH₃CN (40 mg, 0.42 mmol) was added to the mixture at 0° C. The mixture was stirred at RT for 5 h. The resulting reaction mixture was diluted with water (2 mL), concentrated under reduced pressure and the crude was purified by prep-HPLC (0.05% HCl in H₂O/MeCN) to give title product (3 mg, Y: 5.7%) as a yellow solid. ESI-MS (M+H)+: 373.0. ¹H NMR (400 MHz, MeOD-d₄) δ 9.39 (d, J=7.3 Hz, 2H), 8.50 (dd, J=9.1, 3.4 Hz, 1H), 8.20 (s, 1H), 8.15 (d, J=9.7 Hz, 1H), 7.84-7.82 (m, 2H), 7.77 (dd, J=8.5, 1.4 Hz, 1H), 4.80-4.65 (m, 2H), 4.55-4.40 (m, 3H), 3.09 (d, J=9.7 Hz, 3H), 2.67 (s, 3H).

### Example 40. 5-{2,7-dimethylpyrazolo[1,5-a]pyridin-5-yl}-2-[6-(1-methylazetidin-3-yl)pyridazin-3-yl]phenol (Compound 40)

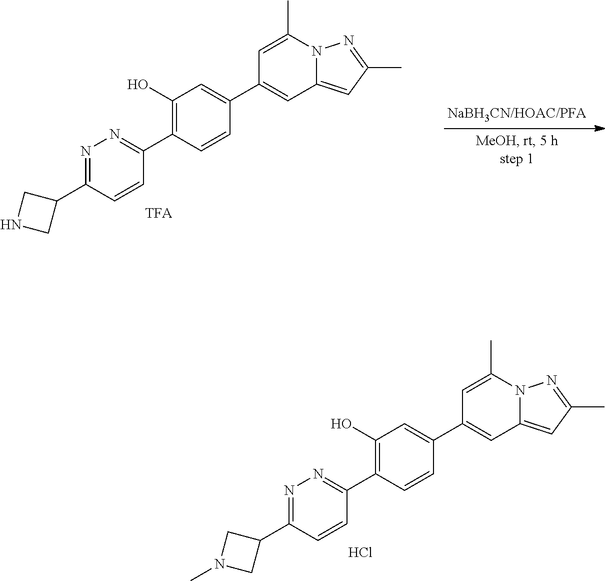

### Step 1: 5-(2,7-dimethylpyrazolo[1,5-a]pyridin-5-yl)-2-(6-(1-methylazetidin-3-yl)pyridazin-3-yl)phenol hydrochloride

To a solution of 2-(6-(azetidin-3-yl)pyridazin-3-yl)-5-(2,7-dimethylpyrazolo[1,5-a]pyridin-5-yl)phenol (1:1) (50 mg, 0.135 mmol) in MeOH (10 mL) and HOAc (50 mg) was added (HCHO)ₙ (20.25 mg, 0.675 mol). The resulting mixture was stirred for 1 h at r.t. Then NaBH₃CN (25.5 mg, 0.405 mmol) was added at 0° C. The resulting mixture was stirred for 4 h at r.t. The mixture was concentrated in vacuo and the residue was purified by prep-HPLC (0.05% HCl in water/ACN) to give title compound (5 mg, Y: 9.8%) as a yellow solid. ESI-MS (M+H)+: 386.1. ¹H NMR (400 MHz, DMSO-d₆) δ 11.10 (s, 1H), 10.59 (s, 1H), 8.64-8.52 (m, 1H), 8.16 (dd, J=8.8, 3.8 Hz, 1H), 8.02-7.98 (m, 1H), 7.89 (s, 1H), 7.45-7.43 (m, 2H), 7.17 (s, 1H), 6.52 (s, 1H), 4.62-4.52 (m, 2H), 4.44-4.26 (m, 3H), 2.93 (s, 3H), 2.72 (s, 3H), 2.44 (s, 3H).

### Example 41. 5-{2,7-dimethylpyrazolo[1,5-a]pyridin-5-yl}-2-[6-(1-ethylazetidin-3-yl)pyridazin-3-yl]phenol (Compound 41)

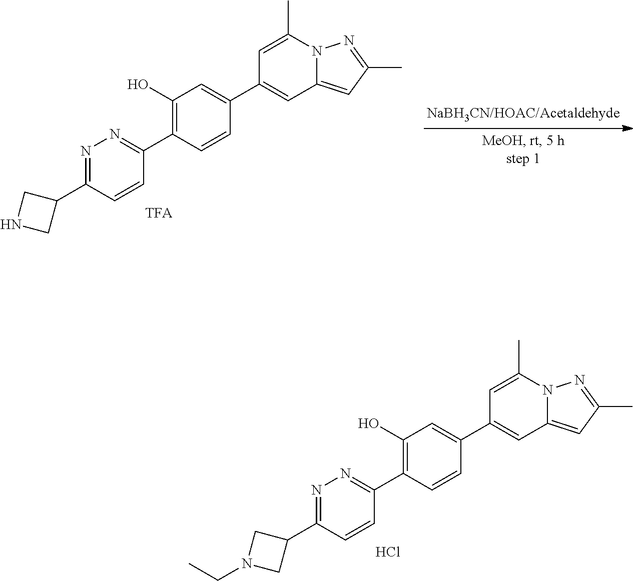

### Step 1: 5-(2,7-dimethylpyrazolo[1,5-a]pyridin-5-yl)-2-(6-(I-ethylazetidin-3-yl)pyridazin-3-yl)phenol hydrochloride

To a solution of 2-(6-(azetidin-3-yl)pyridazin-3-yl)-5-(2,7-dimethylpyrazolo[1,5-a]pyridin-5-yl)phenol (1:1) (50 mg, 0.135 mmol) in MeOH (10 mL) and HOAc (50 mg) was added acetaldehyde (30 mg, 0.675 mol). The resulting mixture was stirred for 1 h at rt. Then NaBH₃CN (25.5 mg, 0.405 mmol) was added at 0° C. The resulting mixture was stirred for 4 h at rt. The mixture was diluted with water (1 mL) and concentrated in vacuo. The residue was purified by prep-HPLC (0.05% HCl in water/ACN) to give title compound (1.16 mg, Y: 2.32%) as a yellow solid. ESI-MS (M+H)+: 400.1. H NMR (400 MHz, DMSO-d₆) δ 11.22 (s, 1H), 10.67 (s, 1H), 8.60 (dd, J=12.1, 9.2 Hz, 1H), 8.16 (dd, J=8.8, 5.9 Hz, 1H), 8.02-7.98 (m, 1H), 7.89 (s, 1H), 7.52-7.37 (m, 2H), 7.17 (s, 1H), 6.51 (s, 1H), 4.62-4.13 (m, 5H), 3.47-3.02 (m, 2H), 2.72 (s, 3H), 2.44 (s, 3H), 1.20-1.09 (m, 3H).

### Example 42. 5-{2,8-dimethylimidazo[1,2-b]pyridazin-6-yl}-2-[6-(1-ethylazetidin-3-yl)pyridazin-3-yl]phenol (Compound 42)

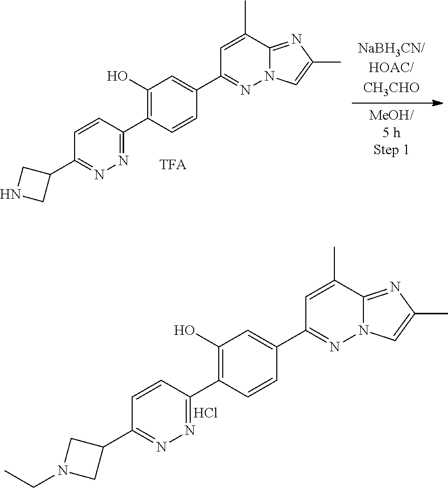

### Step 1: Preparation of 5-(2,8-dimethylimidazo [1,2-b]pyridazin-6-yl)-2-(6-(1-ethylazetidin-3-yl) pyridazin-3-yl) phenol hydrochloride

To a mixture of 2-(6-(azetidin-3-yl) pyridazin-3-yl)-5-(2,8-dimethylimidazo [1,2-b]pyridazin-6-yl) phenol (70 mg, 0.19 mmol) and CH₃CHO (41 mg, 0.94 mmol) in MeOH (16 mL) and HOAc (16 drops) were added NaBH₃CN (36 mg, 0.56 mmol). The mixture was stirred at RT for 5 h. The mixture was concentrated in vacuo. The residue was purified by prep-HPLC (0.05% HCl in water/CH₃CN) to give the title product (22 mg, Y: 28.85%) as a yellow solid. ESI-MS (M+H+): 401.3. ¹H NMR (400 MHz, MeOD-d₄) δ 8.72-8.67 (m, 1H), 8.29 (s, 1H), 8.17-8.12 (m, 1H), 7.85-7.83 (m, 2H), 4.77-4.66 (m, 2H), 4.54-4.52 (m, 3H), 3.45-3.43 (m, 2H), 2.81 (s, 3H), 2.67 (s, 3H), 1.33-1.27 (m, 3H).

### Example 43. 2-[6-(1-ethylazetidin-3-yl)pyridazin-3-yl]-5-{2-methylimidazo[1,2-a]pyrazin-6-yl}phenol (Compound 43)

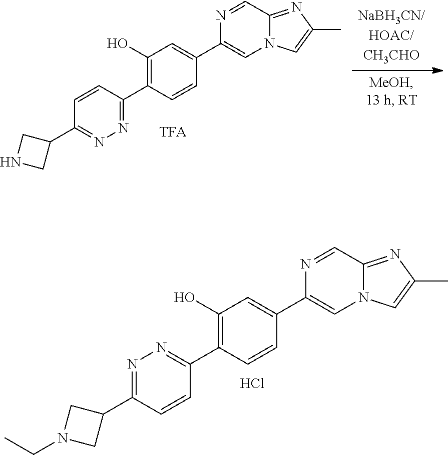

### Step 1: Preparation of 2-(6-(1-ethylazetidin-3-yl)pyridazin-3-yl)-5-(2-methylimidazo[1,2-a]pyrazin-6-yl)phenol

To a solution of 2-(6-(azetidin-3-yl)pyridazin-3-yl)-5-(2-methylimidazo[1,2-a]pyrazin-6-yl)phenol (50 mg, 0.14 mmol) and acetaldehyde (40 mg, 0.70 mmol) in MeOH (25 mL) was added HOAc (0.09 mL) at RT. After stirred at RT for 1 h, NaBH₃CN (40 mg, 0.42 mmol) was added to the mixture at 0° C., and the mixture was stirred at RT for 12 h. The resulting reaction mixture was diluted with water (2 mL) and concentrated under reduced pressure. The residue was purified by prep-HPLC (0.05% HCl in H₂O/CH₃CN) to give title product (6 mg, Y: 11%) as a yellow solid. ESI-MS (M+H)+: 387.0. ¹H NMR (400 MHz, MeOD-d₄) δ 9.40 (d, J=4.9 Hz, 2H), 8.56-8.47 (m, 1H), 8.21 (s, 1H), 8.17-8.11 (m, 1H), 7.87 (dd, J=9.0, 1.6 Hz, 1H), 7.82 (d, J=1.7 Hz, 1H), 7.77 (d, J=8.4 Hz, 1H), 4.73-4.62 (m, 2H), 4.60-4.37 (m, 3H), 3.45-3.43 (m, 2H), 2.67 (s, 3H), 1.32-1.23 (m, 3H).

### Example 44. 2-(6-(azetidin-3-yl)pyridazin-3-yl)-5-(2-methyl-2H-pyrazolo[3,4-c]pyridin-5-yl)phenol hydrochloride (Compound 44)

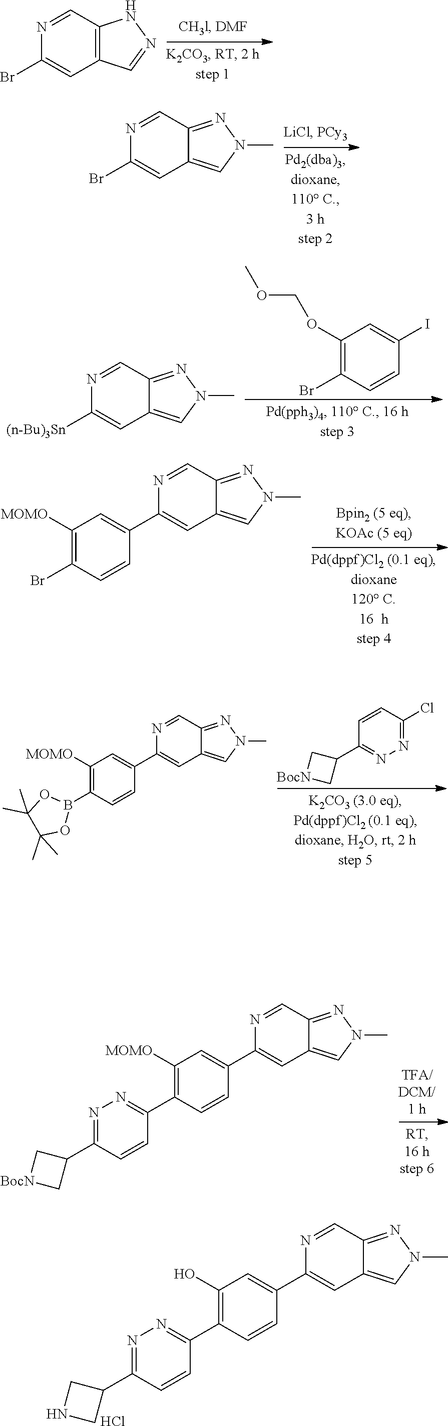

### Step 1: Preparation of 5-bromo-2-methyl-2H-pyrazolo[3,4-c]pyridine

To a mixture of 5-bromo-1H-pyrazolo[3,4-c]pyridine (4.00 g, 20.30 mmol) in DMF (40 mL) were added CH₃I (4.30 g, 30.4 mmol) and K₂CO₃ (8.40 g, 60.9 mmol). The mixture was stirred at RT for 2 h. The reaction mixture was diluted with water (20 mL) and extracted with EtOAc (40 mL). The organic layer was washed with brine (50 mL), dried with Na₂SO₄ and evaporated to give crude title compound. The residue was purified by silica gel column chromatography (PE/EA=3:1 to 1:4) to give title compound (1.50 g, 35.7% yield) as an off-white solid. ESI-MS (M+H+): 212.0. ¹H NMR (400 MHz, CDCl₃) δ 8.94 (s, 1H), 7.88 (s, 1H), 7.65 (d, J=0.9 Hz, 1H), 4.25 (s, 3H).

### Step 2: Preparation of 2-methyl-5-(tributylstannyl)-2H-pyrazolo[3,4-c]pyridine

To a mixture of 5-bromo-2-methyl-2H-pyrazolo[3,4-c]pyridine (1.20 g, 5.68 mmol) and Hexa-n-butylditin (9.80 g, 17.06 mmol) in dioxane (50 mL) were added Pd₂(dba)₃ (520 mg, 0.57 mmol), LiCl (1.20 g, 28.43 mmol) and PCy₃ (318 mg, 1.13 mmol). The mixture was stirred at 110° C. for 3 h. After concentration, the residue was purified by silica gel column chromatography (PE:EA=1:1) to give title product (2.20 g, Y: 87.5%) as a brown solid. ESI-MS (M+H+): 424.2. ¹H NMR (400 MHz, CDCl₃) δ 9.38 (s, 1H), 7.85 (s, 1H), 7.61 (s, 1H), 4.27 (s, 3H), 1.60-1.56 (m, 6H), 1.36-1.31 (m, 6H), 1.19-1.08 (m, 6H), 0.90-0.86 (m, 9H).

### Step 3: Preparation of 5-(4-bromo-3-(methoxymethoxy)phenyl)-2-methyl-2H-pyrazolo[3,4-c]pyridine

To a mixture of 2-methyl-5-(tributylstannyl)-2H-pyrazolo[3,4-c]pyridine (2.20 g, 5.15 mmol) and 1-bromo-4-iodo-2-(methoxymethoxy) benzene (1.20 g, 3.46 mmol) in toluene (20 mL) was added Pd(PPh₃)₄ (400 mg, 0.35 mmol). The mixture was stirred at 110° C. for 16 h. The mixture was filtered and the filtrate was concentrate in vacuo. The residue was purified by silica gel column chromatography (PE:EA=1:3) to give title product (1.1 g, Y: 91%) as a brown solid. ESI-MS (M+H+): 350.0. ¹H NMR (400 MHz, CDCl₃) δ 9.31 (s, 1H), 7.97 (s, 1H), 7.89 (dd, J=1.3, 0.8 Hz, 1H), 7.86 (d, J=1.5 Hz, 1H), 7.63-7.59 (m, 1H), 7.55-7.52 (m, 1H), 5.37 (s, 2H), 4.30 (s, 3H), 3.57 (s, 3H).

### Step 4: Preparation of 5-(3-(methoxymethoxy)-4-(4,4,5,5-tetramethyl-1,3,2-dioxaborolan-2-yl)phenyl)-2-methyl-2H-pyrazolo[3,4-c]pyridine

To a mixture of 5-(4-bromo-3-(methoxymethoxy)phenyl)-2-methyl-2H-pyrazolo[3,4-c]pyridine (1.00 g, 2.88 mmol) and B₂Pin₂ (14.60 g, 57.636 mmol) in dioxane (300 mL) were added Pd(dppf)Cl₂ (210 mg, 0.28 mmol) and KOAc (2.20 g, 23.04 mmol). The mixture was stirred at 120° C. for 16 h. The mixture was filtered and the filtrate was concentrate in vacuo. The residue was purified by Flash chromatography (PE:EA=1:3) to give title product (900 mg, Y: 81%) as a brown solid. ESI-MS (M+H+): 396.1. ¹H NMR (400 MHz, CDCl₃) δ 9.32 (d, J=1.0 Hz, 1H), 7.97-7.92 (m, 2H), 7.79 (d, J=7.7 Hz, 1H), 7.74 (d, J=1.4 Hz, 1H), 7.67 (dd, J=3.3, 1.7 Hz, 1H), 5.32 (s, 2H), 4.29 (s, 3H), 3.57 (s, 3H), 1.37 (s, 12H).

### Step 5: Preparation of tert-butyl 3-(6-(2-(methoxymethoxy)-4-(2-methyl-2H-pyrazolo[3,4-c]pyridin-5-yl)phenyl)pyridazin-3-yl)azetidine-1-carboxylate

To a mixture of 5-(3-(methoxymethoxy)-4-(4,4,5,5-tetramethyl-1,3,2-dioxaborolan-2-yl)phenyl)-2-methyl-2H-pyrazolo[3,4-c]pyridine (500 mg, 1.22 mmol) and tert-butyl 3-(6-chloropyridazin-3-yl) azetidine-1-carboxylate (340 mg, 1.22 mmol) in dioxane: H₂O (20 mL:5 mL) were added Pd(dppf)Cl₂ (92 mg, 0.13 mmol) and K₂CO₃ (522 mg, 3.29 mmol). The mixture was stirred at rt for 2 h. The mixture was filtered and the filtrate was concentrate in vacuo. The residue was purified by silica gel column chromatography (DCM:MeOH=40:1) to give title product (600 mg, Y: 90%) as a yellow solid. ESI-MS (M+H+): 503.0

### Step 6: Preparation of 2-(6-(azetidin-3-yl)pyridazin-3-yl)-5-(2-methyl-2H-pyrazolo[3,4-c]pyridin-5-yl)phenol hydrochloride

A mixture of tert-butyl 3-(6-(2-(methoxymethoxy)-4-(2-methyl-2H-pyrazolo[3,4-c]pyridin-5-yl)phenyl)pyridazin-3-yl)azetidine-1-carboxylate (60 mg, 0.12 mmol) in TFA (15 mL) was stirred at rt for 16 h. The mixture was concentrated in vacuo, the residue was purified by prep-HPLC (0.05% HCl in water/CH₃CN) to give title product (18.67 mg, Y: 38%) as a yellow solid. ESI-MS (M+H+): 359.2 ¹H NMR (400 MHz, MeOD-d₄) δ 9.22 (s, 1H), 8.45 (s, 1H), 8.43 (s, 1H), 8.21 (d, J=1.2 Hz, 1H), 8.05 (d, J=8.3 Hz, 1H), 7.76 (d, J=9.0 Hz, 1H), 7.68-7.62 (m, 2H), 4.56-4.52 (m, 4H), 4.51-4.44 (m, 1H), 4.34 (s, 3H).

### Example 45. 5-(2-methyl-2H-indazol-5-yl)-2-{6-[1-(oxolan-3-yl)azetidin-3-yl]pyridazin-3-yl}phenol (Compound 45)

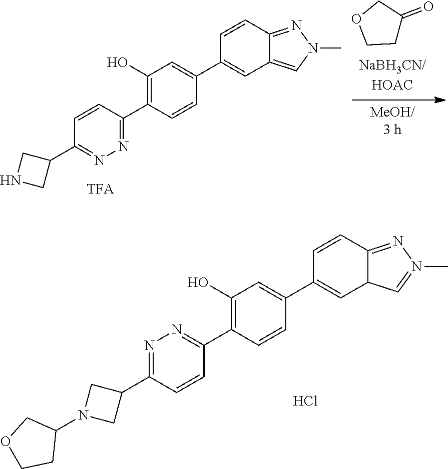

### Step 1: Preparation of 5-(2-methyl-2H-indazol-5-yl)-2-(6-(1-(tetrahydrofuran-3-yl)azetidin-3-yl)pyridazin-3-yl)phenol hydrochloride

To a solution of 2-(6-(azetidin-3-yl)pyridazin-3-yl)-5-(2-methyl-2H-indazol-5-yl)phenol (200 mg, 0.56 mmol) in MeOH (40 mL) were added dihydrofuran-3 (2H)-one (240 mg, 2.8 mmol) and acetic acid (200 mg, 3.36 mmol). The mixture was stirred at RT for 1 h, then sodium cyanoborohydride (106 mg, 1.68 mmol) was added under ice bath and the mixture was stirred at rt for 2 h. The solution was concentrated in vacuo. The crude was purified by prep-HPLC (0.05% HCl in water/CH₃CN) to give title compound (78 mg, yield: 33%) as a yellow solid. ESI-MS (M+H)+: 428.0. ¹H NMR (400 MHz, MeOD-d₄) δ 8.80 (d, J=8.3 Hz, 1H), 8.73 (s, 1H), 8.28 (d, J=9.0 Hz, 1H), 8.24 (s, 1H), 8.02 (d, J=8.4 Hz, 1H), 7.99-7.96 (m, 1H), 7.82 (d, J=9.0 Hz, 1H), 7.51-7.48 (m, 1H), 7.44 (d, J=1.7 Hz, 1H), 4.78-4.69 (m, 2H), 4.65-4.53 (m, 3H), 4.38 (s, 3H), 4.36-4.28 (m, 1H), 4.16-4.07 (m, 2H), 3.80-3.71 (m, 2H), 2.48-2.36 (m, 1H), 2.08-1.98 (m, 1H).

### Example 46. 2-[6-(1-ethylazetidin-3-yl)pyridazin-3-yl]-5-{2-methyl-2H-pyrazolo[3,4-c]pyridin-5-yl}phenol (Compound 46)

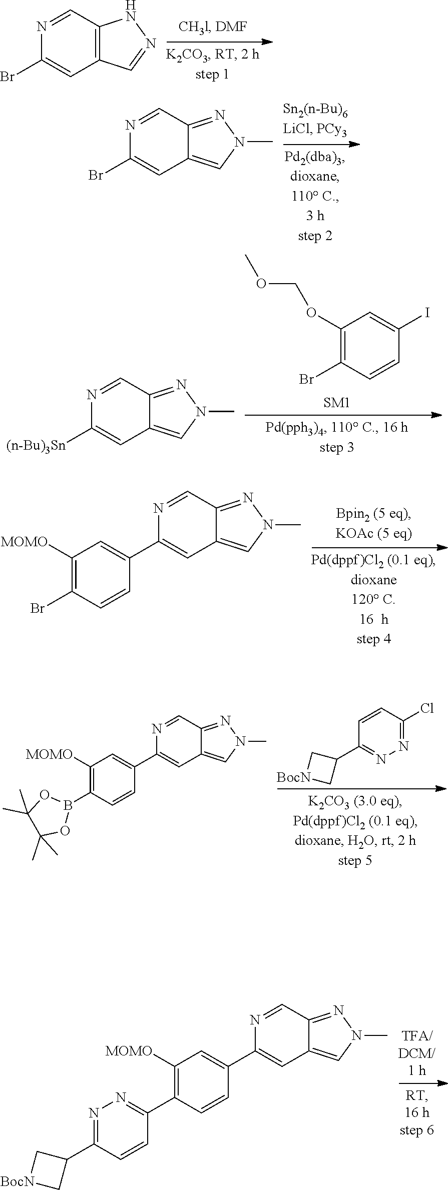

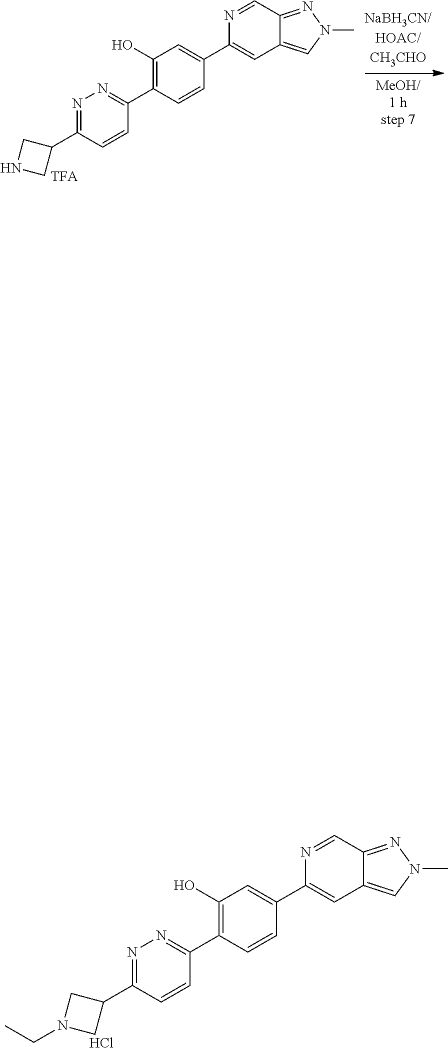

### Step 1: Preparation of 5-bromo-2-methyl-2H-pyrazolo[3,4-c]pyridine

To a mixture of 5-bromo-3a,7a-dihydro-1H-pyrazolo[3,4-c]pyridine (4.00 g, 20.30 mmol) in DMF (40 mL) were added CH₃I (4.30 g, 30.4 mmol) and K₂CO₃ (8.40 g, 60.9 mmol). The mixture was stirred at RT for 2 h. The reaction mixture was diluted with water (20 mL) and extracted with EtOAc (40 mL). The organic layer was washed with brine (50 mL), dried with Na₂SO₄ and evaporated to give crude title compound. The residue was purified by silica gel column chromatography (PE/EA=3:1 to 1:4) to give title compound (1.50 g, 35.7% yield) as an off-white solid. ESI-MS (M+H+): 212.0. ¹H NMR (400 MHz, CDCl₃) δ 8.94 (s, 1H), 7.88 (s, 1H), 7.65 (d, J=0.9 Hz, 1H), 4.25 (s, 3H).

### Step 2: Preparation of 2-methyl-5-(tributylstannyl)-2H-pyrazolo[3,4-c]pyridine

To a mixture of 5-bromo-2-methyl-2H-pyrazolo[3,4-c]pyridine (1.20 g, 5.68 mmol) and Hexa-n-butylditin (9.80 g, 17.06 mmol) in dioxane (50 mL) were added Pd₂(dba)₃ (520 mg, 0.57 mmol), LiCl (1.20 g, 28.43 mmol) and PCy₃ (318 mg, 1.13 mmol). The mixture was stirred at 110° C. for 3 h. After concentration, the residue was purified by flash chromatography (PE:EA=1:1) to give title product (2.20 g, Y: 87.5%) as a brown solid. ESI-MS (M+H+): 424.2. ¹H NMR (400 MHz, CDCl₃) δ 9.38 (s, 1H), 7.85 (s, 1H), 7.61 (s, 1H), 4.27 (s, 3H), 1.60-1.56 (m, 6H), 1.36-1.31 (m, 6H), 1.19-1.08 (m, 6H), 0.90-0.86 (m, 9H).

### Step 3: Preparation of 5-(4-bromo-3-(methoxymethoxy)phenyl)-2-methyl-2H-pyrazolo[3,4-c]pyridine

To a mixture of 2-methyl-5-(tributylstannyl)-2H-pyrazolo[3,4-c]pyridine (2.20 g, 5.15 mmol) and 1-bromo-4-iodo-2-(methoxymethoxy) benzene (1.20 g, 3.46 mmol) in toluene (20 mL) was added Pd(PPh₃) (400 mg, 0.35 mmol). The mixture was stirred at 110° C. for 16 h. LCMS showed the starting material was consumed completely. The mixture was filtered and the filtrate was concentrated in vacuo. The residue was purified by silica gel column chromatography (PE:EA=1:3) to give title product (1.1 g, Y: 91%) as a brown solid. ESI-MS (M+H+): 350.0. ¹H NMR (400 MHz, CDCl₃) δ 9.31 (s, 1H), 7.97 (s, 1H), 7.89 (dd, J=1.3, 0.8 Hz, 1H), 7.86 (d, J=1.5 Hz, 1H), 7.63-7.59 (m, 1H), 7.55-7.52 (m, 1H), 5.37 (s, 2H), 4.30 (s, 3H), 3.57 (s, 3H).

### Step 4: Preparation of 5-(3-(methoxymethoxy)-4-(4,4,5,5-tetramethyl-1,3,2-dioxaborolan-2-yl)phenyl)-2-methyl-2H-pyrazolo[3,4-c]pyridine

To a mixture of 5-(4-bromo-3-(methoxymethoxy)phenyl)-2-methyl-2H-pyrazolo[3,4-c]pyridine (1.00 g, 2.88 mmol) and B₂pin₂ (14.60 g, 57.636 mmol) in dioxane (300 mL) were added Pd(dppf)Cl₂ (210 mg, 0.28 mmol) and KOAc (2.20 g, 23.04 mmol). The mixture was stirred at 120° C. for 16 h. The mixture was filtered and the filtrate was concentrated in vacuo. The residue was purified by flash chromatography (PE:EA=1:3) to give title product (900 mg, Y: 81%) as a brown solid. ESI-MS (M+H+): 396.1. ¹H NMR (400 MHz, CDCl₃) δ 9.32 (d, J=1.0 Hz, 1H), 7.97-7.92 (m, 2H), 7.79 (d, J=7.7 Hz, 1H), 7.74 (d, J=1.4 Hz, 1H), 7.67 (dd, J=3.3, 1.7 Hz, 1H), 5.32 (s, 2H), 4.29 (s, 3H), 3.57 (s, 3H), 1.37 (s, 12H).

### Step 5: Preparation of tert-butyl 3-(6-(2-(methoxymethoxy)-4-(2-methyl-2H-pyrazolo[3,4-c]pyridin-5-yl)phenyl)pyridazin-3-yl)azetidine-1-carboxylate

To a mixture of 5-(3-(methoxymethoxy)-4-(4,4,5,5-tetramethyl-1,3,2-dioxaborolan-2-yl)phenyl)-2-methyl-2H-pyrazolo[3,4-c]pyridine (500 mg, 1.22 mmol) and tert-butyl 3-(6-chloropyridazin-3-yl) azetidine-1-carboxylate (340 mg, 1.22 mmol) in dioxane: H₂O (20 mL: 5 mL) were added Pd(dppf)Cl₂ (92 mg, 0.13 mmol) and K₂CO₃ (522 mg, 3.29 mmol). The mixture was stirred at rt for 2 h. LCMS showed the starting material was consumed completely. The mixture was filtered and the filtrate was concentrated in vacuo. The residue was purified by silica gel column chromatography (DCM:MeOH=40:1) to give title product (600 mg, Y: 90%) as a yellow solid. ESI-MS (M+H+): 503.0.

### Step 6: Preparation of 2-(6-(azetidin-3-yl)pyridazin-3-yl)-5-(2-methyl-2H-pyrazolo[3,4-c]pyridin-5-yl)phenol

A mixture of tert-butyl 3-(6-(2-(methoxymethoxy)-4-(2-methyl-2H-pyrazolo[3,4-c]pyridin-5-yl)phenyl)pyridazin-3-yl)azetidine-1-carboxylate (600 mg, 1.20 mmol) in TFA (15 mL) was stirred at rt for 16 h. LCMS showed the starting material was consumed completely. The mixture was concentrated in vacuo and the residue (50 mg) was purified by prep HPLC (0.05% FA in water/CH₃CN) to give title product (18.67 mg, Y: 36%) as a yellow solid. ESI-MS (M+H+): 359.2 ¹H NMR (400 MHz, MeOD-d₄) δ 9.22 (s, 1H), 8.45 (d, J=9.4 Hz, 2H), 8.21 (d, J=1.2 Hz, 1H), 8.05 (d, J=8.3 Hz, 1H), 7.76 (d, J=9.0 Hz, 1H), 7.68-7.62 (m, 2H), 4.56-4.52 (m, 4H), 4.51-4.44 (m, 1H), 4.34 (s, 3H).

### Step 7: Preparation of 2-(6-(1-ethylazetidin-3-yl)pyridazin-3-yl)-5-(2-methyl-2H-pyrazolo[3,4-c]pyridin-5-yl)phenol hydrochloride

To a mixture of 2-(6-(azetidin-3-yl)pyridazin-3-yl)-5-(2-methyl-2H-pyrazolo[3,4-c]pyridin-5-yl)phenol (50 mg, 0.11 mmol) and CH₃CHO (60 mg, 1.34 mmol) in MeOH (16 mL) and HOAc (16 drops) was added NaBH₃CN (50 mg, 0.86 mmol). The mixture was stirred at RT for 1 h. LCMS showed the starting material was consumed completely. Filtered and concentrated in vacuo. The residue was purified by prep HPLC (0.05% HCl in water/CH₃CN) to give title product (4 mg, Y: 9.5%) as a yellow solid. ESI-MS (M+H+): 387.2. ¹H NMR (400 MHz, MeOD-d₄) δ 9.71 (s, 1H), 8.90 (s, 1H), 8.66 (s, 1H), 8.64-8.57 (m, 1H), 8.25-8.19 (m, 1H), 7.98 (d, J=9.0 Hz, 1H), 7.59-7.51 (m, 2H), 4.74-4.65 (m, 2H), 4.58-4.54 (m, 1H), 4.54 (s, 3H), 4.52-4.45 (m, 2H), 3.48-3.40 (m, 2H), 1.33-1.27 (m, 3H).

### Example 47. 2-[6-(1-ethylazetidin-3-yl)pyridazin-3-yl]-5-{imidazo[1,2-b]pyridazin-6-yl}phenol (Compound 47)

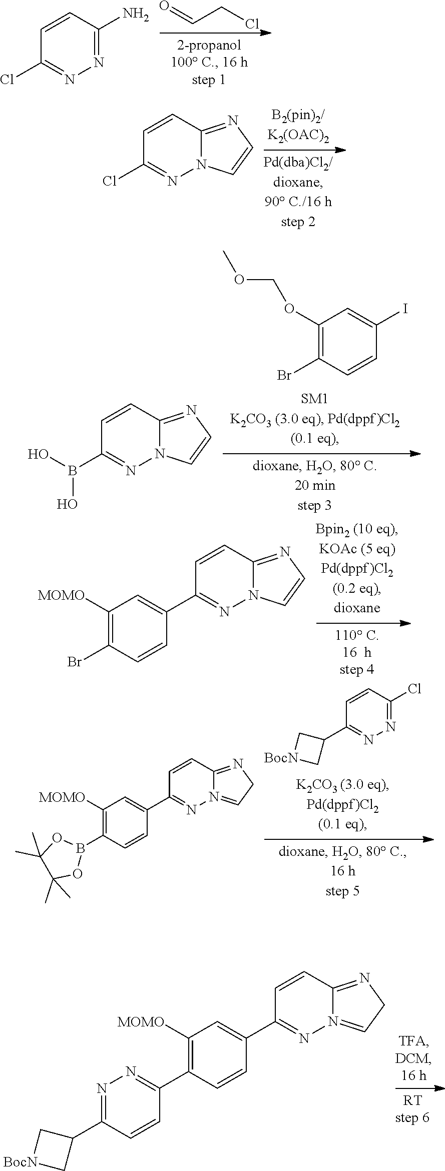

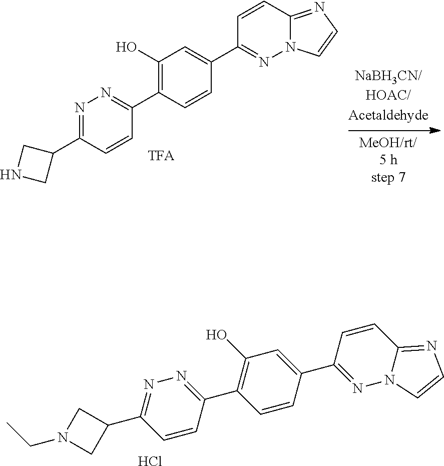 [SMILES: Oc1cc(-c2ccc3nccn3n2)ccc1-c1ccc(C2CNC2)nn1 ; CCN1CC(c2ccc(-c3ccc(-c4ccc5nccn5n4)cc3O)nn2)C1 ; Cl ; "TFA"]

### Step 1: Preparation of 6-chloroimidazo[1,2-b]pyridazine

A mixture of 6-chloropyridazin-3-amine (10 g, 0.0775 mol) and 2-chloroacetaldehyde (30.22 g, 0.387 mol) in 2-propanol (100 mL) was stirred at 100° C. for 16 h. The reaction mixture was evaporated to give crude title compound. The crude was purified by silica gel column chromatography (PE/EA=5:1) to give title compound (10 g, 83% yield) as a white solid. ESI-MS (M+H)+: 154.2. ¹H NMR (400 MHz, DMSO-d₆) δ 8.57 (s, 1H), 8.42 (d, J=9.6 Hz, 1H), 8.15 (s, 1H), 7.69 (d, J=9.5 Hz, 1H).

### Step 2: Preparation of imidazo[1,2-b]pyridazin-6-ylboronic acid

A mixture of 6-chloroimidazo[1,2-b]pyridazine (4.5 g, 29.4 mmol), B₂pin₂ (14.9 g, 58.8 mmol), KOAc (8.6 g, 88.2 mmol) and Pd(dppf)Cl₂ (1 g, 1.47 mmol) in 1,4-dioxane (270 mL) was stirred at 90° C. for 16 h. The reaction mixture was filtered and the filtrate was concentrated in vacuo to give title compound (4.0 g, crude) as a black oil. ESI-MS (M+H)+: 164.0.

### Step 3: Preparation of 6-(4-bromo-3-(methoxymethoxy)phenyl)imidazo[1,2-b]pyridazine

A mixture of imidazo[1,2-b]pyridazin-6-ylboronic acid (2.7 g, 16.56 mmol), 1-bromo-4-iodo-2-(methoxymethoxy)benzene (5.66 g, 16.56 mmol), K₂CO₃ (6.8 g, 49.68 mmol) and Pd(dppf)Cl₂ (1.2 g, 1.656 mmol) in 1,4-dioxane/H₂O (270 mL/27 mL) was stirred at 80° C. for 20 min. The reaction mixture was diluted with water (150 mL) and extracted with EtOAc (150 mL×3). The organic layer was washed with brine, dried over Na₂SO₄ and evaporated to give crude title compound. The crude was purified by silica gel column chromatography (PE/EA=5:1) to give title compound (2.5 g, 45% yield) as a black oil. ESI-MS (M+H)+: 335.8. ¹H NMR (400 MHz, CDCl₃) δ 7.99-7.92 (m, 2H), 7.71 (dd, J=6.3, 3.1 Hz, 2H), 7.62 (d, J=8.3 Hz, 1H), 7.41-7.34 (m, 2H), 5.30 (s, 2H), 3.50 (s, 3H).

### Step 4: Preparation of 6-(3-(methoxymethoxy)-4-(4,4,5,5-tetramethyl-1,3,2-dioxaborolan-2-yl)phenyl)imidazo[1,2-b]pyridazine

A mixture of 6-(4-bromo-3-(methoxymethoxy)phenyl)imidazo[1,2-b]pyridazine (2.5 g, 7.5 mmol), B₂Pin₂ (19 g, 75 mmol), KOAc (3.67 g, 37.5 mmol) and Pd(dppf)Cl₂ (548 mg, 0.75 mmol) in 1,4-dioxane (150 mL) was stirred at 110° C. for 16 h. The reaction mixture was diluted with water (200 mL) and extracted with EtOAc (200 mL×3). The organic layer was washed with brine, dried over Na₂SO₄ and evaporated to give crude title compound. The crude was purified by silica gel column chromatography (PE/EA=1:1) to give title compound (2 g, 74% yield) as a black oil. ESI-MS (M+H)+: 382.1.

### Step 5: Preparation of tert-butyl 3-(6-(4-(imidazo[1,2-b]pyridazin-6-yl)-2-(methoxymethoxy)phenyl)pyridazin-3-yl)azetidine-1-carboxylate

A mixture of 6-(3-(methoxymethoxy)-4-(4,4,5,5-tetramethyl-1,3,2-dioxaborolan-2-yl)phenyl)imidazo[1,2-b]pyridazine. (2 g, 5.24 mmol), tert-butyl 3-(6-chloropyridazin-3-yl)azetidine-1-carboxylate (1.4 g, 5.24 mmol), K₂CO₃ (2.17 g, 15.72 mmol) and Pd(dppf)Cl₂ (383 mg, 0.524 mmol) in 1,4-dioxane/H₂O (200 mL/20 mL) was stirred at 80° C. for 16 h. The reaction mixture was diluted with water (100 mL) and extracted with EtOAc (150 mL×3). The organic layer was washed with brine, dried over Na₂SO₄ and evaporated to give crude title compound. The crude was purified by silica gel column chromatography (PE/EA=5:1) to give title compound (1.4 g, 56% yield) as a black solid. ESI-MS (M+H)+: 489.1.

### Step 6: Preparation of 2-(6-(azetidin-3-yl)pyridazin-3-yl)-5-(imidazo[1,2-b]pyridazin-6-yl)phenol (1:1)

To a solution of tert-butyl 3-(6-(4-(imidazo[1,2-b]pyridazin-6-yl)-2-(methoxymethoxy)phenyl)pyridazin-3-yl)azetidine-1-carboxylate (1.4 g, 2.87 mmol) in DCM (15 mL) was added TFA (15 ml) at room temperature. The reaction mixture was stirred for 16 h. The reaction mixture was diluted with DCM (50 mL) concentrated in vacuo to give crude title compound. The crude was dissolved with water (5 mL), the aqueous solution washed with EA (20 mL*5) and freeze-dry to give title compound (1.2 g, Y: 61%) as a red solid. ESI-MS (M+H)+: 345.0.

### Step 7: Preparation of 2-(6-(1-ethylazetidin-3-yl)pyridazin-3-yl)-5-(imidazo[1,2-b]pyridazin-6-yl)phenol hydrochloride

To a solution of 2-(6-(azetidin-3-yl)pyridazin-3-yl)-5-(imidazo[1,2-b]pyridazin-6-yl)phenol (1:1) (190 mg, 0.55 mmol) in MeOH (40 mL) and HOAc (190 mg) was added Acetaldehyde (121 mg, 2.75 mol). The resulting mixture was stirred for 1 h at rt. Then aNaBH₃CN (104 mg, 1.65 mmol) was added at 0° C. The resulting mixture was stirred for 5 h at rt. The mixture was evaporated to give crude title compound. The crude was purified by prep-HPLC (0.05% HCl in water/ACN) to give title compound (10 mg, Y: 4.9%) as a yellow solid. ESI-MS (M+H)+: 373.2. ¹H NMR (400 MHz, MeOD-d₄) δ 8.66-8.60 (m, 2H), 8.56 (d, J=9.7 Hz, 1H), 8.50 (d, J=9.7 Hz, 1H), 8.31 (d, J=2.2 Hz, 1H), 8.20 (dd, J=8.3, 4.8 Hz, 1H), 8.02 (d, J=9.0 Hz, 1H), 7.89 (s, 1H), 7.86 (d, J=8.3 Hz, 1H), 4.79-4.64 (m, 2H), 4.61-4.45 (m, 3H), 3.51-3.40 (m, 2H), 1.32-1.30 (m, 3H).

### Example 48. 2-[6-(1-ethylazetidin-3-yl)pyridazin-3-yl]-5-(7-methoxy-2-methyl-2H-indazol-5-yl)phenol (Compound 48)

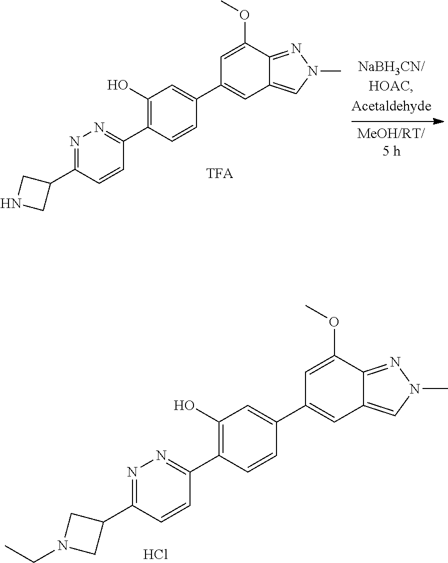

### Step 1: Preparation of 2-(6-(I-ethylazetidin-3-yl) pyridazin-3-yl)-5-(7-methoxy-2-methyl-2H-indazol-5-yl) phenol hydrochloride

To a solution of 2-(6-(azetidin-3-yl) pyridazin-3-yl)-5-(7-methoxy-2-methyl-2H-indazol-5-yl)phenol (1:1) (100 mg, 0.258 mmol) in MeOH (20 mL) and HOAc (100 mg) was added Acetaldehyde (56.8 mg, 1.29 mmol). The resulting mixture was stirred for 1 h at r.t. Then NaBH₃CN (48.8 mg, 0.774 mmol) was added at 0° C. The resulting mixture was stirred for 5 h at rt. The mixture was diluted with water (1 mL) and evaporated to give crude title compound. The crude was purified by prep-HPLC (0.05% HCl in water/ACN) to give title compound (17 mg, Y: 15.88%) as a yellow solid. ESI-MS (M+H)+: 416.2. ¹H NMR (400 MHz, MeOD-d₄) δ 8.65 (d, J=9.2 Hz, 1H), 8.48 (d, J=2.2 Hz, 1H), 8.10 (dd, J=9.0, 4.8 Hz, 1H), 7.91 (dd, J=8.3, 6.4 Hz, 1H), 7.63 (s, 1H), 7.40-7.35 (m, 1H), 7.32 (d, J=1.7 Hz, 1H), 7.12 (d, J=2.4 Hz, 1H), 4.68-4.55 (m, 2H), 4.51-4.32 (m, 3H), 4.23 (s, 3H), 4.04 (s, 3H), 3.40-3.29 (m, 2H), 1.21-1.19 (m, 3H).

### Example 49. 5-(7-methoxy-2-methyl-2H-indazol-5-yl)-2-{6-[1-(oxan-4-yl)azetidin-3-yl]pyridazin-3-yl}phenol (Compound 49)

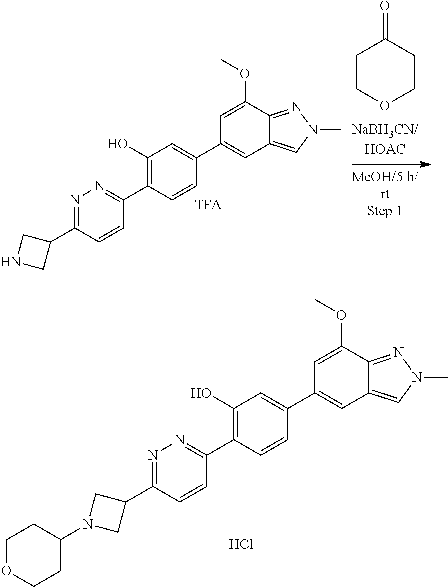 [SMILES: COc1cc(-c2ccc(-c3ccc(C4CNC4)nn3)c(O)c2)cc2cn(C)nc12 ; O=C1CCOCC1 ; COc1cc(-c2ccc(-c3ccc(C4CN(C5CCOCC5)C4)nn3)c(O)c2)cc2cn(C)nc12 ; Cl]

### Step 1: Preparation of 5-(7-methoxy-2-methyl-2H-indazol-5-yl)-2-(6-(1-(tetrahydro-2H-pyran-4-yl) azetidin-3-yl) pyridazin-3-yl) phenol hydrochloride

To a mixture of 2-(6-(azetidin-3-yl) pyridazin-3-yl)-5-(7-methoxy-2-methyl-2H-indazol-5-yl) phenol (100 mg, 0.26 mmol) and tetrahydro-4H-pyran-4-one (130 mg, 1.3 mmol) in MeOH (10 mL) and HOAc (20 drops) was added NaBH₃CN (40 mg, 0.63 mmol). The mixture was stirred at RT for 5 h. LCMS showed the starting material was consumed completely. The mixture was added water (1 mL) and concentrated in vacuo. The residue was purified by prep-HPLC (0.05% HCl in water/ACN) to give title product (39 mg, Y: 30%) as a yellow solid. ESI-MS (M+H+): 472.3. ¹H NMR (400 MHz, MeOD-d₄) δ 8.83-8.57 (m, 2H), 8.43-8.20 (m, 1H), 7.95-7.88 (m, 1H), 7.75-7.65 (m, 1H), 7.47-7.33 (m, 2H), 7.27-7.17 (m, 1H), 4.70-4.48 (m, 5H), 4.31-4.25 (m, 3H), 4.08-3.96 (m, 5H), 3.66-3.51 (m, 1H), 3.36 (t, J=11.6 Hz, 2H), 1.98-1.96 (m, 2H), 1.58-1.44 (m, 2H).

### Example 50. 2-[6-(1-ethylazetidin-3-yl)pyridazin-3-yl]-5-{2-methyl-2H-pyrazolo[4,3-b]pyridin-5-yl}phenol (Compound 50)

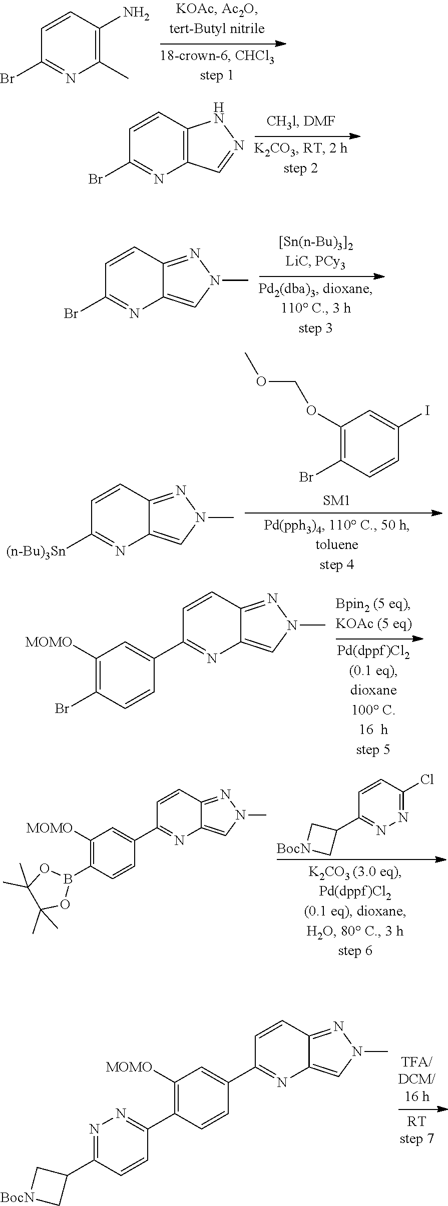

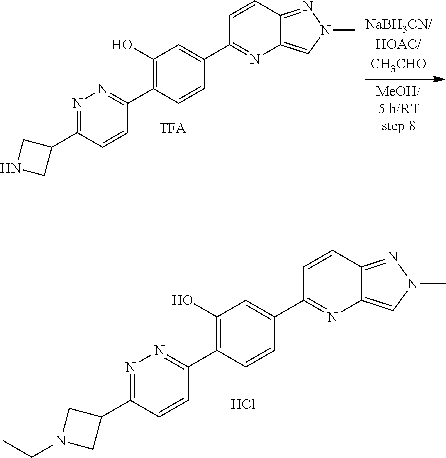

### Step 1: Preparation of 5-bromo-1H-pyrazolo [4,3-b]pyridine

To a solution of 6-bromo-2-methylpyridin-3-amine (15 g, 80.64 mmol), KOAc (9.4 g, 96.774 mmol) in CHCl₃ (250 mL) was added Ac₂O (33 g, 322.58 mmol), the mixture was stirred for 1 h at RT. Then the reaction mixture was stirred at 65° C. for 2 h. tert-Butyl nitrite (16.6 g, 161.29 mmol) and 18-crown-6 (17 g, 64.512 mmol) were added, the reaction mixture was stirred at 80° C. for 16 h. After cooling to rt d, sat·K₂CO₃ (200 mL) was added and the mixture was stirred at rt for 2 h. The mixture was extracted with EA (300 mL*3) and combined the organic layers and concentrated. The residue was purified by column chromatography (PE/EA=3:1) to give title product (7.3 g, 45.6%) as a yellow solid, ESI-MS (M+H)+: 199.9. ¹H NMR (400 MHz, DMSO-d₆) δ 13.57 (s, 1H), 8.29 (s, 1H), 8.02 (d, J=8.7 Hz, 1H), 7.52 (d, J=8.7 Hz, 1H).

### Step 2: Preparation of 5-bromo-2-methyl-2H-pyrazolo[4,3-b]pyridine

A mixture of 5-bromo-1H-pyrazolo[4,3-b]pyridine (7.3 g, 37 mmol), CH₃I (7.8 g, 55.5 mmol), K₂CO₃ (15.3 g, 111 mmol) in DMF (150 mL) was stirred at RT for 2 h. The mixture was added water (150 mL), extracted with EA (200 mL×3). The organic layer was washed with brine, dried over Na₂SO₄ and concentrated in vacuo to give crude title compound. The crude was purified by silica gel column chromatography (PE/EA=5:1) to give title compound (2.4 g, 30.88% yield) as a yellow solid. ESI-MS (M+H)+: 212.0. ¹H NMR (400 MHz, CDCl₃) δ 8.10 (s, 1H), 7.89 (d, J=8.9 Hz, 1H), 7.32 (d, J=9.0 Hz, 1H), 4.25 (s, 3H).

### Step 3: Preparation of 2-methyl-5-(tributylstannyl)-2H-pyrazolo[4,3-b]pyridine

A mixture of 5-bromo-2-methyl-2H-pyrazolo[4,3-b]pyridine (2.4 g, 11.37 mmol), LiCl (2.387 g, 56.85 mmol), Pcy₃ (636.72 mg, 2.274 mmol) and Pd₂(dba)₃ (1.04 g, 1.137 mmol) in 1,4-dioxane (240 mL) was charged with N₂ for three times and added [Sn(n-Bu)₃]₂ (19.783 g, 34.11 mmol) was added. The mixture was stirred at 110° C. for 3 h. The reaction mixture was diluted with KF solution (200 mL) and extracted with EtOAc (200 mL×3). The organic layer was washed with brine, dried over Na₂SO₄ and evaporated to give crude title compound. The crude was purified by silica gel column chromatography (PE/EA=3:1) to give title compound (3.4 g, 70.8% yield) as a green oil. ESI-MS (M+H)+: 423.5. ¹H NMR (400 MHz, CDCl₃) δ 8.19 (s, 1H), 7.84 (dd, J=8.6, 0.8 Hz, 1H), 7.45-7.12 (m, 1H), 4.24 (s, 3H), 1.66-1.52 (m, 6H), 1.43-1.27 (m, 6H), 1.23-1.13 (m, 6H), 0.91-0.83 (m, 9H).

### Step 4: Preparation of 5-(4-bromo-3-(methoxymethoxy)phenyl)-2-methyl-2H-pyrazolo[4,3-b]pyridine

A mixture of 2-methyl-5-(tributylstannyl)-2H-pyrazolo[4,3-b]pyridine (3.4 g, 8.03 mmol), 1-bromo-4-iodo-2-(methoxymethoxy)benzene (2.746 g, 8.03 mmol), Pd(PPh₃)₄ (924 mg, 0.8 mmol) in toluene (340 mL) was stirred at 110° C. for 50 h. The reaction mixture was diluted with water (150 mL) and extracted with EtOAc (150 mL×3). The organic layer was washed with brine, dried over Na₂SO₄ and evaporated to give crude title compound. The crude was purified by silica gel column chromatography (PE/EA=2:1) to give title compound (3 g, 45% yield) as a yellow solid. ESI-MS (M+H)+: 350.0. ¹H NMR (400 MHz, CDCl₃) δ 8.21 (s, 1H), 8.09 (d, J=9.0 Hz, 1H), 7.86 (d, J=1.8 Hz, 1H), 7.68 (s, 1H), 7.54 (dd, J=8.2, 1.9 Hz, 1H), 7.45 (dd, J=10.4, 5.4 Hz, 1H), 5.38 (s, 2H), 4.28 (s, 3H), 3.57 (s, 3H).

### Step 5: Preparation of 5-(3-(methoxymethoxy)-4-(4,4,5,5-tetramethyl-1,3,2-dioxaborolan-2-yl)phenyl)-2-methyl-2H-pyrazolo[4,3-b]pyridine

A mixture of 5-(4-bromo-3-(methoxymethoxy)phenyl)-2-methyl-2H-pyrazolo[4,3-b]pyridine (3 g, 8.64 mmol), B₂pin₂ (10.97 g, 43.2 mmol), KOAc (4.233 g, 43.2 mmol) and Pd(dppf)Cl₂ (705 mg, 0.864 mmol) in 1,4-dioxane (150 mL) was stirred at 100° C. for 16 h. The reaction mixture was diluted with water (200 mL) and extracted with EtOAc (200 mL×3). The organic layer was washed with brine, dried over Na₂SO₄ and evaporated to give crude title compound. The crude was purified by silica gel column chromatography (PE/EA=1:1) to give title compound (3.2 g, 56% yield) as a black oil. ESI-MS (M+H)+: 396.2. ¹H NMR (400 MHz, CDCl₃) δ 8.22 (s, 1H), 8.08 (d, J=9.1 Hz, 1H), 7.81 (d, J=7.7 Hz, 1H), 7.73 (d, J=1.3 Hz, 1H), 7.73-7.69 (m, 1H), 7.66 (dd, J=7.7, 1.3 Hz, 1H), 5.32 (s, 2H), 4.28 (s, 3H), 3.56 (s, 3H), 1.37 (s, 12H).

### Step 6: Preparation of tert-butyl 3-(6-(2-(methoxymethoxy)-4-(2-methyl-2H-pyrazolo[4,3-b]pyridin-5-yl)phenyl)pyridazin-3-yl)azetidine-1-carboxylate

A mixture of 5-(3-(methoxymethoxy)-4-(4,4,5,5-tetramethyl-1,3,2-dioxaborolan-2-yl)phenyl)-2-methyl-2H-pyrazolo[4,3-b]pyridine (270 mg, 0.68 mmol), tert-butyl 3-(6-chloropyridazin-3-yl)azetidine-1-carboxylate (274 mg, 1.02 mmol), K₂CO₃ (281 mg, 2.04 mmol) and Pd(dppf)Cl₂ (55.5 mg, 0.068 mmol) in 1,4-dioxane/H₂O (25 mL/2.5 mL) was stirred at 80° C. for 3 h. The reaction mixture was diluted with water (100 mL) and extracted with EtOAc (80 mL×3). The organic layer was washed with brine, dried over Na₂SO₄ and evaporated to give crude title compound. The crude was purified by silica gel column chromatography (PE/EA=1:1) to give title compound (340 mg, 95% yield) as a yellow solid. ESI-MS (M+H)+: 503.1. ¹H NMR (400 MHz, CDCl₃) δ 8.24 (s, 1H), 8.15-8.13 (m, 1H), 8.11 (t, J=6.4 Hz, 2H), 7.98 (d, J=1.5 Hz, 1H), 7.83 (dd, J=8.1, 1.6 Hz, 1H), 7.77 (d, J=9.1 Hz, 1H), 7.52 (d, J=8.8 Hz, 1H), 5.36 (s, 2H), 4.43 (t, J=8.6 Hz, 2H), 4.35-4.25 (m, 5H), 4.21-4.10 (m, 1H), 3.49 (s, 3H), 1.48 (s, 9H).

### Step 7: Preparation of 2-(6-(azetidin-3-yl)pyridazin-3-yl)-5-(2-methyl-2H-pyrazolo[4,3-b]pyridin-5-yl)phenol (1:1)

To a solution of tert-butyl 3-(6-(2-(methoxymethoxy)-4-(2-methyl-2H-pyrazolo[4,3-b]pyridin-5-yl)phenyl)pyridazin-3-yl)azetidine-1-carboxylate (340 mg, 0.677 mmol) in DCM (12 mL) was added TFA (12 ml) at room temperature. The reaction mixture was stirred at RT for 16 h. The reaction mixture was concentrated to give crude title compound. The crude was dissolved with water (25 mL), the aqueous solution washed with EA (20 mL*3) and freeze-dry to give title compound (300 mg, Y: 60%) as a yellow solid. ESI-MS (M+H)+: 359.2. ¹H NMR (400 MHz, MeOD-d₄) δ 8.50 (s, 1H), 8.46 (d, J=9.1 Hz, 1H), 8.20 (dd, J=9.1, 0.8 Hz, 1H), 8.09 (d, J=8.2 Hz, 1H), 7.90 (d, J=9.1 Hz, 1H), 7.77 (d, J=9.1 Hz, 1H), 7.73-7.66 (m, 2H), 4.57-4.42 (m, 5H), 4.31 (s, 3H).

### Step 8: Preparation of 2-(6-(1-ethylazetidin-3-yl)pyridazin-3-yl)-5-(2-methyl-2H-pyrazolo[4,3-b]pyridin-5-yl)phenol hydrochloride

To a solution of 2-(6-(azetidin-3-yl)pyridazin-3-yl)-5-(2-methyl-2H-pyrazolo[4,3-b]pyridin-5-yl)phenol (70 mg, 0.195 mmol) in MeOH (14 mL) and HOAc (70 mg) was added acetaldehyde (42.9 mg, 0.975 mol). The resulting mixture was stirred for 1 h at r.t. Then NaBH₃CN (36.8 mg, 0.585 mmol) was added at 0° C. The resulting mixture was stirred for 5 h at r.t. The mixture was evaporated to give crude title compound. The crude was purified by prep-HPLC (0.05% HCl in water/ACN) to give title compound (20 mg, Y: 26.6%) as a yellow solid. ESI-MS (M+H)+: 387.2. ¹H NMR (400 MHz, MeOD-d₄) δ 8.92 (d, J=9.0 Hz, 1H), 8.83 (s, 1H), 8.70-8.46 (m, 1H), 8.32-8.26 (m, 1H), 8.23 (dd, J=9.0, 1.4 Hz, 1H), 7.95 (d, J=9.0 Hz, 1H), 7.75 (t, J=1.9 Hz, 1H), 7.73-7.68 (m, 1H), 4.76-4.64 (m, 2H), 4.58-4.47 (m, 3H), 4.45 (s, 3H), 3.48-3.40 (m, 2H), 1.41-1.21 (m, 3H).

### Example 51. 5-{2-methyl-2H-pyrazolo[4,3-b]pyridin-5-yl}-2-{6-[1-(oxan-4-yl)azetidin-3-yl]pyridazin-3-yl}phenol (Compound 51)

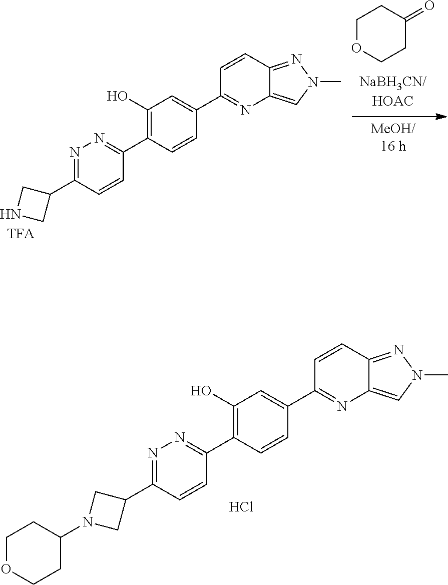

### Step 1: Preparation of 5-(2-methyl-2H-pyrazolo[4,3-b]pyridin-5-yl)-2-(6-(1-(tetrahydro-2H-pyran-4-yl)azetidin-3-yl)pyridazin-3-yl)phenol hydrochloride

To a solution of 2-(6-(azetidin-3-yl)pyridazin-3-yl)-5-(2-methyl-2H-pyrazolo[4,3-b]pyridin-5-yl)phenol (93 mg, 0.195 mmol) in MeOH (14 mL) and HOAc (70 mg) was added tetrahydro-4H-pyran-4-one (97.5 mg, 0.975 mol). The resulting mixture was stirred for 1 h at r.t. Then NaBH₃CN (36.8 mg, 0.585 mmol) was added at 0° C. The resulting mixture was stirred for 5 h at r.t. The mixture was evaporated to give crude title compound. The crude was purified by prep-HPLC (0.05% HCl in water/ACN) to give title compound (42 mg, Y: 48.8%) as a yellow solid. ESI-MS (M+H)+: 443.3. ¹H NMR (400 MHz, MeOD-d₄) δ 8.89 (d, J=9.7 Hz, 1H), 8.81 (s, 1H), 8.64-8.55 (m, 1H), 8.33-8.26 (m, 1H), 8.21 (d, J=9.1 Hz, 1H), 7.94 (d, J=9.1 Hz, 1H), 7.77-7.73 (m, 1H), 7.72-7.67 (m, 1H), 4.77-4.53 (m, 5H), 4.44 (s, 3H), 4.15-4.02 (m, 2H), 3.70-3.59 (m, 1H), 3.52-3.40 (m, 2H), 2.16-1.98 (m, 2H), 1.70-1.43 (m, 2H).

### Example 52. 2-[6-(1-ethylazetidin-3-yl)pyridazin-3-yl]-5-{7-methoxy-2-methyl-2H-pyrazolo[3,4-c]pyridin-5-yl}phenol (Compound 52)

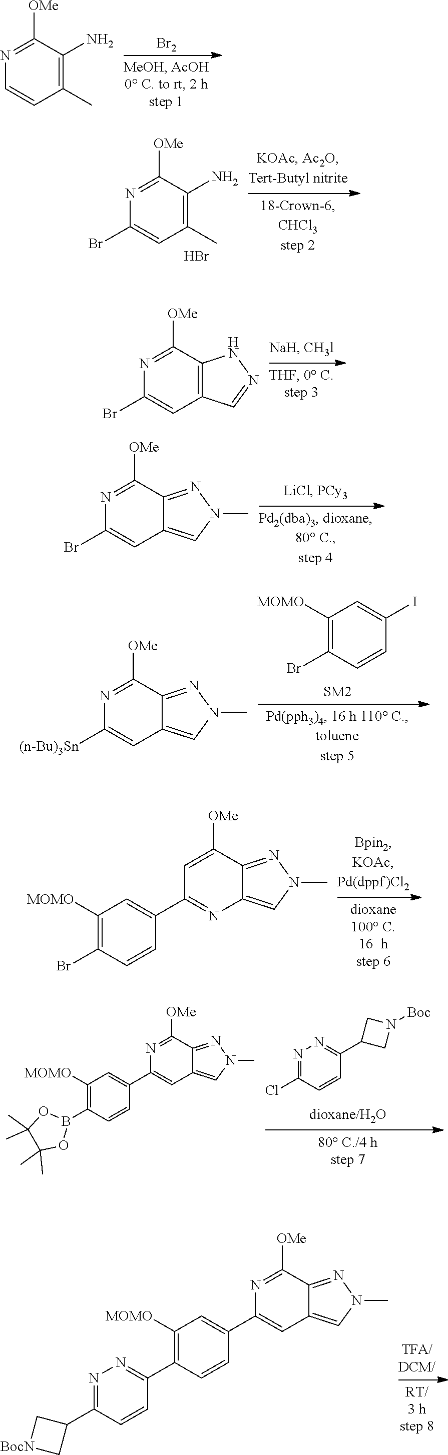

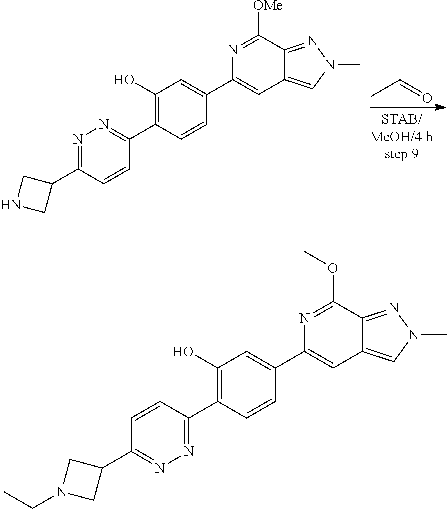

### Step 1: Preparation of 6-bromo-2-methoxy-4-methylpyridin-3-amine

To a solution of 2-methoxy-6-methylaniline (25 g, 0.181 mol) in MeOH (400 mL) was added Br₂ (29 g, 0.181 mol) in AcOH (50 mL) dropwised at RT, the mixture was stirred for 2 h at RT. The reaction mixture was diluted with EtOAc (500 mL) and stirred for 5 min. The solid was collected by filiation, washed with EtOAc (50 mL×2) to afford title compound (30 g, 76.7% yield) as a pink solid. ESI-MS (M+H)+: 217.0

### Step 2: Preparation of 5-bromo-7-methoxy-1H-pyrazolo[3,4-c]pyridine

To a solution of 4-bromo-2-methoxy-6-methylaniline hydrobromide (1.0 g, 4.63 mmol), KOAc (544 mg, 5.55 mmol) in CHCl₃ (10 mL) was added Ac₂O (1.9 g, 19 mmol), the mixture was stirred for 1 h at RT. The reaction mixture was stirred at 70° C. for 2 h. Then isopentynitrate (963 mg, 9.2 mmol) and 18-crown-6 (98 mg, 0.46 mmol) was added, the reaction mixture was stirred at 65° C. for 16 h. After cooling to rt, sat. K₂CO₃ (20 mL) was added and the mixture was stirred at rt for 2 h. The mixture was exacted with EA (30 mL*3) and the organic layers were concentrated. The crude was purified by flash to provide title product (400 mg, 38.1%) as a yellow solid, ESI-MS (M+H)+: 228.0.

### Step 3: Preparation of 5-bromo-7-methoxy-2-methyl-2H-indazole

To a solution of 5-bromo-7-methoxy-1H-pyrazolo[3,4-c]pyridine (2.8 g, 12.3 mmol) in THF (60 mL) was added NaH (600 mg, 24.6 mmol) and at 0° C. under N₂ atmosphere. The mixture was stirred at rt for 30 min under N₂ atmosphere. To the mixture was added CH₃I (6.6 g, 46.9 mmol) under N₂ atmosphere. The mixture was stirred at r.t for 2 h under N₂ atmosphere. The mixture was diluted with H₂O (20 mL) and extracted with EA (50 mL*3). The organic layer was washed with brine, dried over Na₂SO₄ and evaporated to give crude. The crude was purified by silica gel column chromatography (PE:EA=10:1-3:2) to give title product (2.0 g, yield 68%) as an orange solid. ESI-MS (M+H)+: 244.0. ¹H NMR (400 MHz, CDCl₃) δ 7.79 (s, 1H), 7.27 (s, 1H), 4.23 (s, 3H), 4.16 (s, 3H).

### Step 4: Preparation of 2-methyl-5-(tributylstannyl)-2H-pyrazolo[4,3-b]pyridine

To a mixture of 5-bromo-7-methoxy-2-methyl-2H-pyrazolo[3,4-c]pyridine (500 mg, 2.075 mmol), Pd₂(dba)₃ (190 mg, 0.207 mmol), LiCl (435 mg, 10.373 mmol), Pcy₃ (115 mg, 0.415 mmol) in dioxane (30 mL) was added hexabutylditin (3.62 g, 6.22 mmol), the reaction mixture was charged with N₂ for three times and stirred at 80° C. for 3 h. Then sat. KF (50 mL) was added t and the mixture was stirred at r.t for 1 h. The mixture was extracted with EA (60 mL*3) and combined organic layers was concentrated in vacuo. The residue was purified by silica gel chromatography (PE/EA=4:1) to give title product (850 mg, 97.1%) as a yellow solid. ESI-MS (M+H)+: 454.2 ¹H NMR (400 MHz, DMSO-d₆) δ 8.29 (s, 1H), 7.29 (s, 1H), 4.21 (s, 3H), 4.03 (s, 3H), 1.67-1.57 (m, 6H), 1.39-1.32 (m, 6H), 1.15-1.03 (m, 6H), 0.89 (t, J=7.2 Hz, 9H).

### Step 5: Preparation of 5-(4-bromo-3-(methoxymethoxy)phenyl)-7-methoxy-2-methyl-2H-pyrazolo[3,4-c]pyridine

To a mixture of 2-methyl-5-(tributylstannyl)-2H-pyrazolo[4,3-b]pyridine (850 mg, 1.876 mmol), Pd(PPh₃)₄ (217 mg, 0.188 mmol) in toluene (30 mL) was added 1-bromo-4-iodo-2-(methoxymethoxy)benzene (963 mg, 2.815 mmol), the mixture was charged with N₂ for three times and stirred at 110° C. for 16 h. The reaction mixture was diluted with water (30 mL) and extracted with EtOAc (60 mL×3). The organic layer was washed with brine, dried over Na₂SO₄ and evaporated to give crude title compound. The crude was purified silica column chromatography (PE/EA=2:1) to give title compound (350 mg, 49.5% yield) as a white solid. ESI-MS (M+H)+: 379.8 ¹H NMR (400 MHz, CDCl₃) δ 7.90 (s, 1H), 7.84 (s, 1H), 7.58 (d, J=0.9 Hz, 2H), 7.50 (s, 1H), 5.35 (s, 2H), 4.24 (s, 3H), 4.22 (s, 3H), 3.57 (s, 3H).

### Step 6: Preparation of 7-methoxy-5-(3-(methoxymethoxy)-4-(4,4,5,5-tetramethyl-1,3,2-dioxaborolan-2-yl)phenyl)-2-methyl-2H-pyrazolo[3,4-c]pyridine

To a mixture of 5-(4-bromo-3-(methoxymethoxy)phenyl)-7-methoxy-2-methyl-2H-pyrazolo[3,4-c]pyridine (300 mg, 0.796 mmol), B₂pin₂ (1.42 g, 5.57 mmol) in 1,4-dioxane (30 mL) was added Pd(dppf)Cl₂ (33 mg, 0.04 mmol), AcOK (234 mg, 2.387 mmol), the mixture was charged with N₂ for three times and stirred at 100° C. for 16 h under N₂. The reaction mixture was purified by silica gel chromatography (PE/EA=4:1) to give the title product (300 mg, 76%) as a brown solid. ESI-MS (M+H)+: 426.3 ¹H NMR (400 MHz, CDCl₃) δ 7.86 (d, J=2.5 Hz, 1H), 7.80-7.78 (m, 1H), 7.72-7.68 (m, 1H), 7.56 (s, 1H), 7.53-7.47 (m, 1H), 5.30 (s, 2H), 4.25 (s, 3H), 4.23 (s, 3H), 3.57 (s, 3H), 1.37 (s, 12H).

### Step 7: Preparation of tert-butyl 3-(6-(4-(7-methoxy-2-methyl-2H-pyrazolo[3,4-c]pyridin-5-yl)-2-(methoxymethoxy)phenyl)pyridazin-3-yl)azetidine-1-carboxylate

To a mixture of 7-methoxy-5-(3-(methoxymethoxy)-4-(4,4,5,5-tetramethyl-1,3,2-dioxaborolan-2-yl)phenyl)-2-methyl-2H-pyrazolo[3,4-c]pyridine (250 mg, 0.588 mmol), tert-butyl 3-(6-chloropyridazin-3-yl)azetidine-1-carboxylate (237 mg, 0.882 mmol) in 1,4-dioxane (30 mL) and H₂O (3 mL) were added Pd(dppf)Cl₂ (24 mg, 0.029 mmol), K₂CO₃ (244 mg, 1.765 mmol), the mixture was charged with N₂ for three times and stirred at 80° C. for 3 h under N₂. After concentration, the crude was purified by silica gel chromatography (PE/EA=1:1) to give title product (160 mg, 51%) as a brown solid. ESI-MS (M+H)+: 533.3 ¹H NMR (400 MHz, CDCl₃) δ 8.09 (dd, J=8.5, 4.1 Hz, 2H), 8.06 (d, J=1.5 Hz, 1H), 7.90 (s, 1H), 7.86 (dd, J=8.2, 1.6 Hz, 1H), 7.63 (s, 1H), 7.50 (d, J=8.9 Hz, 1H), 5.33 (s, 2H), 4.44-4.43 (m, 3H), 4.32-4.28 (m, 2H), 4.28-4.27 (m, 3H), 4.26 (s, 3H), 3.49 (s, 3H), 1.48 (s, 9H).

### Step 8: synthesis of 2-(6-(azetidin-3-yl)pyridazin-3-yl)-5-(7-methoxy-2-methyl-2H-pyrazolo[3,4-c]pyridin-5-yl)phenol trifluoroacetate

To a solution of tert-butyl 3-(6-(4-(7-methoxy-2-methyl-2H-pyrazolo[3,4-c]pyridin-5-yl)-2-(methoxymethoxy)phenyl)pyridazin-3-yl)azetidine-1-carboxylate (100 mg, 0.188 mmol) in DCM (4 mL) was added TFA (2 mL). The mixture was stirred at RT for 5 h. The mixture was concentrated to give 2-(6-(azetidin-3-yl)pyridazin-3-yl)-5-(7-methoxy-2-methyl-2H-pyrazolo[3,4-c]pyridin-5-yl)phenol trifluoroacetate (60 mg, TFA salt) as a yellow solid. ESI-MS (M+H)+: 389.2

### Step 9: synthesis of 2-(6-(1-ethylazetidin-3-yl)pyridazin-3-yl)-5-(7-methoxy-2-methyl-2H-pyrazolo[3,4-c]pyridin-5-yl)phenol

To a solution of 2-(6-(azetidin-3-yl)pyridazin-3-yl)-5-(7-methoxy-2-methyl-2H-pyrazolo[3,4-c]pyridin-5-yl)phenol (60 mg, 0.155 mmol) in MeOH (5 mL) was added acetaldehyde (20 mg, 0.464 mol). The resulting mixture was stirred for 2 h at r.t. Then sodium cyanoborohydride (164 mg, 0.773 mol) was added. The resulting mixture was stirred for 4 h at r.t. The mixture was concentrated in vacuo and the residue was purified by prep-HPLC (0.05% NH₃·H₂O in water/ACN) to give 2-(6-(1-ethylazetidin-3-yl)pyridazin-3-yl)-5-(7-methoxy-2-methyl-2H-pyrazolo[3,4-c]pyridin-5-yl)phenol (2 mg, Y: 3%) as a yellow solid and 5-(4-(6-(1-ethylazetidin-3-yl)pyridazin-3-yl)-3-hydroxyphenyl)-2-methyl-2H-pyrazolo[3,4-c]pyridin-7-ol (9 mg, Y: 14.5%) as a yellow solid. ESI-MS (M+H)+: 417.2 ¹H NMR (400 MHz, MeOD-d₄) δ 8.37 (d, J=9.2 Hz, 1H), 8.24 (d, J=4.7 Hz, 1H), 7.95 (d, J=8.5 Hz, 1H), 7.85-7.80 (m, 1H), 7.78-7.74 (m, 2H), 7.71 (d, J=8.4, 1.6 Hz, 1H), 4.23 (d, J=1.5 Hz, 6H), 4.11-4.04 (m, 1H), 3.86 (t, J=8.2 Hz, 2H), 3.55 (t, J=7.9 Hz, 2H), 2.65 (q, J=7.2 Hz, 2H), 1.04 (t, J=7.2 Hz, 3H).

### Example 53. 5-{4-[6-(1-ethylazetidin-3-yl)pyridazin-3-yl]-3-hydroxyphenyl}-2-methyl-2H-pyrazolo[3,4-c]pyridin-7-ol (Compound 53)

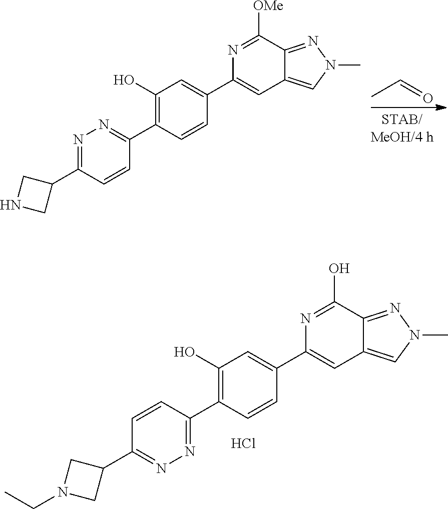

### Step 1: synthesis of 5-(4-(6-(1-ethylazetidin-3-yl)pyridazin-3-yl)-3-hydroxyphenyl)-2-methyl-2H-pyrazolo[3,4-c]pyridin-7-ol hydrochloride

To a solution of 2-(6-(azetidin-3-yl)pyridazin-3-yl)-5-(7-methoxy-2-methyl-2H-pyrazolo[3,4-c]pyridin-5-yl)phenol trifluoroacetate (60 mg, 0.155 mmol) in MeOH (5 mL) was added acetaldehyde (20 mg, 0.464 mmol). The resulting mixture was stirred for 2 h at r.t. Then sodium cyanoborohydride (164 mg, 0.773 mmol). The resulting mixture was stirred for 4 h at r.t. The mixture was added water (1 mL) and concentrated in vacuo, the residue was purified by prep-HPLC (0.05% HCl in water/ACN) and freeze-drying to give 5-(4-(6-(1-ethylazetidin-3-yl)pyridazin-3-yl)-3-hydroxyphenyl)-2-methyl-2H-pyrazolo[3,4-c]pyridin-7-ol (9 mg, Y: 14.5%) as a yellow solid. ESI-MS (M+H)+: 403.3 ¹H NMR (400 MHz, MeOD-d₄) δ 8.78 (dd, J=9.0, 3.0 Hz, 1H), 8.33 (t, J=8.2 Hz, 1H), 8.15 (s, 1H), 7.99-7.94 (m, 1H), 7.41-7.37 (m, 1H), 7.36-7.33 (m, 1H), 6.98 (d, J=1.0 Hz, 1H), 4.78-4.70 (m, 2H), 4.68-4.54 (m, 2H), 4.53-4.47 (m, 1H), 4.19 (s, 3H), 3.50-3.41 (m, 2H), 1.34-1.28 (m, 3H).

### Example 54. 5-(7-ethoxy-2-methyl-2H-indazol-5-yl)-2-{6-[1-(oxan-4-yl)azetidin-3-yl]pyridazin-3-yl}phenol (Compound 54)

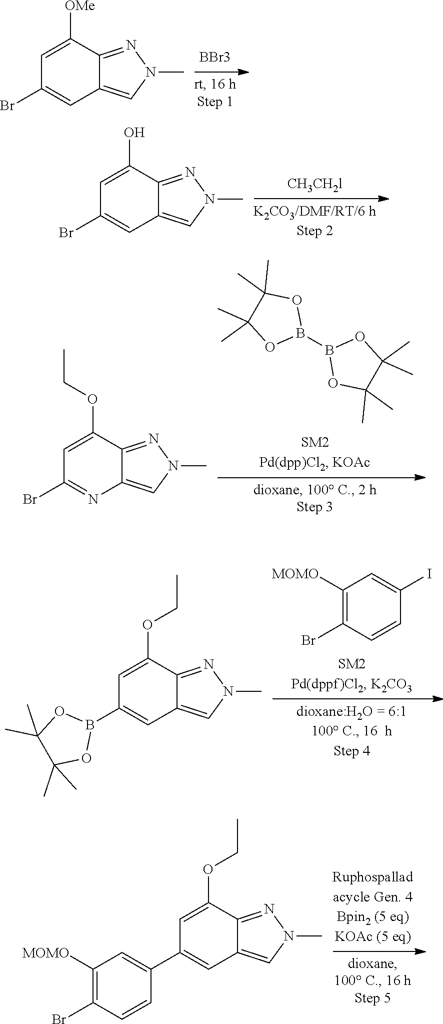

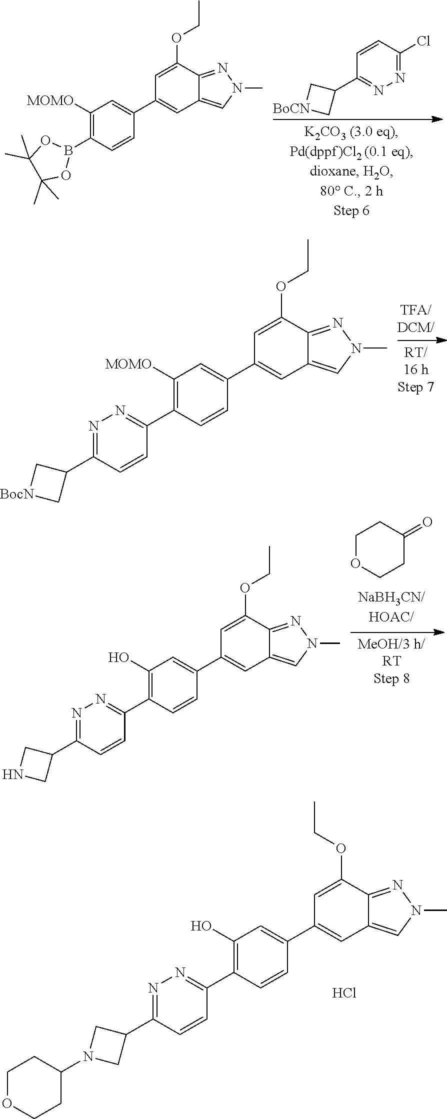

### Step 1: Preparation of 5-bromo-2-methyl-2H-indazol-7-ol

To a solution of 5-bromo-7-methoxy-2-methyl-2H-indazole (3 g, 12.45 mmol) in DCM (50 mL) was added BBr₃ (1 mol/L, 80 mL) under ice bath. The mixture was stirred at RT for 16 h. MeOH (50 mL) was added to quench BBr₃ and basified with saturated sodium bicarbonate aqueous solution and extracted with DCM (300 mL×3). The organic layer was washed with brine, dried over sodium sulfate, filtered and concentrated in vacuo to get title product (2.8 g, crude) as a brown solid. ESI-MS (M+H)+: 227.0.

### Step 2: Preparation of 5-bromo-7-ethoxy-2-methyl-2H-indazole

To a solution of 5-bromo-2-methyl-2H-indazol-7-ol (2.567 g, 11.36 mmol) in DMF (50 mL) were added iodoethane (3.544 g, 22.72 mmol) and K₂CO₃ (4.7 g, 34.08 mmol). The mixture was stirred at RT for 6 h. The solution was diluted with water (60 mL), extracted with EA(60×3 mL). The organic layer was washed with brine, dried over sodium sulfate, filtered and concentrated in vacuo to get the title product (3 g, crude) as a black solid. ESI-MS (M+H)+: 257.1. ¹H NMR (400 MHz, DMSO-d₆) δ 8.25 (s, 1H), 7.45 (d, J=1.5 Hz, 1H), 6.66 (d, J=1.5 Hz, 1H), 4.21-4.15 (m, 2H), 4.13 (s, 3H), 1.40 (t, J=7.0 Hz, 3H).

### Step 3: Preparation of 7-ethoxy-2-methyl-5-(4,4,5,5-tetramethyl-1,3,2-dioxaborolan-2-yl)-2H-indazole

To a mixture of 5-bromo-7-ethoxy-2-methyl-2H-indazole (2.8 g, 11.024 mmol), B₂pin₂ (7 g, 27.560 mmol) in 1,4-dioxane (150 mL) were added KOAc (3.781 g, 38.584 mmol) and Pd(dppf)Cl₂ (804 mg, 1.102 mmol), the mixture was charged with N₂ for three times and stirred at 100° C. for 2 h under N₂. The reaction mixture was used to next step without purification further. ESI-MS (M+H)+: 302.8.

### Step 4: Preparation of 5-(4-bromo-3-(methoxymethoxy) phenyl)-7-ethoxy-2-methyl-2H-indazole

To a mixture of 7-ethoxy-2-methyl-5-(4,4,5,5-tetramethyl-1,3,2-dioxaborolan-2-yl)-2H-indazole (3.33 g, 11.024 mmol) in 1,4-dioxane (150 mL) were added 1-bromo-4-iodo-2-(methoxymethoxy) benzene (5.7 g, 16.536 mmol), Pd(dppf)Cl₂ (804 mg, 1.102 mmol), K₂CO₃ (3.042 g, 22.048 mmol) and water (25 mL), the mixture was charged with N₂ for three times and stirred at 100° C. for 16 h. The reaction mixture was diluted with water (150 mL) and extracted with EtOAc (200 mL×3). The organic layer was washed with brine, dried over Na₂SO₄ and evaporated to give crude title compound. The crude was purified by silica gel column chromatography (PE:EA=5:1-1:1) to give title product (2 g, crude) as brown oily, then the residue was re-purified by C₁₈-Flash (0.05% FA in H₂O/CH₃CN) to give title compound (888 mg, yield: 21%) as a yellow solid. ESI-MS (M+H)+: 393.1. ¹H NMR (400 MHz, CDCl₃) δ 7.89 (s, 1H), 7.58 (d, J=8.2 Hz, 1H), 7.39 (d, J=2.0 Hz, 1H), 7.35 (d, J=1.1 Hz, 1H), 7.14 (d, J=8.2, 2.0 Hz, 1H), 6.74 (s, 1H), 5.33 (s, 2H), 4.32 (q, J=7.0 Hz, 2H), 4.24 (s, 3H), 3.57 (s, 3H), 1.60 (t, J=7.0 Hz, 3H).

### Step 5: Preparation of 7-ethoxy-5-(3-(methoxymethoxy)-4-(4,4,5,5-tetramethyl-1,3,2-dioxaborolan-2-yl)phenyl)-2-methyl-2H-indazole

To a mixture of 5-(4-bromo-3-(methoxymethoxy) phenyl)-7-ethoxy-2-methyl-2H-indazole (700 mg, 1.795 mmol), B₂pin₂ (2.28 g, 8.974 mmol) in 1,4-dioxane (50 mL) were added KOAc (879 mg, 8.974 mmol) and Ruphospallad a cycle G4 (180 mg, 0.180 mmol), the mixture was charged with N₂ for three times and stirred at 100° C. for 16 h under N₂. The reaction mixture was diluted with water (100 mL) and extracted with EtOAc (100 mL×3). The organic layer was washed with brine, dried over Na₂SO₄ and evaporated to give crude title compound. The crude was purified by silica gel column chromatography (EA:PE=30%˜100%) to give title product (411 mg, yield: 52%) as a yellow solid. ESI-MS (M+H)+: 439.2. ¹H NMR (400 MHz, CDCl₃) δ 7.88 (s, 1H), 7.77 (d, J=8.1 Hz, 1H), 7.39 (s, 1H), 7.28 (d, J=6.7 Hz, 2H), 6.79 (s, 1H), 5.29 (d, J=10.8 Hz, 2H), 4.34-4.29 (m, 2H), 4.24 (s, 3H), 3.55 (d, J=3.8 Hz, 3H), 1.59 (t, J=3.2 Hz, 3H), 1.37 (s, 12H).

### Step 6: Preparation of tert-butyl 3-(6-(4-(7-ethoxy-2-methyl-2H-indazol-5-yl)-2-(methoxymethoxy) phenyl) pyridazin-3-yl) azetidine-1-carboxylate

To a mixture of 7-ethoxy-5-(3-(methoxymethoxy)-4-(4,4,5,5-tetramethyl-1,3,2-dioxaborolan-2-yl)phenyl)-2-methyl-2H-indazole (360 mg, 0.822 mmol) and tert-butyl 3-(6-chloropyridazin-3-yl)azetidine-1-carboxylate (243 mg, 0.904 mmol) and K₂CO₃ (340 mg, 2.466 mmol) in 1,4-dioxane:H₂O (15 mL:3 mL) was added Pd(dppf)Cl₂ (60 mg, 0.082 mmol). The mixture was stirred at 80° C. under N₂ for 2 h. The mixture was allowed to cooling down to room temperature, concentrated under reduced pressure, added water (20 mL), extracted with EA (20 mL×3). The combined organic layer was washed brine (40 mL), dried over sodium sulfate, filtered and concentrated in vacuo, the residue was purified by silica gel column chromatography (PE:EA=2:1-1:3) to give title product (300 mg, Y: 67%) as a white solid. ESI-MS (M+H)+: 546.3. ¹H NMR (400 MHz, CDCl₃) δ 8.08 (d, J=8.4, 4.1 Hz, 2H), 7.91 (s, 1H), 7.51 (d, J=5.1, 3.7 Hz, 2H), 7.45 (d, J=5.1, 2.5 Hz, 2H), 6.84 (s, 1H), 5.30 (s, 2H), 4.45-4.35 (m, 4H), 4.32-4.27 (m, 2H), 4.25 (s, 3H), 4.17-4.12 (m, 1H), 3.48 (s, 3H), 1.48 (s, 9H), 1.47-1.24 (m, 3H).

### Step 7: Preparation of 2-(6-(azetidin-3-yl) pyridazin-3-yl)-5-(7-ethoxy-2-methyl-2H-indazol-5-yl) phenol

To a solution of tert-butyl 3-(6-(4-(7-ethoxy-2-methyl-2H-indazol-5-yl)-2-(methoxymethoxy) phenyl) pyridazin-3-yl) azetidine-1-carboxylate (300 mg, 0.550 mmol) in DCM (10 mL) was added TFA (5 mL). The resulting mixture was stirred at RT for 16 h. The mixture was diluted with DCM (20 mL) and concentrated in vacuo and lyophilized to give title product (300 mg, crude) as an orange solid. ESI-MS (M+H)+: 402.1.

### Step 8: Preparation of 5-(7-ethoxy-2-methyl-2H-indazol-5-yl)-2-(6-(1-(tetrahydro-2H-pyran-4-yl) azetidin-3-yl) pyridazin-3-yl) phenol hydrochloride

To a solution of 2-(6-(azetidin-3-yl) pyridazin-3-yl)-5-(7-ethoxy-2-methyl-2H-indazol-5-yl) phenol (50 mg, crude) in MeOH (10 mL) were added tetrahydro-4H-pyran-4-one (62 mg, 0.623 mmol) and acetic acid (38 mg, 0.623 mmol). The mixture was stirred at RT for 1 h, then sodium cyanoborohydride (24 mg, 0.375 mmol) was added under ice bath and the mixture was stirred at RT for 2 h, then water (2 mL) was added and the solution was concentrated in vacuo. The crude was purified by prep-HPLC (0.05% HCl in water/CH₃CN) to give title compound (15 mg, yield: 25%) as an orange solid. ESI-MS (M+H)+: 486.2. ¹H NMR (400 MHz, MeOD-d₄) δ 8.61 (d, J=9.0 Hz, 1H), 8.49 (s, 1H), 8.02 (d, J=9.2 Hz, 2H), 7.67 (s, 1H), 7.40-7.39 (m, 2H), 7.15 (s, 1H), 4.78-4.54 (m, 5H), 4.39 (d, J=7.0 Hz, 2H), 4.30 (s, 3H), 4.13-4.06 (m, 2H), 3.69-3.60 (m, 1H), 3.45 (d, J=11.7 Hz, 2H), 2.11-2.03 (m, 2H), 1.61-1.53 (m, 5H).

### Example 55. 5-(7-ethoxy-2-methyl-2H-indazol-5-yl)-2-[6-(1-methylazetidin-3-yl)pyridazin-3-yl]phenol (Compound 55)

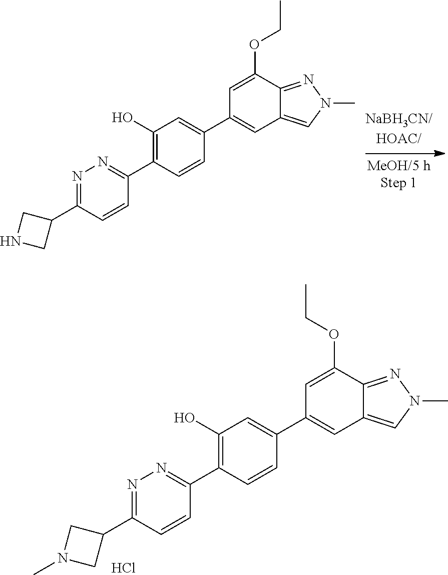

### Step 1: Preparation of 5-(7-ethoxy-2-methyl-2H-indazol-5-yl)-2-(6-(I-methylazetidin-3-yl) pyridazin-3-yl) phenol

To a mixture of 2-(6-(azetidin-3-yl) pyridazin-3-yl)-5-(7-ethoxy-2-methyl-2H-indazol-5-yl) phenol (50 mg, 0.12 mmol) and (HCHO)ₙ (20 mg, 0.67 mmol) in MeOH (5 mL) and HOAc (1 drops) was added NaBH₃CN (25 mg, 0.4 mmol). The mixture was stirred at RT for 5 h. Filtered and concentrated in vacuo. The residue was purified by prep-HPLC (0.05% HCl in water/ACN) to give title product (15 mg, Y: 31%) as a yellow solid. ESI-MS (M+H+): 416.2. ¹H NMR (400 MHz, MeOD-d₄) δ 8.80-8.67 (m, 1H), 8.58 (s, 1H), 8.28-8.14 (m, 1H), 8.05-7.91 (m, 1H), 7.70 (s, 1H), 7.49-7.35 (m, 2H), 7.17 (s, 1H), 4.84-4.75 (m, 2H), 4.62-4.45 (m, 3H), 4.39 (q, J=7.0 Hz, 2H), 4.33 (s, 3H), 3.10 (d, J=5.9 Hz, 3H), 1.57 (t, J=6.9 Hz, 3H).

### Example 56. 5-(7-ethoxy-2-methyl-2H-indazol-5-yl)-2-[6-(1-ethylazetidin-3-yl)pyridazin-3-yl]phenol (Compound 56)

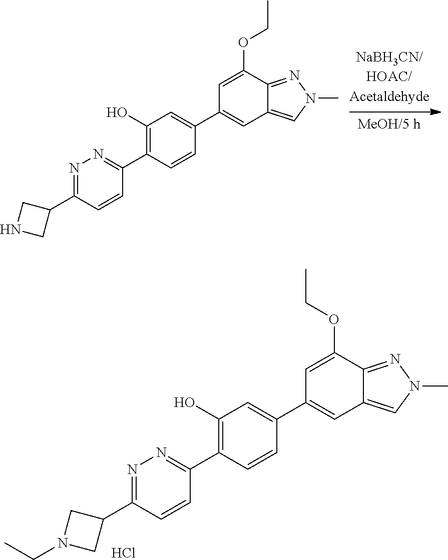

### Step 1: Preparation of 5-(7-ethoxy-2-methyl-2H-indazol-5-yl)-2-(6-(I-ethylazetidin-3-yl)pyridazin-3-yl)phenol hydrochloride

To a solution of 2-(6-(azetidin-3-yl)pyridazin-3-yl)-5-(7-ethoxy-2-methyl-2H-indazol-5-yl)phenol (50 mg, 0.125 mmol) in MeOH (10 mL) and HOAc (50 mg) was added acetaldehyde (27.5 mg, 0.625 mmol). The resulting mixture was stirred for 1 h at rt. Then added NaBH₃CN (24 mg, 0.375 mmol) at 0° C. The resulting mixture was stirred for 5 h at rt. The mixture was evaporated to give crude title compound. The crude was purified by prep-HPLC (0.05% HCl in water/ACN) to give title compound (12 mg, Y: 22.6%) as a yellow solid. ESI-MS (M+H)+: 430.2. ¹H NMR (400 MHz, MeOD-d₄) δ 8.71 (d, J=9.1 Hz, 1H), 8.59 (s, 1H), 8.16 (d, J=8.6 Hz, 1H), 8.01 (dd, J=8.3, 6.1 Hz, 1H), 7.71 (s, 1H), 7.44 (d, J=8.3 Hz, 1H), 7.40 (d, J=1.6 Hz, 1H), 7.21 (s, 1H), 4.84-4.67 (m, 2H), 4.61-4.45 (m, 3H), 4.40 (q, J=6.9 Hz, 2H), 4.33 (s, 3H), 3.45-3.43 (m, 2H), 1.58 (t, J=7.0 Hz, 3H), 1.31-1.29 (m, 3H).

### Example 57. 5-{7-methoxy-2-methyl-2H-pyrazolo[3,4-c]pyridin-5-yl}-2-{6-[1-(propan-2-yl)azetidin-3-yl]pyridazin-3-yl}phenol (Compound 57)

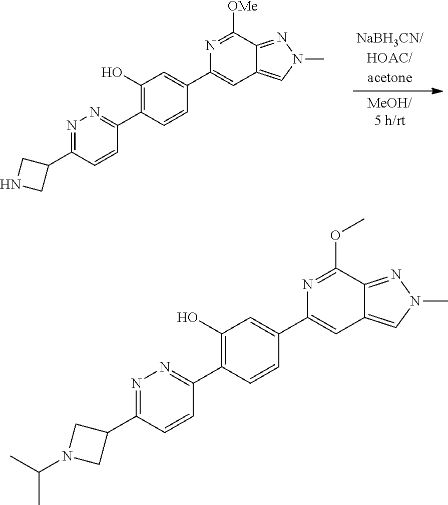

### Step 1: Preparation of 2-(6-(1-isopropylazetidin-3-yl)pyridazin-3-yl)-5-(7-methoxy-2-methyl-2H-pyrazolo[3,4-c]pyridin-5-yl)phenol

To a solution of 2-(6-(azetidin-3-yl)pyridazin-3-yl)-5-(7-methoxy-2-methyl-2H-pyrazolo[3,4-c]pyridin-5-yl)phenol (40 mg, 0.11 mmol) in MeOH (6 mL) and HOAc (40 mg) was added acetone (32 mg, 0.55 mmol). The resulting mixture was stirred for 1 h at rt. Then added NaBH₃CN (20.8 mg, 0.33 mmol) at 0° C. The resulting mixture was stirred for 5 h at rt. The mixture was evaporated to give crude title compound. The crude was purified by prep-HPLC (0.05% NH₃·H2O in water/ACN) to give title compound (3 mg, Y: 6.38%) as a yellow solid. ESI-MS (M+H)+: 431.2. ¹H NMR (400 MHz, CDCl₃) δ 13.49 (s, 1H), 8.09 (d, J=9.0 Hz, 1H), 7.91 (s, 1H), 7.86 (s, 1H), 7.79 (d, J=8.6 Hz, 1H), 7.72 (d, J=7.4 Hz, 1H), 7.64 (s, 2H), 4.28 (s, 3H), 4.25 (s, 3H), 4.21-4.10 (m, 3H), 3.89-3.72 (m, 2H), 3.11-2.69 (m, 1H), 1.19 (br.s, 6H).

### Example 58. 5-{7-methoxy-2-methyl-2H-pyrazolo[3,4-c]pyridin-5-yl}-2-{6-[1-(oxan-4-yl)azetidin-3-yl]pyridazin-3-yl}phenol (Compound 58)

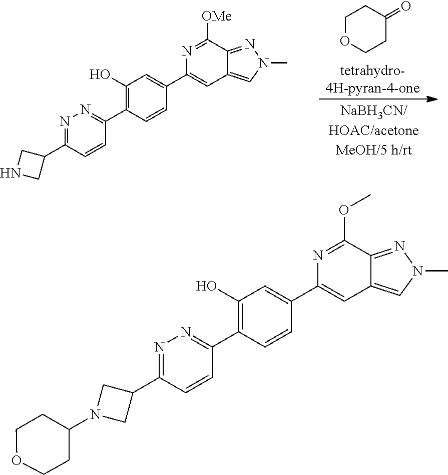 [SMILES: COc1nc(-c2ccc(-c3ccc(C4CNC4)nn3)c(O)c2)cc2cn(C)nc12 ; O=C1CCOCC1 ; COc1nc(-c2ccc(-c3ccc(C4CN(C5CCOCC5)C4)nn3)c(O)c2)cc2cn(C)nc12]

### Step 1: Preparation of 5-(7-methoxy-2-methyl-2H-pyrazolo [3,4-c]pyridin-5-yl)-2-(6-(I-(tetrahydro-2H-pyran-4-yl)azetidin-3-yl)pyridazin-3-yl)phenol

To a solution of 2-(6-(azetidin-3-yl)pyridazin-3-yl)-5-(7-methoxy-2-methyl-2H-pyrazolo[3,4-c]pyridin-5-yl)phenol (40 mg, 0.1 mmol) in MeOH (6 mL) and HOAc (2 drops) was added tetrahydro-4H-pyran-4-one (50 mg, 0.5 mmol). The resulting mixture was stirred for 1 h at RT. Then added NaBH₃CN (18.9 mg, 0.3 mmol) at 0° C. The resulting mixture was stirred for 5 h at RT. The mixture was evaporated to give crude title compound. The crude was purified by prep-HPLC (0.05% NH₃·H₂O in water/ACN) to give title compound (8 mg, Y: 16.9%) as a yellow solid. ESI-MS (M+H)+: 473.2. ¹H NMR (400 MHz, CDCl₃) δ 13.56 (s, 1H), 8.09 (d, J=8.9 Hz, 1H), 7.91 (s, 1H), 7.85 (s, 1H), 7.79 (d, J=5.9 Hz, 1H), 7.71 (d, J=8.7 Hz, 2H), 7.63 (s, 1H), 4.27 (s, 3H), 4.25 (s, 3H), 4.12-4.06 (m, 1H), 4.04-3.98 (m, 2H), 3.96-3.89 (m, 2H), 3.66-3.57 (m, 2H), 3.47-3.33 (m, 2H), 2.63-2.48 (m, 1H), 1.80-1.73 (m, 2H), 1.52-1.43 (m, 2H).

### Example 59. 5-{7-ethyl-2-methyl-2H-pyrazolo[4,3-b]pyridin-5-yl}-2-{6-[1-(oxan-4-yl)azetidin-3-yl]pyridazin-3-yl}phenol (Compound 59)

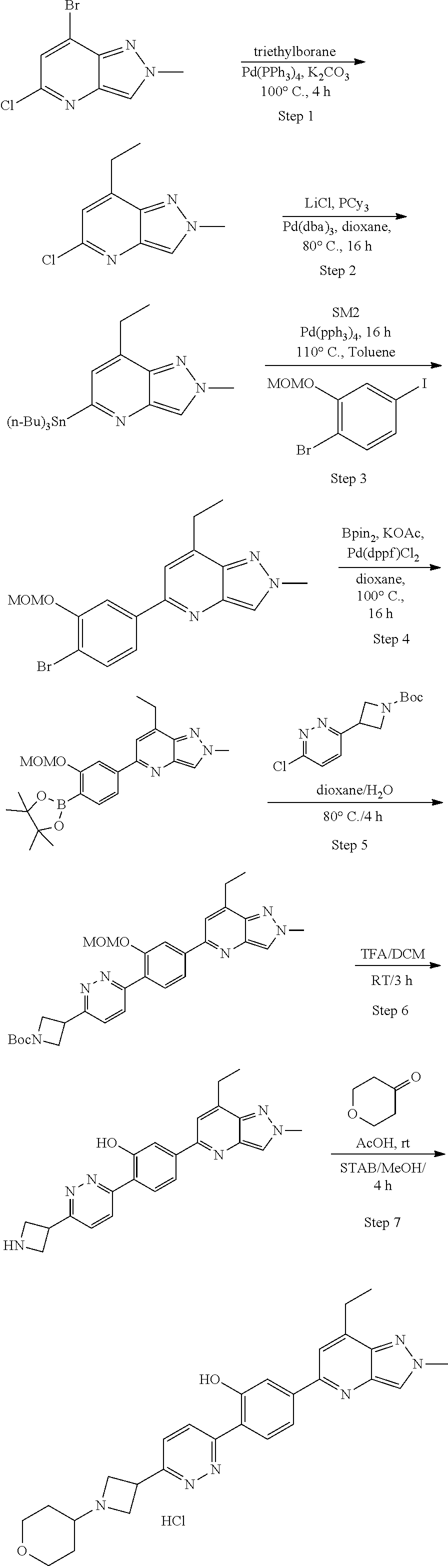

### Step 1: Preparation of 5-chloro-7-ethyl-2-methyl-2H-pyrazolo[4,3-b]pyridine

To a solution of 7-bromo-5-chloro-2-methyl-2H-pyrazolo[4,3-b]pyridine (5.0 g, 27.6 mmol) and trimethylboroxine (4.0 g, 40.8 mmol) in DMF (50 mL), were added K₂CO₃ (11.3 g, 81.6 mmol) Pd (PPh₃)₄ (1.5 g, 1.3 mmol). The mixture was stirred at 100° C. for 4 h under N₂ atmosphere. The mixture was diluted with H₂O (20 mL) and extracted with EA (30 mL×3). The organic layer was washed with brine, dried over Na₂SO₄ and evaporated. The crude was purified by reserve silica gel column chromatography to give title product (800 mg, yield 20.5%) as a yellow solid. ESI-MS (M+H)+: 196.1

### Step 2: Preparation of 2,7-dimethyl-5-(tributylstannyl)-2H-pyrazolo[4,3-b]pyridine

To a mixture of 5-chloro-7-ethyl-2-methyl-2H-pyrazolo[4,3-b]pyridine (800 mg, 4.1 mmol), Pd₂(dba)₃ (376.6 mg, 0.41 mmol), LiCl (861.5 g, 20.5 mmol), Pcy₃ (227.3 mg, 0.82 mmol) in dioxane (10 mL) were added hexabutylditin (7.1 g, 12.3 mmol), the reaction mixture was charged with N₂ for three times and stirred at 80° C. for 16 h. Then sat. KF (50 mL) was added and the mixture was stirred at rt for 1 h, extracted with EA (10 mL*3). The combined organic layers was concentrated in vacuo. The residue was purified by silica gel chromatography (PE:EA=8:1) to give title product (1.2 g, 64.9%) as a yellow oil. ESI-MS (M+H)+: 452.4

### Step 3: Preparation of 5-(4-bromo-3-(methoxymethoxy)phenyl)-7-ethyl-2-methyl-2H-pyrazolo[4,3-b]pyridine

To a mixture of 2,7-dimethyl-5-(tributylstannyl)-2H-pyrazolo[4,3-b]pyridine (1.2 g, 2.7 mmol) and 1-bromo-4-iodo-2-(methoxymethoxy) benzene (1.4 g, 4.0 mmol) in dioxane (10 mL) was added Pd(PPh₃)₄ (307.3 mg, 0.27 mmol). The mixture was stirred at 110° C. for 16 h. The mixture was filtered and the filtrate was concentrated in vacuo. The residue was purified by silica gel column chromatography (PE:EA=1:1) to give title product (520 mg, Y: 52%) as a yellow solid. ESI-MS (M+H+): 376.1

### Step 4: Preparation of 7-ethyl-5-(3-(methoxymethoxy)-4-(4,4,5,5-tetramethyl-1,3,2-dioxaborolan-2-yl)phenyl)-2-methyl-2H-pyrazolo[4,3-b]pyridine

To a mixture of 5-(4-bromo-3-(methoxymethoxy)phenyl)-7-ethyl-2-methyl-2H-pyrazolo[4,3-b]pyridine (520 mg, 1.39 mmol) and B₂pin₂ (1.8 g, 7.1 mmol) in dioxane (20 mL) were added Pd(dppf)Cl₂ (101.4 mg, 0.14 mmol) and KOAc (407.7 mg, 4.2 mmol). The mixture was stirred at 100° C. for 16 h. The mixture was filtered and the filtrate was concentrated in vacuo. The residue was purified by flash chromatography (PE:EA=1:1) to give title product (350 mg, Y: 60.3%) as a yellow solid. ESI-MS (M+H+): 424.3.

### Step 5: Preparation of tert-butyl 3-(6-(4-(7-ethyl-2-methyl-2H-pyrazolo[4,3-b]pyridin-5-yl)-2-(methoxymethoxy)phenyl)pyridazin-3-yl)azetidine-1-carboxylate

To a mixture of 7-ethyl-5-(3-(methoxymethoxy)-4-(4,4,5,5-tetramethyl-1,3,2-dioxaborolan-2-yl)phenyl)-2-methyl-2H-pyrazolo[4,3-b]pyridine (350 mg, 0.8 mmol) and tert-butyl 3-(6-chloropyridazin-3-yl) azetidine-1-carboxylate (333.9 mg, 1.24 mmol) in dioxane: H2O (20 mL:5 mL) were added Pd(dppf)Cl₂ (60.5 mg, 0.08 mmol) and K₂CO₃ (342.6 mg, 2.5 mmol). The mixture was stirred at 80° C. for 4 h. The mixture was filtered and the filtrate was concentrated in vacuo. The residue was purified by silica gel column chromatography (DCM:MeOH=40:1) to give title product (100 mg, Y: 23.6%) as a yellow solid. ESI-MS (M+H+): 531.4.

### Step 6: Preparation of 2-(6-(azetidin-3-yl)pyridazin-3-yl)-5-(7-ethyl-2-methyl-2H-pyrazolo[4,3-b]pyridin-5-yl)phenol

A mixture of tert-butyl 3-(6-(4-(7-ethyl-2-methyl-2H-pyrazolo[4,3-b]pyridin-5-yl)-2-(methoxymethoxy)phenyl)pyridazin-3-yl)azetidine-1-carboxylate (100 mg, 0.18 mmol) in TFA (5 mL) was stirred at rt for 3 h. The mixture was concentrated in vacuo to give title product (120 mg, crude) as a yellow solid. ESI-MS (M+H+): 387.1.

### Step 7: Preparation of 5-(7-ethyl-2-methyl-2H-pyrazolo[4,3-b]pyridin-5-yl)-2-(6-(1-(tetrahydro-2H-pyran-4-yl)azetidin-3-yl)pyridazin-3-yl)phenol hydrochloride

To a mixture of 2-(6-(azetidin-3-yl)pyridazin-3-yl)-5-(7-ethyl-2-methyl-2H-pyrazolo[4,3-b]pyridin-5-yl)phenol (65 mg, 0.17 mmol) and tetrahydro-4H-pyran-4-one (84.2 mg, 0.84 mmol) in MeOH (5 mL) and HOAc (30.3 mg, 0.5 mmoL) were added NaBH₃CN (53.1 mg, 0.84 mmol). The mixture was stirred at RT for 4 h. The mixture was filtered and the filtrate was concentrated in vacuo. The residue was purified by prep HPLC (0.05% HCl in water/CH₃CN) to give title product (25 mg, Y: 31.3%) as a yellow solid. ESI-MS (M+H+): 471.2. ¹H NMR (400 MHz, MeOD-d₄) δ 8.79 (s, 1H), 8.67-8.56 (m, 1H), 8.30 (dd, J=8.3, 4.5 Hz, 1H), 8.06 (s, 1H), 8.01-7.90 (m, 1H), 7.75-7.66 (m, 2H), 4.76-4.55 (m, 5H), 4.45 (s, 3H), 4.12-4.04 (m, 2H), 3.70-3.61 (m, 1H), 3.50-3.37 (m, 4H), 2.07-2.04 (m, 2H), 1.56-1.53 (m, 5H).

### Example 60. 5-{7-ethyl-2-methyl-2H-pyrazolo[4,3-b]pyridin-5-yl}-2-[6-(1-ethylazetidin-3-yl)pyridazin-3-yl]phenol (Compound 60)

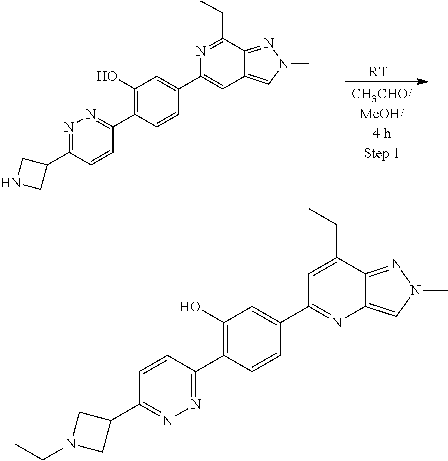

### Step 1: Preparation of 5-(7-ethyl-2-methyl-2H-pyrazolo[4,3-b]pyridin-5-yl)-2-(6-(1-ethylazetidin-3-yl)pyridazin-3-yl)phenol

To a mixture of 2-(6-(azetidin-3-yl)pyridazin-3-yl)-5-(7-ethyl-2-methyl-2H-pyrazolo[4,3-b]pyridin-5-yl)phenol (65 mg, 0.17 mmol) and acetaldehyde (38 mg, 0.85 mmol) in MeOH (5 mL) and HOAc (30.3 mg, 0.5 mmoL) was added NaBH₃CN (53.1 mg, 0.84 mmol). The mixture was stirred at RT for 4 h. The mixture was filtered and the filtrate was concentrated in vacuo. The residue was purified by prep-HPLC (0.05% NH₃·H₂O in water/CH₃CN) to give title product (10 mg, Y: 14.3%) as a yellow solid. ESI-MS (M+H+): 415.1. ¹H NMR (400 MHz, MeOD-d₄) δ 8.47-8.37 (m, 2H), 8.11-8.02 (m, 1H), 7.86 (dd, J=9.1, 1.8 Hz, 1H), 7.68-7.61 (m, 3H), 4.30 (s, 3H), 4.10-4.08 (m, 1H), 3.88-3.82 (m, 2H), 3.57-3.49 (m, 2H), 3.13 (q, J=7.5 Hz, 2H), 2.64 (q, J=7.2 Hz, 2H), 1.47 (t, J=7.6 Hz, 3H), 1.04 (t, J=7.2 Hz, 3H).

### Example 61. 5-{2,7-dimethyl-2H-pyrazolo[4,3-b]pyridin-5-yl}-2-{6-[1-(oxan-4-yl)azetidin-3-yl]pyridazin-3-yl}phenol (Compound 61)

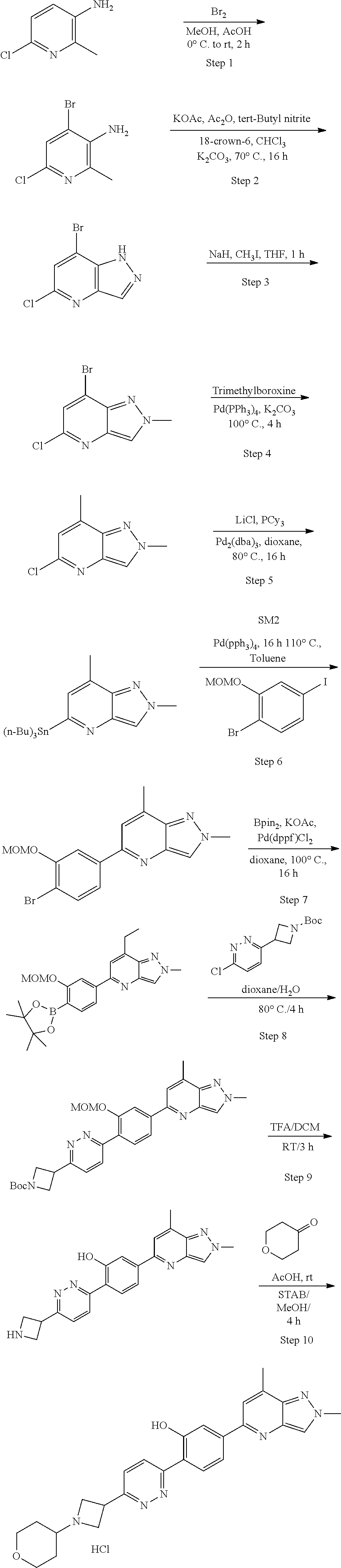

### Step 1: Preparation of 4-bromo-6-chloro-2-methylpyridin-3-amine

To a solution of 6-chloro-2-methylpyridin-3-amine (100 g, 704.2 mol) in MeOH (1000 mL) was added Br₂ (202.8 g, 1267.6 mol) in AcOH (100 mL) dropwised at RT, the mixture was stirred for 2 h at RT. The reaction mixture was diluted with EtOAc (500 mL) and stirred for 5 min. The solid was collected by filiation, washed with EtOAc (1000 mL×2) to afford title compound (140 g, 90.9%) as a yellow solid. ESI-MS (M+H)+: 223.0

### Step 2: Preparation of 7-bromo-5-chloro-1H-pyrazolo[4,3-b]pyridine

To a solution of 4-bromo-6-chloro-2-methylpyridin-3-amine (45.0 g*3, 204.5 mmol), KOAc (24.1 g, 245.5 mmol) in CHCl₃ (500 mL) was added Ac₂O (83.5 g, 818.2 mmol), the mixture was stirred for 1 h at RT. Then the reaction mixture was stirred at 70° C. for 2 h. tert-Butyl nitrite (21.1 g, 204.5 mmol) and 18-crown-6 (5.4 g, 20.5 mmol) was added, the reaction mixture was stirred at 70° C. for 16 h. After cooling to rt, sat. aqueous K₂CO₃ (500 mL) was added and the mixture was stirred at rt for 2 h. The mixture was exacted with EA (500 mL*3) and combined the organic layers was concentrated. The residue was purified by silica gel column chromatography (PE:EA=4:1) to give title product (35.0 g, 25.0%) as a yellow solid, ESI-MS (M+H)+: 234.1.

### Step 3: Preparation of 7-bromo-5-chloro-2-methyl-2H-pyrazolo[4,3-b]pyridine

To a solution of 4-7-bromo-5-chloro-1H-pyrazolo[4,3-b]pyridine (35.0 g, 151.5 mmol) in THF (350 mL) was added NaH (7.27 g, 303.1 mmol) at 0° C. under N₂ atmosphere. The mixture was stirred at rt for 30 min under N₂ atmosphere. To the mixture was added CH₃I (81.8 g, 575.8 mmol). The mixture was stirred at rt for 1 h. The mixture was diluted with H₂O (300 mL) and extracted with EA (500 mL×3). The organic layer was washed with brine, dried over Na₂SO₄ and evaporated to give crude. The crude was purified by silica gel column chromatography (PE:EA=5:1) to give title product (8.0 g, yield 22.2%) as a yellow solid. ESI-MS (M+H)+: 247.9. ¹H NMR (400 MHz, DMSO-d₆) δ 8.79 (d, J=1.7 Hz, 1H), 7.87-7.64 (m, 1H), 4.27 (s, 3H).

### Step 4: Preparation of 5-chloro-2,7-dimethyl-2H-pyrazolo[4,3-b]pyridine

To a solution of 7-bromo-5-chloro-2-methyl-2H-pyrazolo[4,3-b]pyridine (3.0 g, 12.2 mmol) and trimethylboroxine (2.3 g, 18.4 mmol) in DMF (50 mL), were added K₂CO₃ (707.1 mg, 0.61 mmol) and Pd(PPh₃)₄ (957.2 mg, 0.83 mmol). The mixture was stirred at 100° C. for 4 h under N₂ atmosphere. The mixture was diluted with H₂O (20 mL) and extracted with EA (30 mL*3). The organic layer was washed with brine, dried over Na₂SO₄ and evaporated to give crude. The crude was purified by reserve silica gel column chromatography to give title product (1.65 g, yield 75%) as a yellow solid. ESI-MS (M+H)+: 182.1

### Step 5: Preparation of 2,7-dimethyl-5-(tributylstannyl)-2H-pyrazolo[4,3-b]pyridine

To a mixture of 5-chloro-2,7-dimethyl-2H-pyrazolo[4,3-b]pyridine (1.65 g, 9.1 mmol), Pd₂(dba)₃ (836.9 mg, 0.91 mmol), LiCl (1.91 g, 45.6 mmol), Pcy₃ (505.1 mg, 1.8 mmol) in dioxane (20 mL) was added hexabutylditin (15.9 g, 27.3 mmol), the reaction mixture was charged with N₂ for three times and stirred at 80° C. for 16 h. Then sat·KF (50 mL) was added, the mixture was stirred at rt for 1 h and extracted with EA (20 mL*3). The combined organic layers was concentrated in vacuo. The residue was purified by silica gel chromatography (PE:EA=8:1) to give title product (2 g, 51.3%) as a yellow oil. ESI-MS (M+H)+: 438.0

### Step 6: Preparation of 5-(4-bromo-3-(methoxymethoxy)phenyl)-2,7-dimethyl-2H-pyrazolo[4,3-b]pyridine

To a mixture of 2,7-dimethyl-5-(tributylstannyl)-2H-pyrazolo[4,3-b]pyridine (2.0 g, 4.6 mmol) and 1-bromo-4-iodo-2-(methoxymethoxy)benzene (2.4 g, 6.9 mmol) in dioxane (20 mL) was added Pd(PPh₃)₄ (529.9 mg, 0.49 mmol). The mixture was stirred at 110° C. for 16 h. The mixture was filtered and the filtrate was concentrated in vacuo. The residue was purified by silica gel column chromatography (PE:EA=1:1) to give title product (1.3 g, Y: 81.25%) as a yellow solid. ESI-MS (M+H+): 362.2

### Step 7: Preparation of 6-(3-(methoxymethoxy)-4-(4,4,5,5-tetramethyl-1,3,2-dioxaborolan-2-yl) phenyl)-2,8-dimethylimidazo [1,2-b]pyridazine

To a mixture of 5-(4-bromo-3-(methoxymethoxy)phenyl)-2,7-dimethyl-2H-pyrazolo[4,3-b]pyridine (1.3 g, 3.6 mmol) and B₂pin₂ (6.4 g, 25.2 mmol) in dioxane (20 mL) were added Pd(dppf)Cl₂ (263.2 mg, 0.36 mmol) and KOAc (1.41 g, 14.4 mmol). The mixture was stirred at 100° C. for 16 h. The mixture was filtered and the filtrate was concentrated in vacuo. The residue was purified by flash chromatography (PE:EA=1:1) to give title product (500 mg, Y: 29.4%) as a yellow solid. ESI-MS (M+H+): 410.4

### Step 8: Preparation of tert-butyl 3-(6-(4-(2,7-dimethyl-2H-pyrazolo[4,3-b]pyridin-5-yl)-2-(methoxymethoxy)phenyl)pyridazin-3-yl)azetidine-1-carboxylate

To a mixture of 6-(3-(methoxymethoxy)-4-(4,4,5,5-tetramethyl-1,3,2-dioxaborolan-2-yl) phenyl)-2,8-dimethylimidazo [1,2-b]pyridazine (500 mg, 1.22 mmol) and tert-butyl 3-(6-chloropyridazin-3-yl) azetidine-1-carboxylate (493.3 mg, 1.83 mmol) in dioxane: H₂O (20 mL:5 mL) were added Pd(dppf)Cl₂ (89.4 mg, 0.12 mmol) and K₂CO₃ (506.1 mg, 3.67 mmol). The mixture was stirred at 80° C. for 4 h. The mixture was filtered and the filtrate was concentrated in vacuo. The residue was purified by silica gel column chromatography (DCM:MeOH=40:1) to give title product (400 mg, Y: 64.2%) as a yellow solid. ESI-MS (M+H+): 517.4

### Step 9: Preparation of 2-(6-(azetidin-3-yl)pyridazin-3-yl)-5-(2,7-dimethyl-2H-pyrazolo[4,3-b]pyridin-5-yl)phenol

A mixture of 3-(6-(4-(2,7-dimethyl-2H-pyrazolo[4,3-b]pyridin-5-yl)-2-(methoxymethoxy)phenyl)pyridazin-3-yl)azetidine-1-carboxylate (400 mg, 0.78 mmol) in TFA (10 mL) was stirred at rt for 3 h. The mixture was concentrated to give title product (400 mg crude) as a yellow solid. ESI-MS (M+H+): 373.2

### Step 10: Preparation of 5-(2,7-dimethyl-2H-pyrazolo[4,3-b]pyridin-5-yl)-2-(6-(1-(tetrahydro-2H-pyran-4-yl)azetidin-3-yl)pyridazin-3-yl)phenol hydrochloride

To a mixture of 2-(6-(azetidin-3-yl)pyridazin-3-yl)-5-(2,7-dimethyl-2H-pyrazolo[4,3-b]pyridin-5-yl)phenol (120 mg, 0.32 mmol) and tetrahydro-4H-pyran-4-one (161.3 mg, 1.61 mmol) in MeOH (10 mL) and HOAc (58 mg, 0.97 mmoL) was added NaBH₃CN (101.6 mg, 1.6 mmol). The mixture was stirred at RT for 5 h. The mixture was filtered and the filtrate was concentrated in vacuo. The residue was purified by prep-HPLC (0.05% HCl in water/CH₃CN) to give title product (21 mg, Y: 14.5%) as a yellow solid. ESI-MS (M+H+): 457.4. H NMR (400 MHz, DMSO-d₆) δ 8.68 (s, 1H), 8.58 (t, J=9.6 Hz, 1H), 8.17 (t, J=7.9 Hz, 1H), 8.00 (dd, J=38.5, 9.1 Hz, 1H), 7.84-7.80 (m, 2H), 7.77 (d, J=8.3 Hz, 1H), 4.62 (br s, 1H), 4.52-4.43 (m, 4H), 4.25 (s, 3H), 3.97-3.95 (m, 3H), 3.29 (t, J=11.3 Hz, 2H), 2.65 (s, 3H), 1.93 (d, J=11.8 Hz, 2H), 1.57-1.44 (m, 2H).

### Example 62. 5-{2,7-dimethyl-2H-pyrazolo[4,3-b]pyridin-5-yl}-2-[6-(1-ethylazetidin-3-yl)pyridazin-3-yl]phenol (Compound 62)

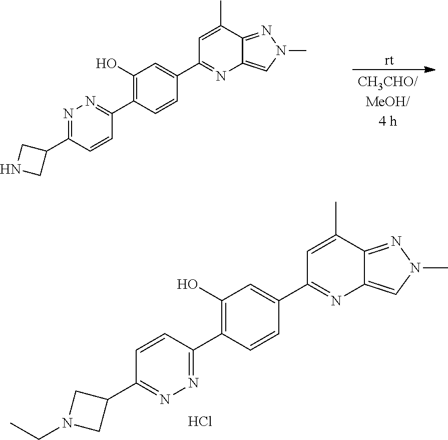

### Step 1: Preparation of 5-(2,7-dimethyl-2H-pyrazolo[4,3-b]pyridin-5-yl)-2-(6-(1-ethylazetidin-3-yl)pyridazin-3-yl)phenol hydrochloride

To a mixture of 2-(6-(azetidin-3-yl)pyridazin-3-yl)-5-(2,7-dimethyl-2H-pyrazolo[4,3-b]pyridin-5-yl)phenol (120 mg, 0.32 mmol) and CH₃CHO (70.8 mg, 1.61 mmol) in MeOH (10 mL) and HOAc (58 mg, 0.97 mmoL) was added NaBH₃CN (101.6 mg, 1.6 mmol). The mixture was stirred at RT for 4 h. The mixture was filtered and the filtrate was concentrated in vacuo. The residue was purified by prep HPLC (0.05% HCl in water/CH₃CN) to give title product (10 mg, Y: 7%) as a yellow solid. ESI-MS (M+H+): 401.2. ¹H NMR (400 MHz, MeOD-d₄) δ 8.82 (s, 1H), 8.62 (t, J=7.7 Hz, 1H), 8.25 (d, J=7.6 Hz, 1H), 8.11-7.99 (m, 2H), 7.77-7.66 (m, 2H), 4.89-4.72 (m, 2H), 4.60-4.47 (m, 3H), 4.44 (s, 3H), 3.51-3.41 (m, 2H), 2.97 (s, 3H), 1.36-1.26 (m, 3H).

### Example 63. 5-(2-methylimidazo[1,2-b]pyridazin-6-yl)-2-(6-(1-(tetrahydro-2H-pyran-4-yl)azetidin-3-yl)pyridazin-3-yl)phenol hydrochloride (Compound 63)

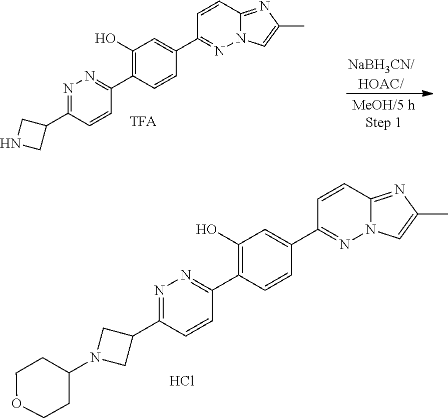 [SMILES: Cc1cn2nc(-c3ccc(-c4ccc(C5CNC5)nn4)c(O)c3)ccc2n1 ; Cc1cn2nc(-c3ccc(-c4ccc(C5CN(C6CCOCC6)C5)nn4)c(O)c3)ccc2n1 ; Cl]

### Step 1: Preparation of 5-(2-methylimidazo[1,2-b]pyridazin-6-yl)-2-(6-(1-(tetrahydro-2H-pyran-4-yl)azetidin-3-yl)pyridazin-3-yl)phenol hydrochloride

A mixture of 2-(6-(azetidin-3-yl)pyridazin-3-yl)-5-(2-methylimidazo[1,2-b]pyridazin-6-yl)phenol 2,2,2-trifluoroacetate (200 mg, 0.42 mmol) and tetrahydro-4H-pyran-4-onein (211.9 mg, 2.1 mmol) MeOH (10 mL) and HOAc (76.2 mg, 1.2 mmoL) were added NaBH₃CN (133.5 mg, 2.1 mmol). The mixture was stirred at RT for 5 h. LCMS showed the starting material was consumed completely. Filtered and concentrated in vacuo. The residue was purified by prep HPLC (0.05% HCl in water/CH₃CN) to give title product (33 mg, Y: 16.5%) as a yellow solid. ESI-MS (M+H+): 443.2. ¹H NMR (400 MHz, MeOD-d₄) δ 8.69 (t, J=8.5 Hz, 1H), 8.49-8.38 (m, 3H), 8.20-8.10 (m, 2H), 7.89-7.82 (m, 2H), 4.76-4.71 (m, 1H), 4.69-4.58 (m, 4H), 4.13-4.04 (m, 2H), 3.74-3.60 (m, 1H), 3.46 (t, J=11.9 Hz, 2H), 2.66 (s, 3H), 2.08-2.04 (m, 2H), 1.66-1.52 (m, 2H).

### Example 64. 5-(2,8-dimethylimidazo[1,2-b]pyridazin-6-yl)-2-(6-(1-(tetrahydro-2H-pyran-4-yl)azetidin-3-yl)pyridazin-3-yl)phenol hydrochloride (Compound 64)

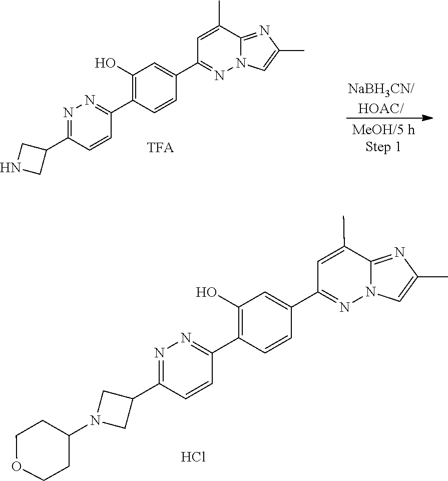 [SMILES: Cc1cn2nc(-c3ccc(-c4ccc(C5CNC5)nn4)c(O)c3)cc(C)c2n1 ; Cc1cn2nc(-c3ccc(-c4ccc(C5CN(C6CCOCC6)C5)nn4)c(O)c3)cc(C)c2n1 ; Cl]

### Step 1: Preparation of 5-(2,8-dimethylimidazo[1,2-b]pyridazin-6-yl)-2-(6-(1-(tetrahydro-2H-pyran-4-yl)azetidin-3-yl)pyridazin-3-yl)phenol hydrochloride

A mixture of 2-(6-(azetidin-3-yl)pyridazin-3-yl)-5-(2,8-dimethylimidazo[1,2-b]pyridazin-6-yl)phenol 2,2,2-trifluoroacetate (200 mg, 0.41 mmol) and tetrahydro-4H-pyran-4-onein (206 mg, 2.06 mmol) MeOH (10 mL) and HOAc (74.1 mg, 1.2 mmoL) were added NaBH₃CN (129.6 mg, 2.06 mmol). The mixture was stirred at RT for 5 h. LCMS showed the starting material was consumed completely. Filtered and concentrated in vacuo. The residue was purified by prep HPLC (0.05% HCl in water/CH₃CN) to give title product (45 mg, Y: 22.4%) as a yellow solid. ESI-MS (M+H+): 457.2. ¹H NMR (400 MHz, MeOD-d₄) δ 8.69 (t, J=8.6 Hz, 1H), 8.34 (s, 1H), 8.29 (s, 1H), 8.18-8.09 (m, 2H), 7.87-7.79 (m, 2H), 4.77-4.70 (m, 1H), 4.69-4.56 (m, 4H), 4.13-4.02 (m, 2H), 3.71-3.59 (m, 1H), 3.46 (t, J=11.9 Hz, 2H), 2.81 (s, 3H), 2.67 (s, 3H), 2.09-2.03 (m, 2H), 1.66-1.54 (m, 2H).

### Example 65. 5-(7-methoxy-2-methyl-2H-pyrazolo[4,3-b]pyridin-5-yl)-2-(6-(1-(tetrahydro-2H-pyran-4-yl)azetidin-3-yl)pyridazin-3-yl)phenol hydrochloride (Compound 65)

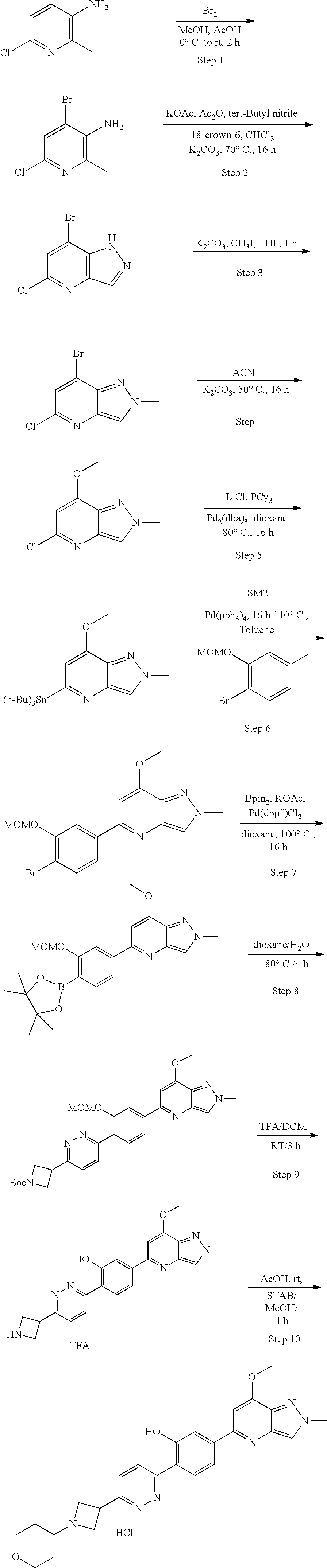

### Step 1: Preparation of 4-bromo-6-chloro-2-methylpyridin-3-amine

To a solution of 6-chloro-2-methylpyridin-3-amine (100 g, 704.2 mmol) in MeOH (1000 mL) was added Br₂ (202.8 g, 1.26 mol) in AcOH (100 mL) dropwised at RT, the mixture was stirred for 2 h at RT. The reaction mixture was diluted with EtOAc (500 mL) and stirred for 5 min. The solid was collected by filiation, washed with EtOAc (1000 mL×2) to afford title compound (140 g, 90.9% yield) as a yellow solid. ESI-MS (M+H)+: 223.0.

### Step 2: Preparation of 7-bromo-5-chloro-1H-pyrazolo[4,3-b]pyridine

To a solution of 4-bromo-6-chloro-2-methylpyridin-3-amine (45.0 g, 204.5 mmol), KOAc (24.1 g, 245.5 mmol) in CHCl₃ (500 mL) was added Ac₂O (83.5 g, 818.2 mmol), the mixture was stirred for 1 h at RT. Then the reaction mixture was stirred at 70° C. for 2 h. Then tert-Butyl nitrite (21.1 g, 204.5 mmol) and 18-crown-6 (5.4 g, 20.5 mmol) was added, the reaction mixture was stirred at 70° C. for 16 h. Then cooled down and sat·K₂CO₃ (500 mL) was added to it and stirred at rt for 2 h. Then extracted with EA (500 mL*3) and combined the organic layers and concentrated. The residue was purified by silica gel column chromatography (PE:EA=4:1) to give title product (35.0 g, 25.0%) as a yellow solid, ESI-MS (M+H)+: 234.1.

### Step 3: Preparation of 7-bromo-5-chloro-2-methyl-2H-pyrazolo[4,3-b]pyridine

To a solution of 4-7-bromo-5-chloro-1H-pyrazolo[4,3-b]pyridine (35.0 g, 151.5 mmol) in THF (350 mL) was added NaH (12.1 g, 303.1 mmol) at 0° C. under N₂ atmosphere. The mixture was stirred at rt for 30 min under N₂ atmosphere. The mixture was added CH₃I (81.8 g, 575.8 mmol) under N₂ atmosphere. The mixture was stirred at rt for 1 h under N₂ atmosphere. The mixture was diluted with H₂O (300 mL) and extracted with EA (500 mL*3). The organic layer was washed with brine, dried over Na₂SO₄ and evaporated to give crude. The crude was purified by silica gel column chromatography (PE:EA=5:1) to give title product (8.0 g, yield 22.2%) as a yellow solid. ESI-MS (M+H)+: 247.9. ¹H NMR (400 MHz, DMSO-d₆) δ 8.79 (d, J=1.7 Hz, 1H), 7.87-7.64 (m, 1H), 4.27 (s, 3H).

### Step 4: Preparation of 5-chloro-7-methoxy-2-methyl-2H-pyrazolo[4,3-b]pyridine

To a solution of 7-bromo-5-chloro-2-methyl-2H-pyrazolo[4,3-b]pyridine (4.3 g, 17.6 mmol) and K₂CO₃ (12.1 g, 87.7 mmol) in ACN (100 mL) and MeOH (100 mL). The mixture was stirred at 50° C. for 16 h under N₂ atmosphere. The mixture was diluted with H₂O (80 mL) and extracted with EA (150 mL*3). The organic layer was washed with brine, dried over Na₂SO₄ and evaporated to give crude. The crude was purified by reserve silica gel column chromatography (PE EA=4:1) to give title product (3.2 g, yield 92.8%) as a white solid. ESI-MS (M+H)+: 198.0. ¹H NMR (400 MHz, CDCl₃) δ 8.00 (s, 1H), 6.55 (s, 1H), 4.22 (s, 3H), 4.07 (s, 3H).

### Step 5: Preparation of 7-methoxy-2-methyl-5-(tributylstannyl)-2H-pyrazolo[4,3-b]pyridine

To a mixture of 5-chloro-7-methoxy-2-methyl-2H-pyrazolo[4,3-b]pyridine (3.2 g, 16.2 mmol), Pd₂(dba)₃ (1.49 g, 1.62 mmol), LiCl (3.41 g, 81.2 mmol), Pcy₃ (0.90 mg, 0.3.2 mmol) in dioxane (100 mL) were added hexabutylditin (28.3 g, 48.7 mmol), the reaction mixture was charged with N₂ for three times and stirred at 80° C. for 16 h. Then sat·KF (100 mL) was added to it and stirred at rt for 1 h, extracted with EA (100 mL*3), combined the organic layers and concentrated in vacuo. The residue was purified by silica gel chromatography (PE:EA=1:1) to give the production (4 g, 57.1%) as a yellow oil. ESI-MS (M+H)+: 454.2.

### Step 6: Preparation of 5-(4-bromo-3-(methoxymethoxy)phenyl)-7-methoxy-2-methyl-2H-pyrazolo[4,3-b]pyridine

A mixture of 7-methoxy-2-methyl-5-(tributylstannyl)-2H-pyrazolo[4,3-b]pyridine (4.0 g, 9.2 mmol) and 1-bromo-4-iodo-2-(methoxymethoxy) benzene (4.7 g, 13.8 mmol) in dioxane (50 mL) were added Pd (PPh₃)₄ (1.06 g, 0.92 mmol). The mixture was stirred at 110° C. for 16 h. LCMS showed the starting material was consumed completely. Filtered and concentrate in vacuo. The residue was purified by silica gel column chromatography (PE:EA=1:1) to give title product (1.3 g, Y: 38.2%) as a yellow solid. ESI-MS (M+H+): 380.2

### Step 7: Preparation of 7-methoxy-5-(3-(methoxymethoxy)-4-(4,4,5,5-tetramethyl-1,3,2-dioxaborolan-2-yl)phenyl)-2-methyl-2H-pyrazolo[4,3-b]pyridine

A mixture of 5-(4-bromo-3-(methoxymethoxy)phenyl)-7-methoxy-2-methyl-2H-pyrazolo[4,3-b]pyridine (1.3 g, 3.4 mmol) and B₂pin₂ (6.1 g, 25.2 mmol) in dioxane (40 mL) were added Pd (dppf) C₁₂ (252 mg, 0.34 mmol) and KOAc (1.69 g, 17.2 mmol). The mixture was stirred at 100° C. for 16 h. LCMS showed the starting material was consumed completely. Filtered and concentrate in vacuo. The residue was purified by flash chromatography (PE:EA=1:1) to give title product (900 mg, Y: 62.3%) as a yellow solid. ESI-MS (M+H+): 426.3

### Step 8: Preparation of tert-butyl 3-(6-(4-(7-methoxy-2-methyl-2H-pyrazolo[4,3-b]pyridin-5-yl)-2-(methoxymethoxy)phenyl)pyridazin-3-yl)azetidine-1-carboxylate

A mixture of 7-methoxy-5-(3-(methoxymethoxy)-4-(4,4,5,5-tetramethyl-1,3,2-dioxaborolan-2-yl)phenyl)-2-methyl-2H-pyrazolo[4,3-b]pyridine (900 mg, 2.1 mmol) and tert-butyl 3-(6-chloropyridazin-3-yl) azetidine-1-carboxylate (854.5 mg, 3.18 mmol) in dioxane: H2O (20 mL:5 mL) were added Pd (dppf) C₁₂ (154.8 mg, 0.21 mmol) and K₂CO₃ (876.7 mg, 6.4 mmol). The mixture was stirred at 80° C. for 4 h. LCMS showed the starting material was consumed completely. Filtered and concentrate in vacuo. The residue was purified by silica gel column chromatography (DCM:MeOH=40:1) to give title product (700 mg, Y: 60.8%) as a yellow solid. ESI-MS (M+H+): 533.4

### Step 9: Preparation of 2-(6-(azetidin-3-yl)pyridazin-3-yl)-5-(7-methoxy-2-methyl-2H-pyrazolo[4,3-b]pyridin-5-yl)phenol TFA salt

A mixture of tert-butyl 3-(6-(4-(7-methoxy-2-methyl-2H-pyrazolo[4,3-b]pyridin-5-yl)-2-(methoxymethoxy)phenyl)pyridazin-3-yl)azetidine-1-carboxylate (700 mg, 1.32 mmol) in TFA (10 mL). The mixture was stirred at rt for 3 h. LCMS showed the starting material was consumed completely. The mixture was concentrated in vacuo and freeze-drying to give title product (600 mg, Y: 90.2%) as a yellow solid. ESI-MS (M+H+): 389.3. ¹H NMR (400 MHz, DMSO-d₆) δ 8.72 (s, 1H), 8.60 (d, J=9.1 Hz, 1H), 8.23 (d, J=8.4 Hz, 1H), 7.94 (d, J=9.1 Hz, 1H), 7.83-7.75 (m, 2H), 7.44 (s, 1H), 4.50-4.44 (m, 1H), 4.43-4.36 (m, 4H), 4.30-4.24 (m, 6H).

### Step 10: Preparation of 5-(7-methoxy-2-methyl-2H-pyrazolo[4,3-b]pyridin-5-yl)-2-(6-(1-(tetrahydro-2H-pyran-4-yl)azetidin-3-yl)pyridazin-3-yl)phenol hydrochloride

A mixture of 2-(6-(azetidin-3-yl)pyridazin-3-yl)-5-(7-methoxy-2-methyl-2H-pyrazolo[4,3-b]pyridin-5-yl)phenol TFA salt (120 mg, 0.24 mmol) and tetrahydro-4H-pyran-4-onein (120 mg, 1.2 mmol) MeOH (10 mL) and HOAc (43 mg, 0.72 mmoL) were added NaBH₃CN (75.3 mg, 1.2 mmol). The mixture was stirred at RT for 5 h. LCMS showed the starting material was consumed completely. Filtered and concentrate in vacuo. The residue was purified by prep HPLC (0.05% HCl in water/CH₃CN) to give the solid and Freeze-drying to give title product (21 mg, Y: 14.5%) as a yellow solid. ESI-MS (M+H+): 473.2. ¹H NMR (400 MHz, MeOD-d₄) δ 8.72 (s, 1H), 8.67 (t, J=8.8 Hz, 1H), 8.31-8.23 (m, 1H), 8.13-8.05 (m, 1H), 7.72-7.64 (m, 2H), 7.55 (s, 1H), 4.74-4.63 (m, 4H), 4.49 (s, 3H), 4.39 (s, 3H), 4.12-4.06 (m, 2H), 3.77 (dd, J=10.0, 4.6 Hz, 1H), 3.70-3.64 (m, 1H), 3.45 (t, J=11.9 Hz, 2H), 2.09-2.04 (m, 2H), 1.64-1.55 (m, 2H).

### Example 66. 2-(6-(1-ethylazetidin-3-yl)pyridazin-3-yl)-5-(7-methoxy-2-methyl-2H-pyrazolo[4,3-b]pyridin-5-yl)phenol hydrochloride (Compound 66)

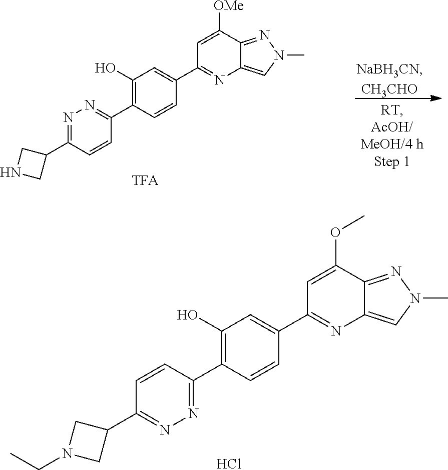

### Step 1: Preparation of 2-(6-(I-ethylazetidin-3-yl)pyridazin-3-yl)-5-(7-methoxy-2-methyl-2H-pyrazolo[4,3-b]pyridin-5-yl)phenol hydrochloride

A mixture of 2-(6-(azetidin-3-yl)pyridazin-3-yl)-5-(7-methoxy-2-methyl-2H-pyrazolo[4,3-b]pyridin-5-yl)phenol TFA (120 mg, 0.24 mmol) and CH₃CHO (52.6 mg, 1.2 mmol) in MeOH (10 mL) and HOAc (43 mg, 0.72 mmoL) were added NaBH₃CN (75.3 mg, 1.2 mmol). The mixture was stirred at RT for 5 h. LCMS showed the starting material was consumed completely. Filtered and concentrate in vacuo. The residue was purified by prep HPLC (0.05% HCl in water/CH₃CN) to give title product (45 mg, Y: 41.6%) as a yellow solid. ESI-MS (M+H+): 417.1. ¹H NMR (400 MHz, MeOD-d₄) δ 8.71 (s, 1H), 8.68-8.62 (m, 1H), 8.29-8.22 (m, 1H), 8.10-8.00 (m, 1H), 7.73-7.64 (m, 2H), 7.54 (s, 1H), 4.76-4.67 (m, 2H), 4.59-4.52 (m, 2H), 4.49 (s, 3H), 4.48-4.41 (m, 1H), 4.39 (s, 3H), 3.44 (q, J=7.2 Hz, 2H), 1.34-1.27 (m, 3H).

### Example 67. 5-(2-methylpyrazolo[1,5-a]pyridin-5-yl)-2-(6-(1-(tetrahydro-2H-pyran-4-yl)azetidin-3-yl)pyridazin-3-yl)phenol 2,2,2-trifluoroacetate (Compound 192)

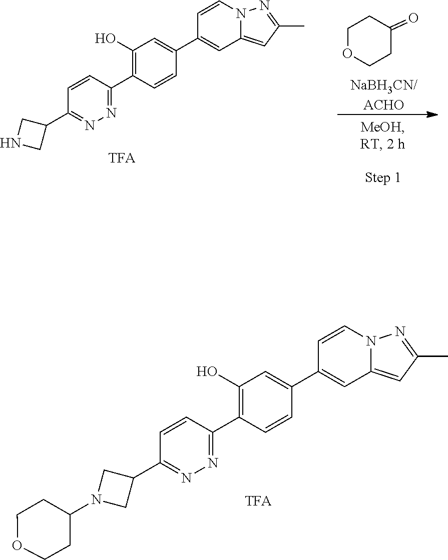 [SMILES: Cc1cc2cc(-c3ccc(-c4ccc(C5CNC5)nn4)c(O)c3)ccn2n1 ; O=C1CCOCC1 ; Cc1cc2cc(-c3ccc(-c4ccc(C5CN(C6CCOCC6)C5)nn4)c(O)c3)ccn2n1 ; "TFA"]

Step 1: Preparation of 5-(2-methylpyrazolo[1,5-a]pyridin-5-yl)-2-(6-(1-(tetrahydro-2H-pyran-4-yl)azetidin-3-yl)pyridazin-3-yl)phenol 2,2,2-trifluoroacetate. To a mixture of 2-(6-(azetidin-3-yl)pyridazin-3-yl)-5-(2-methylpyrazolo[1,5-a]pyridin-5-yl)phenol 2,2,2-trifluoroacetate (150 mg, 0.32 mmol) and tetrahydro-4H-pyran-4-one (210 mg, 2.1 mmoL) in MeOH (5 mL) were added NaBH₃CN (132 mg, 2.1 mmol) and AcOH (75 mg, 1.26 mmoL). The mixture was stirred at RT for 2 h. Filtered and the filtrate was concentrated in vacuo. The residue was purified by prep HPLC (0.05% TFA in water/CH₃CN) to give title product (45 mg, Y: 25.3%) as a yellow solid. ESI-MS (M+H+): 442.1. ¹H NMR (400 MHz, MeOD-d₄) δ 8.48-8.42 (m, 2H), 8.05 (d, J=8.8 Hz, 1H), 7.86 (s, 1H), 7.79 (d, J=9.0 Hz, 1H), 7.42-7.37 (m, 2H), 7.16 (dd, J=7.3, 1.9 Hz, 1H), 6.47 (s, 1H), 4.69-4.60 (m, 4H), 4.53-4.27 (m, 1H), 4.11-4.05 (m, 2H), 3.67-3.59 (m, 1H), 3.46 (t, J=11.3 Hz, 2H), 2.47 (s, 3H), 2.10-2.02 (m, 2H), 1.60-1.48 (m, 2H).

### General Procedure for Parallel Synthesis:

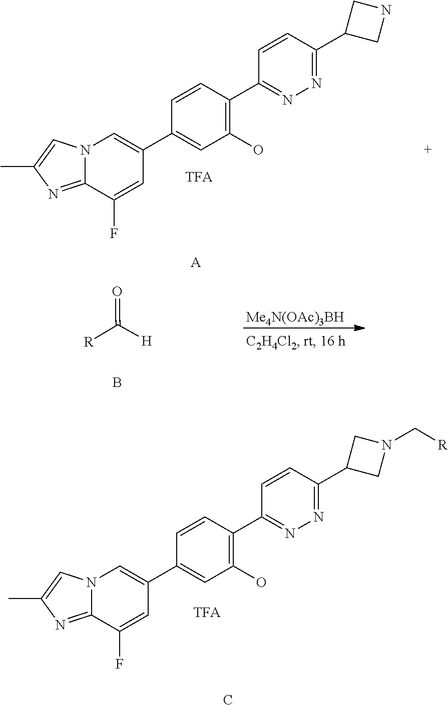

To a mixture of 1 equivalent of amine (A) and 1.3 equivalent aldehyde (B) in 1 mL dichloroethane was added 1.65 equivalents of DIPEA and 6 equivalents of tetramethylammonium triacetoxyborohydride. The reaction mixture was stirred at room temperature for 16 hours. To the mixture was added 0.1 mL water and the solvent was evaporated under high vacuum. The residue was dissolved in DMSO and the resulting solution was purified by HPLC (Deionized Water (phase A) and HPLC-grade Acetonitrile (phase B) were used as an eluent) to obtain final compound (C). In most cases TFA was used as an additive to improve the separation of the products.

Instrument: Agilent 1260 Infinity systems equipped with DAD and mass-detector

Column: Waters Sunfire C18 OBD Prep Column, 100 A, 5 μm, 19 mm×100 mm with SunFire C18 Prep Guard Cartridge, 100 A, 10 μm, 19 mm×10 mm

Compounds 70-128 were prepared as described above

| Compound No. | ESI-MS (M + H)⁺ |
|---|---|
| 70 | 463 |
| 71 | 471.2 |
| 72 | 456.2 |
| 73 | 470.2 |
| 74 | 484.2 |
| 75 | 473.2 |
| 76 | 462 |
| 77 | 484.2 |
| 78 | 463 |
| 79 | 471.2 |
| 80 | 470.1 |
| 81 | 484.2 |
| 82 | 469.2 |
| 83 | 472.2 |
| 84 | 449.2 |
| 85 | 475 |
| 86 | 489 |
| 87 | 499 |
| 88 | 474.2 |
| 89 | 446.1 |
| 90 | 457.2 |
| 91 | 498 |
| 92 | 462 |
| 93 | 475.2 |
| 94 | 469 |
| 95 | 475 |
| 96 | 485 |
| 97 | 476.2 |
| 98 | 455 |
| 99 | 474.2 |
| 100 | 470.1 |
| 101 | 458.2 |
| 102 | 502 |
| 103 | 460.2 |
| 104 | 463.2 |
| 105 | 444.2 |
| 106 | 498.2 |
| 107 | 498.2 |
| 108 | 448 |
| 109 | 490.2 |
| 110 | 461.2 |
| 111 | 476.2 |
| 112 | 480 |
| 113 | 500.2 |
| 114 | 471.2 |
| 115 | 496.2 |
| 116 | 499.2 |
| 117 | 471 |
| 118 | 474.2 |
| 119 | 460.2 |
| 120 | 485.2 |
| 121 | 483.2 |
| 122 | 488.4 |
| 123 | 485.2 |
| 124 | 500.2 |
| 125 | 484.2 |
| 126 | 500.2 |
| 127 | 500.2 |
| 128 | 496.2 |

### General Procedure for Parallel Synthesis:

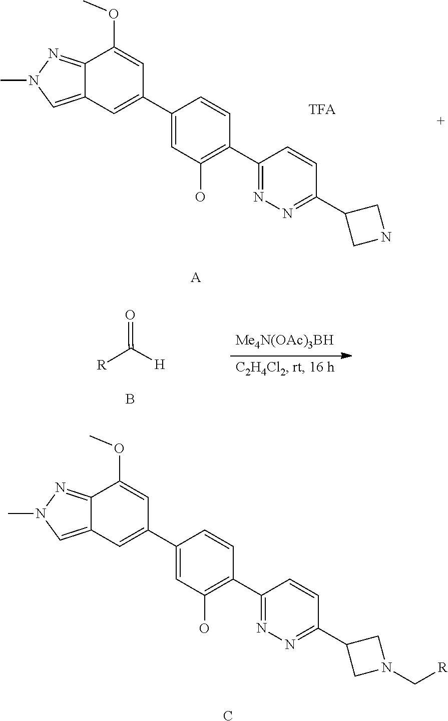

To a mixture of 1 equivalent of amine (A) and 1.3 equivalent aldehyde (B) in 1 mL dichloroethane was added 1.65 equivalents of DIPEA and 6 equivalents of tetramethylammonium triacetoxyborohydride. The reaction mixture was stirred at room temperature for 16 hours. To the mixture was added 0.1 mL water and the solvent was evaporated under high vacuum. The residue was dissolved in DMSO and the resulting solution was purified by HPLC (Deionized Water (phase A) and HPLC-grade Acetonitrile (phase B) were used as an eluent) to obtain final compound (C). In most cases TFA was used as an additive to improve the separation of the products.

Instrument: Agilent 1260 Infinity systems equipped with DAD and mass-detector

Column: Waters Sunfire C18 OBD Prep Column, 100 A, 5 μm, 19 mm×100 mm with SunFire C18 Prep Guard Cartridge, 100 A, 10 μm, 19 mm×10 mm

Compounds 129-191 were prepared as described above.

| Compound No. | ESI-MS (M + H)⁺ |
|---|---|
| 129 | 479 |
| 130 | 479.2 |
| 131 | 482 |
| 132 | 500.2 |
| 133 | 510 |
| 134 | 511.2 |
| 135 | 470 |
| 136 | 458.2 |
| 137 | 456.2 |
| 138 | 493.2 |
| 139 | 497.2 |
| 140 | 486.2 |
| 141 | 485.2 |
| 142 | 488.2 |
| 143 | 460.2 |
| 144 | 500.2 |
| 145 | 486.2 |
| 146 | 500.1 |
| 147 | 486.2 |
| 148 | 488.2 |
| 149 | 509.2 |
| 150 | 493.2 |
| 151 | 486.2 |
| 152 | 495.2 |
| 153 | 509.2 |
| 154 | 500.2 |
| 155 | 486.2 |
| 156 | 500.2 |
| 157 | 470 |
| 158 | 500.2 |
| 159 | 495 |
| 160 | 472.2 |
| 161 | 508.2 |
| 162 | 483.2 |
| 163 | 486.2 |
| 164 | 495 |
| 165 | 492 |
| 166 | 460.2 |
| 167 | 502.2 |
| 168 | 500.2 |
| 169 | 483.2 |
| 170 | 483.2 |
| 171 | 512.2 |
| 172 | 512.2 |
| 173 | 508.2 |
| 174 | 488.2 |
| 175 | 508.2 |
| 176 | 489 |
| 177 | 486.2 |
| 178 | 488.2 |
| 179 | 514.2 |
| 180 | 494.2 |
| 181 | 508.2 |
| 182 | 485.2 |
| 183 | 502 |
| 184 | 508.2 |
| 185 | 482.2 |
| 186 | 496.2 |
| 187 | 500.2 |
| 188 | 512.2 |
| 189 | 510 |
| 190 | 496.2 |
| 191 | 510.2 |

TABLE 1
List of Compounds

| | |
|---|---|
| 1 | 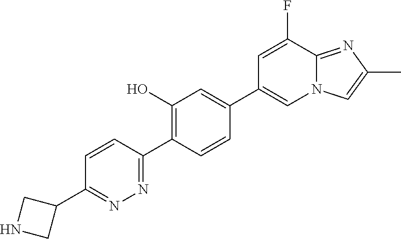 [SMILES: Cc1cn2cc(-c3ccc(-c4ccc(C5CNC5)nn4)c(O)c3)cc(F)c2n1] |
| 2 | 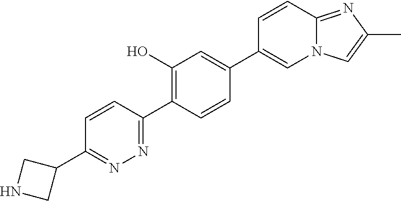 [SMILES: Cc1cn2cc(-c3ccc(-c4ccc(C5CNC5)nn4)c(O)c3)ccc2n1] |
| 3 | 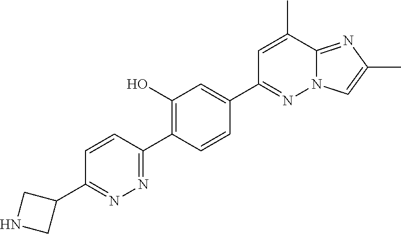 [SMILES: Cc1cn2nc(-c3ccc(-c4ccc(C5CNC5)nn4)c(O)c3)cc(C)c2n1] |
| 4 | 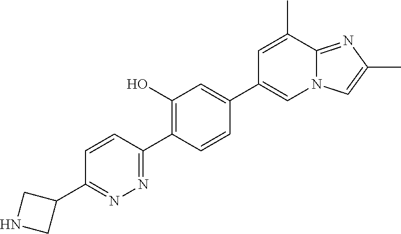 [SMILES: Cc1cn2cc(-c3ccc(-c4ccc(C5CNC5)nn4)c(O)c3)cc(C)c2n1] |
| 5 | 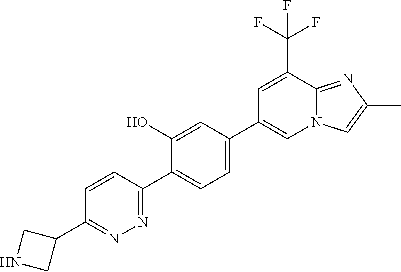 [SMILES: Cc1cn2cc(-c3ccc(-c4ccc(C5CNC5)nn4)c(O)c3)cc(C(F)(F)F)c2n1] |
| 6 | 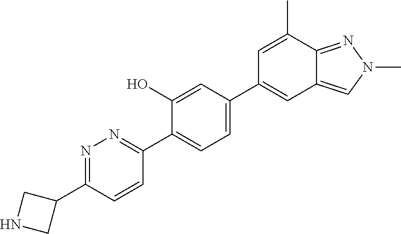 [SMILES: Cc1cc(-c2ccc(-c3ccc(C4CNC4)nn3)c(O)c2)cc2cn(C)nc12] |
| 7 | 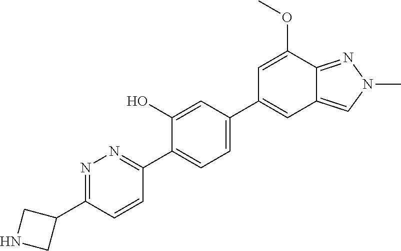 [SMILES: COc1cc(-c2ccc(-c3ccc(C4CNC4)nn3)c(O)c2)cc2cn(C)nc12] |
| 8 | 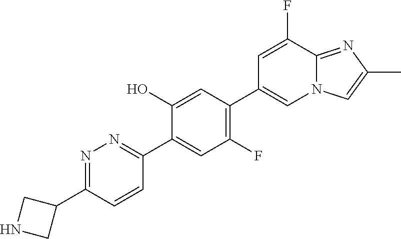 [SMILES: Cc1cn2cc(-c3cc(O)c(-c4ccc(C5CNC5)nn4)cc3F)cc(F)c2n1] |
| 9 | 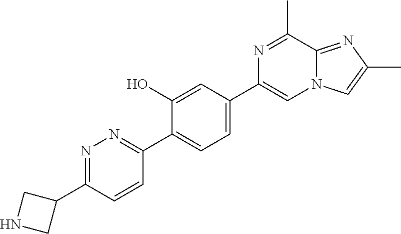 [SMILES: Cc1cn2cc(-c3ccc(-c4ccc(C5CNC5)nn4)c(O)c3)nc(C)c2n1] |
| 10 | 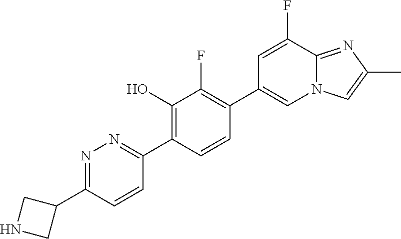 [SMILES: Cc1cn2cc(-c3ccc(-c4ccc(C5CNC5)nn4)c(O)c3F)cc(F)c2n1] |
| 11 | 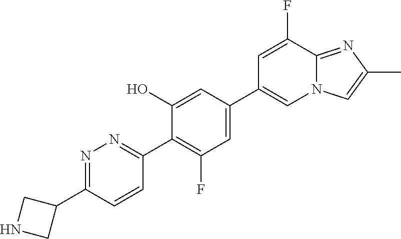 [SMILES: Cc1cn2cc(-c3cc(O)c(-c4ccc(C5CNC5)nn4)c(F)c3)cc(F)c2n1] |
| 12 | 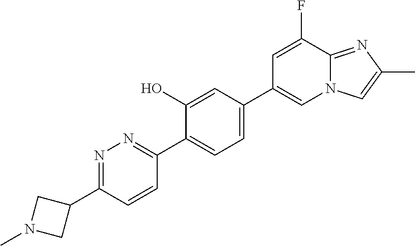 |
| 13 | 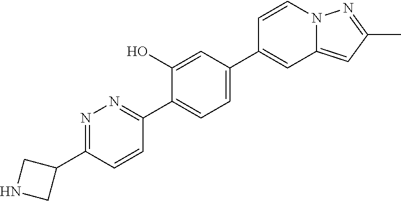 [SMILES: Cc1cc2cc(-c3ccc(-c4ccc(C5CNC5)nn4)c(O)c3)ccn2n1] |
| 14 | 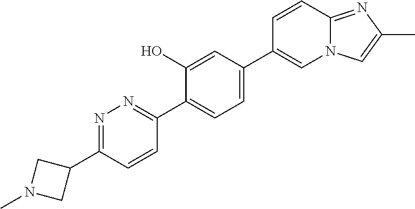 |
| 15 | 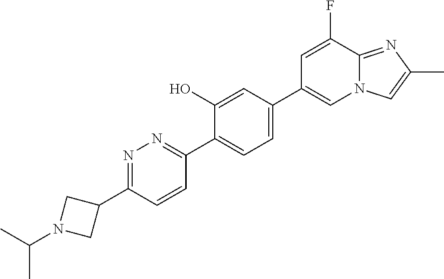 [SMILES: Cc1cn2cc(-c3ccc(-c4ccc(C5CN(C(C)C)C5)nn4)c(O)c3)cc(F)c2n1] |
| 16 | 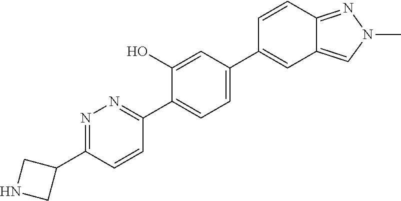 [SMILES: Cn1cc2cc(-c3ccc(-c4ccc(C5CNC5)nn4)c(O)c3)ccc2n1] |
| 17 | 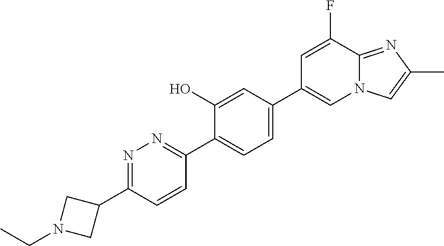 [SMILES: CCN1CC(c2ccc(-c3ccc(-c4cc(F)c5nc(C)cn5c4)cc3O)nn2)C1] |
| 18 | 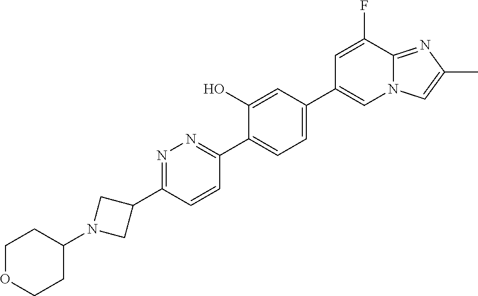 [SMILES: Cc1cn2cc(-c3ccc(-c4ccc(C5CN(C6CCOCC6)C5)nn4)c(O)c3)cc(F)c2n1] |
| 19 | 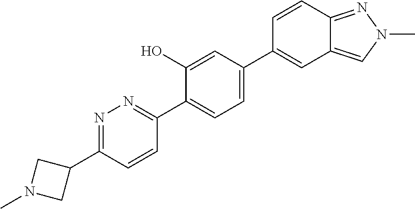 |
| 20 | 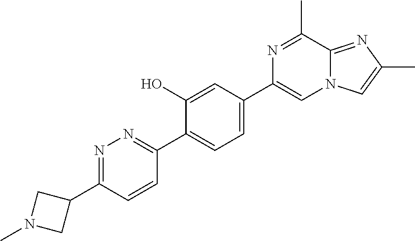 |
| 21 | 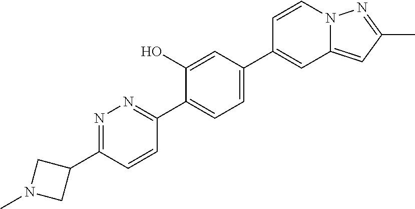 |
| 22 | 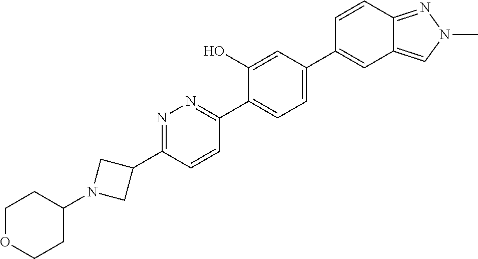 [SMILES: Cn1cc2cc(-c3ccc(-c4ccc(C5CN(C6CCOCC6)C5)nn4)c(O)c3)ccc2n1] |
| 23 | 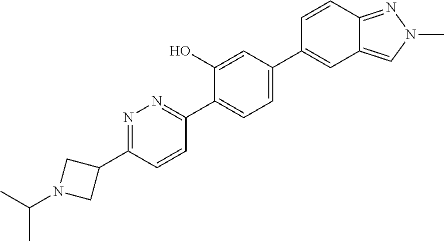 [SMILES: CC(C)N1CC(c2ccc(-c3ccc(-c4ccc5nn(C)cc5c4)cc3O)nn2)C1] |
| 24 | 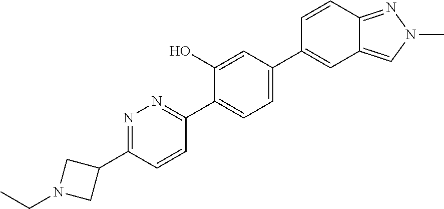 [SMILES: CCN1CC(c2ccc(-c3ccc(-c4ccc5nn(C)cc5c4)cc3O)nn2)C1] |
| 25 | 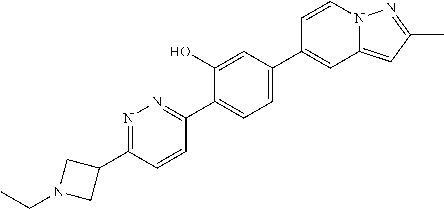 [SMILES: CCN1CC(c2ccc(-c3ccc(-c4ccn5nc(C)cc5c4)cc3O)nn2)C1] |
| 26 | 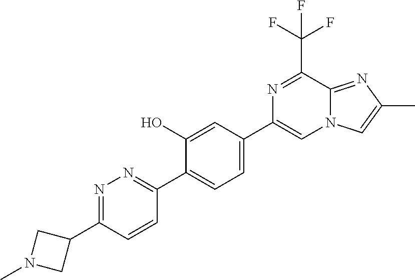 |
| 27 | 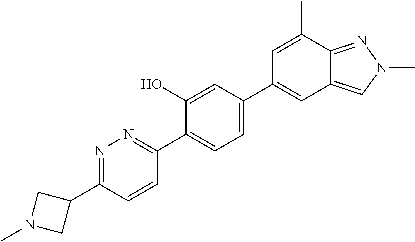 |
| 28 | 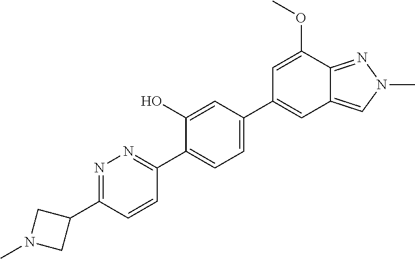 |
| 29 | 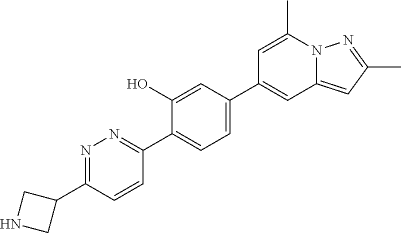 [SMILES: Cc1cc2cc(-c3ccc(-c4ccc(C5CNC5)nn4)c(O)c3)cc(C)n2n1] |
| 30 | 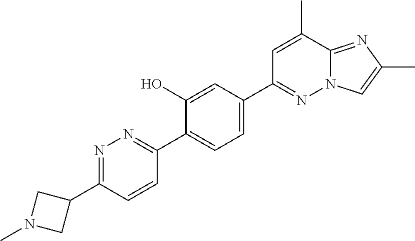 |
| 31 | 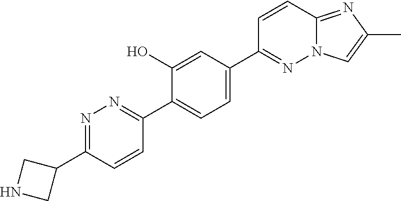 [SMILES: Cc1cn2nc(-c3ccc(-c4ccc(C5CNC5)nn4)c(O)c3)ccc2n1] |
| 32 | 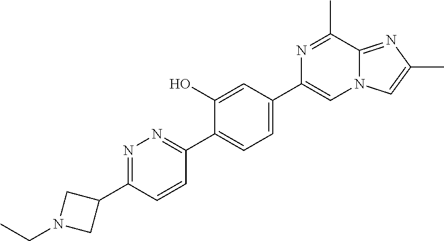 [SMILES: CCN1CC(c2ccc(-c3ccc(-c4cn5cc(C)nc5c(C)n4)cc3O)nn2)C1] |
| 33 | 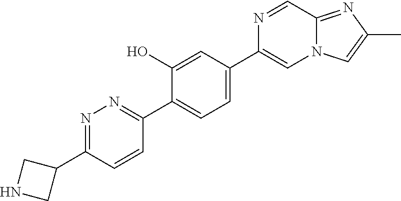 [SMILES: Cc1cn2cc(-c3ccc(-c4ccc(C5CNC5)nn4)c(O)c3)ncc2n1] |
| 34 | 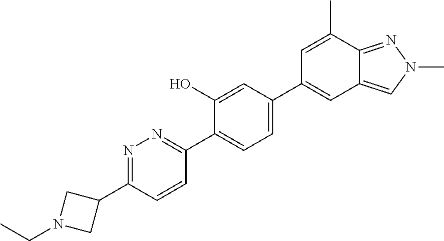 [SMILES: CCN1CC(c2ccc(-c3ccc(-c4cc(C)c5nn(C)cc5c4)cc3O)nn2)C1] |
| 35 | 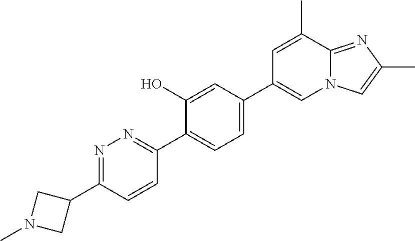 |
| 36 | 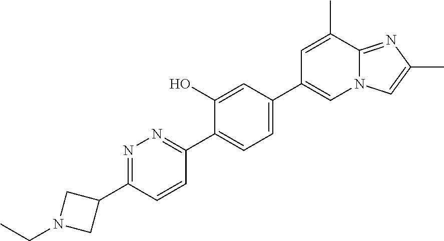 [SMILES: CCN1CC(c2ccc(-c3ccc(-c4cc(C)c5nc(C)cn5c4)cc3O)nn2)C1] |
| 37 | 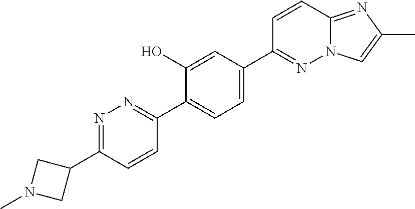 |
| 38 | 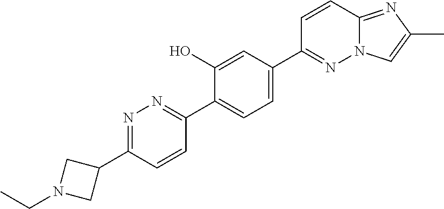 [SMILES: CCN1CC(c2ccc(-c3ccc(-c4ccc5nc(C)cn5n4)cc3O)nn2)C1] |
| 39 | 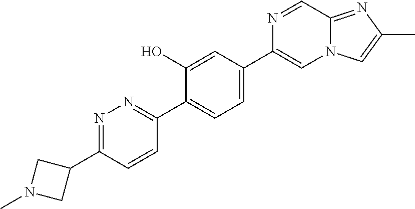 |
| 40 | 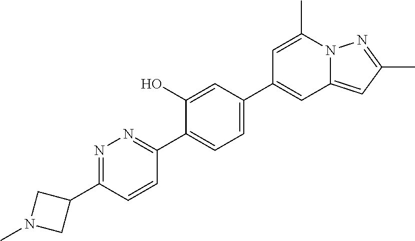 |
| 41 | 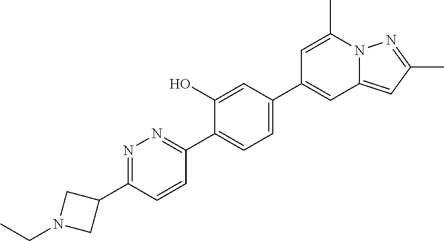 [SMILES: CCN1CC(c2ccc(-c3ccc(-c4cc(C)n5nc(C)cc5c4)cc3O)nn2)C1] |
| 42 | 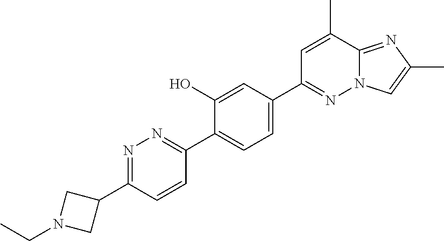 [SMILES: CCN1CC(c2ccc(-c3ccc(-c4cc(C)c5nc(C)cn5n4)cc3O)nn2)C1] |
| 43 | 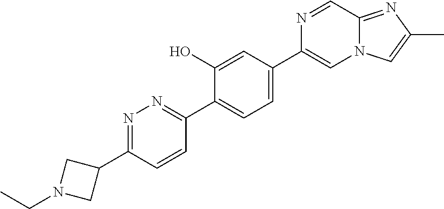 [SMILES: CCN1CC(c2ccc(-c3ccc(-c4cn5cc(C)nc5cn4)cc3O)nn2)C1] |
| 44 | 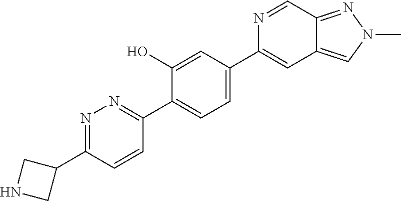 [SMILES: Cn1cc2cc(-c3ccc(-c4ccc(C5CNC5)nn4)c(O)c3)ncc2n1] |
| 45 | 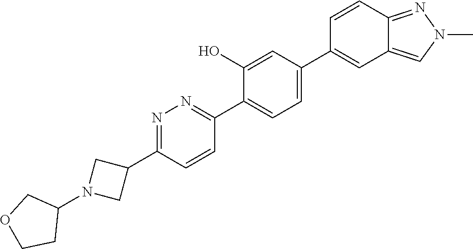 [SMILES: Cn1cc2cc(-c3ccc(-c4ccc(C5CN(C6CCOC6)C5)nn4)c(O)c3)ccc2n1] |
| 46 | 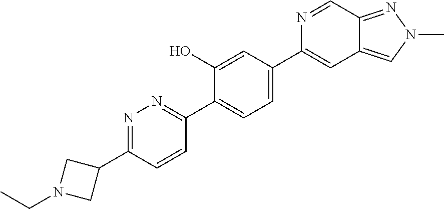 [SMILES: CCN1CC(c2ccc(-c3ccc(-c4cc5cn(C)nc5cn4)cc3O)nn2)C1] |
| 47 | 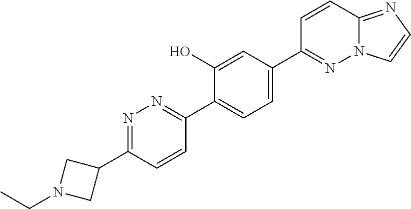 [SMILES: CCN1CC(c2ccc(-c3ccc(-c4ccc5nccn5n4)cc3O)nn2)C1] |
| 48 | 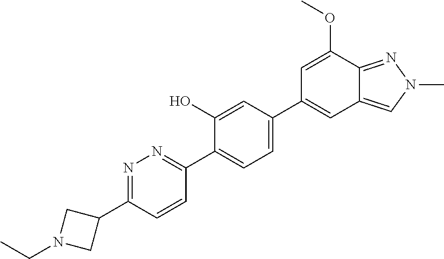 [SMILES: CCN1CC(c2ccc(-c3ccc(-c4cc(OC)c5nn(C)cc5c4)cc3O)nn2)C1] |
| 49 | 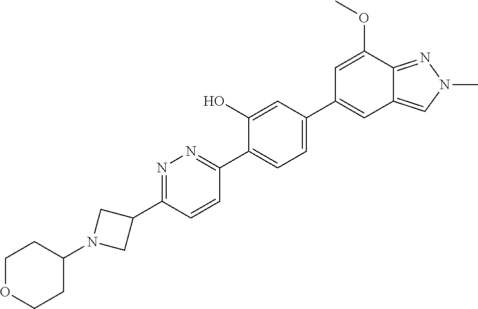 [SMILES: COc1cc(-c2ccc(-c3ccc(C4CN(C5CCOCC5)C4)nn3)c(O)c2)cc2cn(C)nc12] |
| 50 | 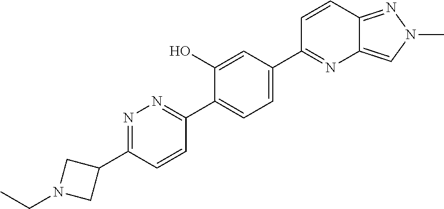 [SMILES: CCN1CC(c2ccc(-c3ccc(-c4ccc5nn(C)cc5n4)cc3O)nn2)C1] |
| 51 | 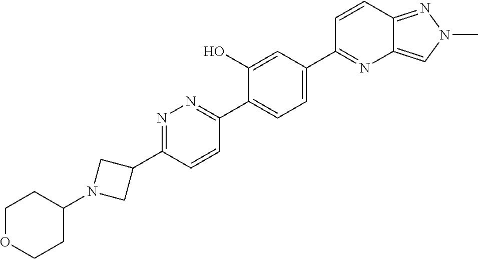 [SMILES: Cn1cc2nc(-c3ccc(-c4ccc(C5CN(C6CCOCC6)C5)nn4)c(O)c3)ccc2n1] |
| 52 | 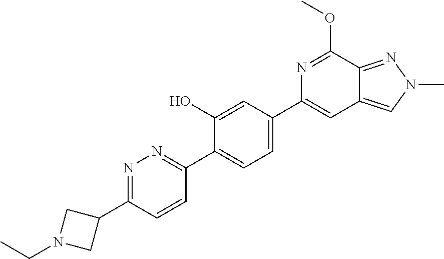 [SMILES: CCN1CC(c2ccc(-c3ccc(-c4cc5cn(C)nc5c(OC)n4)cc3O)nn2)C1] |
| 53 | 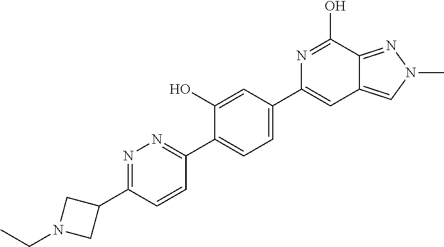 [SMILES: CCN1CC(c2ccc(-c3ccc(-c4cc5cn(C)nc5c(O)n4)cc3O)nn2)C1] |
| 54 | 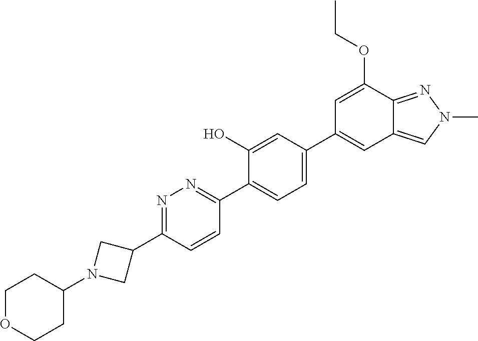 [SMILES: CCOc1cc(-c2ccc(-c3ccc(C4CN(C5CCOCC5)C4)nn3)c(O)c2)cc2cn(C)nc12] |
| 55 | 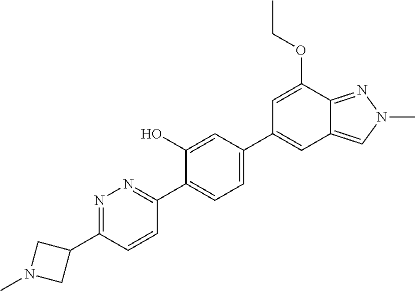 |
| 56 | 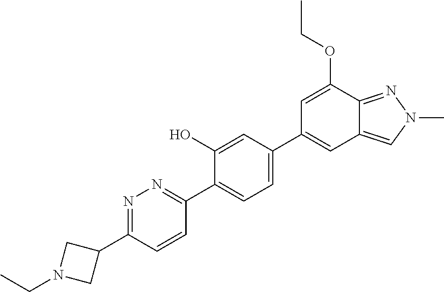 [SMILES: CCOc1cc(-c2ccc(-c3ccc(C4CN(CC)C4)nn3)c(O)c2)cc2cn(C)nc12] |
| 57 | 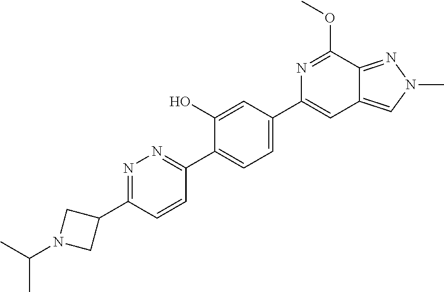 [SMILES: COc1nc(-c2ccc(-c3ccc(C4CN(C(C)C)C4)nn3)c(O)c2)cc2cn(C)nc12] |
| 58 | 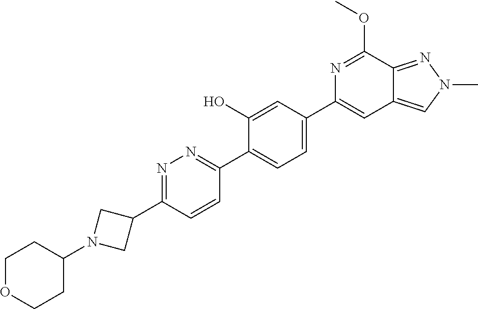 [SMILES: COc1nc(-c2ccc(-c3ccc(C4CN(C5CCOCC5)C4)nn3)c(O)c2)cc2cn(C)nc12] |
| 59 | 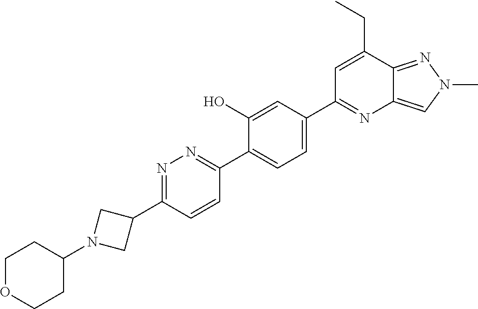 [SMILES: CCc1cc(-c2ccc(-c3ccc(C4CN(C5CCOCC5)C4)nn3)c(O)c2)nc2cn(C)nc12] |
| 60 | 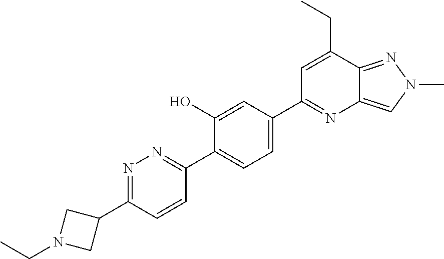 [SMILES: CCc1cc(-c2ccc(-c3ccc(C4CN(CC)C4)nn3)c(O)c2)nc2cn(C)nc12] |
| 61 | 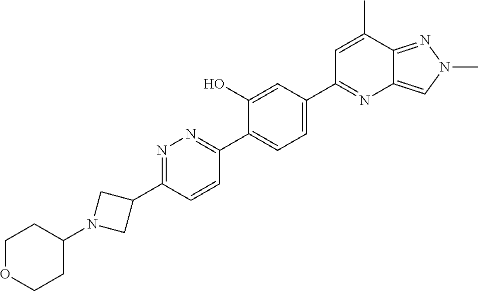 [SMILES: Cc1cc(-c2ccc(-c3ccc(C4CN(C5CCOCC5)C4)nn3)c(O)c2)nc2cn(C)nc12] |
| 62 | 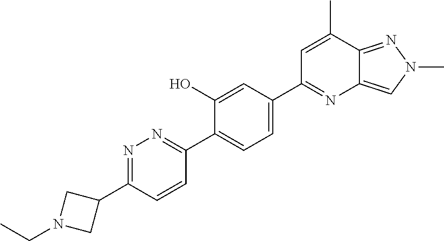 [SMILES: CCN1CC(c2ccc(-c3ccc(-c4cc(C)c5nn(C)cc5n4)cc3O)nn2)C1] |
| 63 | 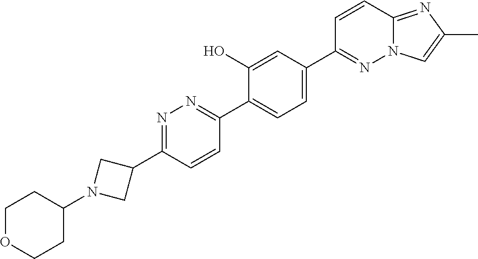 [SMILES: Cc1cn2nc(-c3ccc(-c4ccc(C5CN(C6CCOCC6)C5)nn4)c(O)c3)ccc2n1] |
| 64 | 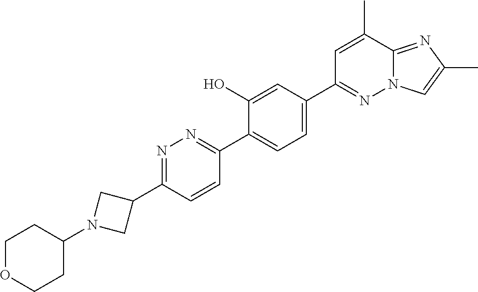 [SMILES: Cc1cn2nc(-c3ccc(-c4ccc(C5CN(C6CCOCC6)C5)nn4)c(O)c3)cc(C)c2n1] |
| 65 | 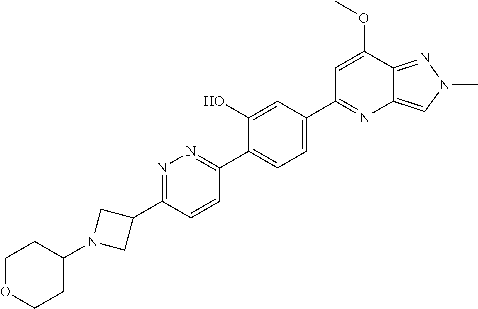 [SMILES: COc1cc(-c2ccc(-c3ccc(C4CN(C5CCOCC5)C4)nn3)c(O)c2)nc2cn(C)nc12] |
| 66 | 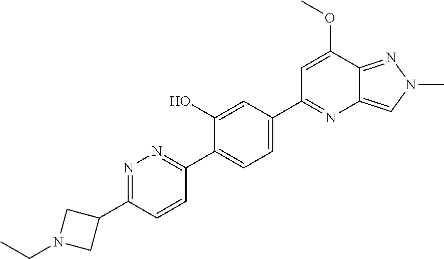 [SMILES: CCN1CC(c2ccc(-c3ccc(-c4cc(OC)c5nn(C)cc5n4)cc3O)nn2)C1] |
| 70 | 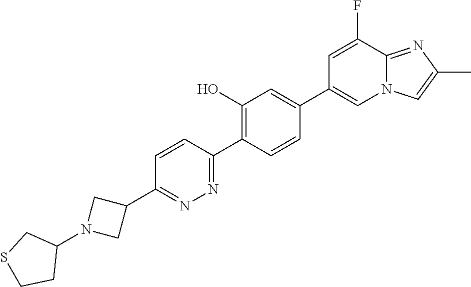 [SMILES: Cc1cn2cc(-c3ccc(-c4ccc(C5CN(C6CCSC6)C5)nn4)c(O)c3)cc(F)c2n1] |
| 71 | 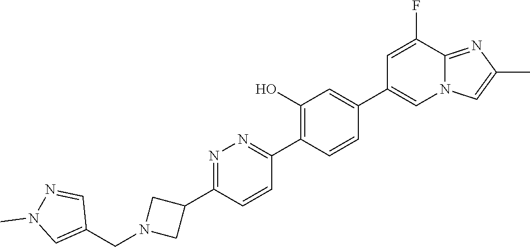 |
| 72 | 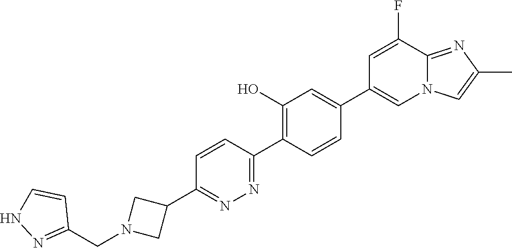 [SMILES: Cc1cn2cc(-c3ccc(-c4ccc(C5CN(Cc6cc[nH]n6)C5)nn4)c(O)c3)cc(F)c2n1] |
| 73 | 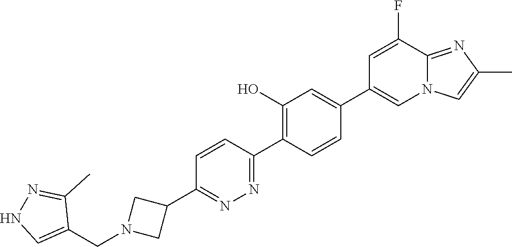 [SMILES: Cc1cn2cc(-c3ccc(-c4ccc(C5CN(Cc6c[nH]nc6C)C5)nn4)c(O)c3)cc(F)c2n1] |
| 74 | 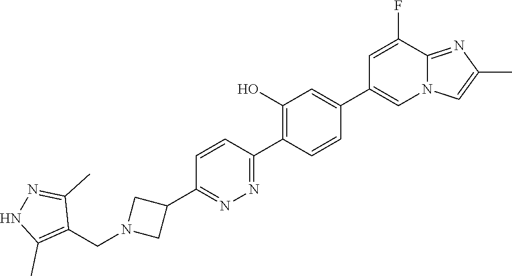 [SMILES: Cc1cn2cc(-c3ccc(-c4ccc(C5CN(Cc6c(C)n[nH]c6C)C5)nn4)c(O)c3)cc(F)c2n1] |
| 75 | 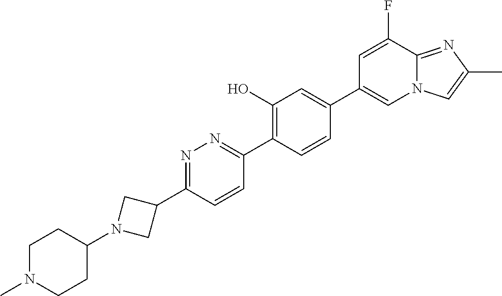 |
| 76 | 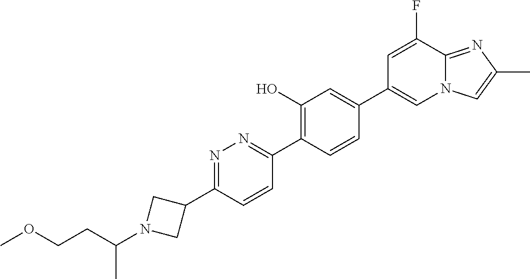 |
| 77 | 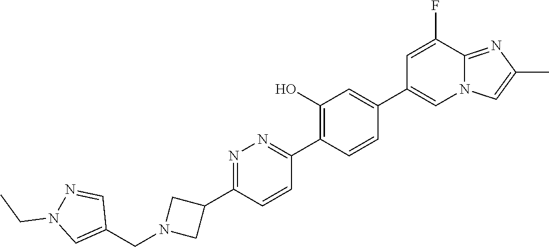 [SMILES: CCn1cc(CN2CC(c3ccc(-c4ccc(-c5cc(F)c6nc(C)cn6c5)cc4O)nn3)C2)cn1] |
| 78 | 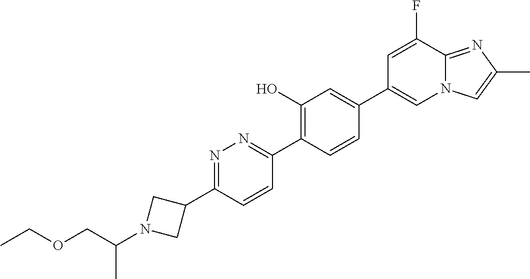 [SMILES: CCOCC(C)N1CC(c2ccc(-c3ccc(-c4cc(F)c5nc(C)cn5c4)cc3O)nn2)C1] |
| 79 | 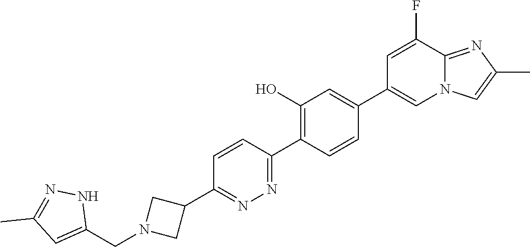 |
| 80 | 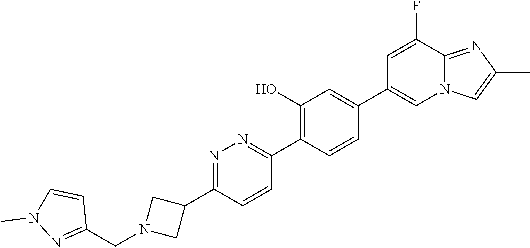 |
| 81 | 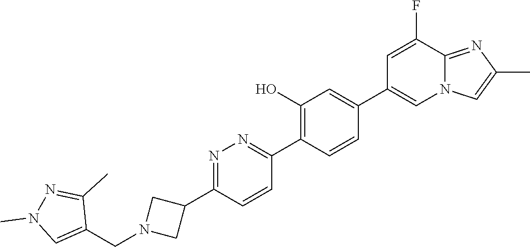 |
| 82 | 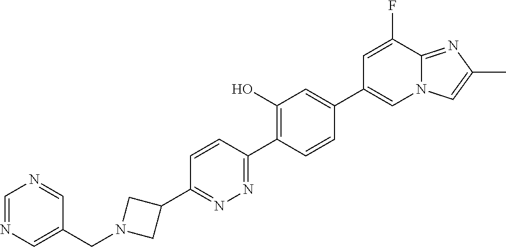 [SMILES: Cc1cn2cc(-c3ccc(-c4ccc(C5CN(Cc6cncnc6)C5)nn4)c(O)c3)cc(F)c2n1] |
| 83 | 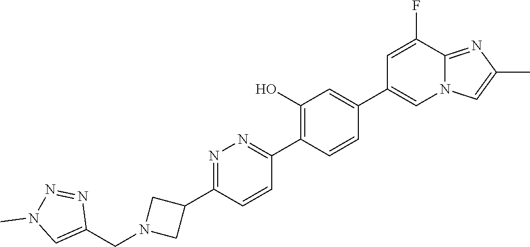 |
| 84 | 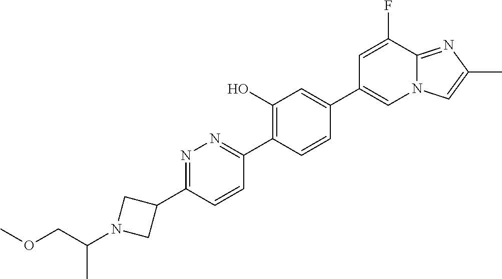 |
| 85 | 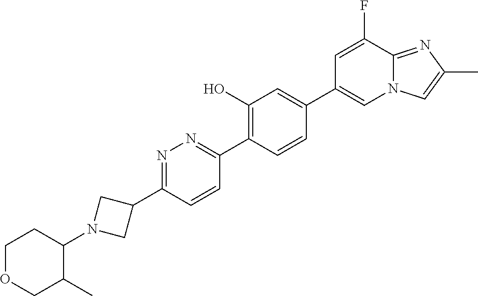 [SMILES: Cc1cn2cc(-c3ccc(-c4ccc(C5CN(C6CCOCC6C)C5)nn4)c(O)c3)cc(F)c2n1] |
| 86 | 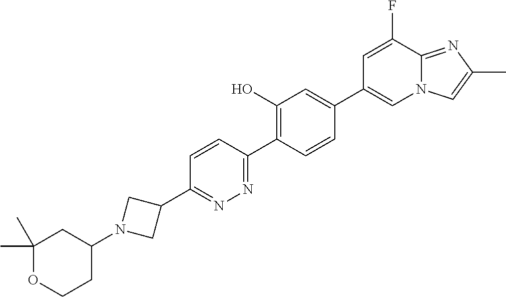 |
| 87 | 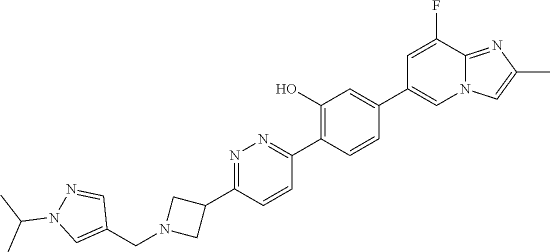 [SMILES: Cc1cn2cc(-c3ccc(-c4ccc(C5CN(Cc6cnn(C(C)C)c6)C5)nn4)c(O)c3)cc(F)c2n1] |
| 88 | 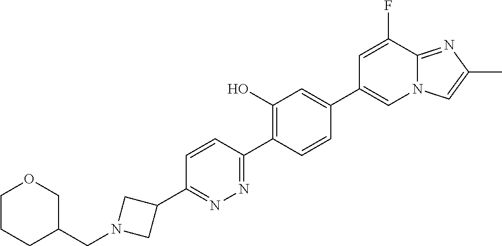 [SMILES: Cc1cn2cc(-c3ccc(-c4ccc(C5CN(CC6CCCOC6)C5)nn4)c(O)c3)cc(F)c2n1] |
| 89 | 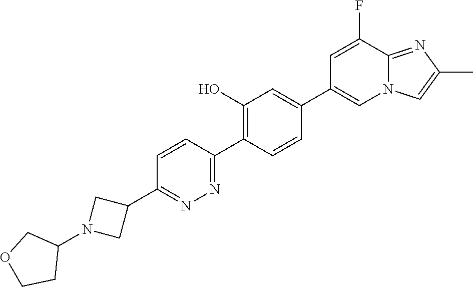 [SMILES: Cc1cn2cc(-c3ccc(-c4ccc(C5CN(C6CCOC6)C5)nn4)c(O)c3)cc(F)c2n1] |
| 90 | 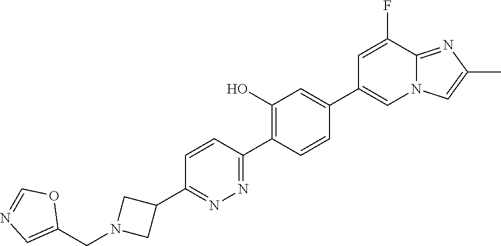 [SMILES: Cc1cn2cc(-c3ccc(-c4ccc(C5CN(Cc6cnco6)C5)nn4)c(O)c3)cc(F)c2n1] |
| 91 | 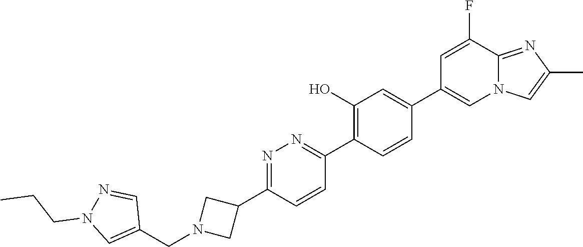 [SMILES: CCCn1cc(CN2CC(c3ccc(-c4ccc(-c5cc(F)c6nc(C)cn6c5)cc4O)nn3)C2)cn1] |
| 92 | 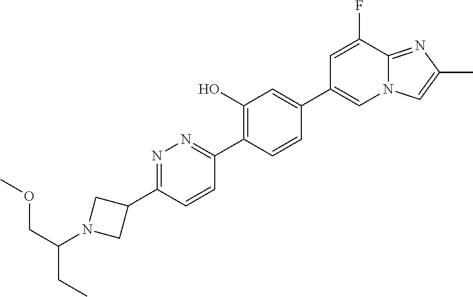 |
| 93 | 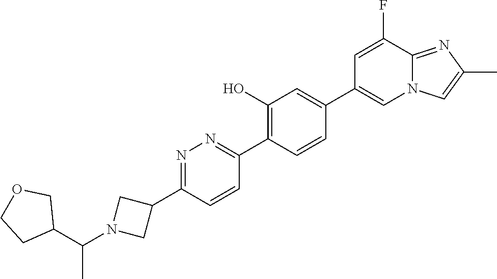 [SMILES: Cc1cn2cc(-c3ccc(-c4ccc(C5CN(C(C)C6CCOC6)C5)nn4)c(O)c3)cc(F)c2n1] |
| 94 | 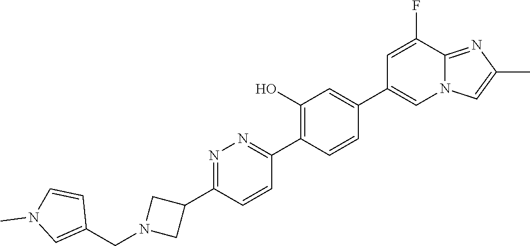 |
| 95 | 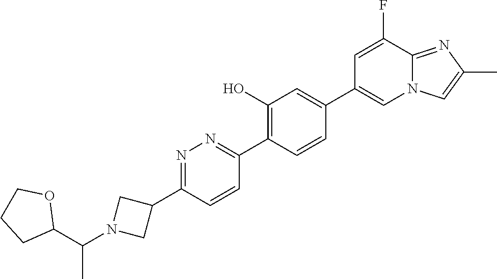 [SMILES: Cc1cn2cc(-c3ccc(-c4ccc(C5CN(C(C)C6CCCO6)C5)nn4)c(O)c3)cc(F)c2n1] |
| 96 | 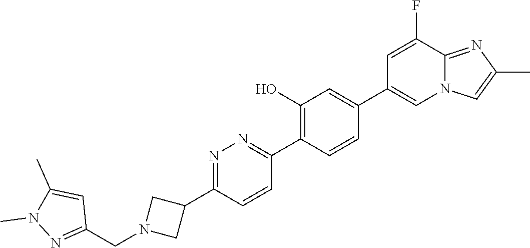 |
| 97 | 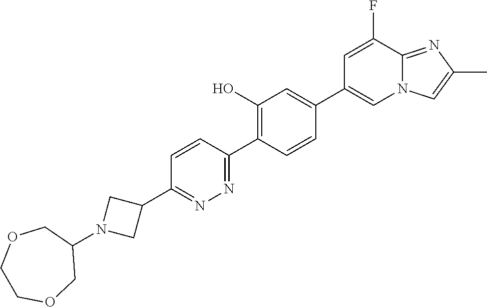 [SMILES: Cc1cn2cc(-c3ccc(-c4ccc(C5CN(C6COCCOC6)C5)nn4)c(O)c3)cc(F)c2n1] |
| 98 | 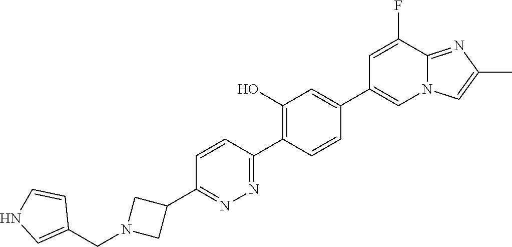 [SMILES: Cc1cn2cc(-c3ccc(-c4ccc(C5CN(Cc6cc[nH]c6)C5)nn4)c(O)c3)cc(F)c2n1] |
| 99 | 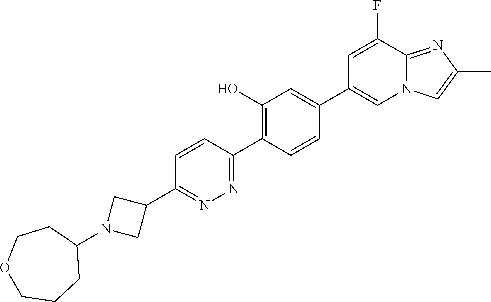 [SMILES: Cc1cn2cc(-c3ccc(-c4ccc(C5CN(C6CCCOCC6)C5)nn4)c(O)c3)cc(F)c2n1] |
| 100 | 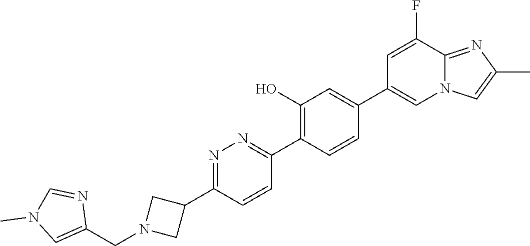 |
| 101 | 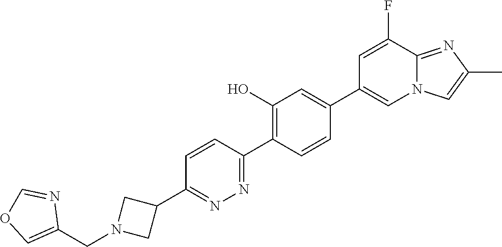 [SMILES: Cc1cn2cc(-c3ccc(-c4ccc(C5CN(Cc6cocn6)C5)nn4)c(O)c3)cc(F)c2n1] |
| 102 | 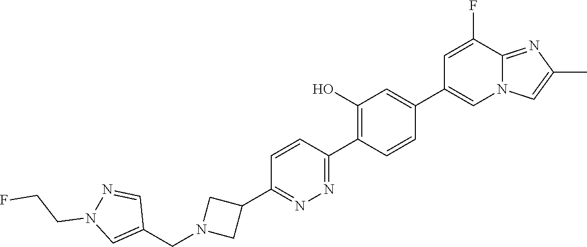 [SMILES: Cc1cn2cc(-c3ccc(-c4ccc(C5CN(Cc6cnn(CCF)c6)C5)nn4)c(O)c3)cc(F)c2n1] |
| 103 | 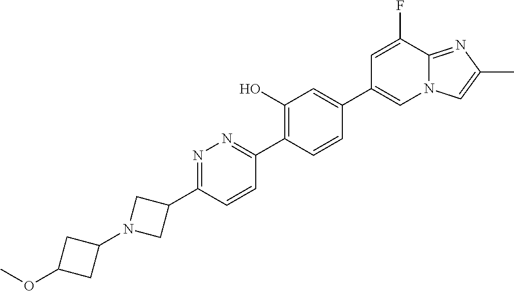 |
| 104 | 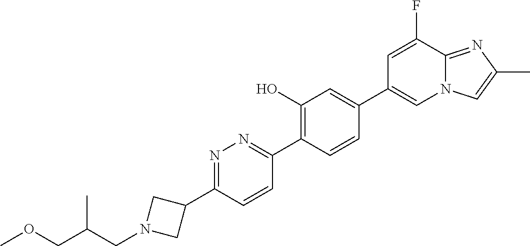 |
| 105 | 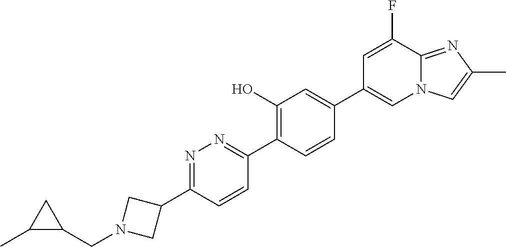 |
| 106 | 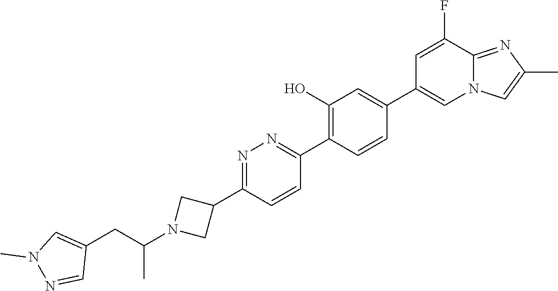 |
| 107 | 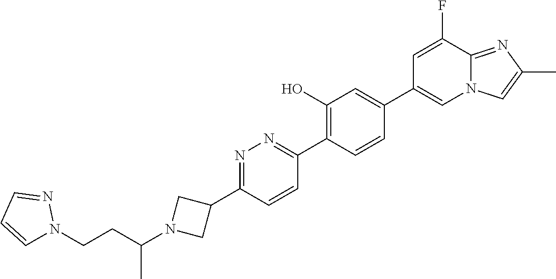 [SMILES: Cc1cn2cc(-c3ccc(-c4ccc(C5CN(C(C)CCn6cccn6)C5)nn4)c(O)c3)cc(F)c2n1] |
| 108 | 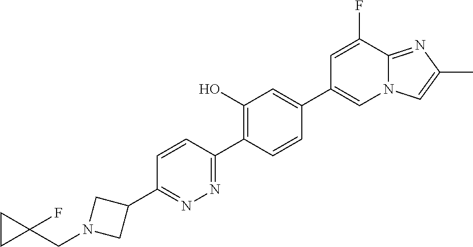 [SMILES: Cc1cn2cc(-c3ccc(-c4ccc(C5CN(CC6(F)CC6)C5)nn4)c(O)c3)cc(F)c2n1] |
| 109 | 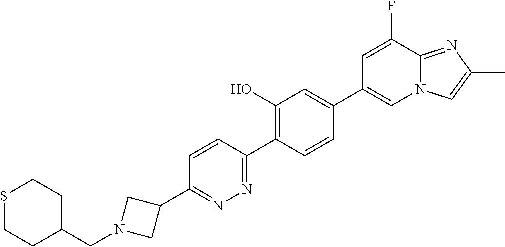 [SMILES: Cc1cn2cc(-c3ccc(-c4ccc(C5CN(CC6CCSCC6)C5)nn4)c(O)c3)cc(F)c2n1] |
| 110 | 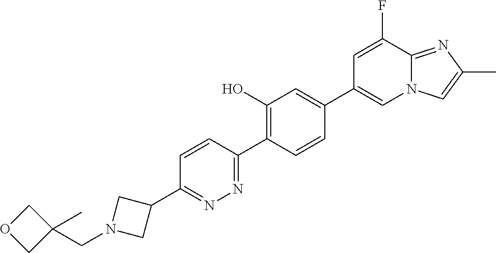 [SMILES: Cc1cn2cc(-c3ccc(-c4ccc(C5CN(CC6(C)COC6)C5)nn4)c(O)c3)cc(F)c2n1] |
| 111 | 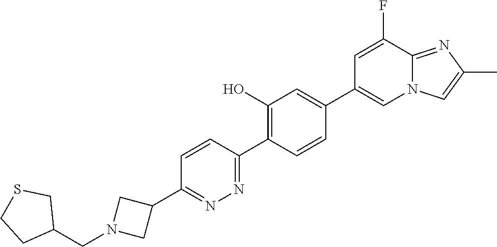 [SMILES: Cc1cn2cc(-c3ccc(-c4ccc(C5CN(CC6CCSC6)C5)nn4)c(O)c3)cc(F)c2n1] |
| 112 | 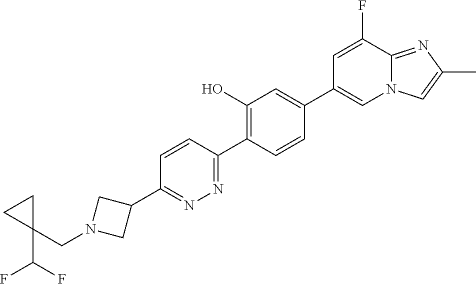 [SMILES: Cc1cn2cc(-c3ccc(-c4ccc(C5CN(CC6(C(F)F)CC6)C5)nn4)c(O)c3)cc(F)c2n1] |
| 113 | 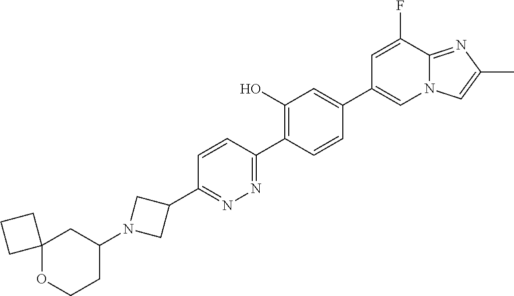 [SMILES: Cc1cn2cc(-c3ccc(-c4ccc(C5CN(C6CCOC7(CCC7)C6)C5)nn4)c(O)c3)cc(F)c2n1] |
| 114 | 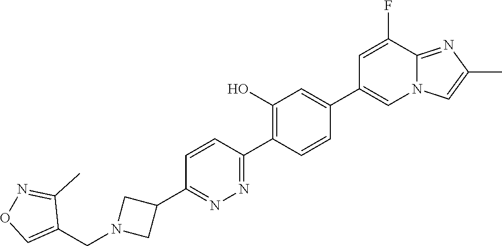 [SMILES: Cc1cn2cc(-c3ccc(-c4ccc(C5CN(Cc6conc6C)C5)nn4)c(O)c3)cc(F)c2n1] |
| 115 | 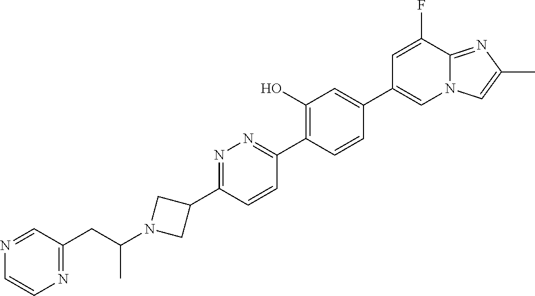 [SMILES: Cc1cn2cc(-c3ccc(-c4ccc(C5CN(C(C)Cc6cnccn6)C5)nn4)c(O)c3)cc(F)c2n1] |
| 116 | 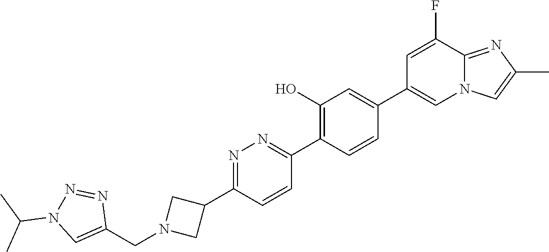 [SMILES: Cc1cn2cc(-c3ccc(-c4ccc(C5CN(Cc6cn(C(C)C)nn6)C5)nn4)c(O)c3)cc(F)c2n1] |
| 117 | 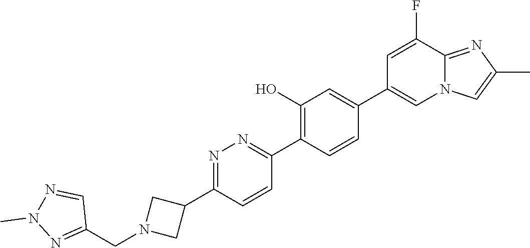 |
| 118 | 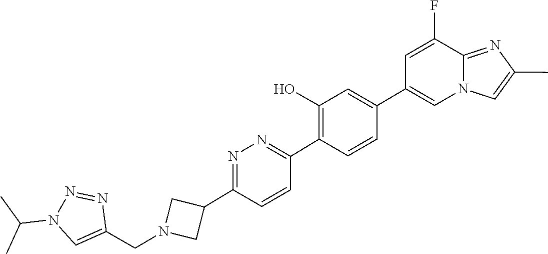 [SMILES: Cc1cn2cc(-c3ccc(-c4ccc(C5CN(Cc6cn(C(C)C)nn6)C5)nn4)c(O)c3)cc(F)c2n1] |
| 119 | 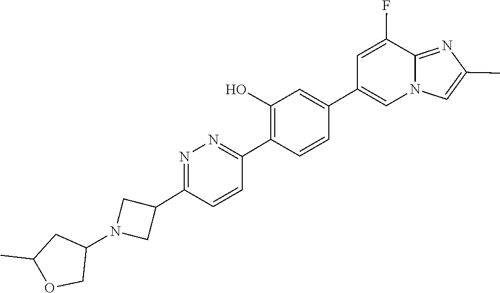 |
| 120 | 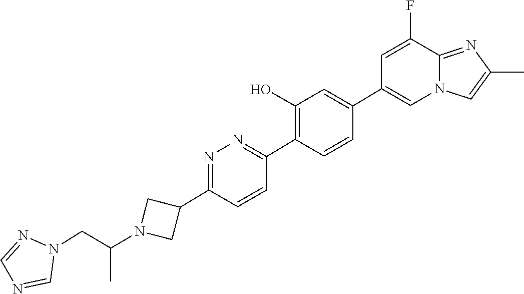 [SMILES: Cc1cn2cc(-c3ccc(-c4ccc(C5CN(C(C)Cn6cncn6)C5)nn4)c(O)c3)cc(F)c2n1] |
| 121 | 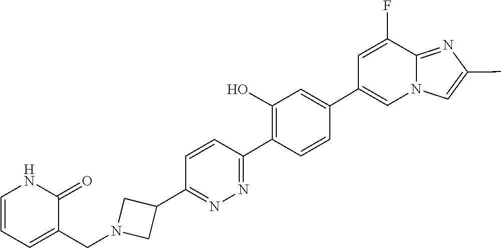 [SMILES: Cc1cn2cc(-c3ccc(-c4ccc(C5CN(Cc6ccc[nH]c6=O)C5)nn4)c(O)c3)cc(F)c2n1] |
| 122 | 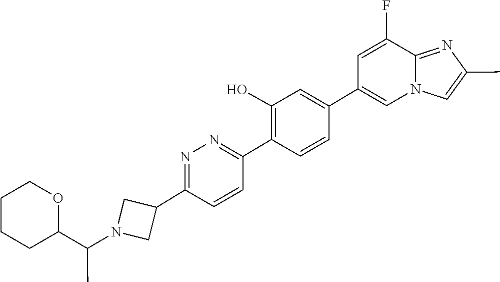 [SMILES: Cc1cn2cc(-c3ccc(-c4ccc(C5CN(C(C)C6CCCCO6)C5)nn4)c(O)c3)cc(F)c2n1] |
| 123 | 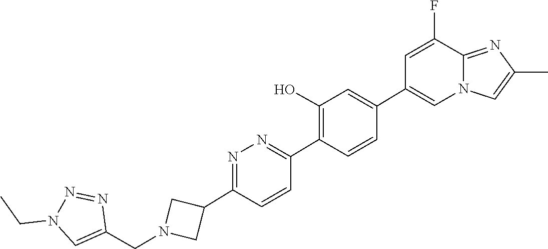 [SMILES: CCn1cc(CN2CC(c3ccc(-c4ccc(-c5cc(F)c6nc(C)cn6c5)cc4O)nn3)C2)nn1] |
| 124 | 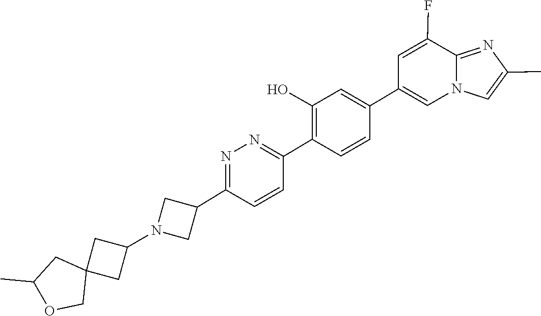 |
| 125 | 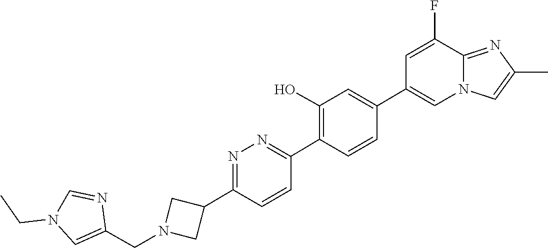 [SMILES: CCn1cnc(CN2CC(c3ccc(-c4ccc(-c5cc(F)c6nc(C)cn6c5)cc4O)nn3)C2)c1] |
| 126 | 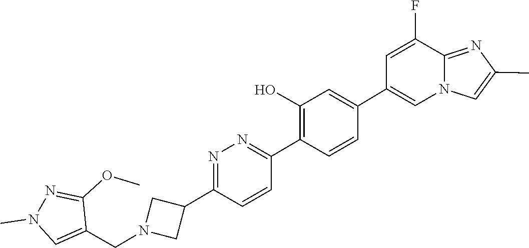 |
| 127 | 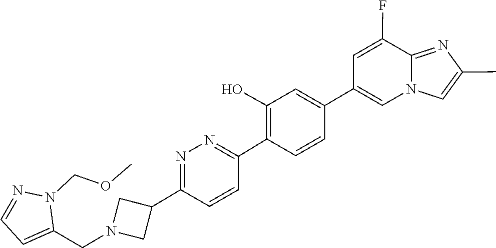 [SMILES: COCn1nccc1CN1CC(c2ccc(-c3ccc(-c4cc(F)c5nc(C)cn5c4)cc3O)nn2)C1] |
| 128 | 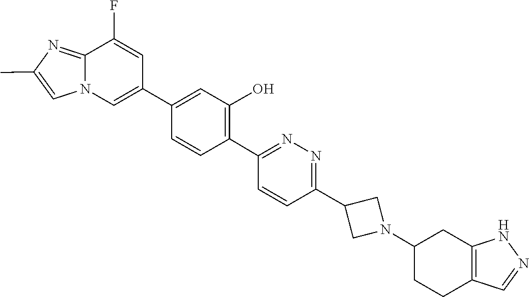 [SMILES: Cc1cn2cc(-c3ccc(-c4ccc(C5CN(C6CCc7cn[nH]c7C6)C5)nn4)c(O)c3)cc(F)c2n1] |
| 129 | 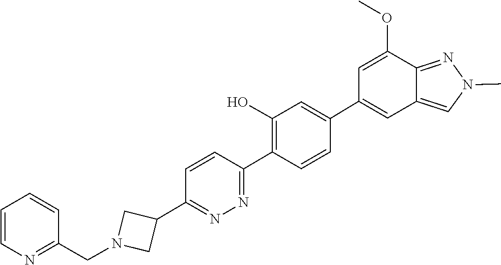 [SMILES: COc1cc(-c2ccc(-c3ccc(C4CN(Cc5ccccn5)C4)nn3)c(O)c2)cc2cn(C)nc12] |
| 130 | 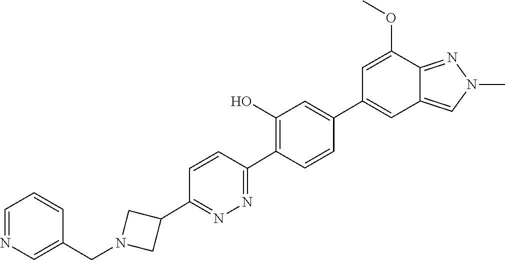 [SMILES: COc1cc(-c2ccc(-c3ccc(C4CN(Cc5cccnc5)C4)nn3)c(O)c2)cc2cn(C)nc12] |
| 131 | 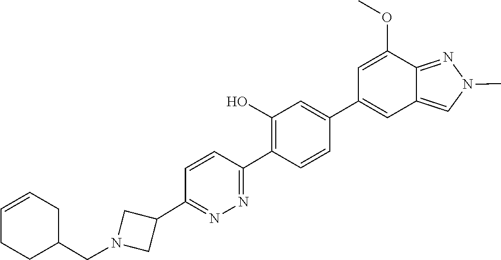 [SMILES: COc1cc(-c2ccc(-c3ccc(C4CN(CC5CC=CCC5)C4)nn3)c(O)c2)cc2cn(C)nc12] |
| 132 | 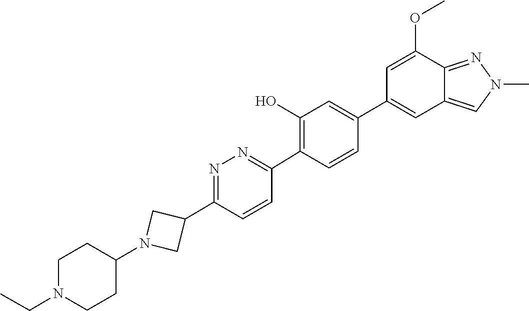 [SMILES: CCN1CCC(N2CC(c3ccc(-c4ccc(-c5cc(OC)c6nn(C)cc6c5)cc4O)nn3)C2)CC1] |
| 133 | 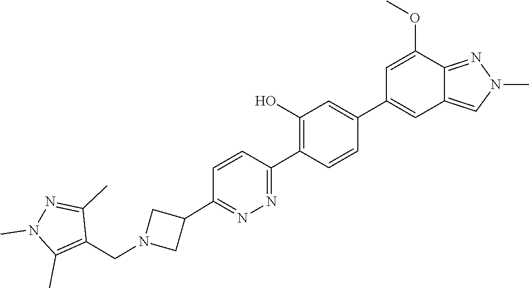 |
| 134 | 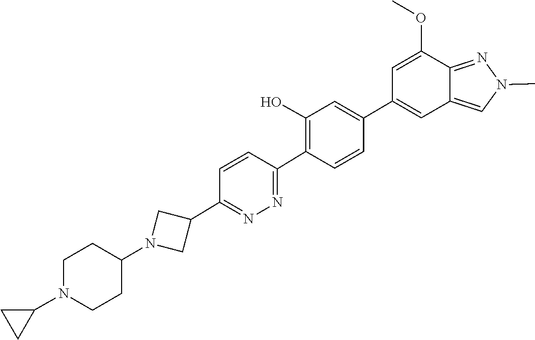 [SMILES: COc1cc(-c2ccc(-c3ccc(C4CN(C5CCN(C6CC6)CC5)C4)nn3)c(O)c2)cc2cn(C)nc12] |
| 135 | 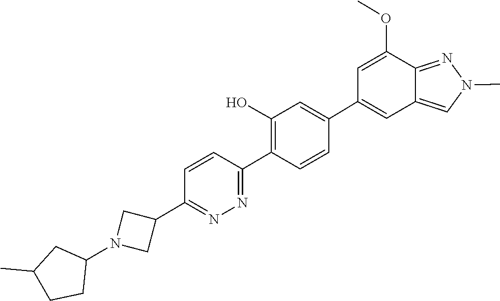 |
| 136 | 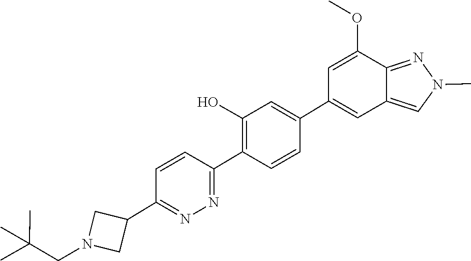 [SMILES: COc1cc(-c2ccc(-c3ccc(C4CN(CC(C)(C)C)C4)nn3)c(O)c2)cc2cn(C)nc12] |
| 137 | 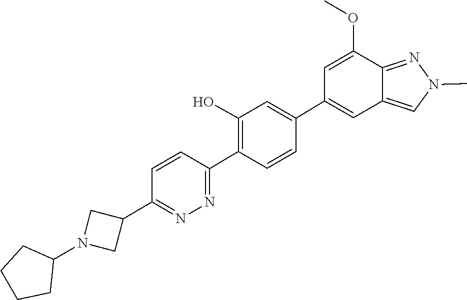 [SMILES: COc1cc(-c2ccc(-c3ccc(C4CN(C5CCCC5)C4)nn3)c(O)c2)cc2cn(C)nc12] |
| 138 | 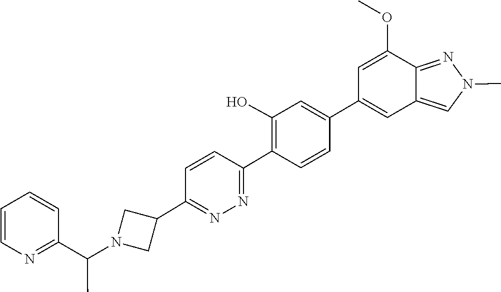 [SMILES: COc1cc(-c2ccc(-c3ccc(C4CN(C(C)c5ccccn5)C4)nn3)c(O)c2)cc2cn(C)nc12] |
| 139 | 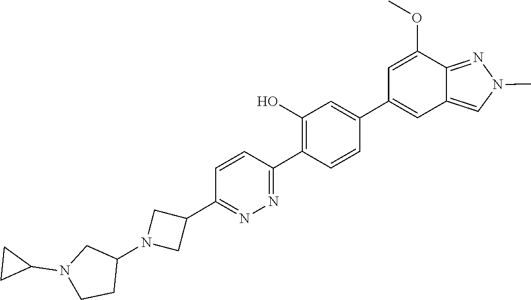 [SMILES: COc1cc(-c2ccc(-c3ccc(C4CN(C5CCN(C6CC6)C5)C4)nn3)c(O)c2)cc2cn(C)nc12] |
| 140 | 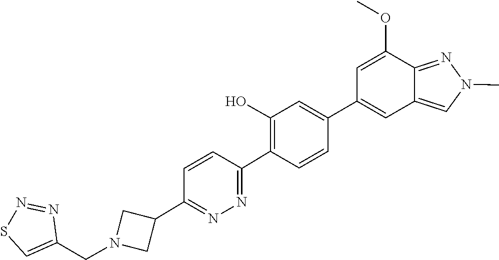 [SMILES: COc1cc(-c2ccc(-c3ccc(C4CN(Cc5csnn5)C4)nn3)c(O)c2)cc2cn(C)nc12] |
| 141 | 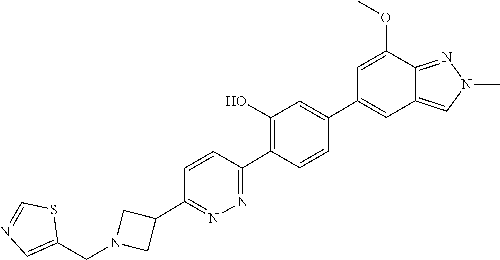 [SMILES: COc1cc(-c2ccc(-c3ccc(C4CN(Cc5cncs5)C4)nn3)c(O)c2)cc2cn(C)nc12] |
| 142 | 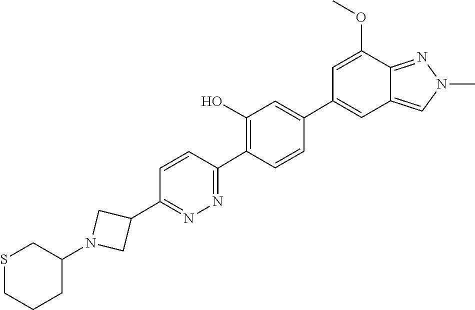 [SMILES: COc1cc(-c2ccc(-c3ccc(C4CN(C5CCCSC5)C4)nn3)c(O)c2)cc2cn(C)nc12] |
| 143 | 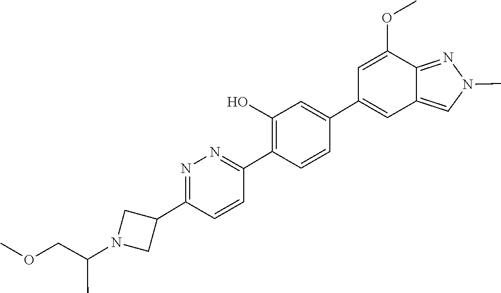 |
| 144 | 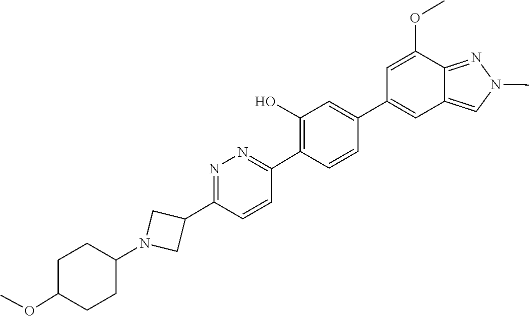 |
| 145 | 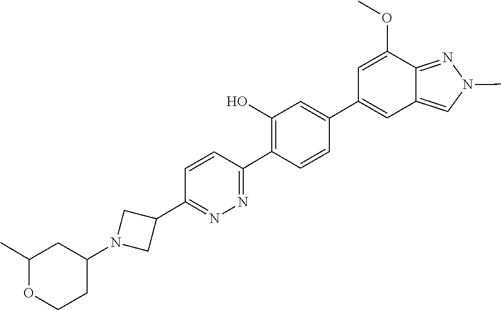 |
| 146 | 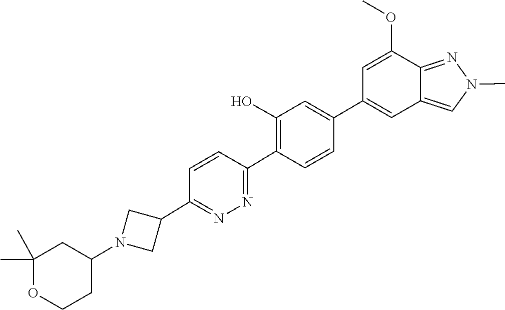 |
| 147 | 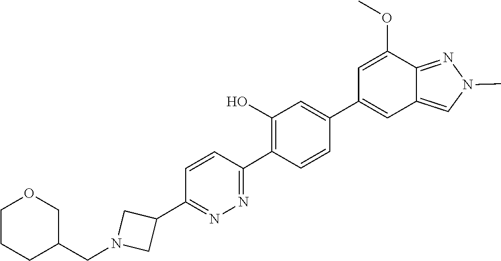 [SMILES: COc1cc(-c2ccc(-c3ccc(C4CN(CC5CCCOC5)C4)nn3)c(O)c2)cc2cn(C)nc12] |
| 148 | 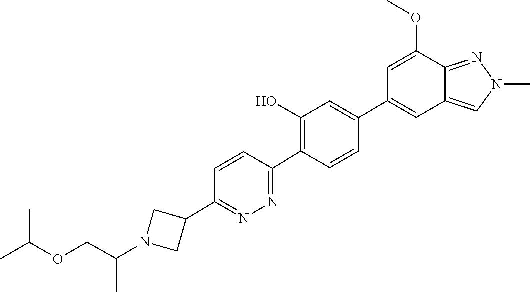 [SMILES: COc1cc(-c2ccc(-c3ccc(C4CN(C(C)COC(C)C)C4)nn3)c(O)c2)cc2cn(C)nc12] |
| 149 | 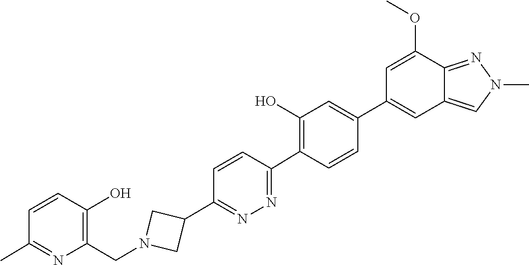 |
| 150 | 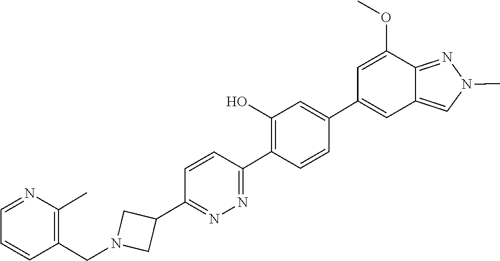 [SMILES: COc1cc(-c2ccc(-c3ccc(C4CN(Cc5cccnc5C)C4)nn3)c(O)c2)cc2cn(C)nc12] |
| 151 | 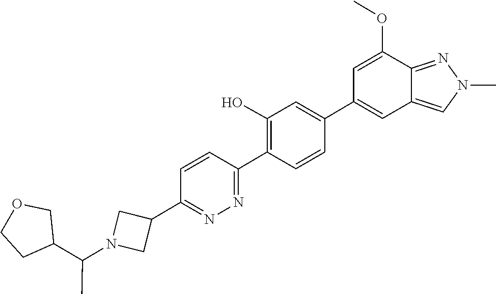 [SMILES: COc1cc(-c2ccc(-c3ccc(C4CN(C(C)C5CCOC5)C4)nn3)c(O)c2)cc2cn(C)nc12] |
| 152 | 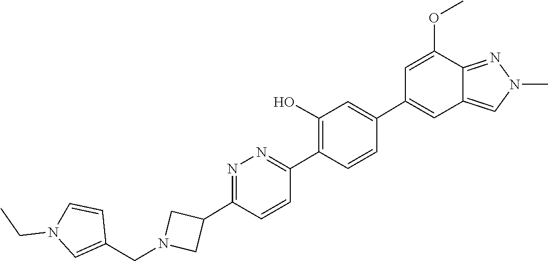 [SMILES: CCn1ccc(CN2CC(c3ccc(-c4ccc(-c5cc(OC)c6nn(C)cc6c5)cc4O)nn3)C2)c1] |
| 153 | 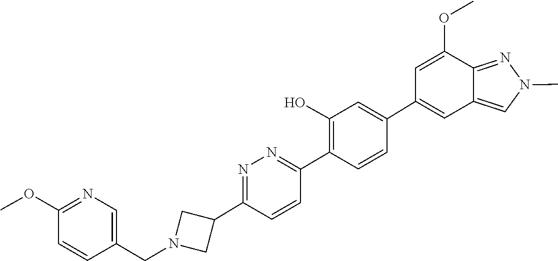 |
| 154 | 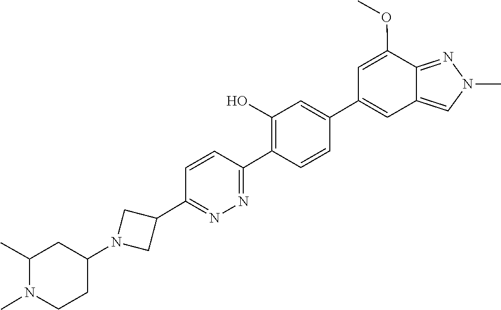 |
| 155 | 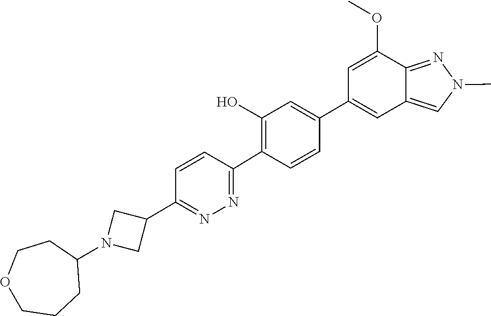 [SMILES: COc1cc(-c2ccc(-c3ccc(C4CN(C5CCCOCC5)C4)nn3)c(O)c2)cc2cn(C)nc12] |
| 156 | 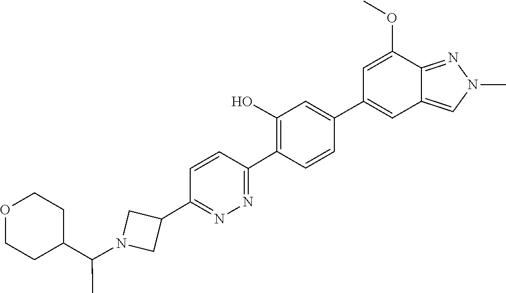 [SMILES: COc1cc(-c2ccc(-c3ccc(C4CN(C(C)C5CCOCC5)C4)nn3)c(O)c2)cc2cn(C)nc12] |
| 157 | 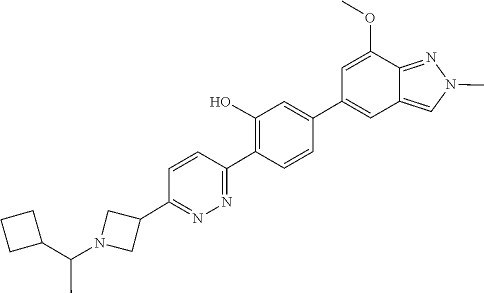 [SMILES: COc1cc(-c2ccc(-c3ccc(C4CN(C(C)C5CCC5)C4)nn3)c(O)c2)cc2cn(C)nc12] |
| 158 | 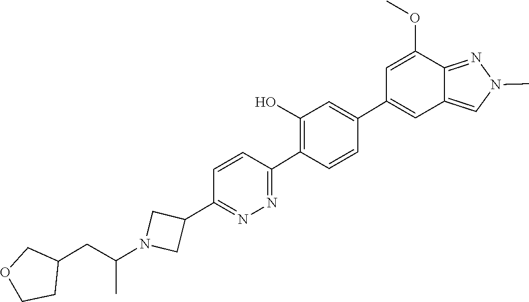 [SMILES: COc1cc(-c2ccc(-c3ccc(C4CN(C(C)CC5CCOC5)C4)nn3)c(O)c2)cc2cn(C)nc12] |
| 159 | 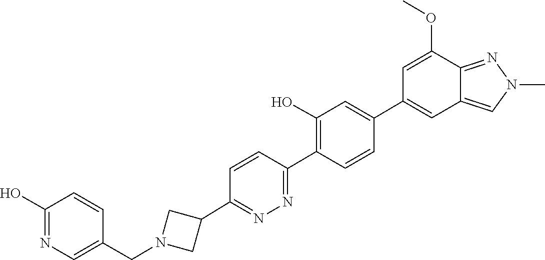 [SMILES: COc1cc(-c2ccc(-c3ccc(C4CN(Cc5ccc(O)nc5)C4)nn3)c(O)c2)cc2cn(C)nc12] |
| 160 | 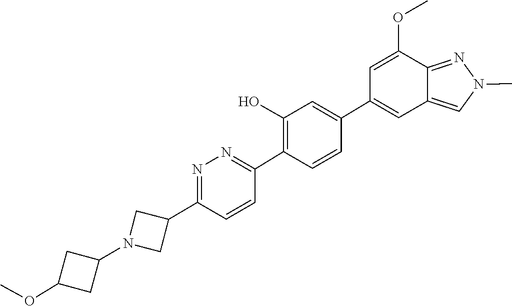 |
| 161 | 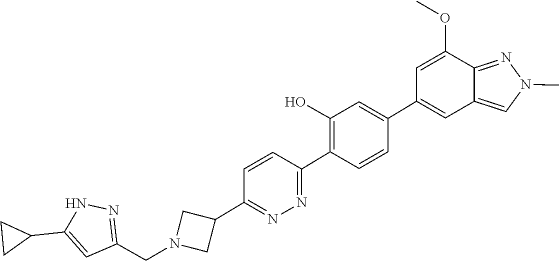 [SMILES: COc1cc(-c2ccc(-c3ccc(C4CN(Cc5cc(C6CC6)[nH]n5)C4)nn3)c(O)c2)cc2cn(C)nc12] |
| 162 | 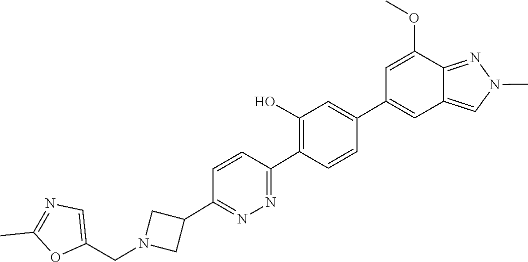 |
| 163 | 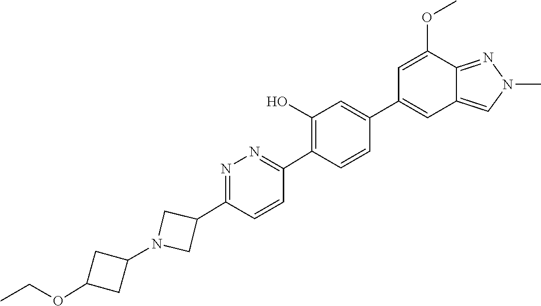 [SMILES: CCOC1CC(N2CC(c3ccc(-c4ccc(-c5cc(OC)c6nn(C)cc6c5)cc4O)nn3)C2)C1] |
| 164 | 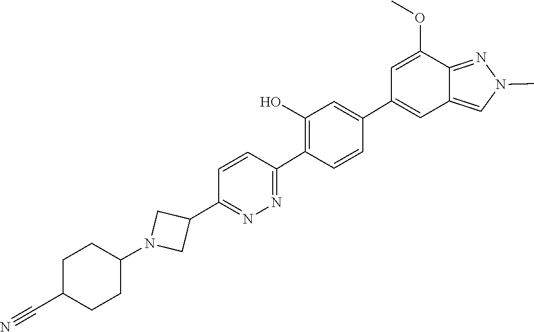 [SMILES: COc1cc(-c2ccc(-c3ccc(C4CN(C5CCC(C#N)CC5)C4)nn3)c(O)c2)cc2cn(C)nc12] |
| 165 | 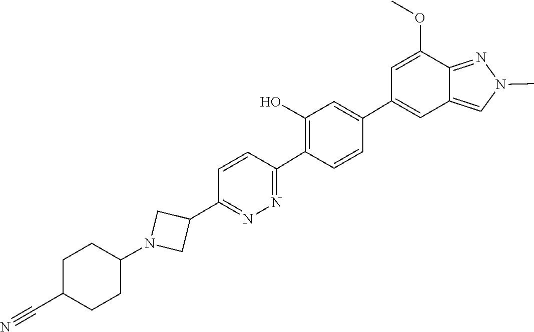 [SMILES: COc1cc(-c2ccc(-c3ccc(C4CN(C5CCC(C#N)CC5)C4)nn3)c(O)c2)cc2cn(C)nc12] |
| 166 | 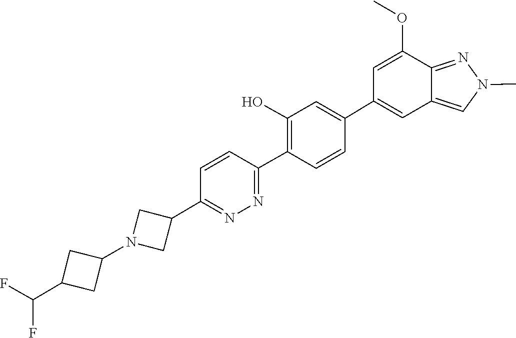 [SMILES: COc1cc(-c2ccc(-c3ccc(C4CN(C5CC(C(F)F)C5)C4)nn3)c(O)c2)cc2cn(C)nc12] |
| 167 | 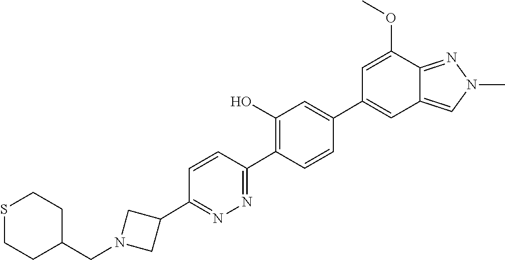 [SMILES: COc1cc(-c2ccc(-c3ccc(C4CN(CC5CCSCC5)C4)nn3)c(O)c2)cc2cn(C)nc12] |
| 168 | 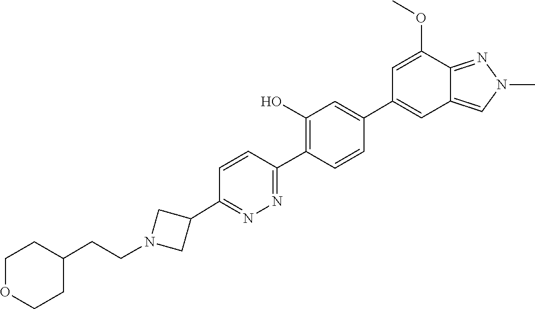 [SMILES: COc1cc(-c2ccc(-c3ccc(C4CN(CCC5CCOCC5)C4)nn3)c(O)c2)cc2cn(C)nc12] |
| 169 | 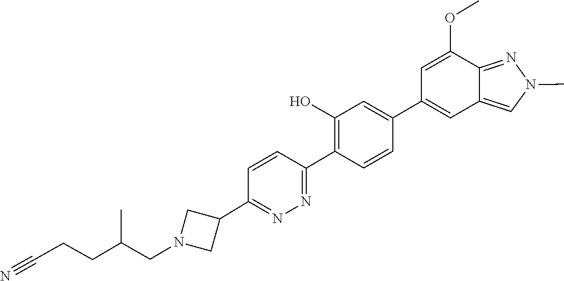 [SMILES: COc1cc(-c2ccc(-c3ccc(C4CN(CC(C)CCC#N)C4)nn3)c(O)c2)cc2cn(C)nc12] |
| 170 | 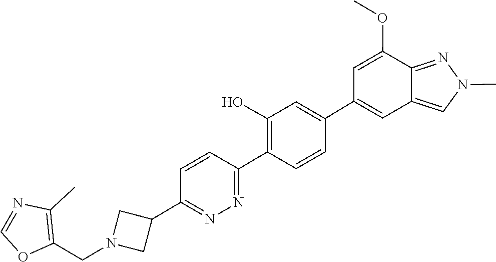 [SMILES: COc1cc(-c2ccc(-c3ccc(C4CN(Cc5ocnc5C)C4)nn3)c(O)c2)cc2cn(C)nc12] |
| 171 | 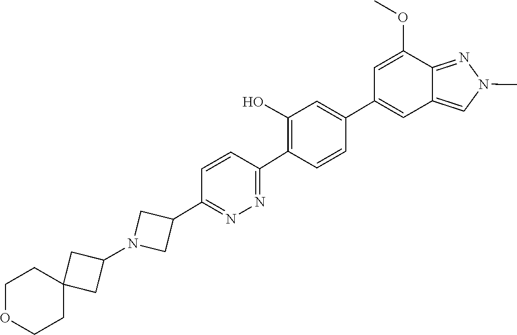 [SMILES: COc1cc(-c2ccc(-c3ccc(C4CN(C5CC6(CCOCC6)C5)C4)nn3)c(O)c2)cc2cn(C)nc12] |
| 172 | 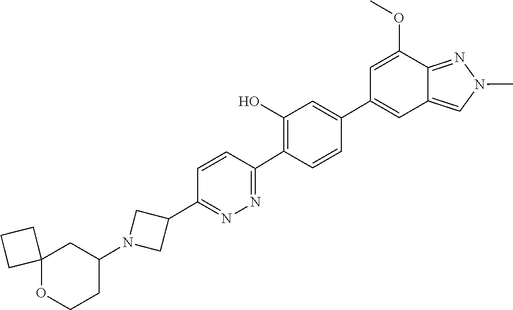 [SMILES: COc1cc(-c2ccc(-c3ccc(C4CN(C5CCOC6(CCC6)C5)C4)nn3)c(O)c2)cc2cn(C)nc12] |
| 173 | 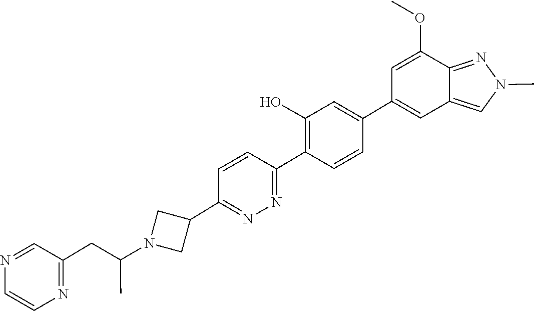 [SMILES: COc1cc(-c2ccc(-c3ccc(C4CN(C(C)Cc5cnccn5)C4)nn3)c(O)c2)cc2cn(C)nc12] |
| 174 | 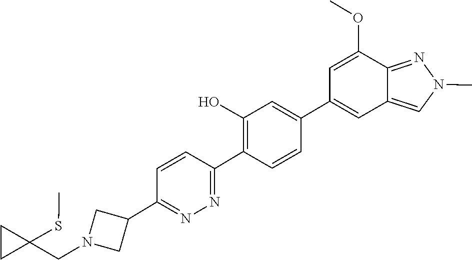 [SMILES: COc1cc(-c2ccc(-c3ccc(C4CN(CC5(SC)CC5)C4)nn3)c(O)c2)cc2cn(C)nc12] |
| 175 | 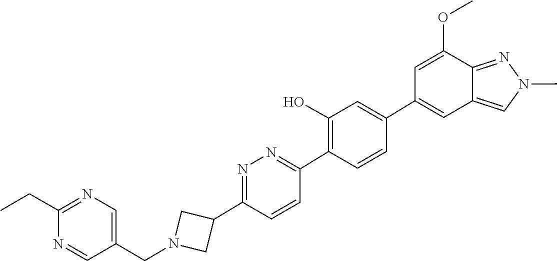 [SMILES: CCc1ncc(CN2CC(c3ccc(-c4ccc(-c5cc(OC)c6nn(C)cc6c5)cc4O)nn3)C2)cn1] |
| 176 | 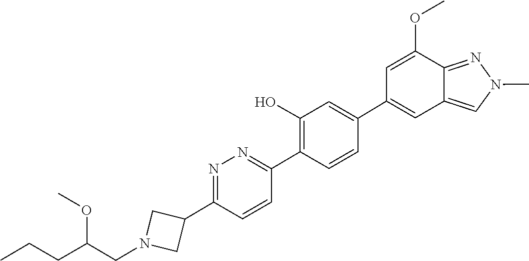 [SMILES: CCCC(CN1CC(c2ccc(-c3ccc(-c4cc(OC)c5nn(C)cc5c4)cc3O)nn2)C1)OC] |
| 177 | 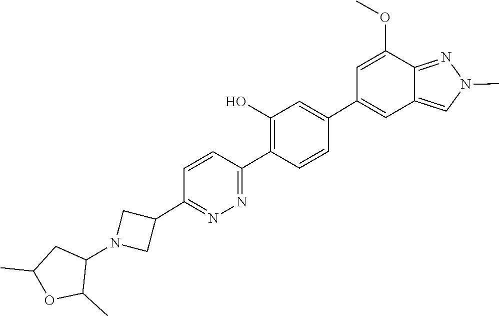 |
| 178 | 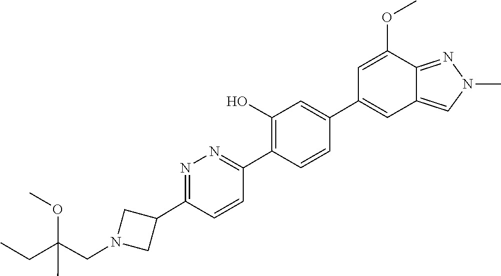 [SMILES: CCC(C)(CN1CC(c2ccc(-c3ccc(-c4cc(OC)c5nn(C)cc5c4)cc3O)nn2)C1)OC] |
| 179 | 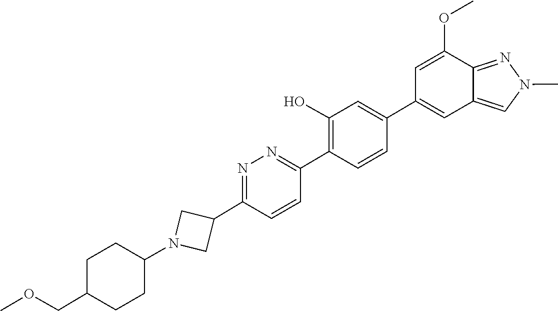 |
| 180 | 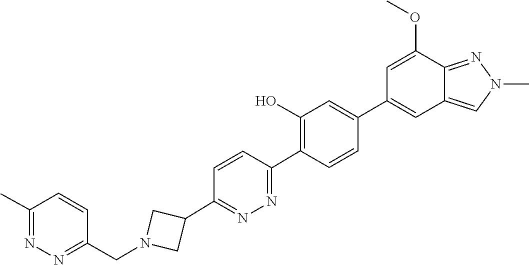 |
| 181 | 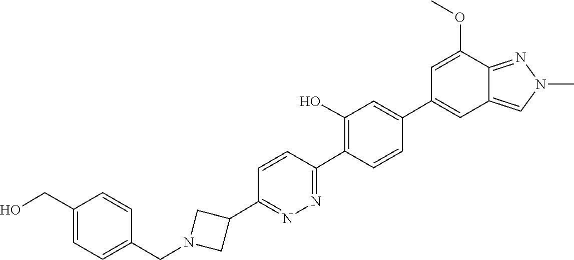 [SMILES: COc1cc(-c2ccc(-c3ccc(C4CN(Cc5ccc(CO)cc5)C4)nn3)c(O)c2)cc2cn(C)nc12] |
| 182 | 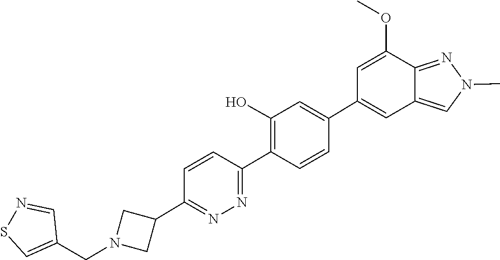 [SMILES: COc1cc(-c2ccc(-c3ccc(C4CN(Cc5cnsc5)C4)nn3)c(O)c2)cc2cn(C)nc12] |
| 183 | 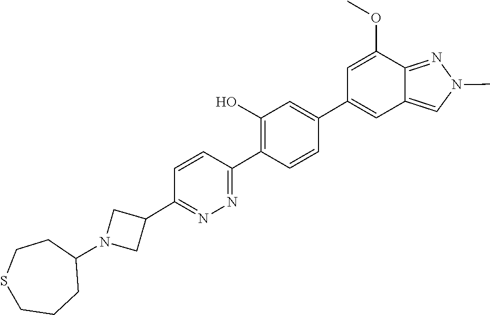 [SMILES: COc1cc(-c2ccc(-c3ccc(C4CN(C5CCCSCC5)C4)nn3)c(O)c2)cc2cn(C)nc12] |
| 184 | 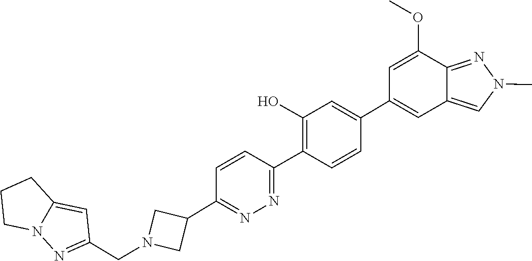 [SMILES: COc1cc(-c2ccc(-c3ccc(C4CN(Cc5cc6n(n5)CCC6)C4)nn3)c(O)c2)cc2cn(C)nc12] |
| 185 | 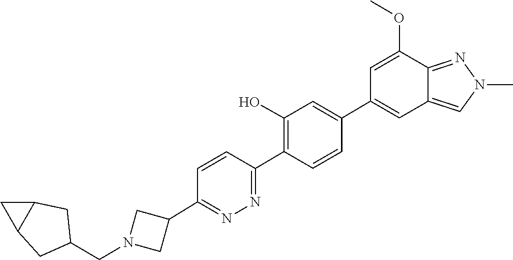 [SMILES: COc1cc(-c2ccc(-c3ccc(C4CN(CC5CC6CC6C5)C4)nn3)c(O)c2)cc2cn(C)nc12] |
| 186 | 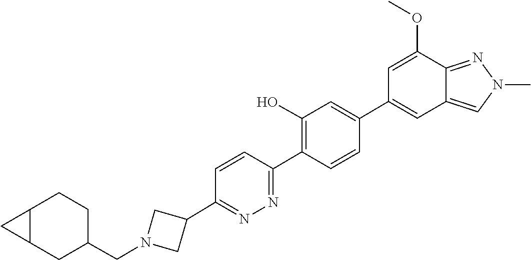 [SMILES: COc1cc(-c2ccc(-c3ccc(C4CN(CC5CCC6CC6C5)C4)nn3)c(O)c2)cc2cn(C)nc12] |
| 187 | 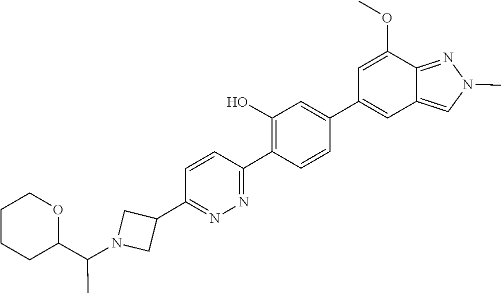 [SMILES: COc1cc(-c2ccc(-c3ccc(C4CN(C(C)C5CCCCO5)C4)nn3)c(O)c2)cc2cn(C)nc12] |
| 188 | 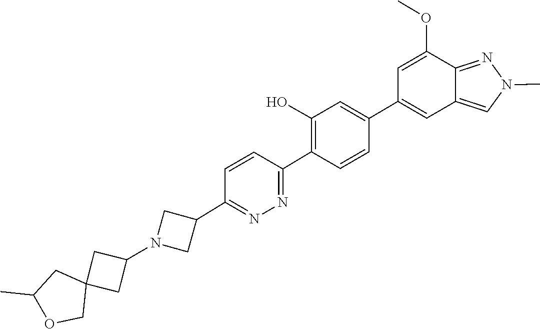 |
| 189 | 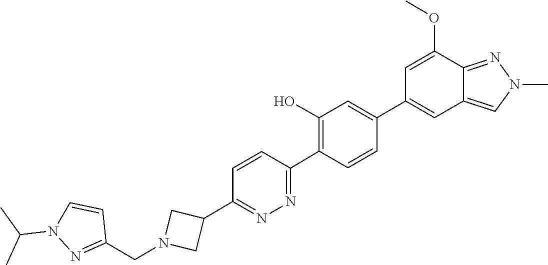 [SMILES: COc1cc(-c2ccc(-c3ccc(C4CN(Cc5ccn(C(C)C)n5)C4)nn3)c(O)c2)cc2cn(C)nc12] |
| 190 | 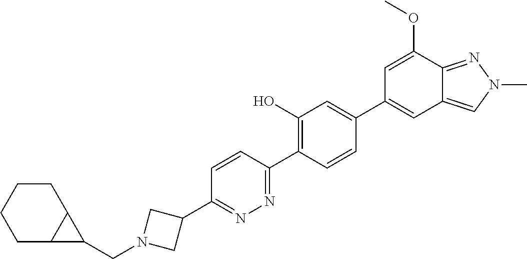 [SMILES: COc1cc(-c2ccc(-c3ccc(C4CN(CC5C6CCCCC65)C4)nn3)c(O)c2)cc2cn(C)nc12] |
| 191 | 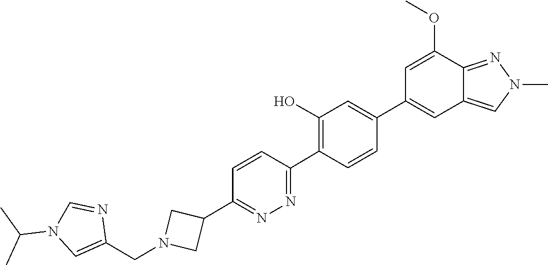 [SMILES: COc1cc(-c2ccc(-c3ccc(C4CN(Cc5cn(C(C)C)cn5)C4)nn3)c(O)c2)cc2cn(C)nc12] |
| 192 | 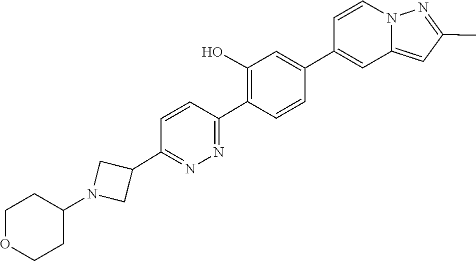 [SMILES: Cc1cc2cc(-c3ccc(-c4ccc(C5CN(C6CCOCC6)C5)nn4)c(O)c3)ccn2n1] |

### Biological Activity of the Compounds of the Present Disclosure

### Mini-Gene Reporter Assay-HTT

Mini-gene reporter constructs for each target site were constructed by first PCR amplifying the region-of-interest including the sequence of the alternatively skipped, sequences of the immediate upstream and downstream introns, and sequences of the immediate upstream and downstream exons. Then the 3′end of the amplified sequence was ligated to a firefly luciferase reporter gene and cloned into the pcDNA3.1 vector backbone. The final reporter construct was transiently transfected into HEK293 cells using Lipofectamine 3000 transfection reagents. Compounds that can induce the inclusion of the skipped exon in the reporter construct would increase the reporter firefly luciferase activity.

Test compounds were diluted in duplicates and dispensed into an assay plate. Transient mini-gene reporter cell line stock was resuspended and dispensed into the assay plate. Assay plates were then centrifuged and incubated. A luciferase reagent (e.g. ONE-Glo firefly luciferase reagent from Promega®) was added to the assay plate and the luminescence (RLU) signals recorded. EC50 values were determined by curve fitting in Levenberg-Marquardt algorithm.

In Table 2 below, A indicates a EC50 (nM)<50 nM, B indicates a EC50 (nM) 50 nM to <500 nM, C indicates a EC50 (nM) 500 nM to <1000 nM, and D indicates a EC50 (nM) ≥1000 nM.

TABLE 2
HTT Assay Data

| | EC₅₀ (nM) |
|---|---|
| Compound No. | HTT |
| 1 | B |
| 2 | B |
| 3 | B |
| 4 | D |
| 5 | B |
| 6 | C |
| 7 | B |
| 8 | D |
| 9 | B |
| 10 | A |
| 11 | C |
| 12 | B |
| 13 | D |
| 14 | C |
| 15 | B |
| 16 | B |
| 17 | B |
| 18 | C |
| 19 | D |
| 20 | C |
| 21 | D |
| 22 | C |
| 23 | D |
| 24 | B |
| 25 | D |
| 26 | D |
| 27 | B |
| 28 | B |
| 29 | C |
| 30 | D |
| 31 | C |
| 32 | D |
| 33 | C |
| 34 | B |
| 35 | C |
| 36 | D |
| 37 | B |
| 38 | B |
| 39 | B |
| 40 | C |
| 41 | D |
| 42 | D |
| 43 | C |
| 44 | C |
| 45 | C |
| 46 | C |
| 47 | C |
| 48 | B |
| 49 | C |
| 50 | B |
| 51 | C |
| 52 | B |
| 53 | C |
| 54 | D |
| 55 | D |
| 56 | C |
| 57 | B |
| 58 | B |
| 59 | D |
| 60 | D |
| 61 | D |
| 62 | C |
| 63 | C |
| 64 | C |
| 65 | D |
| 66 | D |
| 192 | D |

In Table 3 below, A indicates a 00 efficacy at 5 μM>750, B indicates a 00 efficacy at 5 μM, of 25% to 75% and C indicates a % efficacy at 5 μM<25%.

TABLE 3
HTT Minigene % Efficacy at 5 μM Assay Data

| | % Efficacy |
|---|---|
| Compound | at 5 μM |
| 70 | B |
| 71 | A |
| 72 | A |
| 73 | A |
| 74 | B |
| 75 | B |
| 76 | A |
| 77 | A |
| 78 | A |
| 79 | B |
| 80 | B |
| 81 | B |
| 82 | B |
| 83 | B |
| 84 | A |
| 85 | A |
| 86 | A |
| 87 | B |
| 88 | B |
| 89 | A |
| 90 | B |
| 91 | B |
| 92 | A |
| 93 | A |
| 94 | B |
| 95 | A |
| 96 | C |
| 97 | B |
| 98 | A |
| 99 | A |
| 100 | B |
| 101 | B |
| 102 | A |
| 103 | B |
| 104 | B |
| 105 | A |
| 106 | A |
| 107 | B |
| 108 | A |
| 109 | A |
| 110 | B |
| 111 | A |
| 112 | B |
| 113 | B |
| 114 | C |
| 115 | B |
| 116 | B |
| 117 | B |
| 118 | B |
| 119 | B |
| 120 | C |
| 121 | B |
| 122 | B |
| 123 | B |
| 124 | B |
| 125 | B |
| 126 | B |
| 127 | C |
| 128 | B |
| 129 | C |
| 130 | B |
| 131 | A |
| 132 | C |
| 133 | C |
| 134 | C |
| 135 | B |
| 136 | A |
| 137 | A |
| 138 | B |
| 139 | B |
| 140 | C |
| 141 | B |
| 142 | B |
| 143 | A |
| 144 | A |
| 145 | B |
| 146 | B |
| 147 | A |
| 148 | B |
| 149 | B |
| 150 | B |
| 151 | A |
| 152 | C |
| 153 | C |
| 154 | B |
| 155 | A |
| 156 | A |
| 157 | A |
| 158 | A |
| 159 | B |
| 160 | B |
| 161 | B |
| 162 | B |
| 163 | B |
| 164 | A |
| 165 | B |
| 166 | A |
| 167 | B |
| 168 | A |
| 169 | A |
| 170 | C |
| 171 | B |
| 172 | B |
| 173 | B |
| 174 | B |
| 175 | B |
| 176 | B |
| 177 | B |
| 178 | C |
| 179 | B |
| 180 | B |
| 181 | B |
| 182 | B |
| 183 | A |
| 184 | B |
| 185 | B |
| 186 | B |
| 187 | B |
| 188 | B |
| 189 | C |
| 190 | B |
| 191 | B |

### RT-qPCR Assay to Quantify Changes in HTT Splice Isoforms

SK—N—SH cells were seeded at 8.5e3 per well in a 96 well plate, in 199 mL (×4 plates, quadruplicate) and treated with the indicated compounds at different concentrations with a final DMSO concentration of 0.5%. Vehicle controls were set up by diluting DMSO 1:200. Cells were returned to a humidified incubator at 37° C., 5% CO₂ and treated for approximately 96 hours.

RNA was isolated from each well using QIAGEN RNeasy mini columns (cat #74004) as per the manufacturer's instructions. Each condition was done in quadruplicate as mentioned above to allow for adequate starting template for downstream analyses. These quadruplicates were pooled (by condition) into single replicates at the cell lysis step. Reverse-transcription was done using the Applied Biosystems High-Capacity cDNA Reverse Transcription Kit (cat #4368814), per the manufacturer's instructions, using approximately half of the harvested RNA. The cDNA was diluted 1:10 in nuclease-free water, and qPCR was done using Applied Biosystems PowerUp SYBR Green Master Mix (cat #A25742) per the manufacturer's instructions. Cycling conditions are primers are detailed below. Each sample was run in technical duplicates.

### Primer Pairs:

Inclusion:
HTT_F1 + HTT_R, Exclusion: HTT_F2 + HTT_R, and
Reference: GAPDH_F, GAPDH_R
HTT_F1 (exon inclusion):
CAACCCTTGAGAGGCAAGC
HTT_F2 (exon skipping):
CAACCCTTGAGGCCCTGT
HTT_R:
CACAAACTCTGTGGAGGAGAC
GAPDH_F:
CAACGGATTTGGTCGTATTGG
GAPDH_R:
TGATGGCAACAATATCCACTTTACT

qPCR:

| | | | |
|---|---|---|---|
| | 95° C. | 1 | m |
| 40x | 95° C. | 10 | s |
| | 60° C. | 30 | s |

Melt curve

Data were analyzed using the 2-DDCT method. GAPDH was used as the reference gene, and vehicle (i.e. DMSO only) treated cells were used as the reference/untreated cell populations. Relative expression of the HTT transcripts was plotted using Graphpad Prism.

In Table 3 below, A indicates a EC50 (nM)<50 nM, B indicates a EC50 (nM) 50 nM to <500 nM, C indicates a EC50 (nM) 500 nM to <1000 nM, and D indicates a EC50 (nM) >1000 nM.

TABLE 3
qPCR Assay Data

| | EC₅₀ (nM) | EC₅₀ (nM) |
|---|---|---|
| | HTT (exclusion | HTT (inclusion |
| Compound No. | transcript) | transcript) |
| 12 | A | C |
| 15 | A | A |
| 22 | A | NA |
| 23 | A | D |
| 26 | B | C |
| 28 | A | C |
| 36 | B | NA |
| 38 | A | D |

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A compound of Formula (I): 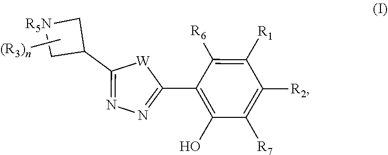
or a pharmaceutically acceptable salt, solvate, or prodrug thereof, wherein:
W is —S— or —HC═CH—;
R₁ is H, halogen, hydroxyl, cyano, C₁-C₆alkyl, C₂-C₆alkenyl, C₂-C₆alkynyl, C₁-C₆haloalkyl, C₁-C₆alkoxyl, C₁-C₆haloalkoxyl, —(CH₂)₀₋₂—C₃-C₈cycloalkyl, NH₂, NH(C₁-C₆alkyl), N(C₁-C₆alkyl)₂, or —(CH₂)₀₋₂-heterocyclyl, wherein heterocyclyl is a 4- to 7-membered ring and comprises 1, 2, or 3 heteroatoms independently selected from N, O, and S, wherein the alkyl, alkenyl, alkynyl, alkoxyl, cycloalkyl, and heterocyclyl are optionally substituted with one or more C₃-C₈cycloalkyl, aryl, or 4- to 7-membered heterocyclyl comprising 1, 2, or 3 heteroatoms independently selected from N, O, and S;
R₂ is aryl, 5- to 7-membered cycloalkyl, 5-, 6-, or 9-membered heterocyclyl comprising 1, 2, or 3 heteroatoms independently selected from N, O, and S, or 5-, 6-, or 9-membered heteroaryl comprising 2, 3, or 4 heteroatoms independently selected from N, O, and S, wherein the aryl, cycloalkyl, heterocyclyl, or heteroaryl is optionally substituted with one or more R₄;
each R₃ is independently halogen, C₁-C₆alkyl, C₂-C₆alkenyl, C₂-C₆alkynyl, C₁-C₆alkoxyl, C₃-C₈cycloalkyl, NH₂, NH(C₁-C₆alkyl), or N(C₁-C₆alkyl)₂, wherein the alkyl, alkenyl, alkynyl, alkoxyl, and cycloalkyl are optionally substituted with one or more hydroxyl or NH₂;
each R₄ is independently halogen, hydroxyl, cyano, nitro, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or C(O)NH₂, wherein the alkyl, alkenyl, alkynyl, alkoxyl, and cycloalkyl are optionally substituted with one or more hydroxyl, 4- to 7-membered heterocyclyl comprising 1, 2, or 3 heteroatoms independently selected from N, O, and S, NH₂, NH(C₁-C₆alkyl), or N(C₁-C₆alkyl)₂;
R₅ is H, C₁-C₆alkyl, C₂-C₆alkenyl, C₂-C₆alkynyl, C₁-C₆alkoxyl, C₃-C₈cycloalkyl, —CH₂C₃-C₈cycloalkyl, heterocyclyl, —CH₂heterocycyl, —CH₂CH₂heterocycyl, —CH₂-(5-6 membered heteroaryl) wherein heterocyclyl is a 4- to 7-membered ring and comprises 1, 2, or 3 heteroatoms independently selected from N, O, and S, wherein R₅ is optionally substituted with one or more halogen, C₁-C₆alkyl, C₁-C₆haloalkyl, C₁-C₆ heteroalkyl, C₁-C₆alkoxyl, C₃-C₈cycloalkyl, spiro C₃-C₈cycloalkyl, spiro 4-7 membered heterocyclyl, 5-6 membered heteroaryl, oxo, cyano, or hydroxyl;
R₆ is H, halogen, C₁-C₆alkyl or C₁-C₆haloalkyl;
R₇ is H, halogen, C₁-C₆alkyl or C₁-C₆haloalkyl; and
n is 0, 1, 2, 3, 4, or 5.

2. The compound of claim 1 of Formula (Ic): 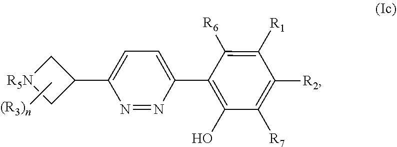
or a pharmaceutically acceptable salt, solvate, or prodrug thereof, wherein:
R₁ is H, halogen, hydroxyl, cyano, C₁-C₆alkyl, C₂-C₆alkenyl, C₂-C₆alkynyl, C₁-C₆haloalkyl, C₁-C₆alkoxyl, C₁-C₆haloalkoxyl, —(CH₂)₀₋₂—C₃-C₈cycloalkyl, NH₂, NH(C₁-C₆alkyl), N(C₁-C₆alkyl)₂, or —(CH₂)₀₋₂-heterocyclyl, wherein heterocyclyl is a 4- to 7-membered ring and comprises 1, 2, or 3 heteroatoms independently selected from N, O, and S, wherein the alkyl, alkenyl, alkynyl, alkoxyl, cycloalkyl, and heterocyclyl are optionally substituted with one or more C₃-C₈cycloalkyl, aryl, or 4- to 7-membered heterocyclyl comprising 1, 2, or 3 heteroatoms independently selected from N, O, and S;
R₂ is aryl, 5- to 7-membered cycloalkyl, 5-, 6-, or 9-membered heterocyclyl comprising 1, 2, or 3 heteroatoms independently selected from N, O, and S, or 5-, 6-, or 9-membered heteroaryl comprising 2, 3, or 4 heteroatoms independently selected from N, O, and S, wherein the aryl, cycloalkyl, heterocyclyl, or heteroaryl is optionally substituted with one or more R₄;
each R₃ is independently halogen, C₁-C₆alkyl, C₂-C₆alkenyl, C₂-C₆alkynyl, C₁-C₆alkoxyl, C₃-C₈cycloalkyl, NH₂, NH(C₁-C₆alkyl), or N(C₁-C₆alkyl)₂, wherein the alkyl, alkenyl, alkynyl, alkoxyl, and cycloalkyl are optionally substituted with one or more hydroxyl or NH₂;
each R₄ is independently halogen, hydroxyl, cyano, nitro, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or C(O)NH₂, wherein the alkyl, alkenyl, alkynyl, alkoxyl, and cycloalkyl are optionally substituted with one or more hydroxyl, 4- to 7-membered heterocyclyl comprising 1, 2, or 3 heteroatoms independently selected from N, O, and S, NH₂, NH(C₁-C₆alkyl), or N(C₁-C₆alkyl)₂;
R₅ is H, C₁-C₆alkyl, C₂-C₆alkenyl, C₂-C₆alkynyl, C₁-C₆alkoxyl, C₃-C₈cycloalkyl, —CH₂C₃-C₈cycloalkyl, heterocyclyl, —CH₂heterocycyl, —CH₂CH₂heterocycyl, —CH₂-(5-6 membered heteroaryl) wherein heterocyclyl is a 4- to 7-membered ring and comprises 1, 2, or 3 heteroatoms independently selected from N, O, and S, wherein R₅ is optionally substituted with one or more halogen, C₁-C₆alkyl, C₁-C₆haloalkyl, C₁-C₆ heteroalkyl, C₁-C₆alkoxyl, C₃-C₈cycloalkyl, spiro C₃-C₈cycloalkyl, spiro 4-7 membered heterocyclyl, 5-6 membered heteroaryl, oxo, cyano, or hydroxyl;
R₆ is H, halogen, C₁-C₆alkyl or C₁-C₆haloalkyl;
R₇ is H, halogen, C₁-C₆alkyl or C₁-C₆haloalkyl; and
n is 0, 1, 2, 3, 4, or 5.

3. The compound of claim 1 of Formula (Id): 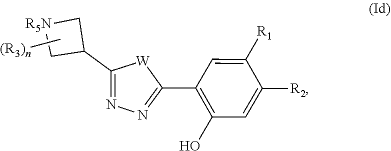
or a pharmaceutically acceptable salt, solvate, or prodrug thereof, wherein:
W is —S— or —HC═CH—;
R₁ is H, halogen, hydroxyl, cyano, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or 4- to 7-membered heterocyclyl comprising 1, 2, or 3 heteroatoms independently selected from N, O, and S, wherein the alkyl, alkenyl, alkynyl, alkoxyl, cycloalkyl, and heterocyclyl are optionally substituted with one or more C₃-C₈cycloalkyl, aryl, or 4- to 7-membered heterocyclyl comprising 1, 2, or 3 heteroatoms independently selected from N, O, and S;
R₂ is aryl, 5- to 7-membered cycloalkyl, 5-, 6-, or 9-membered heterocyclyl comprising 1, 2, or 3 heteroatoms independently selected from N, O, and S, or 5-, 6-, or 9-membered heteroaryl comprising 2, 3, or 4 heteroatoms independently selected from N, O, and S, wherein the aryl, cycloalkyl, heterocyclyl, or heteroaryl is optionally substituted with one or more R₄;
each R₃ is independently halogen, C₁-C₆alkyl, C₂-C₆alkenyl, C₂-C₆alkynyl, C₁-C₆alkoxyl, C₃-C₈cycloalkyl, NH₂, NH(C₁-C₆alkyl), or N(C₁-C₆alkyl)₂, wherein the alkyl, alkenyl, alkynyl, alkoxyl, and cycloalkyl are optionally substituted with one or more hydroxyl or NH₂;
each R₄ is independently halogen, hydroxyl, cyano, nitro, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or C(O)NH₂, wherein the alkyl, alkenyl, alkynyl, alkoxyl, and cycloalkyl are optionally substituted with one or more hydroxyl, 4- to 7-membered heterocyclyl comprising 1, 2, or 3 heteroatoms independently selected from N, O, and S, NH₂, NH(C₁-C₆alkyl), or N(C₁-C₆alkyl)₂;
R₅ is H, C₁-C₆alkyl, C₂-C₆alkenyl, C₂-C₆alkynyl, C₁-C₆alkoxyl, C₃-C₈cycloalkyl, —CH₂C₃-C₈cycloalkyl, heterocyclyl, —CH₂heterocycyl, —CH₂CH₂heterocycyl, —CH₂-(5-6 membered heteroaryl) wherein heterocyclyl is a 4- to 7-membered ring and comprises 1, 2, or 3 heteroatoms independently selected from N, O, and S, wherein R₅ is optionally substituted with one or more halogen, C₁-C₆alkyl, C₁-C₆haloalkyl, C₁-C₆ heteroalkyl, C₁-C₆alkoxyl, C₃-C₈cycloalkyl, spiro C₃-C₈cycloalkyl, spiro 4-7 membered heterocyclyl, 5-6 membered heteroaryl, oxo, cyano, or hydroxyl; and
n is 0, 1, 2, 3, 4, or 5.

4. The compound of claim 1, wherein the compound is of Formula (Ia): 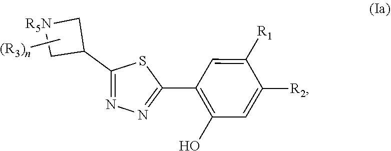
or a pharmaceutically acceptable salt, solvate, or prodrug thereof.

5. The compound of claim 1, wherein the compound is of Formula (Ib): 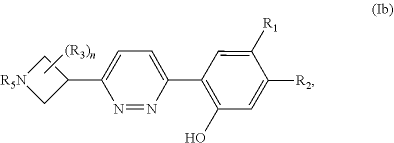
or a pharmaceutically acceptable salt, solvate, or prodrug thereof.

6. The compound of claim 1, wherein the compound is of Formula (Ie): 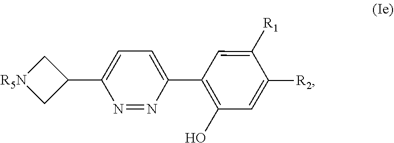
or a pharmaceutically acceptable salt, solvate, or prodrug thereof.

7. The compound of claim 1, wherein the compound is of Formula (If): 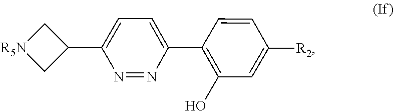
or a pharmaceutically acceptable salt, solvate, or prodrug thereof.

8. The compound according to any one of claims 1-7, wherein R₁ is H or F.

9. The compound according to any one of claims 1-8, wherein R₂ is 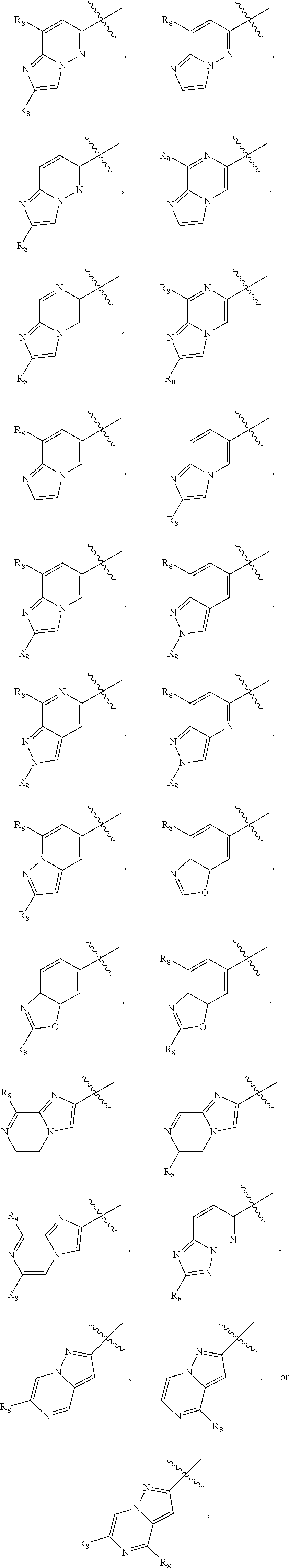
wherein each R₈ is independently R₄.

10. The compound of claim 9, wherein each R₈ is independently halo, C₁₋₆alkyl, C₁₋₆haloalkyl, or C₁₋₆alkoxy; preferably each R₈ is independently F, Me, Et, CF₃, MeO, or EtO.

11. The compound according to any one of claims 1-10, wherein R₂ is

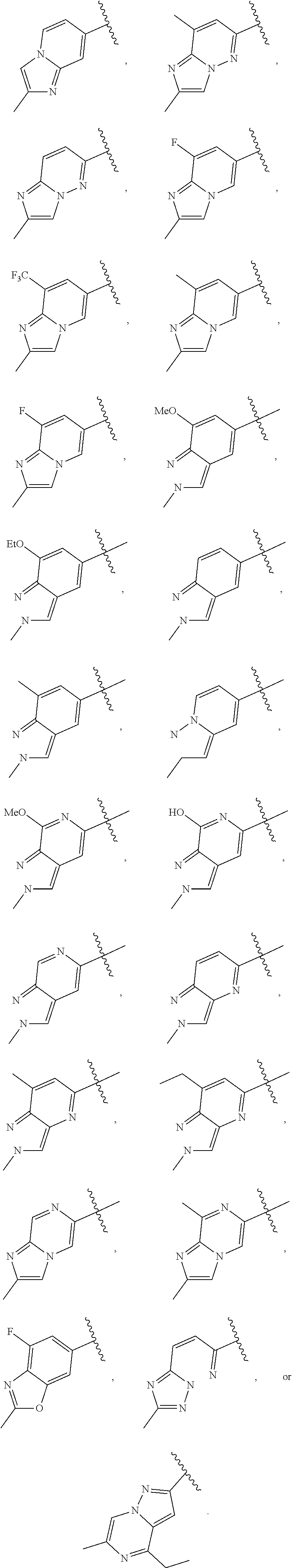

12. The compound according to any one of claims 1-10, wherein R₄ is methyl, ethyl, F, or CF₃.

13. A compound according to any one of claims 1-12, wherein R₅ is H, C₁₋₆alkyl, or heterocyclyl, wherein heterocyclyl is a 4- to 7-membered ring and comprises 1 or 2 heteroatoms independently selected from N and O.

14. A compound according to any one of claims 1-12, wherein R₅ is H.

15. A compound according to any one of claims 1-14, selected from a compound of Table 1.

16. A compound according to any one of claims 1-15, or a pharmaceutically acceptable salt, solvate, or prodrug thereof for use as a therapeutically active substance.

17. A compound according to any one of claims 1-16, or a pharmaceutically acceptable salt, solvate, or prodrug thereof for use as a small molecule splicing modulator.

18. A pharmaceutical composition comprising a compound according to any one of claims 1-17, or a pharmaceutically acceptable salt, solvate, or prodrug thereof and one or more pharmaceutically acceptable excipients.

19. A method of treating a disease in a subject in need thereof, the method comprising administering to the subject a therapeutically effective amount of a compound of any one of claims 1-15 or a pharmaceutically composition of claim 18, wherein the disease is selected from the group consisting of ALS, Alzheimer's disease, argyrophilic grain disease, corticobasal degeneration, cystic fibrosis, dilated cardiomyopathy, Duchenne muscular dystrophy, Ehlers-Danlos syndrome, Fabry's disease, familial dysautonomia, familial hypercholesterolemia, familial persistent hyperinsulinemic hypoglycemia, frontotemporal dementia, FTDP-17, Gacher's disease, globular glial tauopathy, HIV-1, Huntington's disease, Hutchinson-Gilford progeria syndrome, hypercholesterolemia, Leber congenital amaurosis, migraine, multiple sclerosis, myelodysplastic syndromes, NASH, Niemann-Pick's, pain, Parkinson's disease, phenylketonuria, Pick's disease, progressive supranuclear palsy, spinal muscular atrophy, spinocerebellar ataxia type 2, Wilson's disease, Sickle cell anemia, Crohn's disease, ulcerative colitis, psoriasis, and rheumatoid arthritis

20. The method of claim 19, wherein the disease is Huntington's disease.

21. A method of treating a disease in a subject in need thereof, the method comprising administering to the subject a therapeutically effective amount of a compound of any one of claims 1-15 or a pharmaceutical composition of claim 18, wherein the disease is a pulmonary disease selected from the group consisting of chronic obstructive pulmonary disease (COPD), asthma, acute lung injury (ALI), pulmonary fibrosis, and pulmonary arterial hypertension (PAH).
